(19) United States
(12) Patent Application Publication
(10) Pub. No.: US 2024/0093202 A1
(43) Pub. Date: Mar. 21, 2024

(54) Suppression-Replacement Gene Therapy

(71) Applicant: Mayo Foundation for Medical Education and Research, Rochester, MN (US)

(72) Inventors: Michael J. ACKERMAN, Rochester, MN (US); Steven M. DOTZLER, Rochester, MN (US); William GENDRON, Rochester, MN (US); Sahej BAINS, Rochester, MN (US); Changsung John KIM, Rochester, MN (US); David J. TESTER, Rochester, MN (US)

(73) Assignee: Mayo Foundation for Medical Education and Research, Rochester, MN (US)

(21) Appl. No.: 18/270,014

(22) PCT Filed: Dec. 30, 2021
(86) PCT No.: PCT/US21/65682

§ 371 Date: Jun. 28, 2023

Related U.S. Application Data
(60) Provisional application No. 63/132,316, filed on Dec. 30, 2020.
(60) Provisional application No. 63/179,083, filed on Apr. 23, 2021.
(60) Provisional application No. 63/208,556, filed on Jun. 9, 2021.
(60) Provisional application No. 63/270,388, filed on Oct. 21, 2021.

(51) Int. Cl. C12N 15/113; A61K 38/17; A61K 48/00; A61P 9/00; C07K 14/705; C12N 15/86

(52) U.S. Cl. CPC C12N 15/1138; A61K 38/177; A61K 48/005; A61P 9/00; C07K 14/705; C12N 15/86; C12N 2310/14; C12N 2320/34; C12N 2740/16043; C12N 2750/14143; C12N 2840/203

(57) ABSTRACT

Methods and materials for treating a mammal having a congenital disease (e.g., a congenital heart disease such as congenital long QT syndrome) are provided herein. For example, this document provides methods and materials for generating and using nucleic acids to treat a mammal having a congenital disease, where the nucleic acids can suppress expression of mutant disease-related alleles in the mammal while providing a replacement cDNA that does not contain the disease-related mutation(s).

### CROSS-REFERENCE TO RELATED APPLICATIONS

This application claims benefit of priority from U.S. Provisional Application Ser. No. 63/132,316, filed Dec. 30, 2020, U.S. Provisional Application Ser. No. 63/179,083, filed Apr. 23, 2021, U.S. Provisional Application Ser. No. 63/208,556, filed Jun. 9, 2021, and U.S. Provisional Application Ser. No. 63/270,388, filed Oct. 21, 2021. The disclosures of the prior applications are considered part of (and are incorporated by reference in) the disclosure of this application.

### TECHNICAL FIELD

This document relates to methods and materials for treating a mammal having a congenital disease (e.g., a congenital heart disease such as congenital long QT syndrome). For example, this document provides methods and materials for generating and using nucleic acids that can be administered to a mammal having a congenital disease, and can suppress expression of mutant disease-related alleles in the mammal while providing a replacement cDNA that does not contain the disease-related mutation(s).

### BACKGROUND

Congenital long QT syndrome (LQTS) is an autosomal dominant disorder characterized by delayed repolarization of the myocardium that is associated with a prolonged QT interval on electrocardiogram (ECG). Patients with LQTS have increased risk for torsadogenic syncope/seizures and sudden cardiac death (SCD). The prevalence of LQTS is about 1 in 2000, and when untreated, higher risk patients have an estimated 10-year mortality of 50% (Schwartz et al., Circulation, 120:1761-1767 (2009); and Schwartz and Ackerman, Eur. Heart J., 34:3109-3116 (2013)).

LQTS is caused by pathogenic variants in cardiac ion channels or their interacting regulatory proteins (Giudicessi et al., Trends Cardiovasc. Med., 28:453-464 (2018)). Type 1 LQTS (LQT1) is the most common form of LQTS, accounting for about 35% of cases (Ackerman et al., Heart Rhythm., 8:1308-1339 (2011)). LQT1 is caused by loss-of-function variants in KCNQ1, which encodes the α-subunit of the Kv7.1 voltage-gated potassium channel that is responsible for the slow delayed rectifier current (I_{Ks}) during repolarization of the cardiac action potential. Because the KCNQ1-encoded α-subunits tetramerize during Kv7.1 channel assembly, pathogenic missense variants commonly exhibit a dominant-negative effect due to interference with the wild-type (WT) subunits translated from the non-affected allele. Another common form of LQTS is LQT2, which accounts for about 30% of cases. Patients with LQT2 host loss-of-function mutations in the KCNH2-encoded I_{Kr} (Kv11.1) potassium channel that, like KCNQ1, plays a role in cardiac action potential duration (APD) (Tester et al., Heart Rhythm., 2(5):507-517 (2005); Giudicessi et al., Trends Cardiovasc. Med., 28:453-464 (2018); and Ackerman et al., Heart Rhythm., 8:1308-1339 (2011)). Pathogenic variants in KCNQ1 or KCNH2 that lead to a gain-of-function and an abnormal increase in I_{Ks} or I_{Kr} current density, respectively, can lead to short QT syndrome (SQTS). The third most common form of LQTS is LQT3, which accounts for about 10% of cases. Patients with LQT3 host gain-of-function mutations in the SCN5A-encoded I_{Na} (Nav1.5) sodium channel that also plays a role in the cardiac APD (Tester et al., J. Am. Coll. Cardiol. EP, 4:569-579 (2018)). Pathogenic variants in SCN5A that lead to a loss-of-function and a decrease in I_{Na} can cause Brugada syndrome (Wilde and Amin, J. Am. Coll. Cardiol. EP, 4:569-579 (2018)).

Current therapies for management of LQTS include beta-blockers, which provide a first line treatment, as well as more invasive therapies such as left cardiac sympathetic denervation (LCSD) or implantation of a cardioverter defibrillator (ICD). These, however, can have limitations including noncompliance, breakthrough cardiac events, or infection (Rohatgi et al., J. Am. Coll. Cardiol., 70:453-462 (2017); Priori et al., Heart Rhythm., 10:1932-1963 (2013); Al-Khatib et al., Heart Rhythm., 15:e190-e252 (2018); Schwartz et al., Circulation, 109:1826-1833 (2004); Bos et al., Circ. Arrhythm. Electrophysiol., 6:705-711 (2013); Schwartz et al., Circulation, 122:1272-1282 (2010); Homer et al., Heart Rhythm., 7:1616-1622 (2010); and Kleemann et al., Circulation, 115:2474-2480 (2007)), and they do not treat the underlying pathogenic substrate.

RNA interference (RNAi) technology, such as small interfering RNA (siRNA), utilizes endogenous gene silencing to knock down gene expression. Attempts to overcome dominant-negative KCNH2 variants in LQT2 have used allele-specific siRNAs to selectively knock down the mutant allele (Lu et al., Heart Rhythm, 10:128-136 (2013); and Matsa et al., Eur. Heart 1, 35:1078-1087 (2014)). The best possible outcome of this method would be haploinsufficiency, however. In addition, it would be necessary to engineer and validate a separate siRNA for each unique LQT2-causative variant, which would be impractical in KCNQ1, KCNH2, and SCN5A, as there are hundreds of LQT1-, LQT2-, and LQT3-causative variants (Landrum et al., Nucleic Acids Res., 46:D1062-1c:1 D1067 (2018)).

### SUMMARY

This document is based, at least in part, on the development of a dual-component “suppression-and-replacement” KCNQ1 (KCNQ1-SupRep) gene therapy approach for LQT1, in which a KCNQ1 shRNA is used to suppress expression of the endogenous KCNQ1 alleles and a codon-altered “shRNA-immune” copy of KCNQ1 is used for gene replacement. As described herein, the “KCNQ1-SupRep” system was successfully used to rescue the prolonged action potential duration in induced pluripotent stem cell (iPSC) cardiomyocytes derived from fibroblasts or PBMCs from four patients with unique LQT1-causative KCNQ1 variants. This document therefore describes successful preclinical hybrid gene therapy in LQT1, and demonstrates that the system provided herein is capable of complete rescue of KCNQ1 function. Theoretically, KCNQ1-SupRep is applicable to essentially any patient with LQT1, because it targets the whole KCNQ1 gene rather than specific mutations.

This document also is based, at least in part, on the development of a “suppression-and-replacement” KCNH2 (KCNH2-SupRep) gene therapy approach for LQT2, in which a KCNQ2 shRNA is used to suppress expression of the endogenous KCNH2 alleles and a codon-altered “shRNA-immune” copy of KCNH2 is used for gene replacement.

In addition, this document is based, at least in part, on the development of a “suppression-and-replacement” SCN5A (SCN5A-SupRep) gene therapy approach for LQT3, in which a SCN5A shRNA is used to suppress expression of the endogenous SCN5A alleles and a codon-altered “shRNA-immune” copy of SCN5A is used for gene replacement.

Having the ability to reduce the myocardium repolarization time (e.g., by shortening the APD) using the methods and materials described herein can allow clinicians and patients (e.g., LQTS patients) to achieve cardiac function that more closely resembles the function of a healthy heart, when compared to the function of an untreated LQTS patient's heart. In some cases, having the ability to reduce the myocardium repolarization time in LQTS patients using the methods and materials described herein can allow clinicians and patients to reduce LQTS symptoms and/or reverse LQTS progression. For example, delivery of a nucleic acid or virus construct provided herein to heart tissue can rescue cardiac defects and increase survival in LQTS patients.

In one aspect, this document features a nucleic acid construct. The nucleic acid construct can include (a) a first nucleotide sequence encoding an RNAi molecule capable of hybridizing to a target sequence encoding an endogenous KCNQ1 polypeptide within a cell and suppressing expression of the endogenous KCNQ1 polypeptide within the cell, and (b) a second nucleotide sequence encoding a KCNQ1 polypeptide, where the second nucleotide sequence includes a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNQ1 polypeptide from the second nucleotide sequence within the cell. The first nucleotide sequence can include, consist essentially of, or consist of the sequence set forth in SEQ ID NO:12, SEQ ID NO:13, SEQ ID NO:15, or SEQ ID NO:36, and the second nucleotide sequence can include, consist essentially of, or consist of the sequence set forth in SEQ ID NO:9. The first nucleotide sequence can include, consist essentially of, or consist of the sequence set forth in SEQ ID NO:36 and the second nucleotide sequence can include, consist essentially of, or consist of the sequence set forth in SEQ ID NO:9. The first nucleotide sequence can be operably linked to a first promoter, and the second nucleotide sequence can be operably linked to a second promoter. The first and second promoters can be the same or can be different. The first promoter can be a U6 promoter, and the second promoter can be a cytomegalovirus immediate-early (CMV) promoter. The nucleic acid construct can further include a nucleotide sequence encoding a reporter. The reporter can be a fluorescent polypeptide. The nucleotide sequence encoding the reporter can be downstream of the second nucleotide sequence encoding the KCNQ1 polypeptide (e.g., a cDNA encoding the KCNQ1 polypeptide), and can be separated from the second nucleotide sequence by an internal ribozyme entry sequence (IRES) or P2A self-cleaving peptide sequence. The nucleic acid construct can be within a viral vector. The viral vector can be an adeno-associated virus (AAV) vector (e.g., an AAV serotype 9 vector or an AAV2/9 vector). The cell can be a cardiomyocyte.

In another aspect, this document features a virus particle containing a nucleic acid construct described herein (e.g., a nucleic acid construct of the preceding paragraph).

In another aspect, this document features a method for treating a mammal having a congenital cardiac disease. The method can include administering to the mammal a nucleic acid construct containing (a) a first nucleotide sequence encoding an RNAi molecule capable of hybridizing to a target sequence encoding an endogenous KCNQ1 polypeptide within a cell of the mammal and suppressing expression of the endogenous KCNQ1 polypeptide within the cell, and (b) a second nucleotide sequence encoding a KCNQ1 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNQ1 polypeptide from the second nucleotide sequence within the cell. The congenital cardiac disease can be long QT syndrome (LQTS) or short QT syndrome (SQTS). The congenital cardiac disease can be LQT1. The first nucleotide sequence can include, consist essentially of, or consist of the sequence set forth in SEQ ID NO:12, SEQ ID NO:13, SEQ ID NO:15, or SEQ ID NO:36, and the second nucleotide sequence can include, consist essentially of, or consist of the sequence set forth in SEQ ID NO:9. The first nucleotide sequence can include, consist essentially of, or consist of the sequence set forth in SEQ ID NO:36, and the second nucleotide sequence can include, consist essentially of, or consist of the sequence set forth in SEQ ID NO:9. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can further include a nucleotide sequence encoding a reporter. The reporter can be a fluorescent polypeptide. The nucleotide sequence encoding the reporter can be downstream of the second nucleotide sequence encoding the KCNQ1 polypeptide (e.g., a cDNA encoding the KCNQ1 polypeptide), and can be separated from the second nucleotide sequence by an IRES. The nucleic acid construct can be within a viral vector. The viral can be an AAV vector (e.g., an AAV serotype 9 vector or an AAV2/9 vector). The cell can be a cardiomyocyte.

In another aspect, this document features a method for reducing the action potential duration (APD) in cardiac cells within a mammal. The method can include administering to the mammal a nucleic acid construct containing (a) a first nucleotide sequence encoding an RNAi molecule capable of hybridizing to a target sequence encoding an endogenous KCNQ1 polypeptide within cardiac cells of the mammal and suppressing expression of the endogenous KCNQ1 polypeptide within the cardiac cells, and (b) a second nucleotide sequence encoding a KCNQ1 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNQ1 polypeptide from the second nucleotide sequence within the cell. The first nucleotide sequence can include, consist essentially of, or consist of the sequence set forth in SEQ ID NO:12, SEQ ID NO:13, SEQ ID NO:15, or SEQ ID NO:36, and the second nucleotide sequence can include, consist essentially of, or consist of the sequence set forth in SEQ ID NO:9. The first nucleotide sequence can include, consist essentially of, or consist of the sequence set forth in SEQ ID NO:36, and the second nucleotide sequence can include, consist essentially of, or consist of the sequence set forth in SEQ ID NO:9. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can be within a viral vector. The viral vector can be an AAV vector (e.g., an AAV serotype 9 vector or an AAV2/9 vector).

In another aspect, this document features a method for reducing one or more symptoms of LQTS in a mammal. The method can include administering to the mammal a nucleic acid construct containing (a) a first nucleotide sequence encoding an RNAi molecule capable of hybridizing to a target sequence encoding an endogenous KCNQ1 polypeptide within a cell of the mammal and suppressing expression of the endogenous KCNQ1 polypeptide within the cell, and (b) a second nucleotide sequence encoding a KCNQ1 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNQ1 polypeptide from the second nucleotide sequence within the cell. The LQTS can be LQT1. The first nucleotide sequence can include, consist essentially of, or consist of the sequence set forth in SEQ ID NO:12, SEQ ID NO:13, SEQ ID NO:15, or SEQ ID NO:36, and the second nucleotide sequence can include, consist essentially of, or consist of the sequence set forth in SEQ ID NO:9. The first nucleotide sequence can include, consist essentially of, or consist of the sequence set forth in SEQ ID NO:36, and the second nucleotide sequence can include, consist essentially of, or consist of the sequence set forth in SEQ ID NO:9. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can be within a viral vector. The viral vector can be an AAV vector (e.g., an AAV serotype 9 vector or an AAV2/9 vector). The cell can be a cardiomyocyte.

In another aspect, this document features a nucleic acid construct that can include (a) a first nucleotide sequence encoding an RNAi molecule capable of hybridizing to a target sequence encoding an endogenous KCNH2 polypeptide within a cell and suppressing expression of the endogenous KCNH2 polypeptide within the cell, and (b) a second nucleotide sequence encoding a KCNH2 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNH2 polypeptide from the second nucleotide sequence within the cell. The first nucleotide sequence can include, consist essentially of, or consist of the sequence set forth in SEQ ID NO:27, and the second nucleotide sequence can include, consist essentially of, or consist of the sequence set forth in SEQ ID NO:29. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can further include a nucleotide sequence encoding a reporter. The reporter can be a fluorescent polypeptide. The nucleotide sequence encoding the reporter can be downstream of the second nucleotide sequence encoding the KCNH2 polypeptide (e.g., a cDNA encoding the KCNH2 polypeptide), and can be separated from the second nucleotide sequence by an IRES or P2A self-cleaving peptide sequence. The nucleic acid construct can be within a viral vector. The viral vector can be an AAV vector (e.g., an AAV serotype 9 vector or an AAV2/9 vector). The cell can be a cardiomyocyte.

In another aspect, this document features a virus particle containing a nucleic acid construct described herein (e.g., a nucleic acid construct described in the preceding paragraph).

In still another aspect, this document features a method for treating a mammal having a congenital cardiac disease. The method can include administering to the mammal a nucleic acid construct containing (a) a first nucleotide sequence encoding an RNAi molecule capable of hybridizing to a target sequence encoding an endogenous KCNH2 polypeptide within a cell of the mammal and suppressing expression of the endogenous KCNH2 polypeptide within the cell, and (b) a second nucleotide sequence encoding a KCNH2 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNH2 polypeptide from the second nucleotide sequence within the cell. The congenital cardiac disease can be LQTS or SQTS. The congenital cardiac disease can be LQT2. The first nucleotide sequence can include, consist essentially of, or consist of the sequence set forth in SEQ ID NO:27, and the second nucleotide sequence can include, consist essentially of, or consist of the sequence set forth in SEQ ID NO:29. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can further include a nucleotide sequence encoding a reporter. The reporter can be a fluorescent polypeptide. The nucleotide sequence encoding the reporter can be downstream of the second nucleotide sequence encoding the KCNH2 polypeptide (e.g., a cDNA encoding the KCNH2 polypeptide), and can be separated from the second nucleotide sequence by an IRES. The nucleic acid construct can be within a viral vector. The viral vector can be an AAV vector (e.g., an AAV serotype 9 vector or an AAV2/9 vector). The cell can be a cardiomyocyte.

In another aspect, this document features a method for reducing the APD in cardiac cells within a mammal. The method can include administering to the mammal a nucleic acid construct containing (a) a first nucleotide sequence encoding an RNAi molecule capable of hybridizing to a target sequence encoding an endogenous KCNH2 polypeptide within cardiac cells of the mammal and suppressing expression of the endogenous KCNH2 polypeptide within the cardiac cells, and (b) a second nucleotide sequence encoding a KCNH2 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNH2 polypeptide from the second nucleotide sequence within the cell. The first nucleotide sequence can include, consist essentially of, or consist of the sequence set forth in SEQ ID NO:27, and the second nucleotide sequence can include, consist essentially of, or consist of the sequence set forth in SEQ ID NO:29. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can be within a viral vector. The viral vector can be an AAV vector (e.g., an AAV serotype 9 vector or an AAV2/9 vector).

In yet another aspect, this document features a method for reducing one or more symptoms of LQTS in a mammal. The method can include administering to the mammal a nucleic acid construct containing (a) a first nucleotide sequence encoding an RNAi molecule capable of hybridizing to a target sequence encoding an endogenous KCNH2 polypeptide within a cell of the mammal and suppressing expression of the endogenous KCNH2 polypeptide within the cell, and (b) a second nucleotide sequence encoding a KCNH2 polypeptide, where the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and where the RNAi molecule does not suppress expression of the KCNH2 polypeptide from the second nucleotide sequence within the cell. The LQTS can be LQT2. The first nucleotide sequence can include, consist essentially of, or consist of the sequence set forth in SEQ ID NO:27, and the second nucleotide sequence can include, consist essentially of, or consist of the sequence set forth in SEQ ID NO:29. The first nucleotide sequence can be operably linked to a first promoter, and the second nucleotide sequence can be operably linked to a second promoter. The first and second promoters can be the same or can be different. The first promoter can be a U6 promoter, and the second promoter can be a CMV promoter. The nucleic acid construct can be within a viral vector. The viral vector can be an AAV vector (e.g., an AAV serotype 9 vector or an AAV2/9 vector). The cell can be a cardiomyocyte.

In another aspect, this document features a nucleic acid construct for treating a congenital heart disease caused by an endogenous cardiac polypeptide containing one or more mutations causative of the congenital heart disease, where the construct can include (a) a first nucleotide sequence encoding an RNAi molecule capable of hybridizing to a target sequence encoding the endogenous cardiac polypeptide within a cell and suppressing expression of the endogenous cardiac polypeptide within the cell, and (b) a second nucleotide sequence encoding a replacement version of the endogenous cardiac polypeptide that lacks the one or more mutations causative of the congenital heart disease, wherein the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and wherein the RNAi molecule does not suppress expression of the replacement version of the endogenous cardiac polypeptide that lacks the one or more mutations causative of the congenital heart disease from the second nucleotide sequence within the cell. The first nucleotide sequence can be operably linked to a first promoter and the second nucleotide sequence can be operably linked to a second promoter. The first and second promoters can be the same, or the first and second promoters can be different. The first promoter can be a U6 promoter and the second promoter can be a CMV promoter. The nucleic acid construct can further include a nucleotide sequence encoding a reporter. The reporter can be a fluorescent polypeptide. The nucleotide sequence encoding the reporter can be downstream of the second nucleotide sequence encoding the cDNA, and can be separated from the second nucleotide sequence by an IRES or P2A self-cleaving peptide sequence. The nucleic acid construct can be within a viral vector. The viral vector can be an AAV vector (e.g., an AAV serotype 9 vector or an AAV2/9 vector). The cell can be a cardiomyocyte.

In another aspect, this document features a virus particle containing the nucleic acid construct described herein (e.g., a nucleic acid construct described in the preceding paragraph).

In still another aspect, this document features a method for treating a mammal having a congenital cardiac disease. The method can include administering to the mammal a nucleic acid construct containing (a) a first nucleotide sequence encoding an RNAi molecule capable of hybridizing to a target sequence encoding the endogenous cardiac polypeptide within a cell and suppressing expression of the endogenous cardiac polypeptide within the cell, and (b) a second nucleotide sequence encoding a replacement version of the endogenous cardiac polypeptide that lacks the one or more mutations causative of the congenital heart disease, wherein the second nucleotide sequence comprises a target sequence identical to the target sequence of the first nucleotide sequence with the exception that the target sequence of the second nucleotide sequence comprises 1 to 13 wobble position variants as compared to the target sequence of the first nucleotide sequence, and wherein the RNAi molecule does not suppress expression of the replacement version of the endogenous cardiac polypeptide that lacks the one or more mutations causative of the congenital heart disease from the second nucleotide sequence within the cell. The first nucleotide sequence can be operably linked to a first promoter and the second nucleotide sequence can be operably linked to a second promoter. The first and second promoters can be the same, or the first and second promoters can be different. The first promoter can be a U6 promoter and the second promoter can be a CMV promoter. The nucleic acid construct can further include a nucleotide sequence encoding a reporter. The reporter can be a fluorescent polypeptide. The nucleotide sequence encoding the reporter can be downstream of the second nucleotide sequence encoding the cDNA, and can be separated from the second nucleotide sequence by an IRES. The nucleic acid construct can be within a viral vector. The viral vector can be an AAV vector (e.g., an AAV serotype 9 vector or an AAV2/9 vector). The cell can be a cardiomyocyte.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a diagram of an exemplary KCNQ1-P2A AAV construct, and FIG. 1B shows the DNA sequence (SEQ ID NO:1029) for the construct. FIG. 1C shows a KCNQ1 target sequence (sh#5; SEQ ID NO:102), a corresponding shIMM KCNQ1 sequence (SEQ ID NO:103), a wild type KCNQ1 nucleotide sequence (SEQ ID NO:1030, with the sh#5 sequence underlined), a corresponding shIMM KCNQ1 nucleotide sequence (SEQ ID NO:1031, with the shIMM sequence underlined), and a KCNQ1 amino acid sequence (SEQ ID NO:1032).

FIG. 2A is a diagram of an exemplary KCNH2-P2A AAV construct, and FIG. 2B shows the DNA sequence (SEQ ID NO:1033) for the construct. The encoded AmpR amino acid sequence (SEQ ID NO:2784) also is shown. FIG. 2C shows a KCNH2 target sequence (RAB_sh#4; SEQ ID NO:27), a corresponding shIMM KCNH2 sequence (SEQ ID NO:29), a wild type KCNH2 nucleotide sequence (SEQ ID NO:1034, with the RAB_sh#4 sequence underlined), a corresponding shIMM KCNH2 nucleotide sequence (SEQ ID NO:1035, with the shIMM sequence underlined), and a KCNH2 amino acid sequence (SEQ ID NO:1036).

FIG. 3A is a diagram of an exemplary SCN5A-P2A Lenti construct, and FIG. 3B shows the DNA sequence (SEQ ID NO:1041) for the construct. FIG. 3C shows a SCN5A target sequence (sh#4; SEQ ID NO:30), a corresponding shIMM SCN5A sequence (SEQ ID NO:32), a wild type SCN5A nucleotide sequence (SEQ ID NO:1042, with the sh#5 sequence underlined), a corresponding shIMM SCN5A nucleotide sequence (SEQ ID NO:1043, with the shIMM sequence underlined), and a SCN5A amino acid sequence (SEQ ID NO:1044).

FIG. 4A is a diagram of an exemplary PKP2-P2A AAV construct, and FIG. 4B shows the DNA sequence (SEQ ID NO:1037) for the construct. FIG. 4C shows a PKP2 target sequence (sh#36; SEQ ID NO:52), a corresponding shIMM PKP2 sequence (SEQ ID NO:993), a wild type PKP2 nucleotide sequence (SEQ ID NO:1038, with the sh#5 sequence underlined), a corresponding shIMM PKP2 nucleotide sequence (SEQ ID NO:1039, with the shIMM sequence underlined), and a PKP2 amino acid sequence (SEQ ID NO:1040).

FIGS. 5A-5C show results obtained from experiments used to test KCNQ1 shRNAs for the KCNQ1-SupRep vector. TSA201 cells were co-transfected with KCNQ1-WT and various KCNQ1 shRNAs or a non-targeting scrambled shRNA control (shCT). FIG. 5A includes a graph (top) plotting KCNQ1 expression for cells co-transfected with four commercial shRNAs (sh#1-4), normalized to GAPDH, measured by qRT-PCR. An image of a representative western blot of KCNQ1 with cofilin housekeeping control also is shown (bottom). FIG. 5B is a graph plotting ImageJ quantification of western blot relative pixel density. KCNQ1 sh#4 was selected for the final KCNQ1-SupRep gene therapy vector, and is referred to as shKCNQ1 in the further studies described herein. Results and representative images were obtained from three independent experiments (defined as three identical repeats of each experiment conducted from start to finish on separate weeks with one biological replicate per treatment group per run). Graphs show mean±S.D. One-way ANOVA with post-hoc Tukey's test for multiple comparisons also was used. *p<0.05. FIG. 5C is a graph plotting knockdown of KCNQ1 in TSA201 cells co-transfected with various custom shRNAs (sh#5-sh#8), normalized to GAPDH, determined using qPCR.

FIGS. 6A and 6B depict the design for the KCNQ1 suppression-replacement (KCNQ1-SupRep) vector. FIG. 6A shows a sequence alignment of the target sequence portion of shKCNQ1 (SEQ ID NO:7) to KCNQ1-WT cDNA (SEQ ID NO:8) (top) and “shRNA-immune” KCNQ1 (KCNQ1-shIMM, bottom) (SEQ ID NO:9), which includes wobble base synonymous variants (underlined). The amino acid sequence shown is KCNQ1 p.V458-P469 (c.1372-1407, NM_000218.2) (SEQ ID NO:10). FIG. 6B is a schematic of representative KCNQ1-SupRep vector maps. (U6) U6 promoter; (CMV) cytomegalovirus promoter; (MHC) alpha-myosin heavy chain promoter, (MLC) myosin light chain 2 promoter, (TnC) cardiac troponin C promoter, (TnT) cardiac troponin T promoter, (E) calsequestrin-2 cardiomyocyte-specific transcriptional cis-regulatory enhancer motif, (IRES) internal ribosome entry site; and (CFP) cyan fluorescent protein.

FIGS. 7A and 7B show that shKCNQ1 knocks down KCNQ1-WT but not KCNQ1-shIMM in TSA201 cells co-transfected with KCNQ1-WT or KCNQ1-shIMM and shCT, shKCNQ1, or KCNQ1-SupRep. FIG. 7A is a graph (top) plotting relative KCNQ1 expression normalized to GAPDH measured by allele-specific qRT-PCR quantifying KCNQ1-WT (white) and KCNQ1-shIMM (grey). Results were confirmed with western blotting (bottom) for KCNQ1 with cofilin as housekeeping control. FIG. 7B is a graph plotting ImageJ quantification of western blot pixel density. Results and representative images were obtained from three independent experiments (defined as three identical repeats of each experiment conducted from start to finish on separate weeks with one biological replicate per treatment group per run). Both graphs show mean±S.D. For relative KCNQ1, one-way ANOVA with post-hoc Tukey's test for multiple comparisons was used in both FIG. 7A and FIG. 7B. For the sample treated with KCNQ1-SupRep in FIG. 7A, an unpaired 2-tailed student's t-test was used to compare the proportion of KCNQ1-WT compared to KCNQ1-shIMM (vertical bracket). *p<0.05.

FIG. 8 is a graph plotting relative KCNQ1 levels, indicating that suppression and replacement of KCNQ1-WT by shKCNQ1 and KCNQ1-SupRep was dose-dependent. TSA201 cells were co-transfected with 100 fmol KCNQ1-WT and a range (0-300 fmol) of shCT, shKCNQ1, or KCNQ1-SupRep. KCNQ1 expression was measured by allele-specific qRT-PCR and normalized to GAPDH. Markers represent the total KCNQ1. For KCNQ1-SupRep treatment when both KCNQ1-WT and -shIMM were present simultaneously, the allele-specific proportions of KCNQ1-WT (light grey shading) and KCNQ1-shIMM (dark grey shading) are shown.

FIG. 9 is a graph plotting relative KCNQ1 levels during activation of the two components of KCNQ1-SupRep showing that both shKCNQ1 and KCNQ1-shIMM activate at essentially the same rate. TSA201 cells were co-transfected with 100 fmol KCNQ1-WT and 100 fmol of shCT, shKCNQ1, KCNQ1-shIMM, or KCNQ1-SupRep and RNA harvested at different time points from 0 hours to 72 hours. KCNQ1 expression was measured by allele-specific qRT-PCR and normalized to GAPDH. Markers represent the total KCNQ1. For KCNQ1-SupRep treatment when both KCNQ1-WT and -shIMM were present simultaneously, the allele-specific proportion of KCNQ1-WT (light grey shading) and KCNQ1-shIMM (dark grey shading) are shown. Cells treated with KCNQ1-WT and shCT have nearly identical total KCNQ1 compared to cells treated with KCNQ1-WT and KCNQ1-SupRep, however in KCNQ1-SupRep, the proportion of KCNQ1-WT (light grey shading) is strongly suppressed while the proportion of KCNQ1-shIMM (dark grey shading) becomes the predominant form of KCNQ1 present.

FIGS. 10A-10C show patch clamp analysis of I_{Ks} in TSA201 cells co-transfected with KCNQ1-WT, KCNQ1-shIMM, or KCNQ1-variants and the Kv7.1 beta-subunit, KCNE1. FIG. 10A shows representative voltage clamp I_{Ks} traces for the indicated constructs, determined from a holding potential of −80 mV and test potentials from −40 mV to +80 mV in 10 mV increments with 4s duration. KCNQ1-shIMM produced WT I_{Ks} current (top). KCNQ1-Y171X, KCNQ1-V254M, and KCNQ1-I567S produced no I_{Ks} current (bottom). FIG. 10B is a graph plotting peak current density in the transfected cells. Error bars represent standard error of the mean (S.E.M.). FIG. 10C is a graph plotting peak current density at the +80 mV depolarization step. Error bars represent standard deviation (S.D.). One-way ANOVA with post-hoc Tukey's test for multiple comparisons also was used. *p<0.05.

FIG. 11 is a series of representative images showing immunofluorescence of TSA201 cells transfected with KCNQ1-WT, KCNQ1-shIMM, or KCNQ1-variants. KCNQ1-shIMM and KCNQ1-WT both trafficked to the cell membrane. KCNQ1-Y171X resulted in a premature stop codon and no expressed protein, while KCNQ1-V254M correctly trafficked to the cell membrane. KCNQ1-I567S created detectable protein, although seemingly at a lower expression level consistent with qPCR and western blot results. DAPI was used to stain nuclei, KCNQ1 (green), and merge. Representative images were obtained from three independent experiments (defined as three identical repeats of this experiment conducted from start to finish on separate weeks with one biological replicate per treatment group per run). Scale bars=20 μm.

FIG. 12 includes a graph (top) and a western blot (bottom) showing that KCNQ1-SupRep knocked down LQT1 disease-causing KCNQ1 variants, including both nonsense and missense variants, and replaced the variants with KCNQ1-shIMM. TSA201 cells were co-transfected with KCNQ1-WT or KCNQ1-variants and shCT, shKCNQ1, or KCNQ1-SupRep. shKCNQ1 knocks down KCNQ1 in a variant-independent manner. KCNQ1-SupRep knocks down KCNQ1 variants via shKCNQ1 and expresses KCNQ1-shIMM, which is knockdown immune. The graph at the top of FIG. 12 demonstrates proportional expression of KCNQ1-WT/variants and KCNQ1-shIMM, detected using allele-specific qRT-PCR to measure KCNQ1-WT/variant (white) and KCNQ1-shIMM (gray). Overall KCNQ1 expression (not allele-specific) was validated by western blotting with cofilin as a housekeeping control (FIG. 12, bottom). Results and representative images were obtained from three independent experiments (defined as three identical repeats of each experiment conducted from start to finish on separate weeks with one biological replicate per treatment group per run). The graph shows mean±S.D. For relative KCNQ1, a separate one-way ANOVA with post-hoc Tukey's test for multiple comparisons was conducted for each KCNQ1 variant to compare the three treatments and avoid extraneous comparisons between variants. In samples treated with KCNQ1-SupRep, an unpaired two-tailed student's t-test was used to compare the proportion of KCNQ1-WT compared to KCNQ1-shIMM (vertical brackets). *p<0.05.

FIGS. 13A-13D show quality control of iPSCs derived from four patients with LQT1, an unrelated healthy control, and two CRISPR-Cas9 corrected isogenic control iPSCs generated from two of the LQT1 patient iPSCs (KCNQ1-V254M and KCNQ1-A344A/sp1). FIG. 13A shows Sanger sequencing confirmation of LQT1-causative KCNQ1 variants in iPSCs derived from patients with LQT1 (middle), from an unrelated healthy control (top), and from isogenic controls (bottom). FIGS. 13B-13D show representative quality control studies completed for all iPSC lines, including normal karyotype (FIG. 13B), bright field image of an iPSC colony with normal morphology (FIG. 13C), and immunofluorescence microscopy (FIG. 13D) for markers of pluripotency including DAPI nuclear stain, Tra-1-60 or SSEA-4, Nanog or Oct-4, and a merged image. Scale bars=2011M. (spl) splice; (*) silent variant introduced during CRISPR-Cas9 correction to prevent reintroduction of double-strand breaks after successful editing of the transfected target cell.

FIG. 14 includes representative images showing immunofluorescence of iPSC-CMs derived from a patient with KCNQ1-V254M mediated LQT1, one week after transduction with lentiviral shCT or KCNQ1-SupRep. The patient-derived iPSC-CMs were stained with three separate antibodies to demonstrate (1) the presence of cardiomyocytes (cardiac troponin T, CTNT), (2) transduction by lentivirus as indicated by the turboGFP reporter (GFP) in shCT or by the CFP reporter in KCNQ1-SupRep, and (3) the presence of KCNQ1 either endogenously or as the result of treatment with KCNQ1-SupRep. The results showed that high purity populations of cardiomyocytes were evenly transduced with lentiviral shCT or KCNQ1-SupRep. With shCT, there was weak staining for KCNQ1, but when cells were treated with KCNQ1-SupRep, KCNQ1 staining was bright, indicating robust expression. Cells were counterstained with DAPI for nuclear stain. The figure shows representative images of iPSC-CMs from one LQT1 variant (KCNQ1-V254M). Immunofluorescence results for iPSC-CMs derived from the unrelated control and other three LQT1 variants (KCNQ1-Y171X, -I567S, and -A344A/spl) are found in FIGS. 15A-15D. Scale bars 50 μm.

FIGS. 15A-15D show immunofluorescence images from the iPSC-CMs not shown in FIG. 14, including the unrelated control (FIG. 15A) and three LQT1 variants (KCNQ1-Y171X, -I567S, and -A344A/spl; FIGS. 15B, 15C, and 15D, respectively). Immunofluorescence images were acquired one week after transduction with lentiviral shCT or KCNQ1-SupRep. The patient-derived iPSC-CMs were stained with three separate antibodies to demonstrate (1) presence of cardiomyocytes (cardiac troponin T; CTNT), (2) transduction by lentivirus as indicated by the turboGFP reporter in shCT (GFP or CFP in KCNQ1-SupRep), and (3) the presence of KCNQ1, either endogenous or as the result of treatment with KCNQ1-SupRep. The results showed high purity populations of cardiomyocytes that were evenly transduced with lentiviral shCT or KCNQ1-SupRep. In shCT, there was weak staining for KCNQ1, but in treatment with KCNQ1-SupRep, KCNQ1 staining was bright and indicated robust expression. Cells were counterstained with DAPI for nuclear stain. Scale bars=50 μm.

FIGS. 16A and 16B show that action potential duration (APD) was shortened in LQT1 iPSC-CMs treated with lentivirus containing KCNQ1-SupRep compared to shCT. FIG. 16A includes a series of representative traces showing three consecutive FluoVolt™ voltage dye optical action potentials paced at 1 Hz for untreated, unrelated healthy control and KCNQ1-Y171X, KCNQ1-V254M, KCNQ14567S, and KCNQ1-A344A/spl iPSC-CMs treated with shCT or KCNQ1-SupRep. FIG. 16B includes a series of graphs plotting APD₉₀ and APD₅₀ values for untreated, unrelated healthy control and KCNQ1-Y171X, KCNQ1-V254M, KCNQ14567S, and KCNQ1-A344A/spl iPSC-CMs treated with shCT or KCNQ1-SupRep. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. The total number of measurements (n) is shown. Box plots show median and interquartile range with whiskers extending to minimum and maximum values. Baseline APD₉₀ and APD₅₀ values were assessed by one-way ANOVA with post-hoc Dunnett's test comparing each KCNQ1 variant treated with shCT to the untreated, unrelated control (TABLE 5). APD shortening due to KCNQ1-SupRep compared to treatment with shCT was assessed by unpaired two-tailed student's t-tests at both the APD₉₀ and APD₅₀ levels separately for each variant. *p<0.0001.

FIGS. 17A and 17B show that CRISPR-Cas9 corrected isogenic controls serve as a marker for “perfect” correction of the cardiac APD. FluoVolt™ voltage dye measurement of the cardiac APD was conducted in isogenic control iPSC-CMs generated from two of the four LQT1 iPSCs (KCNQ1-V254M and KCNQ1-A344A/sp1). Data for treatment with shCT or KCNQ1-SupRep was shown here unchanged from FIGS. 16A and 16B. Both isogenic control iPSC-CMs had significantly shorter APD₉₀ and APD₅₀ than the LQT1 iPSC-CMs treated with shCT, which indicated that correction of the single pathogenic LQT1 variant in KCNQ1 was able to rescue the disease phenotype in vitro. As with the unrelated control, the isogenic controls were measured untreated as to provide the purest signal for a normal APD. Treatment of LQT1 iPSC-CMs with KCNQ1-SupRep resulted in APD shortening, although the degree of shortening was variable. For KCNQ1-V254M, KCNQ1-SupRep undercorrected the prolonged APD₉₀ and overcorrected the APD₅₀. In KCNQ1-A344A/spl, ideal correction for the APD₉₀ was achieved and matched the isogenic control APD₉₀, but overcorrection of the APD₅₀ also occurred. FIG. 17A includes representative traces showing three consecutive action potentials paced at 1 Hz.

FIG. 17B includes a pair of graphs plotting APD₉₀ and APD₅₀ values for untreated, isogenic controls, and KCNQ1-V254M and KCNQ1-A344A/spl iPSC-CMs treated with shCT or KCNQ1-SupRep. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. The total number of measurements (n) is shown. Box plots show median and interquartile range with whiskers extending to minimum and maximum values. A one-way ANOVA with post-hoc Tukey's test comparing all pairs for APD₉₀ and all pairs for APD₅₀ was used for each KCNQ1 variant tested. *p<0.0001, unless indicated by a specific p-value in the figure.

FIGS. 18A-18D show that use of iPSC-CM 3D organoid culture system can achieve results similar to those obtained in standard syncytial monolayer culture. To assess whether culture in 3D organoid or syncytial monolayer yields findings similar to monolayer culture, the iPSC-CMs from one of the four patients with LQT1 (KCNQ1-Y171X) were dissociated and plated into a round mold containing thick collagenous MATRIGEL® to form a spheroid. After 2-3 days, the iPSC-CMs formed a strong beating syncytium in 3D, and were used as the organoid model for this study. The organoids were treated with KCNQ1-SupRep, shCT, or left untreated as control. Seven days post viral transduction, the organoids were assayed by immunofluorescence or FluoVolt™ voltage dye. FIG. 18A is an image of a beating iPSC-CM organoid suspended in media in a 24-well culture plate, with a zoomed in image shown in the inset. FIG. 18B includes representative images of organoids that were fixed, cryosectioned, and stained for immunofluorescence using the cardiomyocyte marker cardiac troponin T (CTNT; top) and the lentiviral transduction marker as indicated by the turboGFP reporter in shCT (GFP; middle) or by the CFP reporter in KCNQ1-SupRep (bottom). FIG. 18C is a representative trace of FluoVolt™ voltage dye in the untreated LQT1 organoid or the LQT1 organoid treated with KCNQ1-SupRep. FIG. 18D is a graph plotting overall APD₉₀ and APD₅₀ values for untreated and KCNQ1-SupRep treated organoids from KCNQ1-Y171X iPSC-CMs. *p<0.0001.

FIGS. 19A-19F provide a summary of the LQT1 and LQT2 transgenic rabbit phenotype. Shown in FIG. 19A are schematic representations of pathogenic variants (KCNQ1-Y315S and KCNH2-G628S) in the KCNQ1-encoded potassium channel subunit (left) and KCNH2-encoded potassium channel subunit polypeptides (right) and the transgenic constructs (bottom). FIG. 19B includes representative electrocardiogram traces showing the differences in QT interval between wild-type (WT), LQT1, and LQT2 rabbits. FIG. 19C is a bar graph showing the significant difference in QT interval duration between WT and LQT1 or LQT2 rabbits. FIG. 19D shows the spontaneous torsades de pointes (TdP) in a oestradiol-treated LQT2 rabbit initiated by short-long-short sequence. FIG. 19E includes representative cellular cardiac action potential traces that demonstrated prolonged action potential durations in LQT1 and LQT2 rabbit cardiomyocytes compared with cardiomyocytes from WT rabbits. FIG. 19F shows IV-curves of I_{Ks} and I_{Kr} currents in cardiomyocytes isolated form WT, LQT1, and LQT2 rabbit hearts, indicating the loss of I_{Ks} in LQT1 rabbits and loss of I_{Kr} in LQT2 rabbits.

FIGS. 20A-20C demonstrate generation and confirmation of KCNH2-G604S and KCNH2-N633S iPSC lines. FIG. 20A is an image of a karyotype, showing that each clone had a normal karyotype for their respective sex. FIG. 20B is an image showing phase-contrast light images of iPSC colonies from each of the patient cell lines used for the study. FIG. 20C contains representative Sanger sequencing chromatograms for the patent cell lines. The boxes indicate the relevant codon, and the stars indicate the exact nucleotide of interest. Scale bars=50 μm.

FIG. 21 is an image showing immunocytochemistry for p.G604S clone #1, p.G604S clone #2, p.N633S clone #1, and p.N633S clone #2. Each of the respective clones for each line was demonstrated to express Nanog and SSEA4 pluripotency markers. Scale bars=20 μm.

FIG. 22 is a graph plotting knockdown of KCNH2 in TSA201 cells with various shRNAs, determined using qPCR.

FIG. 23 is a graph plotting the results of FluoVolt™ studies using CRISPR-Cas9 corrected isogenic controls as a marker for correction of cardiac APD in N633 S iPSC-CMs and isogenic control iPSC-CMs generated from LQT2 iPSCs (N633S). APD_{90B} and APD_{50B} values were determined for isogenic control treated with shCT, and for KCNH2-N633S variant treated with shCT or KCNH2-SupRep. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point, and Bazett corrected APD_{90B} and APD_{50B} values were plotted. The total number of measurements (n) and medians (horizontal black lines) are indicated. A one-way ANOVA with post-hoc Tukey's test comparing all pairs for APD_{90B} and all pairs for APD_{50B} was used.

FIG. 24 is a graph plotting the results of FluoVolt™ voltage dye measurement of cardiac APD in N633S iPSC-CMs and isogenic control iPSC-CMs generated from LQT2 iPSCs (N633S). APD_{90B} and APD_{50B} values for the untreated (UT) KCNH2-N633S variant, the SupRep treated isogenic control, and the untreated (UT) isogenic control are shown. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. Bazett corrected APD_{90B} and APD_{50B} values are shown, and the total number of measurements (n) is indicated. Dot plots show median (horizontal black line). A one-way ANOVA with post-hoc Tukey's test comparing all pairs for APD_{90B} and all pairs for APD_{50B} was used.

FIG. 25 is a graph plotting the results of FluoVolt′ voltage dye measurement of cardiac APD in G604S iPSC-CMs. APD₉₀ and APD₅₀ values for KCNH2-G604S variant treated with shCT and KCNH2-G604S variant treated with KCNH2-SupRep are shown. Treatment of LQT2 iPSC-CMs with KCNH2-SupRep results in significant APD₉₀ and APD₅₀ shortening compared to those treated with shCT. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. The total number of measurements (n) is shown. Dot plots show median (horizontal black line). A student's t-test comparing all pairs for APD_{90B} and all pairs for APD_{50B} was used.

FIG. 26 is a graph plotting APD₉₀ and APD₅₀ values for the KCNH2-G604S variant treated with shCT (1), and KCNH2-SupRep (2), or CRISPR-Cas9 corrected isogenic control treated with shCT (3). Treatment of the KCNH2-G604S iPSC-CMs with KCNH2-SupRep resulted in significant APD₉₀ shortening compared to treatment with shCT. Action potential trace videos were obtained for 20 second durations at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. The total number of measurements (n) is shown. The graph also shows the medians (horizontal black lines). A one-way ANOVA with post-hoc Tukey's test was used to compare all pairs for APD₉₀ and all pairs for APD₅₀.

FIG. 27 is a graph plotting APD₉₀ and APD₅₀ values for the KCNH2-G628S variant treated with shCT (1), KCNH2-SupRep (2), or CRISPR-Cas9 corrected isogenic control treated with shCT (3). Treatment of the KCNH2-G628S iPSC-CMs with KCNH2-SupRep resulted in significant APD₉₀ shortening compared to treatment with shCT. Action potential trace videos were obtained for 20 second durations at 50 fps with 1 Hz pacing. Regions of interest containing flashing cells were identified, and the changes in fluorescence intensity over time were measured to produce optical action potentials from which APD₉₀ values were determined. APD₉₀ values for all action potentials within a 20 second trace were averaged to produce a single data point. The total number of measurements (n) is shown. The graph also shows the medians (horizontal black lines). A one-way ANOVA with post-hoc Tukey's test was used to compare all pairs for APD₉₀.

FIGS. 28A and 28B show that KCNH2-SupRep knocked down LQT2 disease-causing KCNH2 missense variants and replaced them with KCNH2-shIMM. TSA201 cells were co-transfected with KCNH2-WT or KCNH2-variants and shCT, shKCNH2, or KCNH2-SupRep. shKCNH2 knocked down KCNH2 in a variant-independent manner. FIG. 28A is a graph plotting proportional expression of KCNH2-WT/variants and KCNH2-shIMM, which were detected using allele-specific qRT-PCR to measure KCNH2-WT/variant (white) and KCNH2-shIMM (grey). FIG. 28B is an image of a western blot showing overall KCNH2 expression (not allele-specific), with GAPDH as a housekeeping control.

FIGS. 29A and 29B show that shKCNH2 knocked down KCNH2-WT but not KCNH2-shIMM in TSA201 cells co-transfected with KCNH2-WT or KCNH2-shIMM and shCT, shKCNH2, or KCNH2-SupRep. FIG. 29A is a graph plotting relative KCNH2 expression normalized to GAPDH, as measured by allele-specific qRT-PCR to quantify KCNH2-WT (white) and KCNH2-shIMM (grey). Results were confirmed with western blotting (FIG. 29B) for KCNH2, with GAPDH as a housekeeping control.

FIGS. 30A-30D show that KCNH2-AAV-P2A CTnC-EGFP did not generate KCNH2 current in heterologous TSA 201 cells. FIG. 30A is a plot of representative whole cell I_{Kr} tracings from TSA201 cells expressing KCNH2-WT with KCNE2, determined from a holding potential of −80 mV and testing potentials from −40 mV to +60 mV in 10 mV increments with a 3 second duration. FIG. 30B shows representative whole cell outward tracings from TSA201 cells expressing KCNH2-AAV-P2A CTnC-EGFP, determined from a holding potential of −80 mV and testing potentials from −40 mV to +60 mV in 10 mV increments with a 3 second duration. FIG. 30C is a graph plotting current-voltage relationship for KCNH2-pIRES2-EGFP with KCNE2-pIRES2-dsRed2 (n=9) and KCNH2-AAV-P2A CTnC-EGFP (n=8). All values represent mean±SEM. FIG. 30D is a graph plotting peak current density at +10 mV for KCNH2-pIRES2-EGFP with KCNE2-pIRES2-dsRed2 (n=9) and KCNH2-AAV-P2A CTnC-EGFP (n=8). All values represent mean±SEM.

FIGS. 31A-31E show that KCNH2-AAV-P2A CTnC-EGFP generated E-4031 sensitive outward current in H9C2 cells. FIG. 31A includes representative whole cell outward current tracings from empty H9C2 cells (upper panel), H9C2 cells expressing KCNH2-AAV-P2A CTnC-EGFP before E-4031 (middle panel), and H9C2 cells expressing KCNH2-AAV-P2A CTnC-EGFP after E-4031 (lower panel) determined from a holding potential of −80 mV and testing potentials from −40 mV to +60 mV in 10 mV increments with a 3 second duration. FIG. 31B is a graph plotting current-voltage relationship for outward current from empty H9C2 cells and H9C2 cells expressing KCNH2-AAV-P2A CTnC-EGFP (n=9). All values represent mean±SEM. FIG. 31C is a graph plotting peak current density at +60 mV from empty H9C2 cells and H9C2 cells expressing KCNH2-AAV-P2A CTnC-EGFP (n=9). All values represent mean±SEM. FIG. 31D is a graph plotting current-voltage relationship for H9C2 cells expressing KCNH2-AAV-P2A CTnC-EGFP, before and after E-4031 (n=6). All values represent mean±SEM. FIG. 31E is a graph plotting peak current density at +60 mV from H9C2 cells expressing KCNH2-AAV-P2A CTnC-EGFP, before and after E-4031 (n=6). All values represent mean±SEM.

FIG. 32 is a graph plotting APD₉₀ and APD₅₀ values for the KCNH2-N588K variant treated with shCT (1), KCNH2-SupRep (2), or isogenic control treated with shCT (3). Treatment of SQT1 iPSC-CMs with KCNH2-SupRep resulted in significant APD₉₀ prolongation compared to treatment with shCT. Action potential trace videos were obtained for 20 second durations at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point. The total number of measurements (n) is shown. The graph also shows medians (horizontal black line). A one-way ANOVA with post-hoc Tukey's test was used to compare all pairs for APD₉₀ and APD₅₀ was used.

FIGS. 33A-33D show quality control for iPSCs derived from a patient with the SCN5A-F1760C variant. FIG. 33A is a bright field image of an iPSC colony with normal morphology. FIG. 33B shows the Sanger sequencing confirmation (SEQ ID NO:1047) of the LQT3-causing SCN5A-F1760C variant in iPSCs derived from the patient. FIG. 33C is an image showing a normal karyotype for the iPSC line generated from the patient's blood sample. FIG. 33D includes images of immunofluorescence microscopy for markers of pluripotency, including DAPI nuclear stain, Tra-1-60 or SSEA-4, Nanog or Oct-4, and a merged image.

FIG. 34 is a graph plotting knockdown of SCN5A in TSA201 cells with various shRNAs, determined using qPCR.

FIG. 35 is a schematic showing representative SCN5A-SupRep vector maps.

(CMV) cytomegalovirus promoter; (MCS) multiple cloning site; (U6) U6 promoter; (ChlorR) chloramphenicol resistance gene; (Ori) origin of replication; (WPRE) Woodchuck Hepatitis Virus Posttranscriptional Regulatory Element; (GFP) green fluorescent protein; (P2A) a member of 2A self-cleaving peptide family; (HA) tag derived from the human influenza hemagglutinin molecule corresponding to amino acids 98-106.

FIGS. 36A and 36B show that the APD was shortened in LQT3 SCN5A-F1760C iPSC-CMs treated with lentivirus containing SCN5A-SupRep, compared to untreated cells. FIG. 36A includes representative traces showing five consecutive FLUOVOLT™ voltage dye optical action potentials paced at 1 Hz for untreated and SCN5A-SupRep treated SCN5A-F1760C iPSC-CMs. FIG. 36B is a graph plotting APD₉₀ and APD₅₀ values for untreated and SCN5A-SupRep treated SCN5A-F1760C iPSC-CMs. Action potential trace videos were obtained for a 20 second duration at 50 fps with 1 Hz pacing. Regions of interest containing flashing cells were identified, and the changes in fluorescence intensity over time were measured to produce optical action potentials from which APD₉₀ and APD₅₀ values were determined. APD₉₀ and APD₅₀ values for all action potentials within a 20 second trace were averaged to produce a single data point.

FIG. 37 is a graph plotting knockdown of MYH7 in TSA201 cells with various shRNAs, determined using qPCR.

FIG. 38 is a graph plotting knockdown of PKP2 in TSA201 cells with various shRNAs, determined by qRT-PCR.

FIGS. 39A-39D show quality control of iPSCs derived from a patient with a PKP2-c2146-1G>C variant. FIG. 39A includes bright field images of iPSC colonies with normal morphology. FIG. 39B shows Sanger sequencing confirmation of the ACM-causative PKP2-c2146-1G>C variant in iPSCs derived from the patient with ACM. FIG. 39C shows a normal karyotype for clones from the iPSC line generated from the patient's blood sample. FIG. 39D includes images of immunofluorescence microscopy for DAPI nuclear stain and markers of pluripotency, including Tra-1-60 or SSEA-4, Nanog or Oct-4, and a merged image.

FIG. 40 includes a series of graphs showing that calcium transient duration (CTD) and decay were shortened in ACM iPSC-CMs treated with lentivirus containing PKP2-SupRep compared to untreated cells. Given that PKP2-mediated ACM-associated arrhythmic events are often associated with exertion, calcium handling measurements were performed under both baseline and following treatment with the adrenergic agonist, isoproterenol (Iso). Trace videos were obtained for a 20 second duration at 50 fps with 0.5 Hz pacing. Regions of interest containing flashing cells were identified, and the changes in fluorescence intensity over time were measured to produce calcium transient traces from which the values were determined. All values of calcium transients within a second trace were averaged to produce a single data point for all the parameters except for calcium amplitude, where only the first value was taken for analysis.

FIG. 41 is a graph plotting knockdown of DSP in TSA201 cells with various shRNAs, determined by qRT-PCR.

FIG. 42 is a graph plotting knockdown of MYBPC3 in TSA201 cells with various shRNAs, determined by qRT-PCR.

FIG. 43 is a graph plotting knockdown of RBM20 in TSA201 cells with various shRNAs, determined by qRT-PCR.

FIG. 44 is a graph plotting knockdown of CACNA1C in TSA201 cells with various shRNAs, determined by qRT-PCR.

FIG. 45 is a graph plotting knockdown of CALM1 in TSA201 cells with various shRNAs, determined by qRT-PCR.

FIG. 46 is a graph plotting knockdown of CALM2 in TSA201 cells with various shRNAs, determined by qRT-PCR.

FIG. 47 is a graph plotting knockdown of CALM3 in TSA201 cells with various shRNAs, determined by qRT-PCR.

FIG. 48 is a graph plotting knockdown of KCNJ2 in TSA201 cells with various shRNAs, determined by qRT-PCR.

FIG. 49 is a graph plotting knockdown of CASQ2 in TSA201 cells with various shRNAs, determined by qRT-PCR.

FIG. 50 is a graph plotting knockdown of DSG2 in TSA201 cells with various shRNAs, determined by qRT-PCR.

FIG. 51 is a graph plotting knockdown of TNNT2 in TSA201 cells with various shRNAs, determined by qRT-PCR.

FIG. 52 is a graph plotting knockdown of TPM1 in TSA201 cells with various shRNAs, determined by qRT-PCR.

FIG. 53 is a graph plotting knockdown of LMNA in TSA201 cells with various shRNAs, determined by qRT-PCR.

FIG. 54 is a graph plotting knockdown of PLN in TSA201 cells with various shRNAs, determined by qRT-PCR.

### DETAILED DESCRIPTION

This document provides methods and materials for treating a mammal having a congenital disease (e.g., a congenital heart disease such as a LQTS or, more specifically, LQT1, LQT2, or LQT3) through suppression of endogenous causative allele(s) and replacement with/expression of a non-mutant (non-causative), non-suppressed coding sequence. In general, the methods and materials provided herein involve the use of nucleic acid constructs that contain one or more suppressive components (e.g., an RNAi nucleic acid such as a shRNA) designed to suppress the expression of one or more disease-associated alleles (or their transcribed RNAs) within one or more types of cells (e.g., cardiomyocytes) present within a mammal (e.g., the heart of a mammal such as a human having LQTS, or more specifically, LQT1, LQT2, or LQT3), in combination with one or more corrective components (e.g., a nucleic acid encoding a version of the disease-associated allele that encodes a wild type polypeptide and is immune to the suppressive component). The methods and materials provided herein can be used to reduce one or more symptoms or effects of the disease caused by allele(s) targeted by the suppressive component.

In some cases, this document provides a suppression-and-replacement (SupRep) nucleic acid that can be used to treat a mammal having a congenital disorder. Disorders that can be treated according to the methods provided herein include, without limitation, LQTS (e.g., LQT1, LQT2, LQT3, LQT4, LQT5, LQT6, LQT7, LQT8, LQT9, LQT10, LQT11, LQT12, LQT13, LQT14, LQT15, LQT16, or LQT17), Brugada syndrome (BrS), catecholaminergic polymorphic ventricular tachycardia (CPVT), arrhythmogenic cardiomyopathy (ACM), hypertrophic cardiomyopathy (HCM), dilated cardiomyopathy (DCM), SQTS, Timothy syndrome, left ventricular non-compaction cardiomyopathy (LVNC), skeletal myopathy, Andersen-Tawil syndrome (ATS), familial hypercholesterolemia (FH), cardiomyopathies, atrial fibrillation, and Triadin knockout syndrome (TKOS).

The nucleic acids provided herein include two main components—a suppressive gene therapy component that can suppress the expression of a selected disease-associated allele, and a corrective gene therapy component encoding a corrected version of the selected disease-associated allele that is immune to the suppressive gene therapy component.

The suppressive component can be, for example, an RNAi nucleic acid such as a shRNA, siRNA, or a micro RNA (miRNA). The suppressive component can have any appropriate length. For example, the suppressive component can be from about 10 to 40 nucleotides in length (e.g., from about 10 to about 20, from about 15 to about 30, from about 18 to about 22, from about 20 to about 30, or from about 30 to about 40 nucleotides in length).

The suppressive component can be designed to target a region of a disease-associated allele that does not contain the pathogenic mutation(s) (e.g., LQTS-causative mutations) or other genetic polymorphisms. In this manner, the suppressive component can reduce the expression of numerous versions of the endogenous alleles, including wild type alleles, alleles containing disease-associated mutations, or alleles containing other polymorphisms that are not causative of the disorder to be treated.

In some cases, the suppressive component can be designed to target a region of a disease-associated allele that contains one or more pathogenic mutations (e.g., one or more LQTS-causative mutations) or other genetic polymorphisms.

The corrective component can be a nucleic acid that encodes a corrected version of the disease-associated allele that lacks the pathogenic mutation(s), and may encode a wild type polypeptide. The corrective component also contains base substitutions as compared to the endogenous version of the targeted gene, such that the corrective component is immune to (e.g., not suppressed by) the suppressive gene therapy component. For example, the region of the corrective component that would otherwise be targeted by the suppressive component can include from about 1 to about 13 (e.g., from about 1 to about 3, from about 2 to about 4, from about 3 to about 5, from about 4 to about 6, from about 5 to about 7, from about 6 to about 8, from about 7 to about 9, from about 8 to about 10, from about 9 to about 11, from about 10 to about 12, or from about 11 to about 13) wobble base synonymous variants that do not change the amino acid sequence encoded by the corrective component, as compared to the corresponding wild type sequence. In some cases, the region of the corrective component that would otherwise be targeted by the suppressive component can include 1, 2, 3, 4, 5, 6, 7, 8, 9, 10, 11, 12, or 13 wobble base synonymous variants that do not change the amino acid sequence encoded by the corrective component, as compared to the corresponding wild type sequence (e.g., wild type, non-pathogenic sequence). Due to the presence of the synonymous variants, expression of the suppressive component will not reduce the expression of the corrective component.

Other suppressive component/corrective component combinations also can be used. For example, in some cases, the suppressive component can be designed to target the 5′ untranslated region (UTR) or 3′ UTR, since the corrective cDNA does not contain the UTRs but endogenous transcription of mRNA does contain the UTRs. In such cases, the corrective component does not need to contain silent variants since the suppressive component (e.g., RNAi) is targeted to a UTR. In some cases, the suppressive component can target a sequence near the 5′ or 3′ end of the coding sequence, and the corrective component can include a truncated cDNA that does not contain the sequence targeted by the suppressive component.

In some cases, the corrective component may encode a polypeptide that is not 100% identical to the wild type polypeptide at the amino acid sequence level, but has activity at a level sufficient to treat the disorder. Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be encoded within a corrective component of a SupRep construct provided herein include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, a SupRep construct provided herein also can encode or contain a reporter. Any appropriate reporter can be used. In some cases, for example, a fluorescent reporter (e.g., green fluorescent protein, red fluorescent protein, or yellow fluorescent protein) can be used. In some cases, a non-fluorescent tag can be included. Any appropriate non-fluorescent tag can be used, including, without limitation, hemagglutinin, FLAG® tag, His6, and V5.

A non-limiting example of a SupRep construct provided herein is a SupRep KCNQ1 gene therapy vector that can be used for treating of mammals having LQT1. As described in the Examples herein, the therapeutic efficacy of the SupRep KCNQ1 gene therapy vector is supported by results obtained using two in vitro model systems. Again, the SupRep strategy has two components that occur in tandem. First, for KCNQ1 and LQT1, suppression of both endogenous KCNQ1 alleles (the WT allele and the LQT1 mutant-containing allele) occurs via a KCNQ1 shRNA. The second component involves replacement of KCNQ1 via expression of a shRNA-immune (shIMM) KCNQ1 cDNA that contains synonymous variants at the wobble base of each codon within the shRNA's binding sequence. As noted above, these synonymous variants did not alter the WT amino acid sequence, but did prevent knock down (KD) by the shRNA—thereby rendering it “immune” to the shRNA. KCNQ1-SupRep can be mutation-independent, eliminating the need to design multiple RNAi since the shRNA targets the gene itself rather than discrete mutations.

Nucleic acid molecules encoding a suppressive component and a corrective component can be produced by techniques including, without limitation, common molecular cloning, polymerase chain reaction (PCR), chemical nucleic acid synthesis techniques, and combinations of such techniques. For example, PCR can be used with oligonucleotide primers designed to amplify nucleic acid (e.g., genomic DNA or RNA) encoding a selected polypeptide (e.g., KCNQ1).

This document also provides methods for using the SupRep constructs described herein to treat a mammal identified as having a congenital disorder. As described in the Examples herein, for example, a KCNQ1-SupRep gene therapy vector was generated, and its ability to suppress and replace KCNQ1 was validated via heterologous expression in TSA201 cells. In addition, the LQT1 disease phenotype was rescued by shortening of the cardiac action potential duration (APD) in an in vitro cardiac model using patient-specific, induced pluripotent stem cell-derived cardiomyocytes (iPSC-CMs) generated from four patients with distinct LQT1-causative variants. Further, the studies described herein demonstrated that the KCNQ1-SupRep gene therapy approximated a “therapeutic cure,” in terms of APD normalization, when compared to the gold standard of a patient's own corrected isogenic control cells.

Any appropriate mammal can be treated as described herein. For example, mammals including, without limitation, humans, monkeys, dogs, cats, cows, horses, pigs, sheep, rabbits, rats, and mice having a congenital disorder (e.g., a congenital heart disorder such as a LQTS, or more specifically LQT1) can be treated as described herein. In some cases, a mammal (e.g., a human) having a congenital disease (e.g., a congenital cardiac disease such as a LQTS, or more specifically LQT1) can be treated by administering a SupRep nucleic acid construct to the mammal (e.g., to the heart muscle of the mammal) in a manner that suppresses expression of endogenous disease-associated alleles and provides a replacement wild type cDNA (or a cDNA that does not include disease-associated polymorphisms). A mammal can be identified as having a congenital disorder using any appropriate diagnostic technique. Non-limiting examples include, without limitation, genetic screening for one or more disease-associated alleles and assessment of organ (e.g., heart) function deficits (e.g., by electrocardiogram, echocardiogram, exercise stress test, and/or lidocaine challenge).

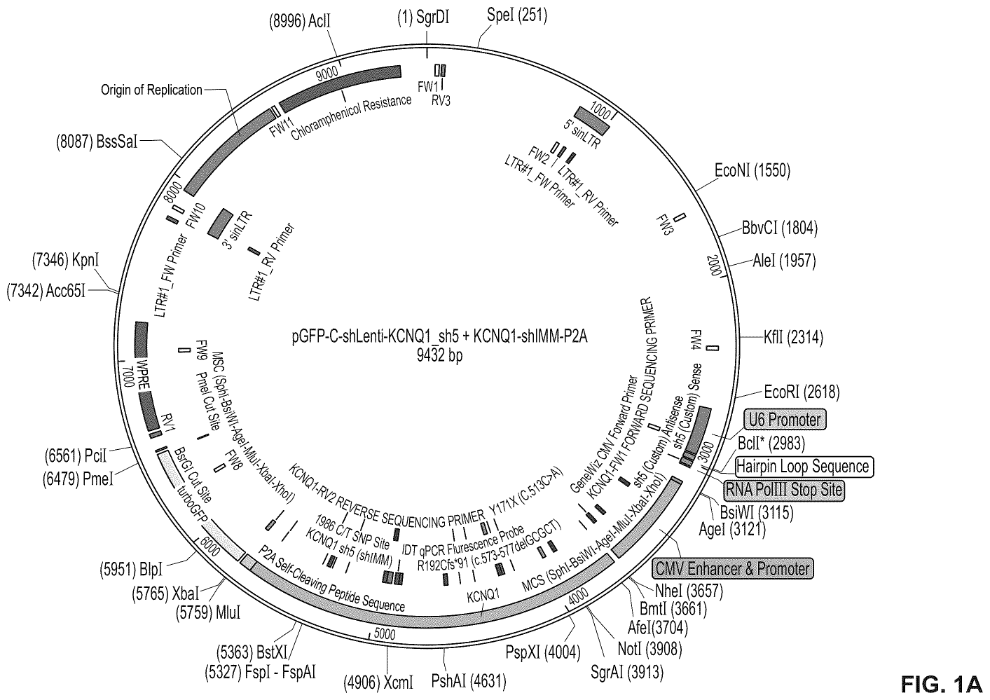

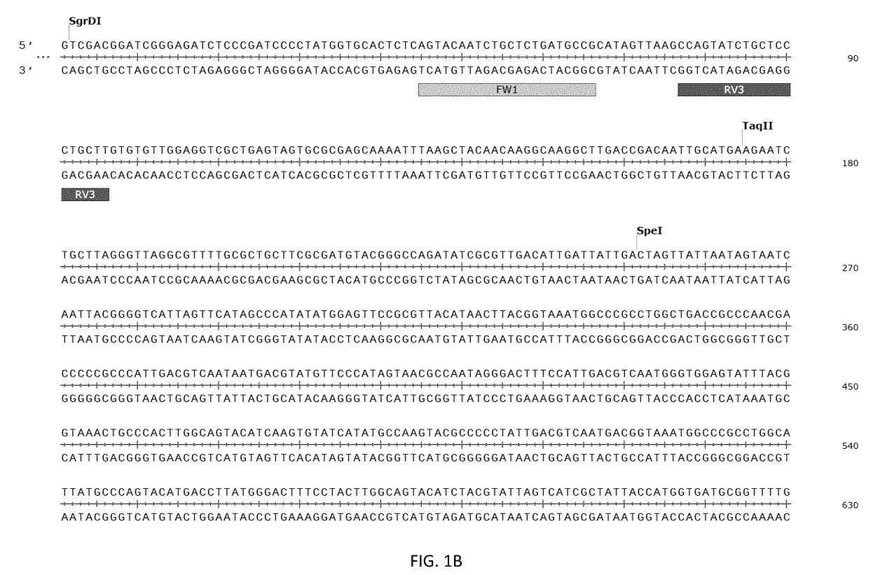

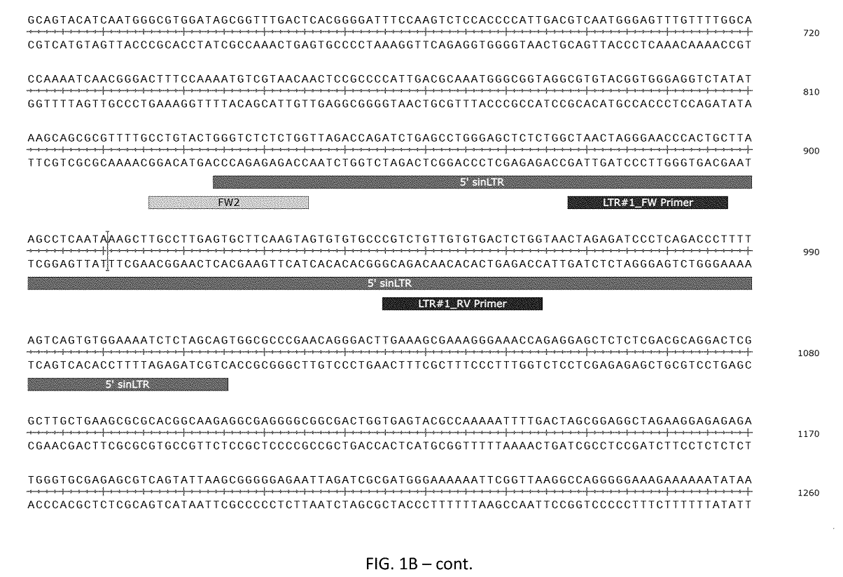

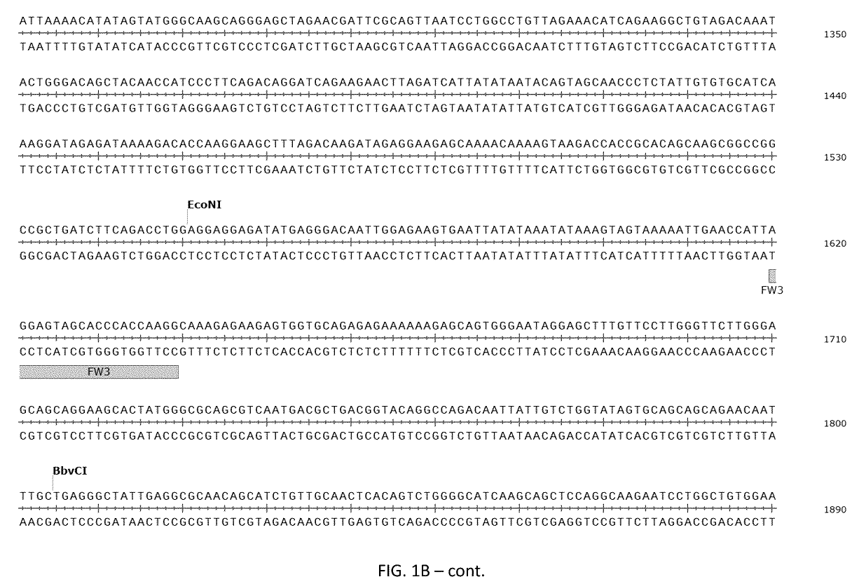

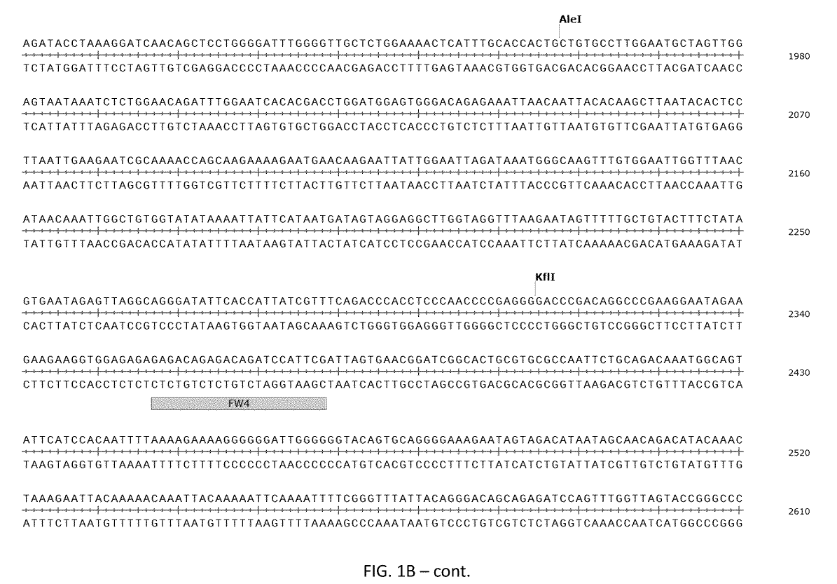

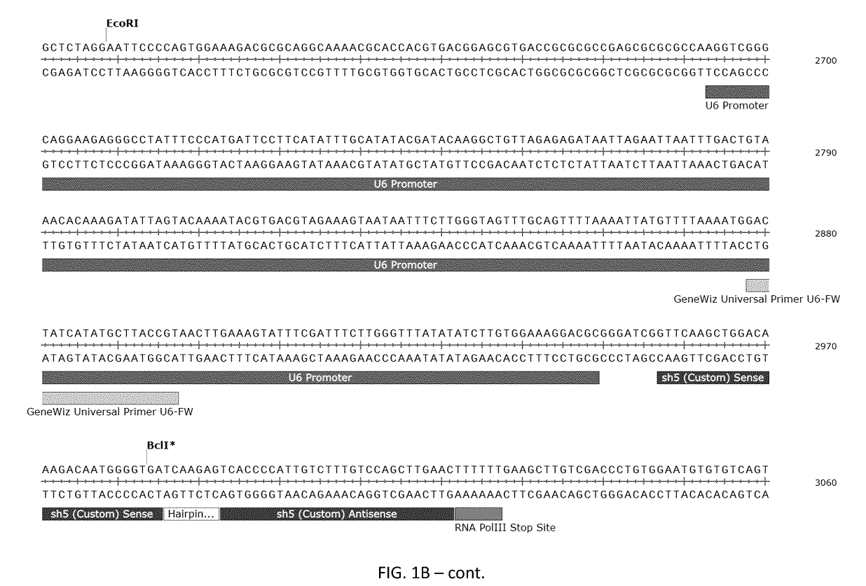

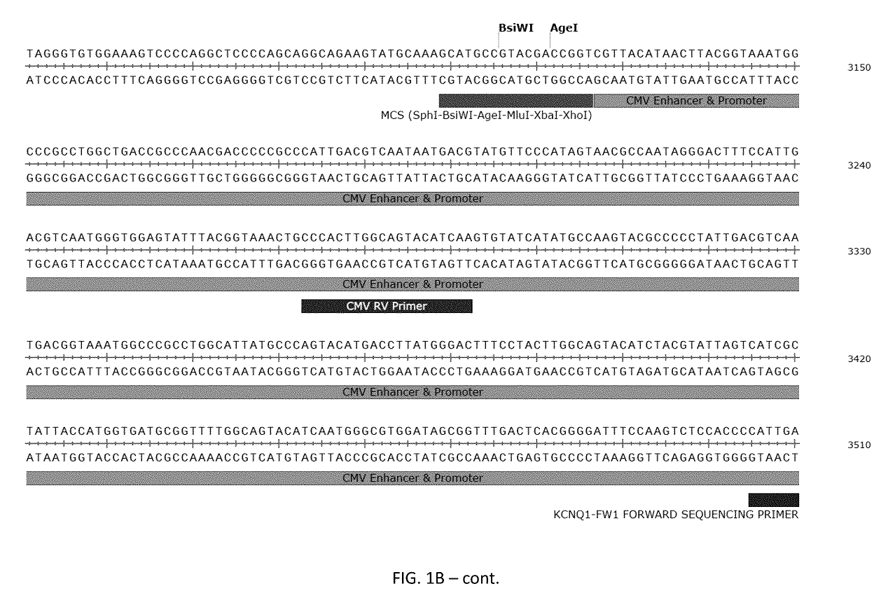

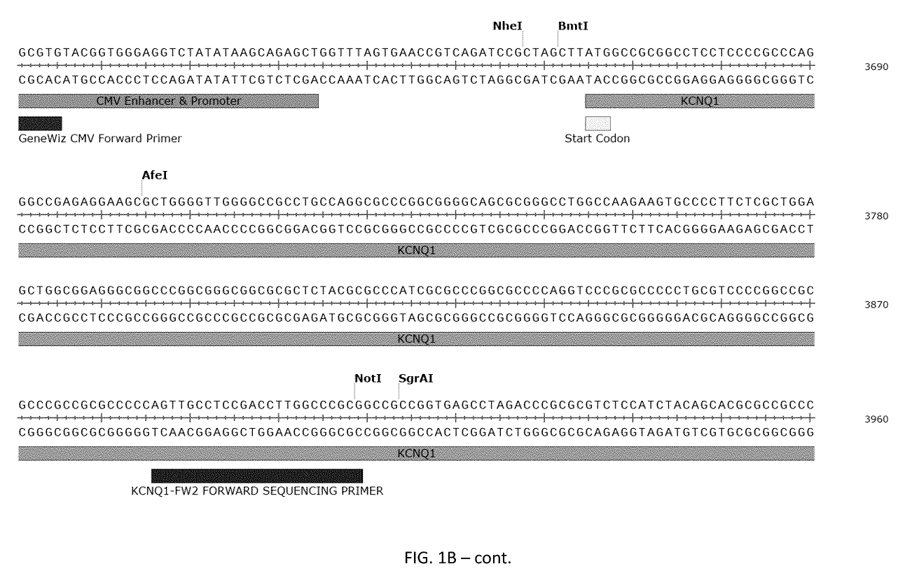

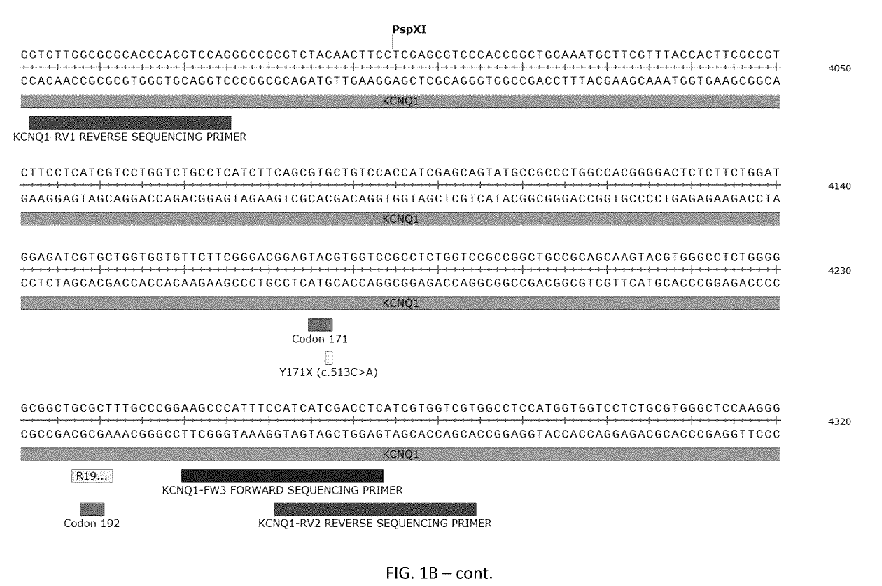

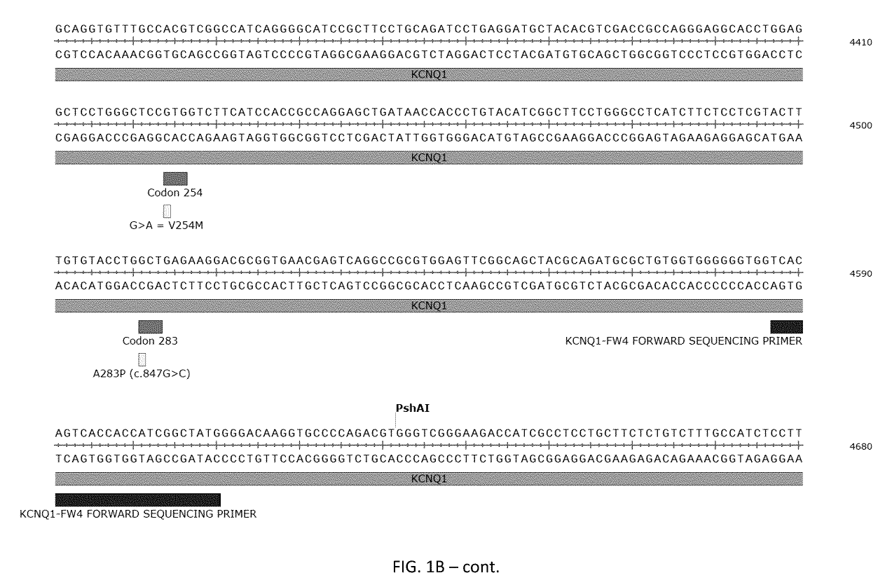

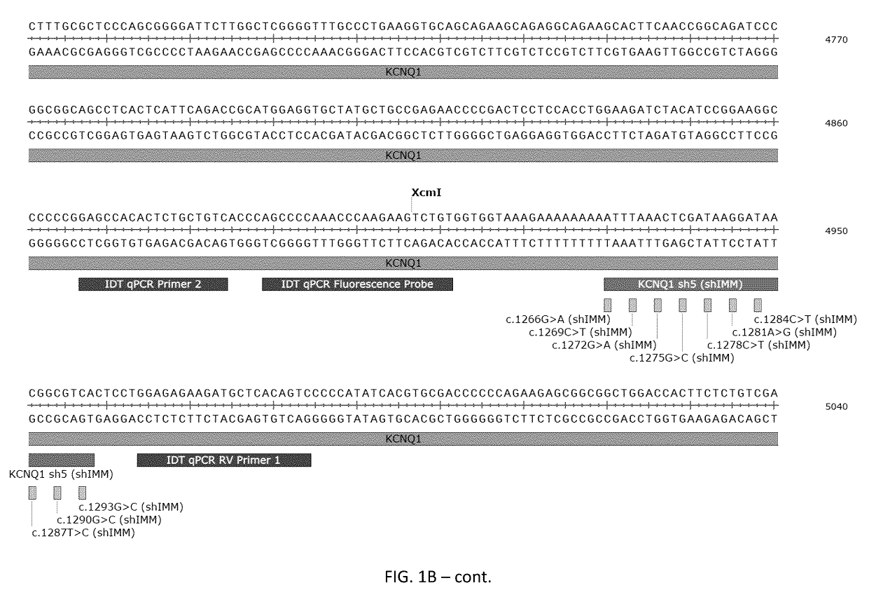

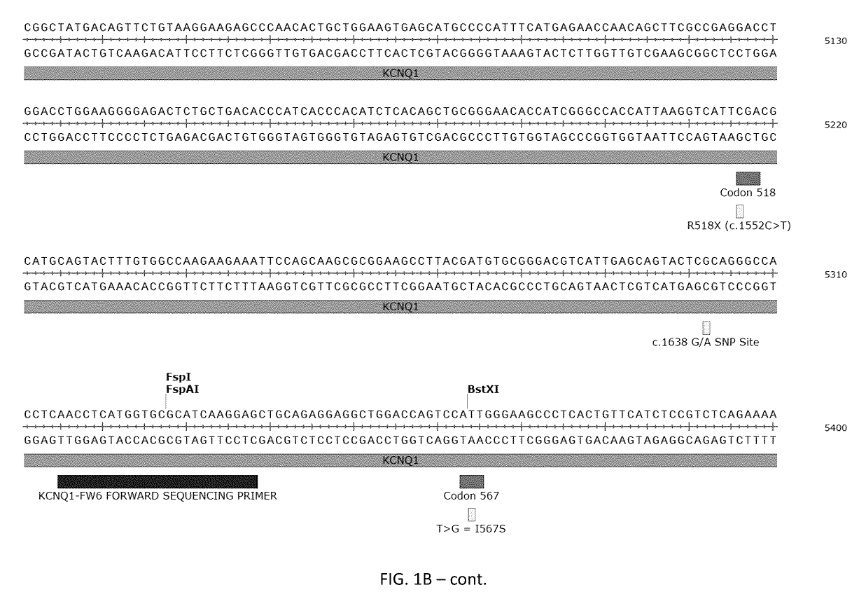

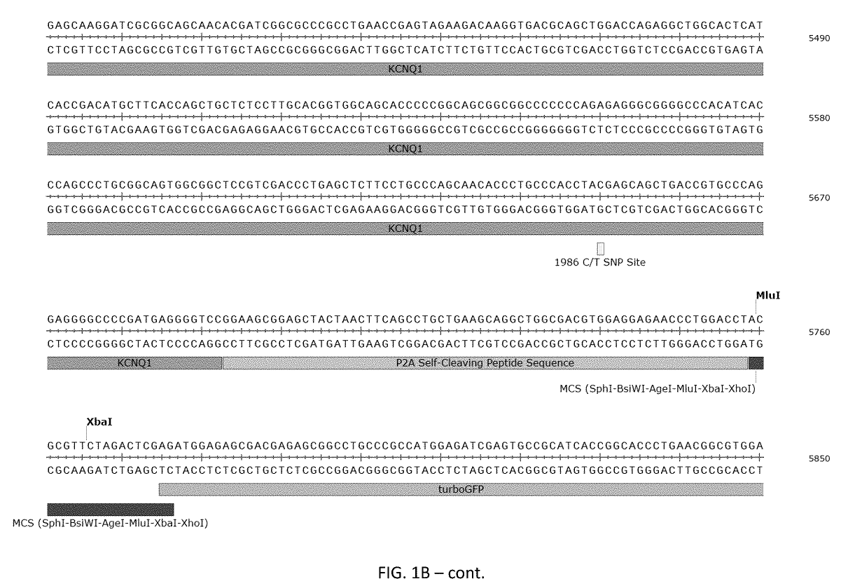

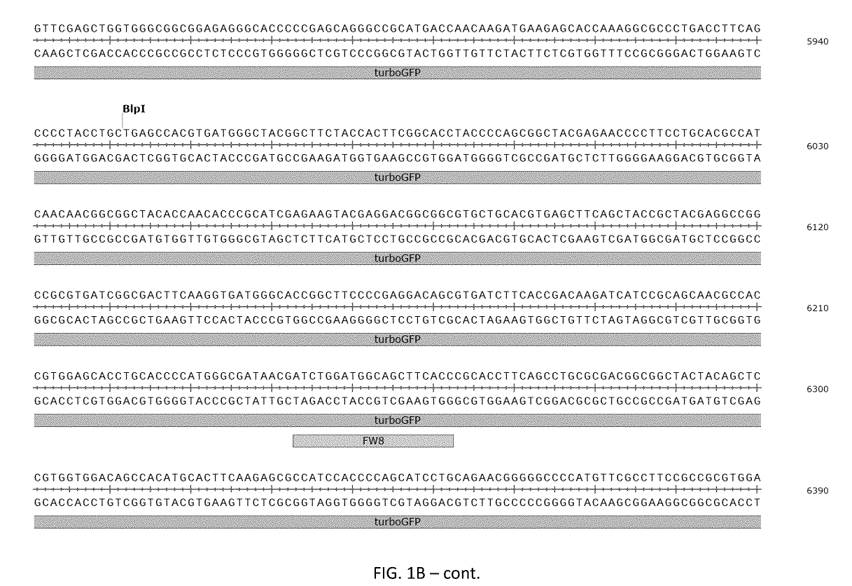

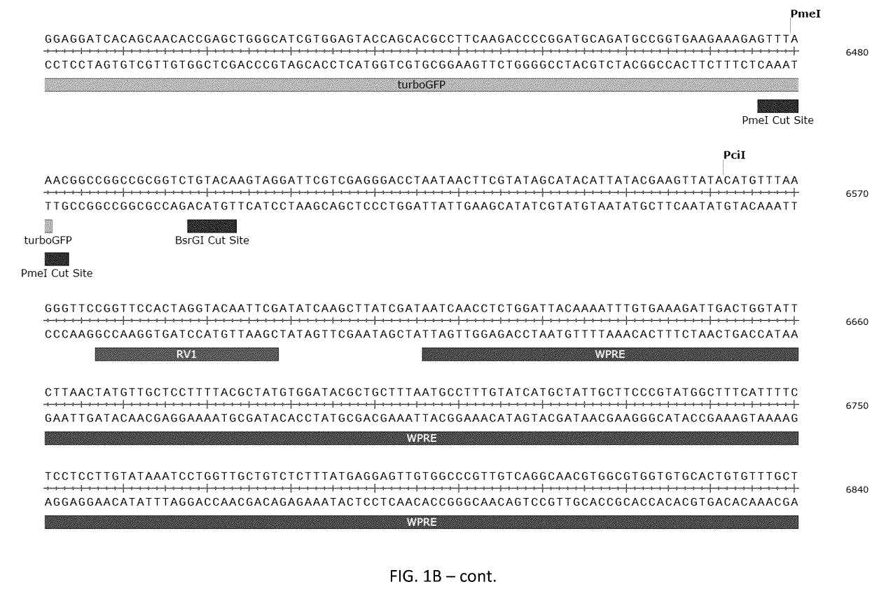

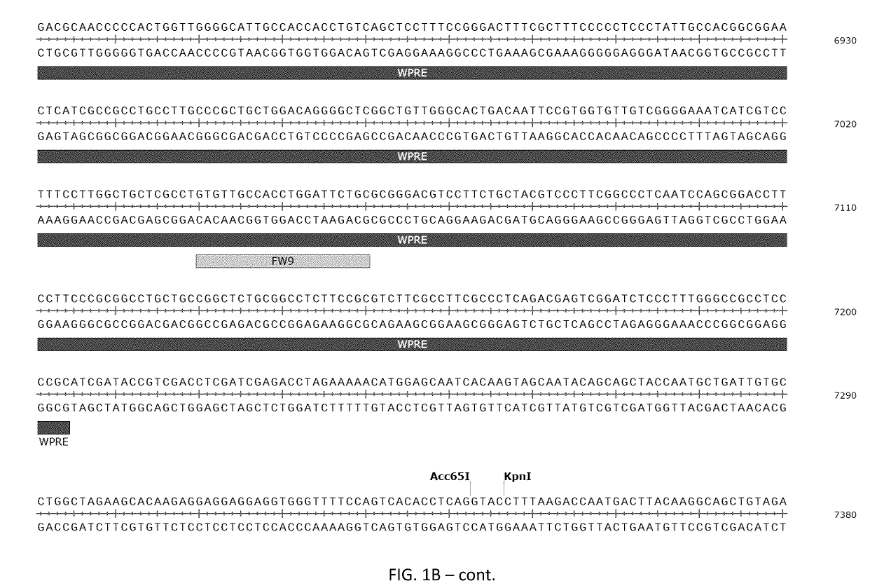

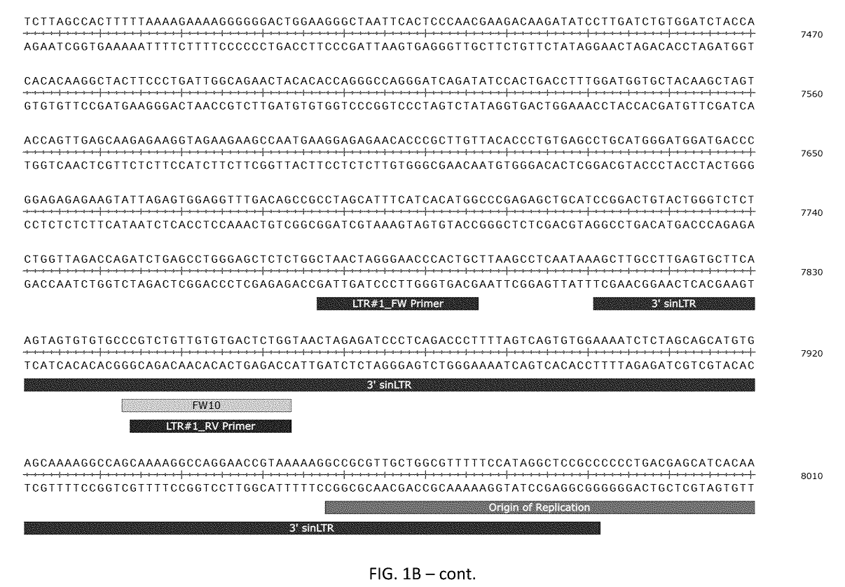

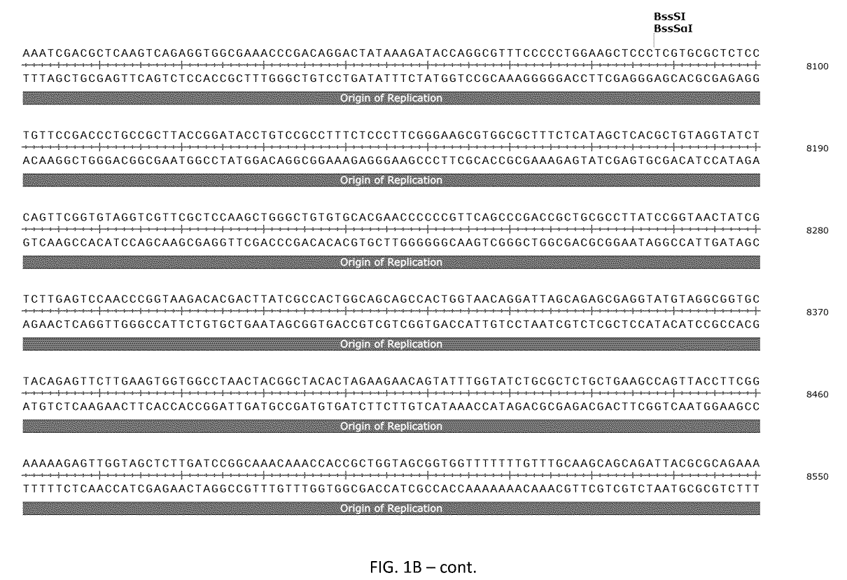

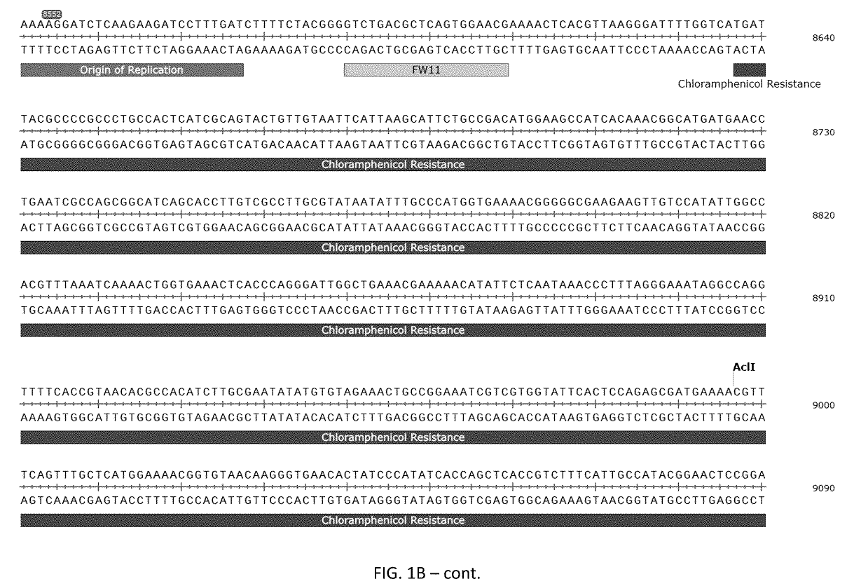

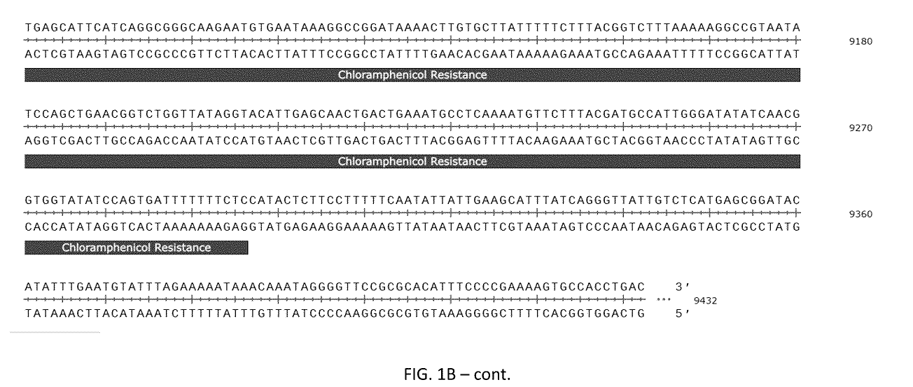

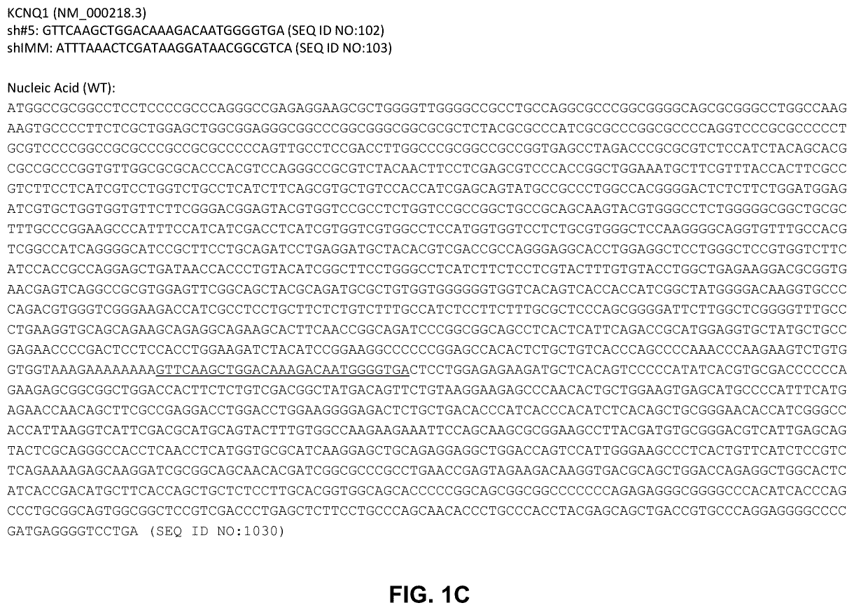

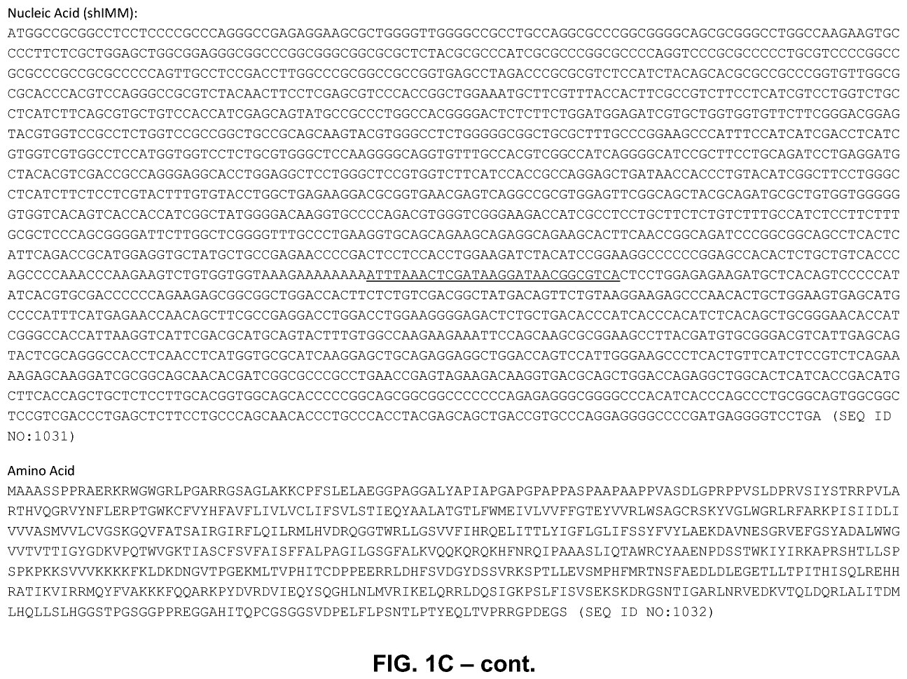

In some cases, the mammal can have LQT1 or SQTS, and the gene to be suppressed and replaced can be KCNQ1. An example of a KCNQ1 construct is shown in FIGS. 1A and 1B. An exemplary KCNQ1 sequence is set forth in NCBI RefSeq accession number NM_000218 (e.g., version NM_000218.2 or NM_00218.3) (FIG. 1C). A KCNQ1 polypeptide can, in some cases, have the amino acid sequence set forth in NCBI RefSeq accession number NP_000209 (e.g., version NP_000209.2) (FIG. 1C).

Examples of shRNA sequences and corresponding shIMM sequences targeted to KCNQ1 are set forth in TABLE 1A.

TABLE 1A
Representative KCNQ1 shRNA and
shIMM sequences

| | SEQ | | SEQ |
|---|---|---|---|
| shRNA Sequence | ID | shIMM Sequence | ID |
| GGCTGGAAATGCTTC | 54 | GGGTGGAAGTGTTTT | 55 |
| GTTTACCACT | | GTATATCATT | |
| GCTGGAAATGCTTCG | 56 | GGTGGAAGTGTTTTG | 57 |
| TTTACCACTT | | TATATCATTT | |
| GGAAATGCTTCGTTT | 58 | GGAAGTGTTTTGTAT | 59 |
| ACCACT | | ATCATT | |
| GGAAATGCTTCGTTT | 60 | GGAAGTGTTTTGTAT | 61 |
| ACCA | | ATCA | |
| GAAATGCTTCGTTTA | 62 | GAAGTGTTTTGTATA | 63 |
| CCACTT | | TCATTT | |
| TTCCTCATCGTCCTG | 64 | TTTCTGATTGTGCTC | 65 |
| GTCTGCCTCATCTT | | GTGTGTCTGATTTT | |
| GCGTGCTGTCCACCA | 66 | GTGTCCTCTCGACGA | 67 |
| TCGAGCAGTATGCC | | TTGAACAATACGCG | |
| GTCCACCATCGAGCA | 68 | CTCGACGATTGAACA | 69 |
| GTAT | | ATAC | |
| TCCACCATCGAGCAG | 70 | TCGACGATTGAACAA | 71 |
| TATGCC | | TACGCG | |
| GTGTTCTTCGGGACG | 72 | GTCTTTTTTGGCACC | 73 |
| GAGTACGTGGTCCG | | GAATATGTCGTGCG | |
| CTCATCGTGGTCGTG | 74 | CTGATTGTCGTGGTC | 75 |
| GCCTCCATGGTGGT | | GCGTCGATGGTCGT | |
| GGGCAGGTGTTTGCC | 76 | GGCCAAGTCTTCGCG | 77 |
| ACGTCGGCCATCAG | | ACCTCCGCGATTAG | |
| ACCGCCAGGGAGGCA | 78 | ATCGGCAAGGTGGGA | 79 |
| CCTGGAGGCTCCTG | | CGTGGAGACTGCTC | |
| TGGTCTTCATCCACC | 80 | TCGTGTTTATTCATC | 81 |
| GCCAGGAGCTGATA | | GGCAAGAACTCATT | |
| TGGTCTTCATCCACC | 82 | TCGTGTTTATTCATC | 83 |
| GCCAGG | | GGCAAG | |
| GCTGATAACCACCCT | 84 | ACTCATTACGACGCT | 85 |
| GTACAT | | CTATAT | |
| ACCACCCTGTACATC | 86 | ACGACGCTCTATATT | 87 |
| GGCTTCCTGGGCCT | | GGGTTTCTCGGGCT | |
| ACCACCCTGTACATC | 88 | ACGACGCTCTATATT | 89 |
| GGCTTC | | GGGTTT | |
| CTGGCTGAGAAGGAC | 90 | CTCGCAGAAAAAGAT | 91 |
| GCGGTGAACGAGTC | | GCCGTCAATGAATC | |
| CTGTGGTGGGGGGTG | 92 | CTCTGGTGGGGCGTC | 93 |
| GTCACAGTCACCAC | | GTGACTGTGACGAC | |
| AGACCATCGCCTCCT | 94 | AAACGATTGCGTCGT | 95 |
| GCTTCTCTGTCTTT | | GTTTTTCAGTGTTC | |
| AGCAGAAGCAGAGGC | 96 | AACAAAAACAAAGAC | 97 |
| AGAAGCACTTCAAC | | AAAAACATTTTAAT | |
| GAAGCAGAGGCAGAA | 98 | AAAACAAAGACAAAA | 99 |
| GCACTT | | ACATTT | |
| CCCAAACCCAAGAAG | 100 | CCGAAGCCGAAAAAA | 101 |
| TCTGTGGTGGTAAA | | TCAGTCGTCGTTAA | |
| GTTCAAGCTGGACAA | 102 | ATTTAAACTCGATAA | 103 |
| AGACAATGGGGTGA | | GGATAACGGCGTCA | |
| GTTCAAGCTGGACAA | 104 | ATTTAAACTCGATAA | 105 |
| AGACAA | | GGATAA | |
| TGGACAAAGACAATG | 106 | TCGATAAGGATAACG | 107 |
| GGGTGA | | GCGTCA | |
| GAGAGAAGATGCTCA | 108 | GTGAAAAAATGCTGA | 109 |
| CAGT | | CTGT | |
| GACAGTTCTGTAAGG | 110 | GATAGCTCAGTTAGA | 111 |
| AAGAGCCCAACACT | | AAAAGTCCTACTCT | |
| GTTCTGTAAGGAAGA | 112 | GCTCAGTTAGAAAAA | 113 |
| GCCCAACACT | | GTCCTACTCT | |
| GCCCAACACTGCTGG | 114 | GTCCTACTCTCCTCG | 115 |
| AAGTGAGCATGCCC | | AGGTCAGTATGCCG | |
| GCCCAACACTGCTGG | 116 | GTCCTACTCTCCTCG | 117 |
| AAGTGA | | AGGTCA | |
| TGAGAACCAACAGCT | 118 | TGAGGACGAATAGTT | 119 |
| TCGCCGAGGACCTG | | TTGCGGAAGATCTC | |
| GGGCCACCATTAAGG | 120 | GCGCGACGATAAAAG | 121 |
| TCAT | | TGAT | |
| GGCCACCATTAAGGT | 122 | CGCGACGATAAAAGT | 123 |
| CATT | | GATA | |
| CGCATGCAGTACTTT | 124 | CGGATGCAATATTTC | 125 |
| GTGGCCAAGAAGAA | | GTCGCGAAAAAAAA | |
| AAGAAATTCCAGCAA | 126 | AAAAAGTTTCAACAG | 127 |
| GCGCGGAAGCCTTA | | GCCCGCAAACCATA | |
| AGGGCCACCTCAACC | 128 | AAGGGCATCTGAATC | 129 |
| TCATGGTGCGCATC | | TGATGGTCCGGATT | |
| GTCCATTGGGAAGCC | 130 | ATCGATAGGCAAACC | 131 |
| CTCACTGTTCATCT | | GTCTCTCTTTATTT | |
| GGAAGCCCTCACTGT | 132 | GCAAACCGTCTCTCT | 133 |
| TCATCT | | TTATTT | |
| GCCTGAACCGAGTAG | 134 | GGCTCAATCGTGTTG | 135 |
| AAGA | | AGGA | |
| GAAGACAAGGTGACG | 136 | GAGGATAAAGTCACC | 137 |
| CAGCTGGACCAGAG | | CAACTCGATCAAAG | |

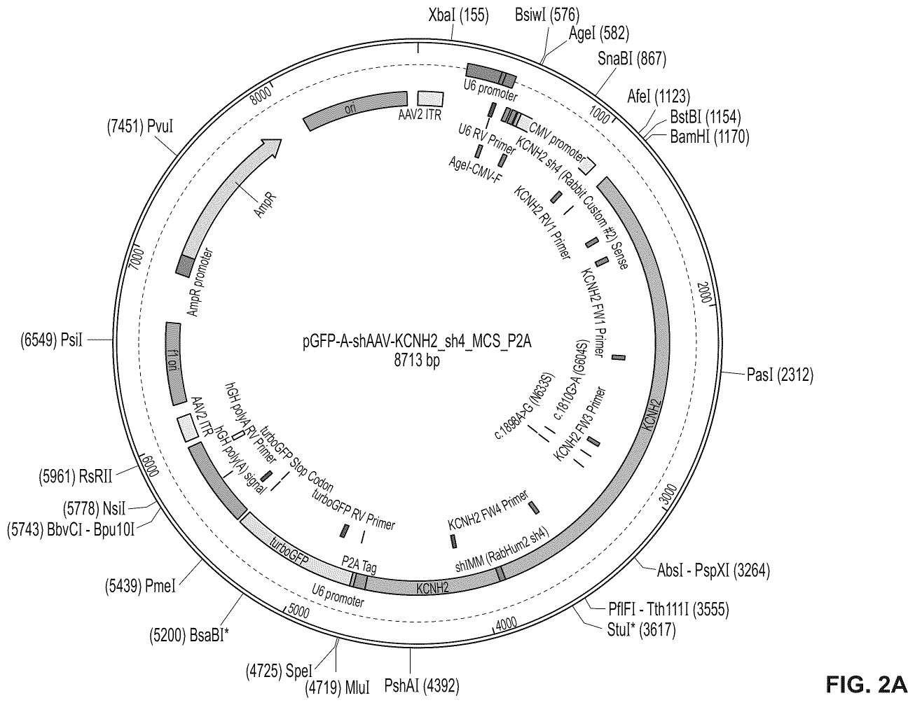

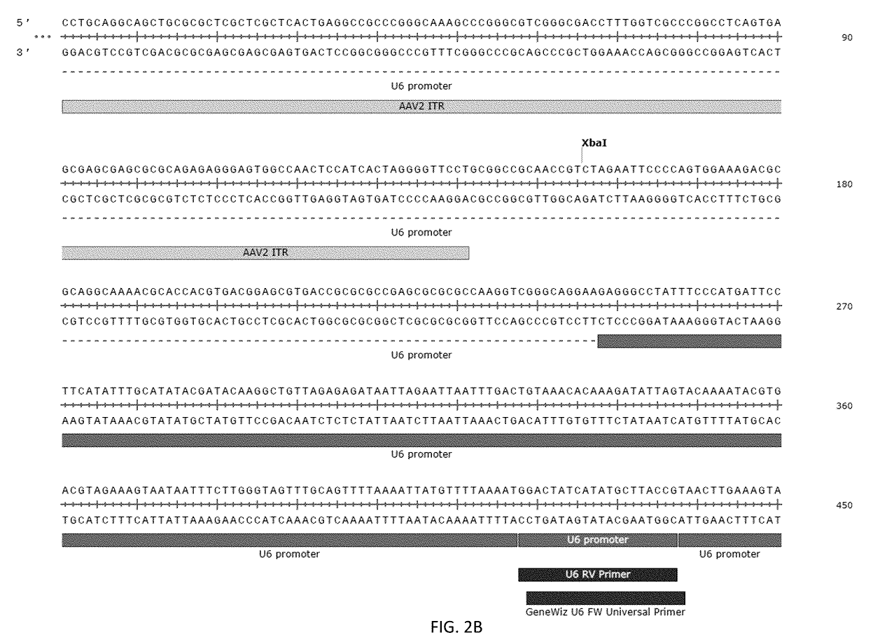

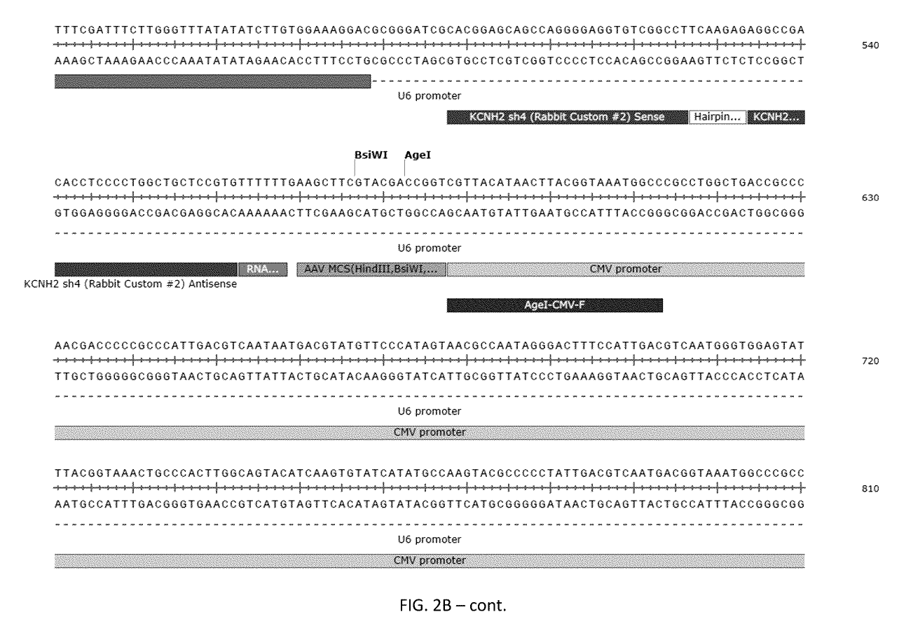

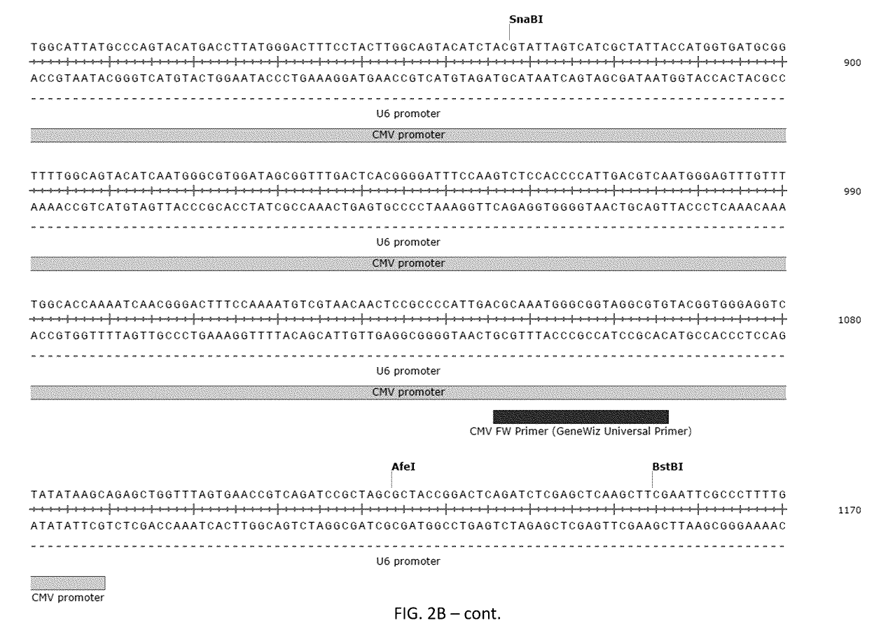

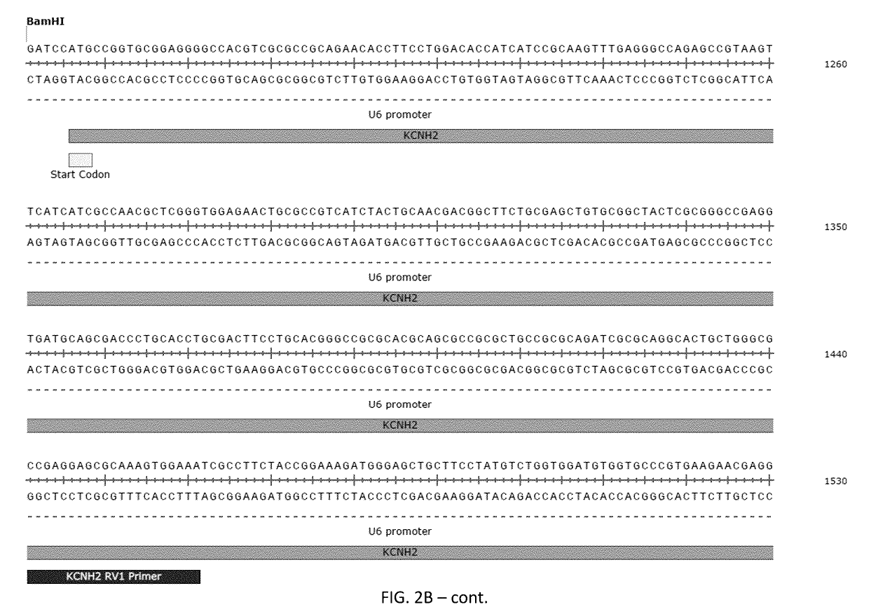

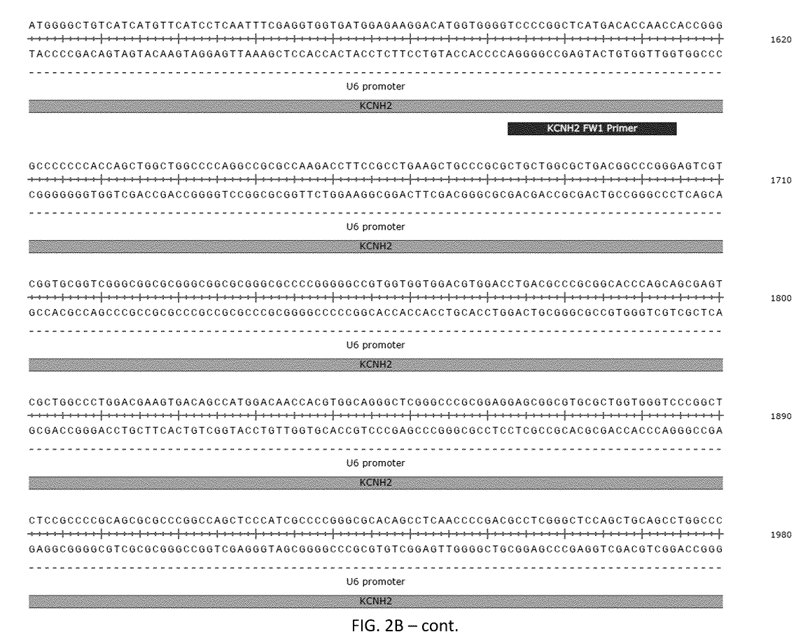

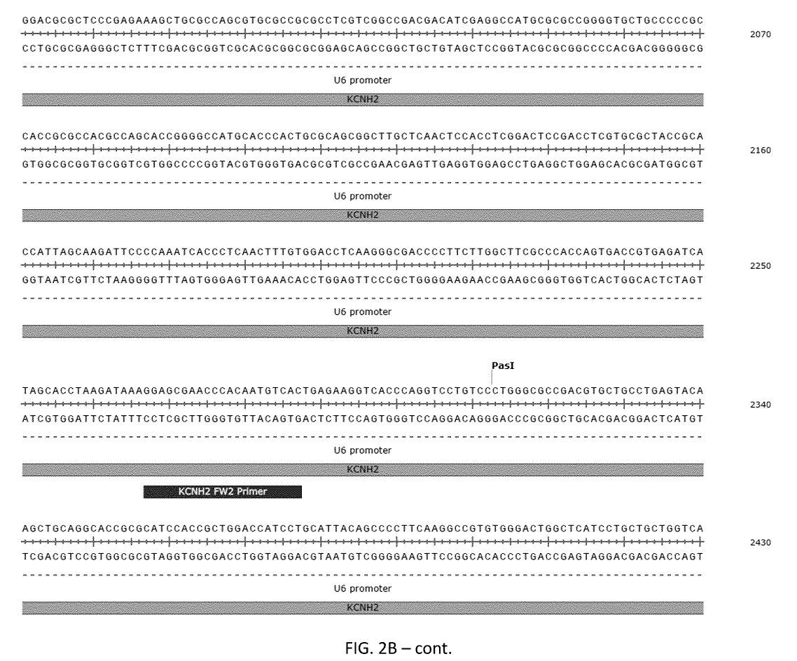

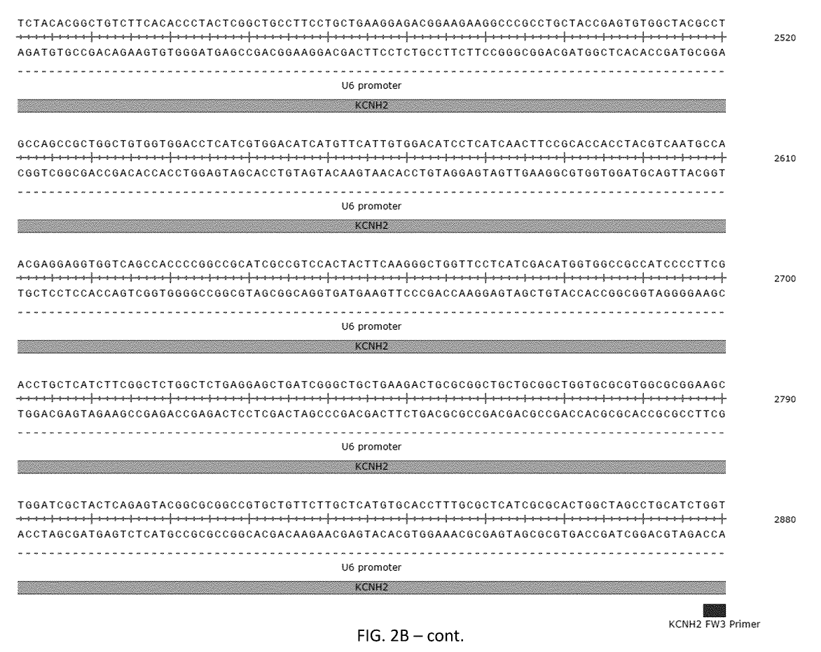

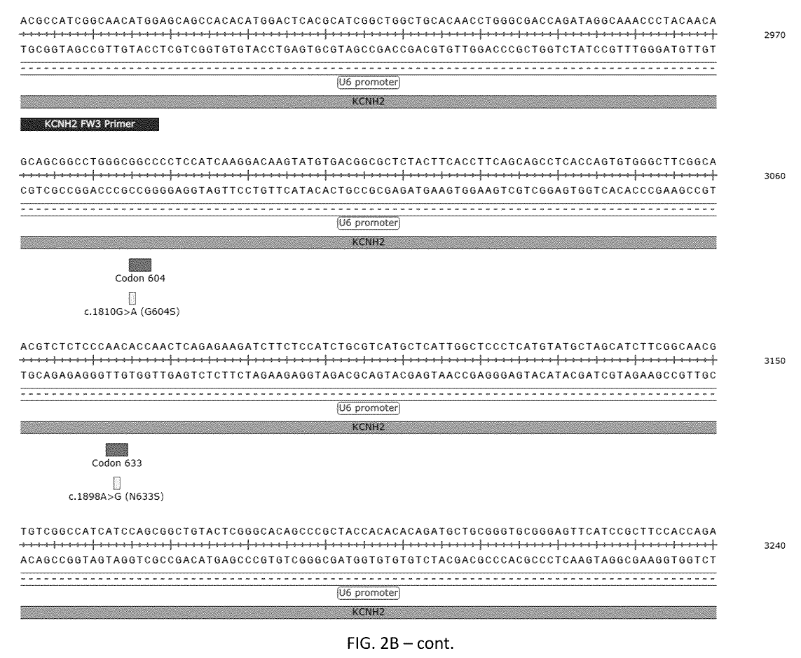

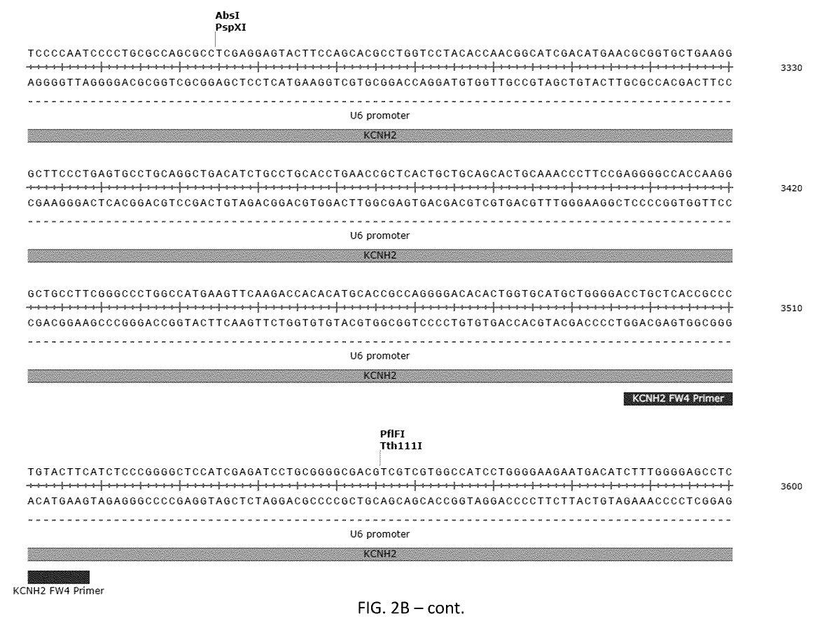

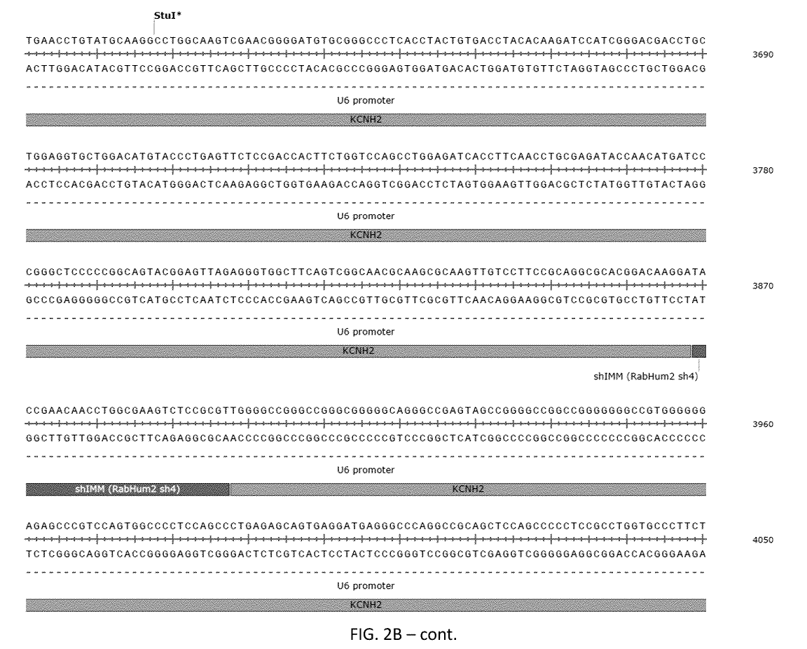

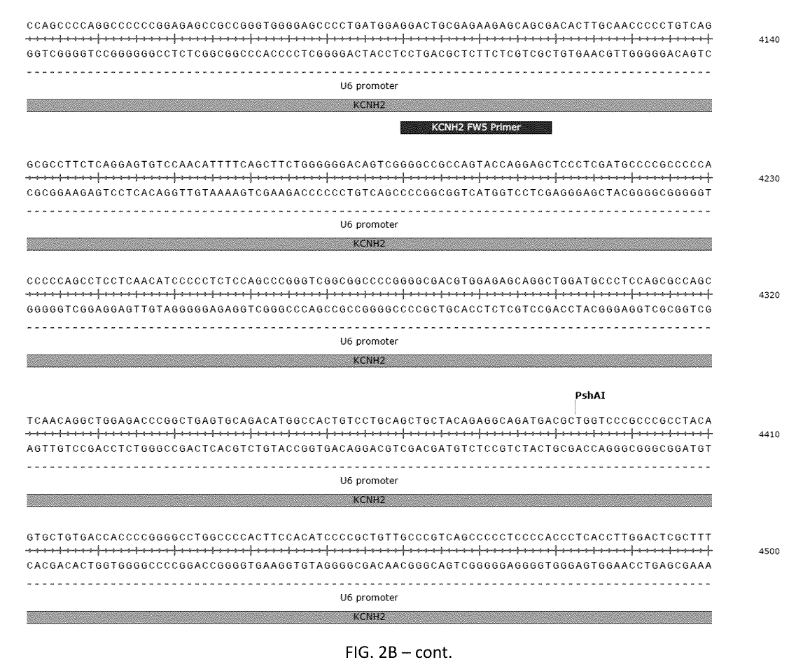

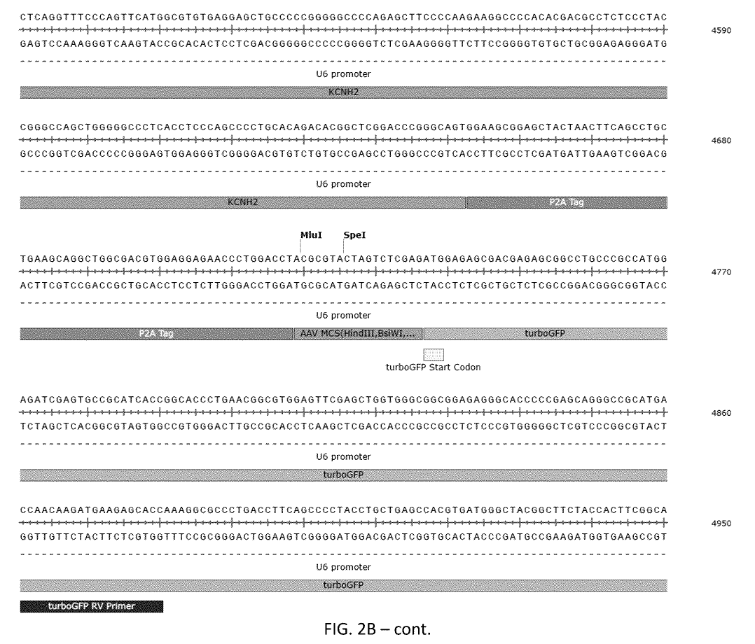

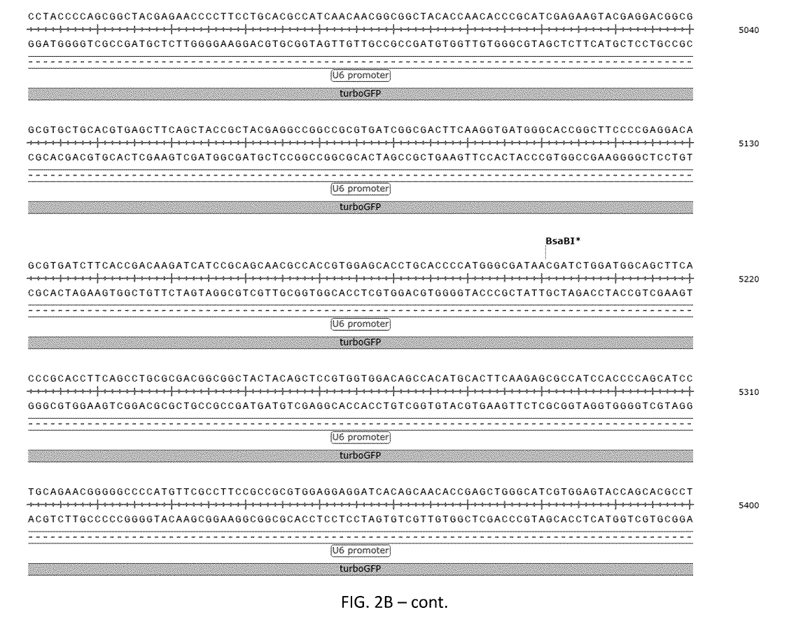

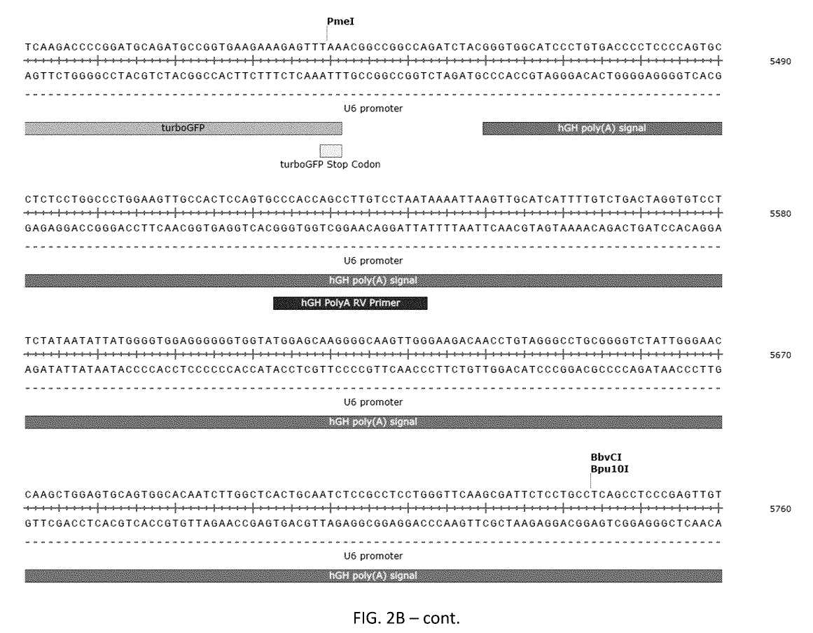

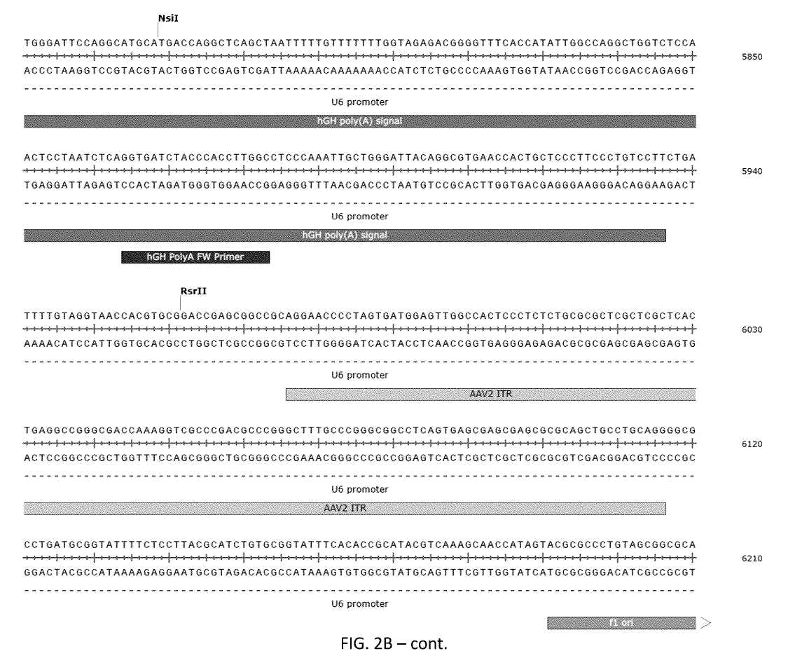

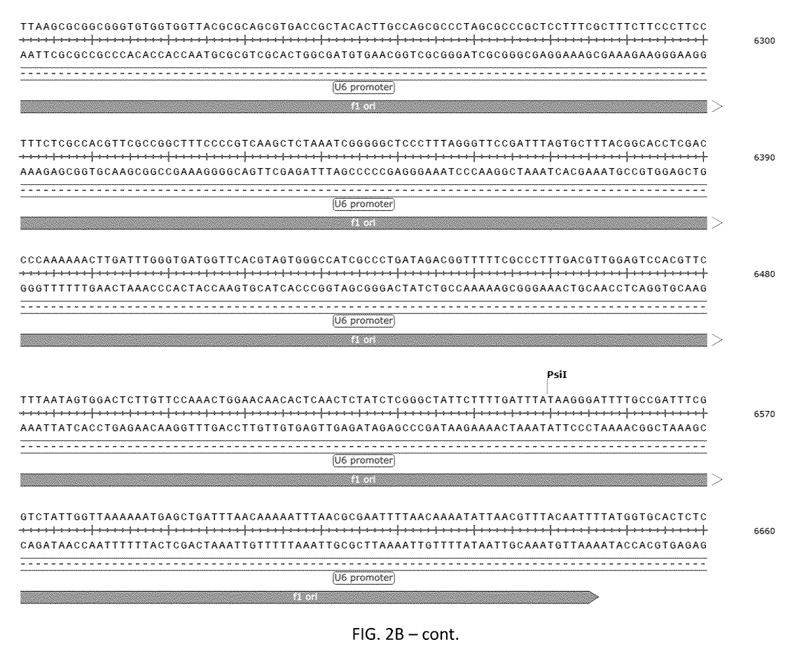

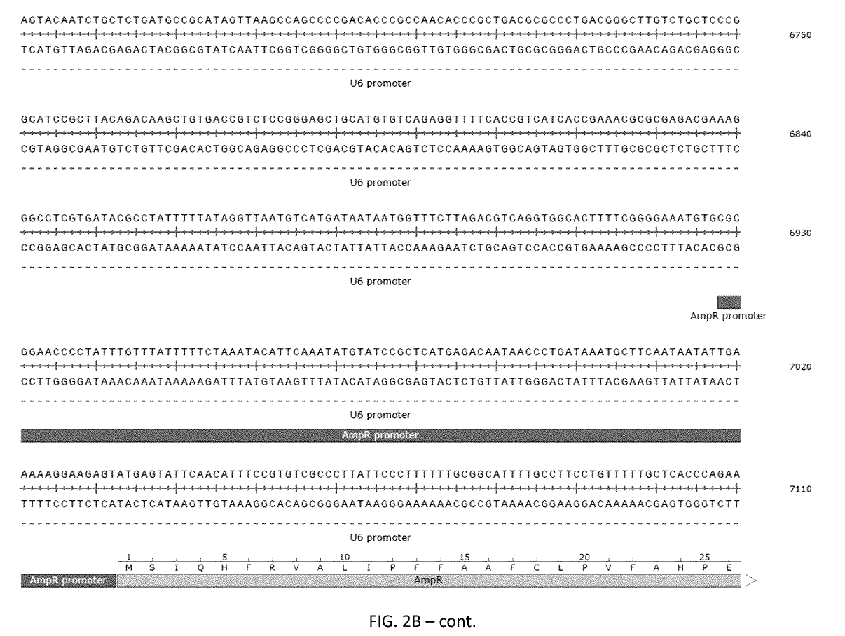

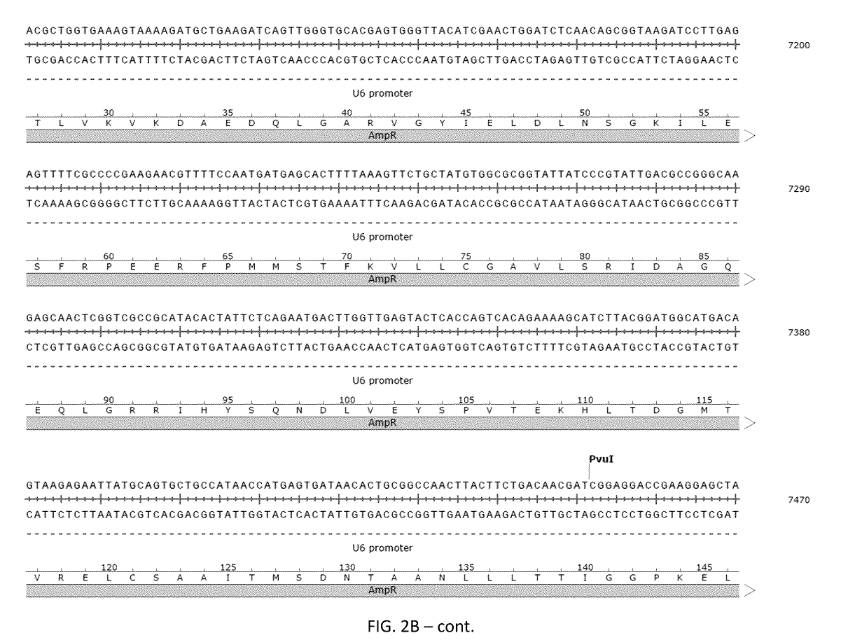

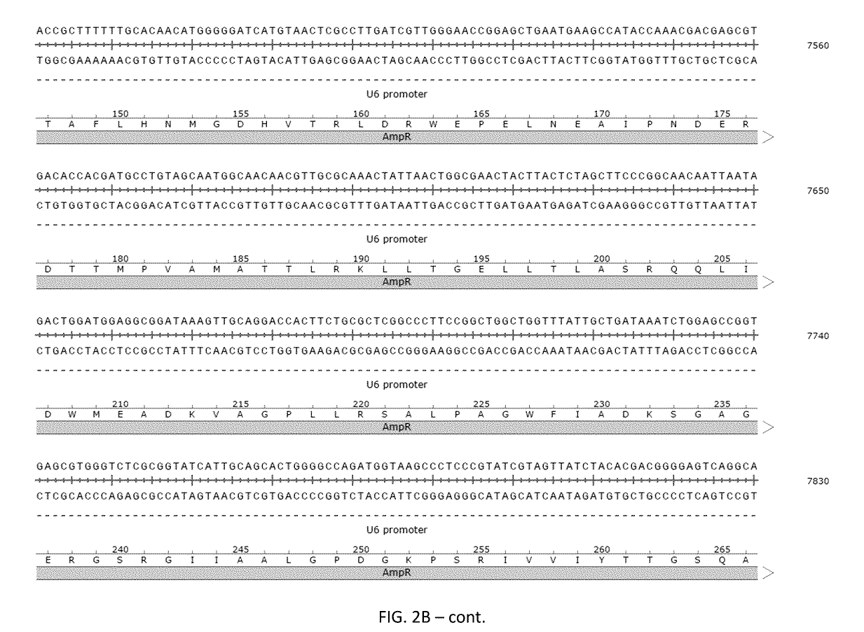

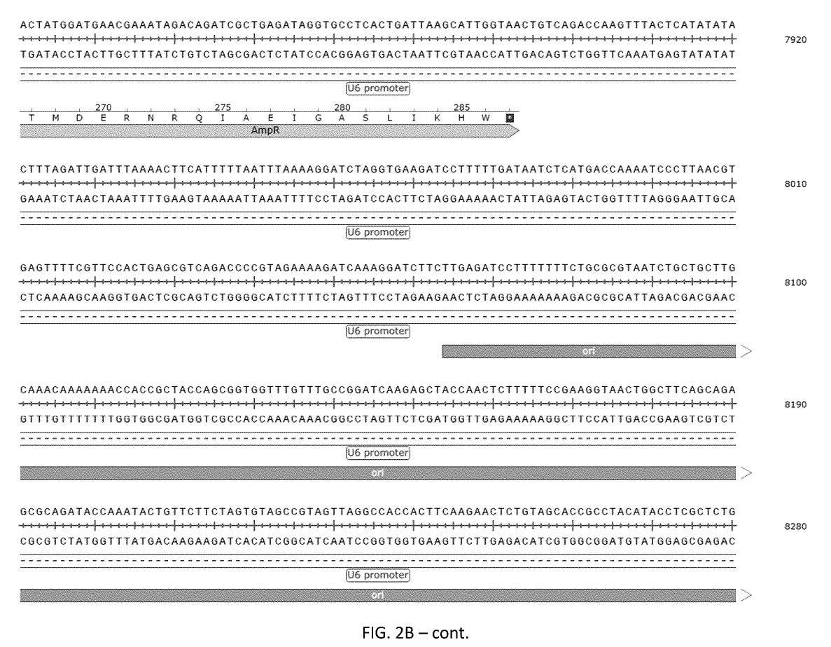

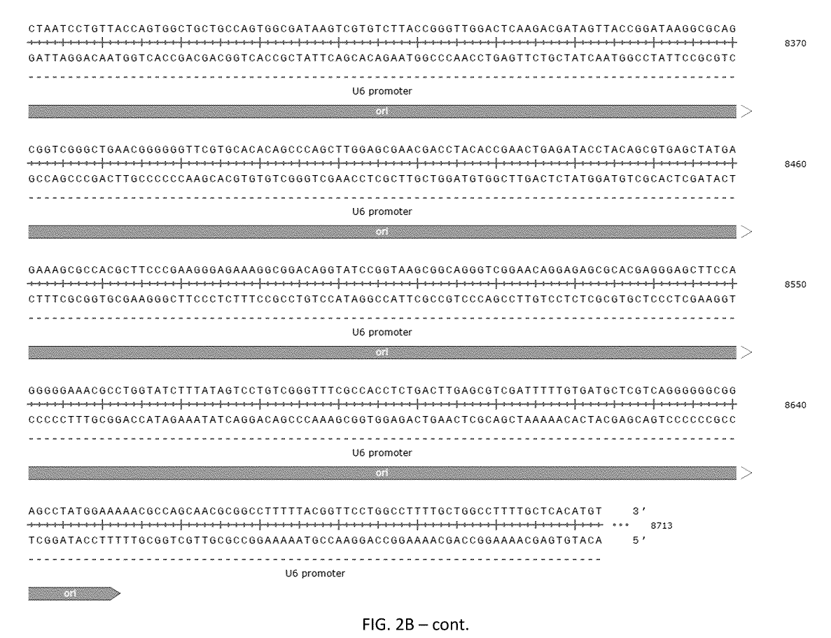

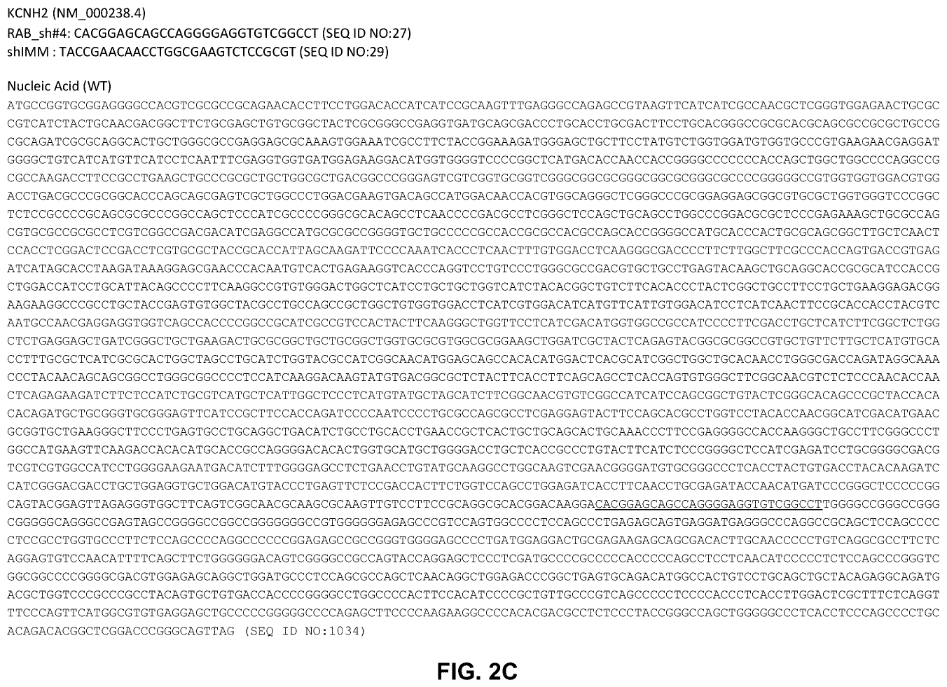

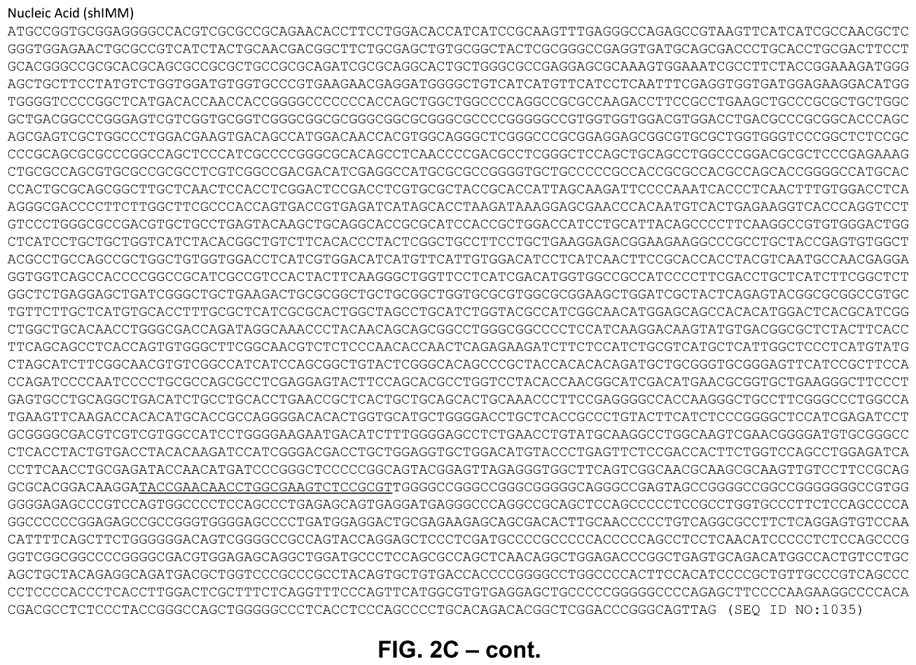

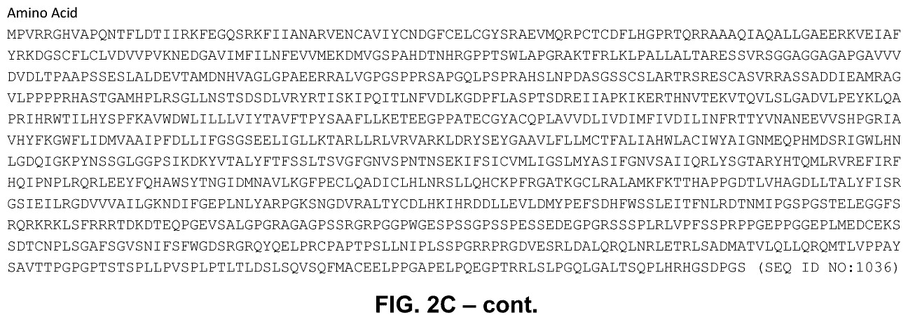

In some cases, the mammal can have LQT2 or SQTS, and the gene to be suppressed and replaced can be KCNH2. An example of a KCNH2 construct is shown in FIGS. 2A and 2B. An exemplary KCNH2 sequence is set forth in NCBI RefSeq accession number NM_000238 (e.g., version NM_000238.4; FIG. 2C). A KCNH2 polypeptide can, in some cases, have the amino acid sequence set forth in NCBI RefSeq accession number NP_000229 (e.g., version NP_000229.1; FIG. 2C).

Examples of shRNA sequences and corresponding shIMM sequences targeted to KCNH2 are set forth in TABLE 1B.

TABLE 1B
Representative KCNH2 shRNA
and shIMM sequences

| | SEQ | | SEQ |
|---|---|---|---|
| shRNA Sequence | ID | shIMM Sequence | ID |
| CACCTTCCTGGACAC | 138 | TACGTTTCTCGATAC | 139 |
| CATCATCCGCAAGT | | GATTATTCGGAAAT | |
| CACCTTCCTGGACAC | 140 | TACGTTTCTCGATAC | 141 |
| CATCAT | | GATTAT | |
| TGGACACCATCATCC | 142 | TCGATACGATTATTC | 143 |
| GCAAGT | | GGAAAT | |
| TGGGCGCCGAGGAGC | 144 | TCGGGGCGGAAGAAC | 145 |
| GCAAAGTGGAAATC | | GGAAGGTCGAGATT | |
| GATGGGAGCTGCTTC | 146 | GACGGCAGTTGTTTT | 147 |
| CTATGT | | CTTTGC | |
| GGAGCTGCTTCCTAT | 148 | GCAGTTGTTTTCTTT | 149 |
| GTCT | | GCCT | |
| GGGCTGTCATCATGT | 150 | GCGCAGTGATTATGT | 151 |
| TCAT | | TTAT | |
| GCTGTCATCATGTTC | 152 | GCAGTGATTATGTTT | 153 |
| ATCCTCAATT | | ATTCTGAACT | |
| TCGTGCGCTACCGCA | 154 | TGGTCCGGTATCGGA | 155 |
| CCATTAGCAAGATT | | CGATAAGTAAAATA | |
| ATCACCCTCAACTTT | 156 | ATTACGCTGAATTTC | 157 |
| GTGGACCTCAAGGG | | GTCGATCTGAAAGG | |
| GTGACCGTGAGATCA | 158 | GCGATCGAGAAATTA | 159 |
| TAGCACCTAAGATA | | TTGCTCCAAAAATT | |
| GATCATAGCACCTAA | 160 | AATTATTGCTCCAAA | 161 |
| GATAAA | | AATTAA | |
| GATCATAGCACCTAA | 162 | AATTATTGCTCCAAA | 163 |
| GATA | | AATT | |
| GAGCGAACCCACAAT | 164 | GAACGTACGCATAAC | 165 |
| GTCA | | GTGA | |
| GTGGGACTGGCTCAT | 166 | CTGGGATTGGCTGAT | 167 |
| CCTGCTGCTGGTCA | | TCTCCTCCTCGTGA | |
| GGTCATCTACACGGC | 168 | CGTGATTTATACCGC | 169 |
| TGTCTT | | AGTGTT | |
| GTGGACATCCTCATC | 170 | GTCGATATTCTGATT | 171 |
| AACT | | AATT | |
| GACATCCTCATCAAC | 172 | GATATTCTGATTAAT | 173 |
| TTCCGCACCACCTA | | TTTCGGACGACGTA | |
| GAAGCTGGATCGCTA | 174 | CAAACTCGACCGGTA | 175 |
| CTCAGA | | TTCTGA | |
| GAAGCTGGATCGCTA | 176 | CAAACTCGACCGGTA | 177 |
| CTCA | | TTCT | |
| GCCCCTCCATCAAGG | 178 | GGCCGTCGATTAAAG | 179 |
| ACAAGTATGT | | ATAAATACGT | |
| CTGACATCTGCCTGC | 180 | CAGATATTTGTCTCC | 181 |
| ACCTGAACCGCTCA | | ATCTCAATCGGTCT | |
| CTGACATCTGCCTGC | 182 | CAGATATTTGTCTCC | 183 |
| ACCTGAACCGCTCA | | ATCTCAATCGGTCT | |
| TGAAGTTCAAGACCA | 184 | TGAAATTTAAAACGA | 185 |
| CACATGCACCGCCA | | CTCACGCTCCCCCT | |
| CTTCTGGTCCAGCCT | 186 | TTTTTGGTCGAGTCT | 187 |
| GGAGATCACCTTCA | | CGAAATTACGTTTA | |
| CACGGAGCAGCCAGG | 27 | TACCGAACAACCTGG | 29 |
| GGAGGTGTCGGCCT | | CGAAGTCTCCGCGT | |
| CACGGAGCAGCCAGG | 188 | TACCGAACAACCTGG | 189 |
| GGAGGT | | CGAAGT | |
| AGCCAGGGGAGGTGT | 190 | AACCTGGCGAAGTCT | 191 |
| CGGCCT | | CCGCGT | |
| CTGCAGCTGCTACAG | 192 | CTCCAACTCCTTCAA | 193 |
| AGGCAGATGACGCT | | AGACAAATGACCCT | |
| CGACGCCTCTCCCTA | 194 | CGTCGGCTGTCGCTT | 195 |
| CCGGGCCAGCTGGG | | CCCGGGCAACTCGG | |
| CGACGCCTCTCCCTA | 196 | CGTCGGCTGTCGCTT | 197 |
| CCGGGCCAGCTGGG | | CCCGGGCAACTCGG | |

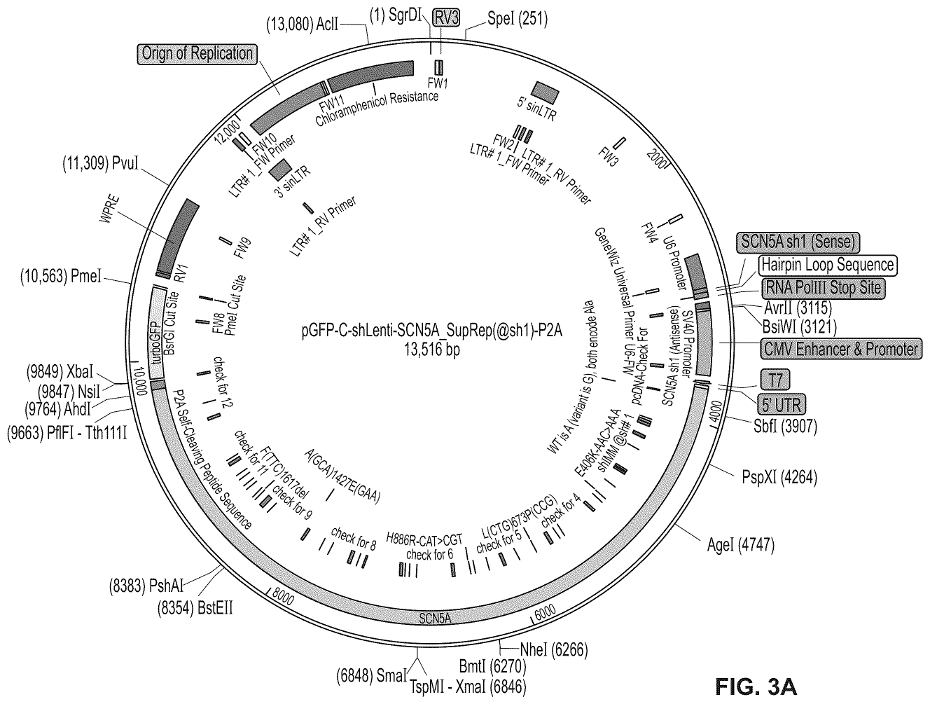

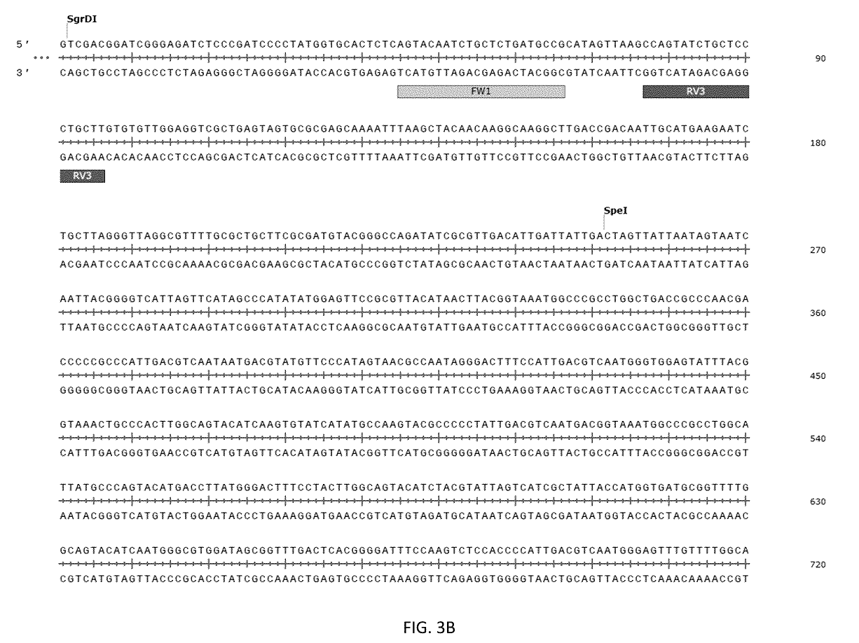

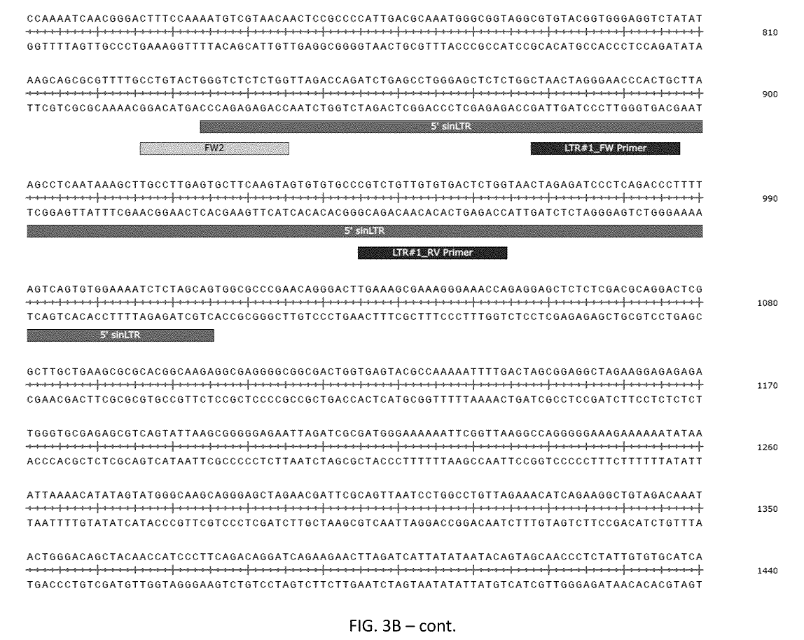

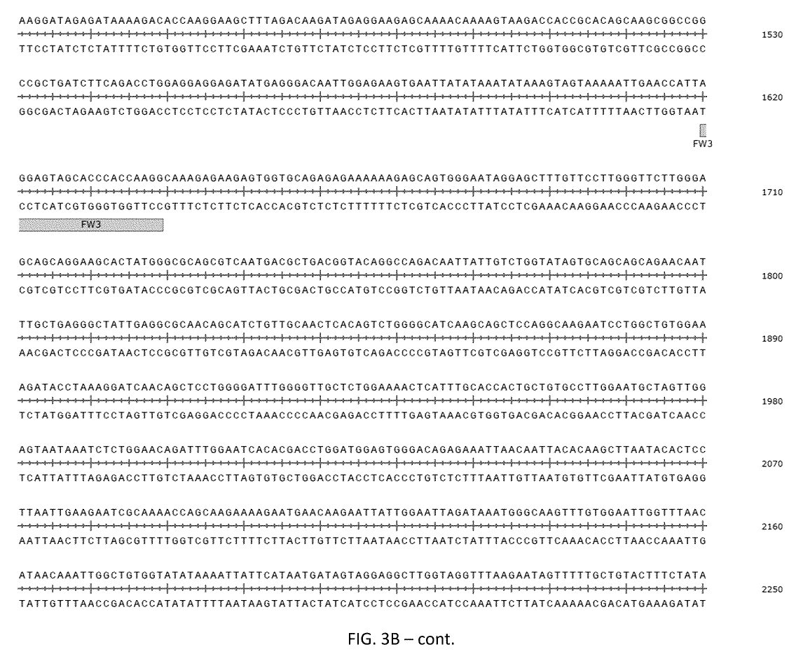

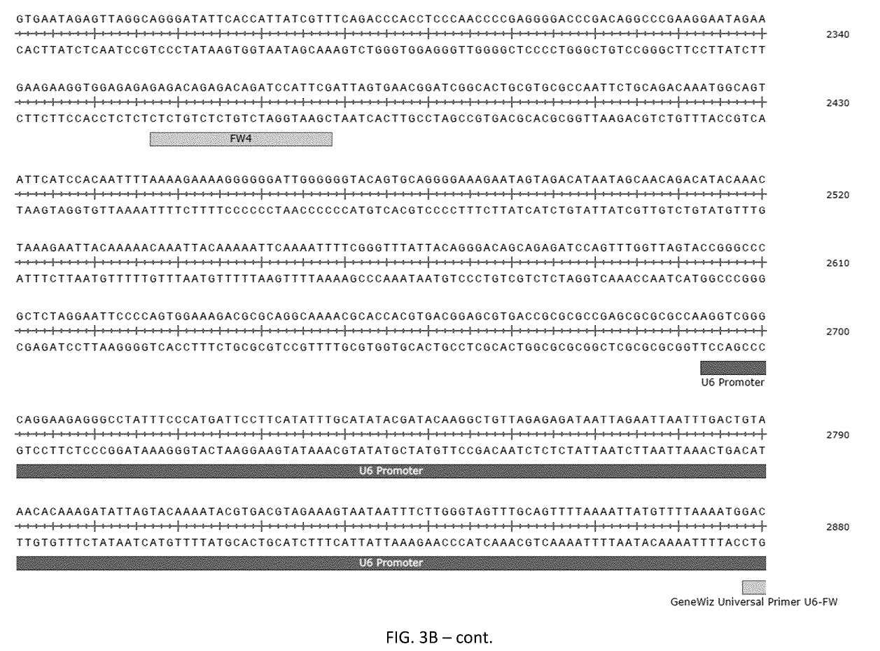

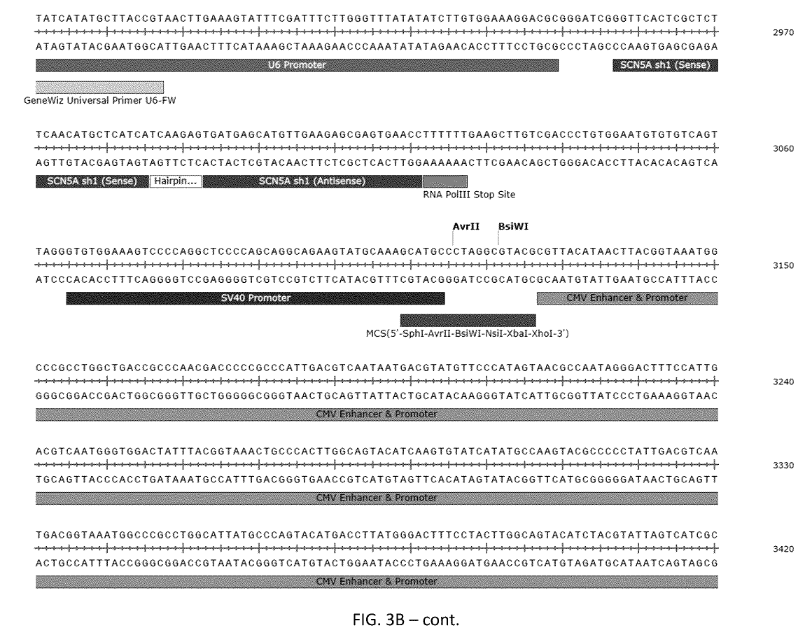

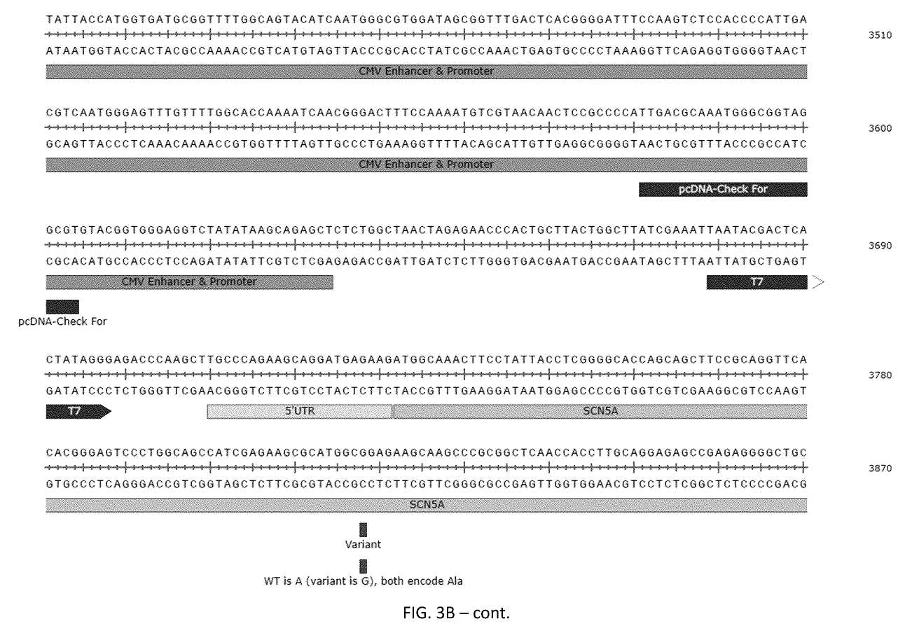

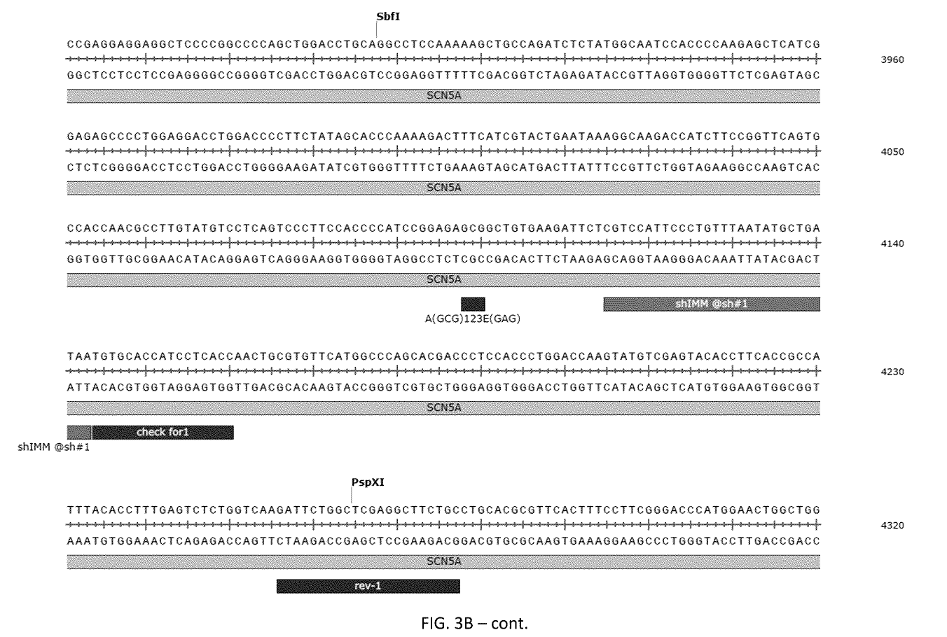

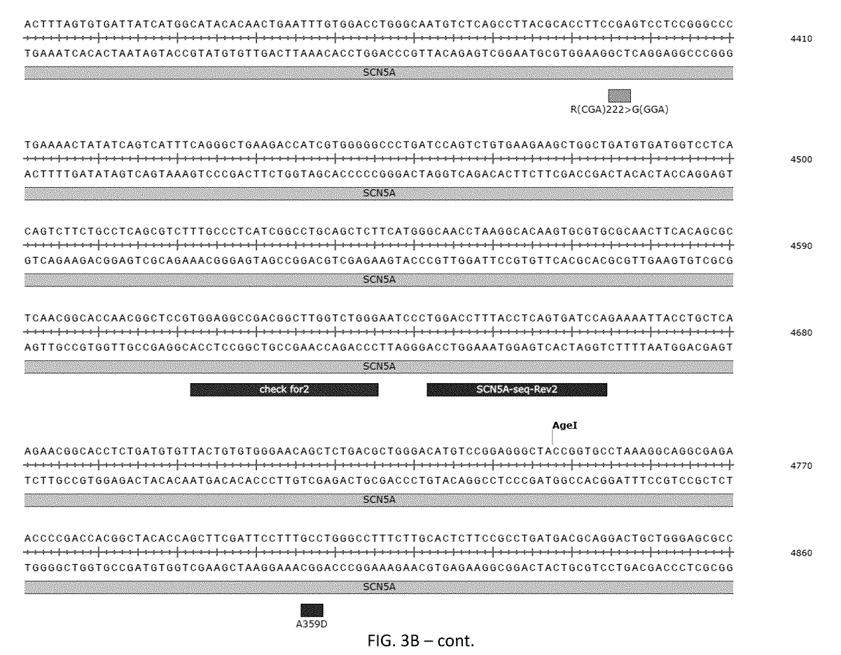

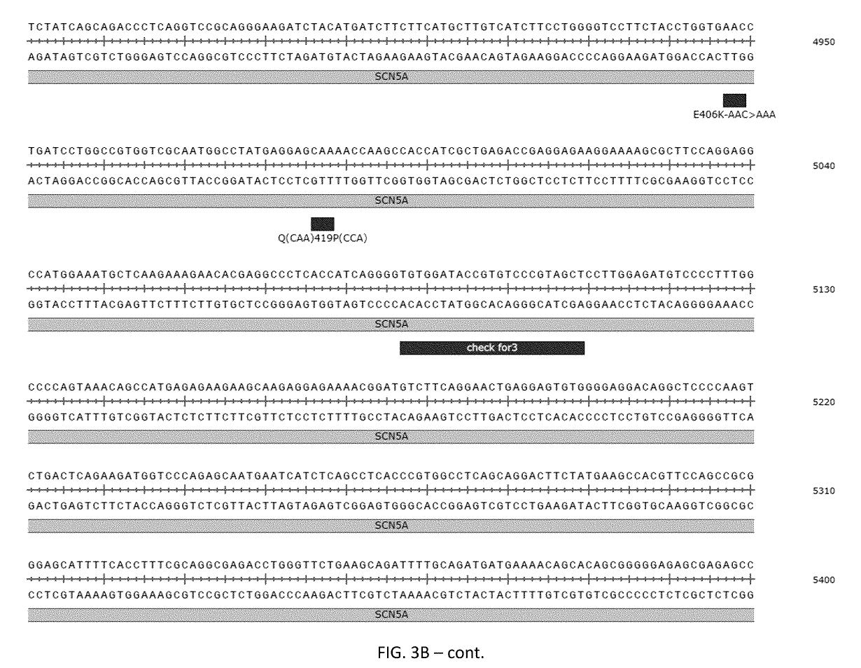

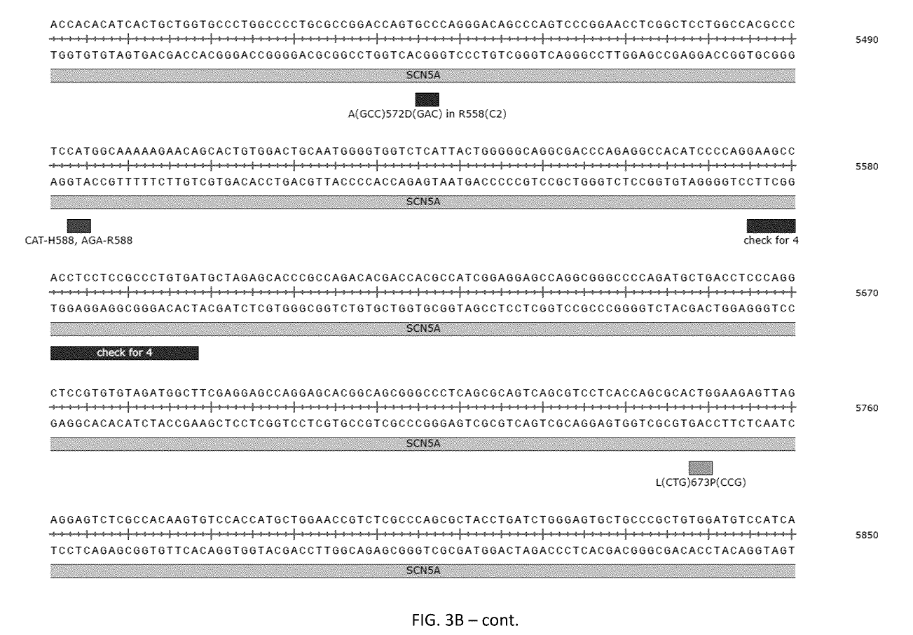

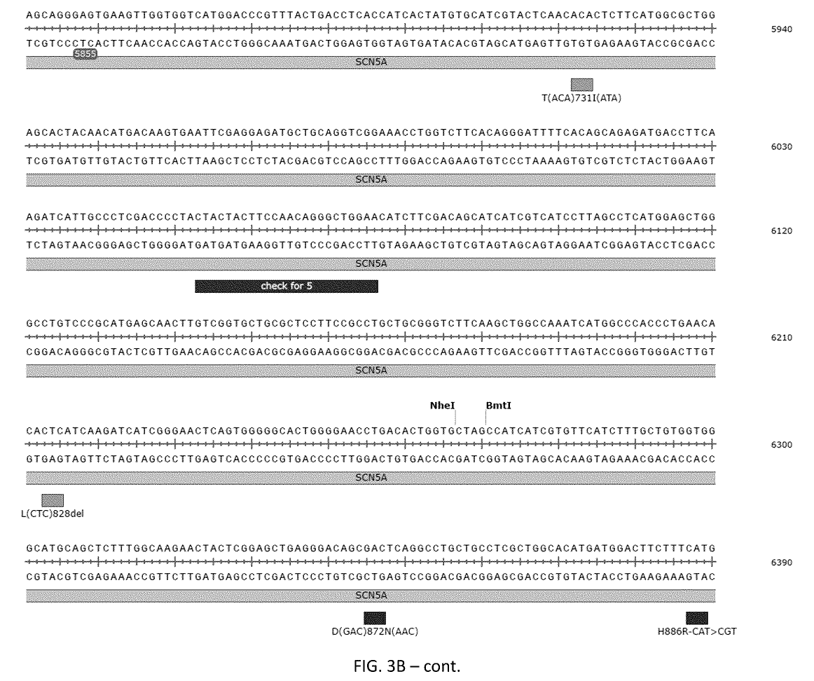

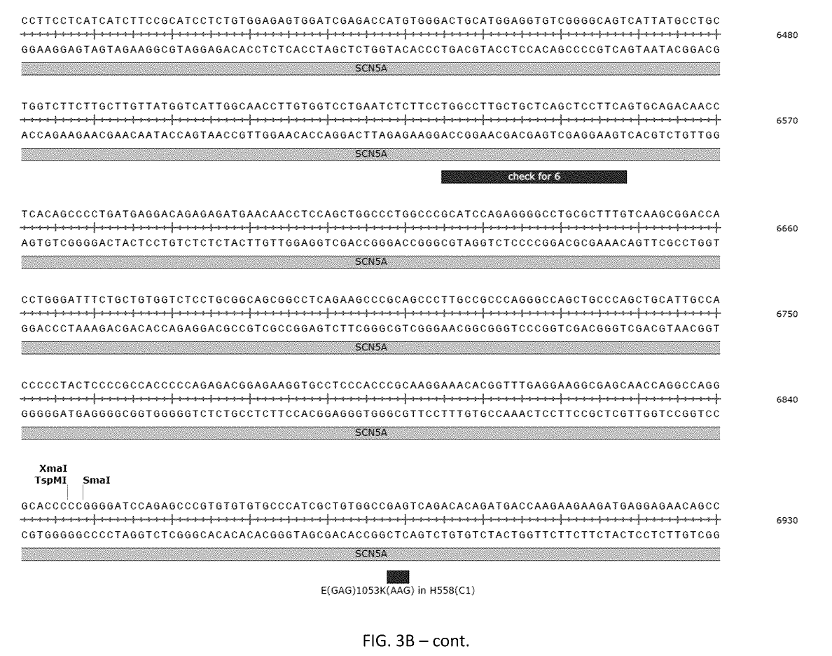

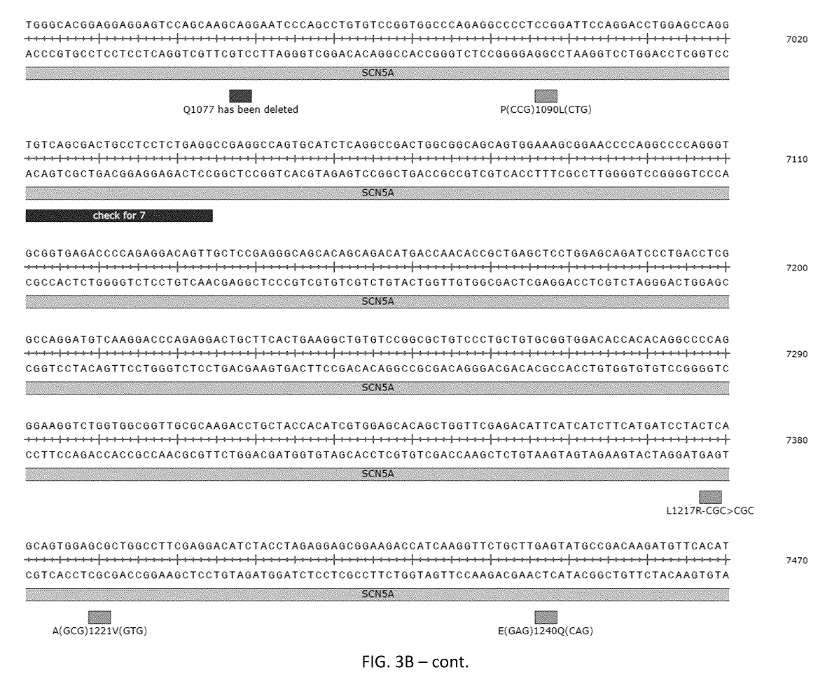

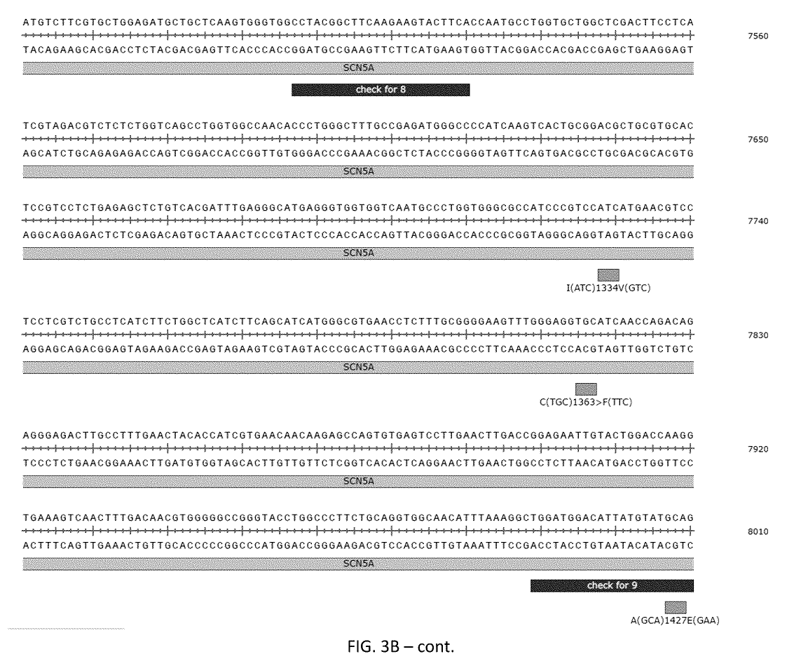

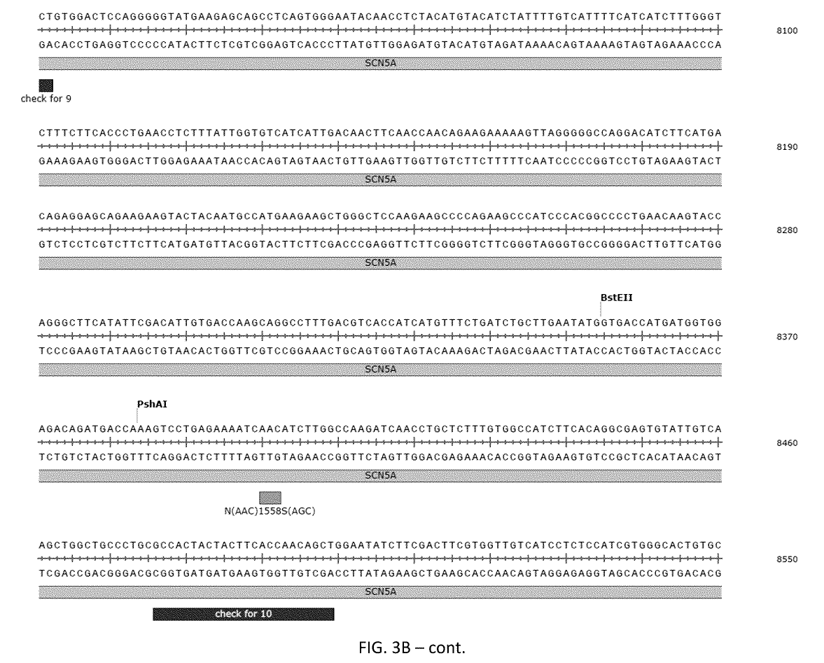

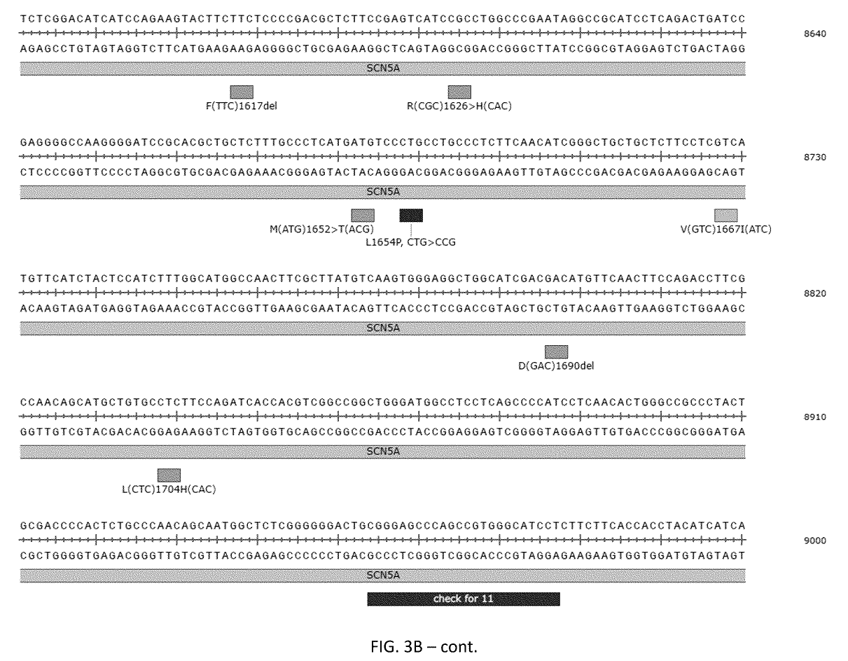

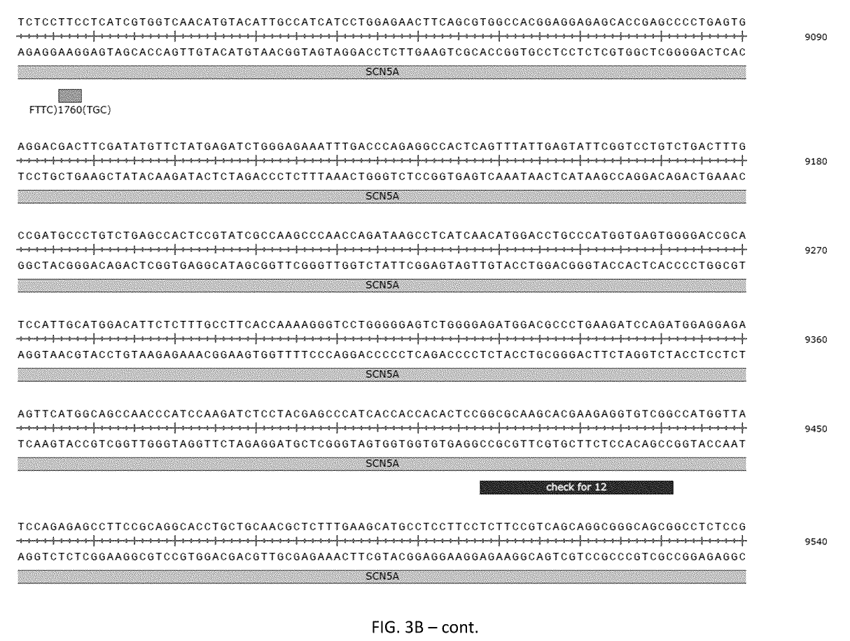

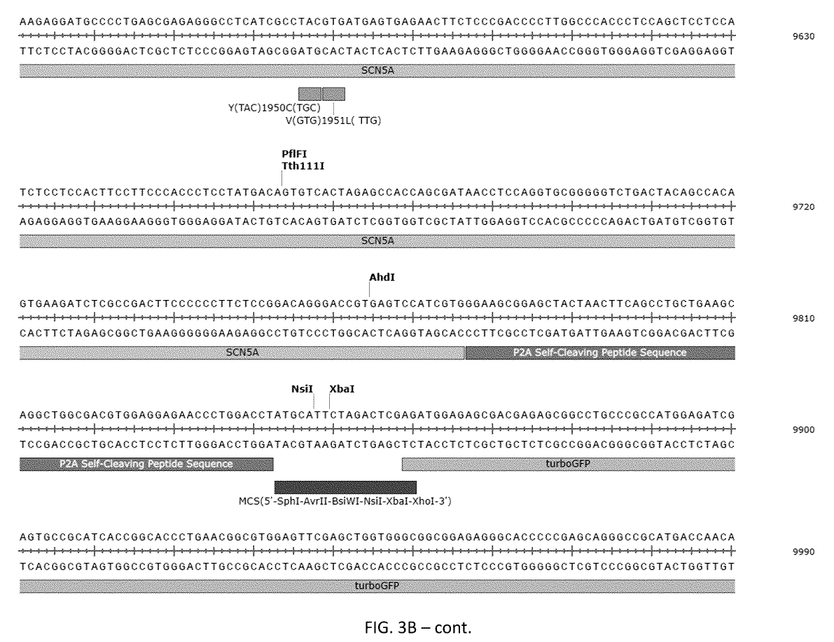

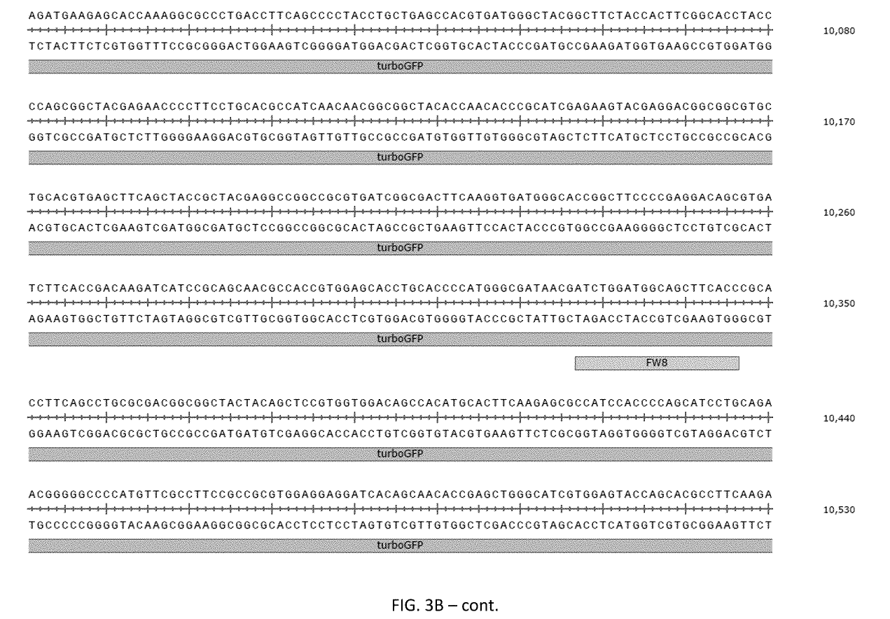

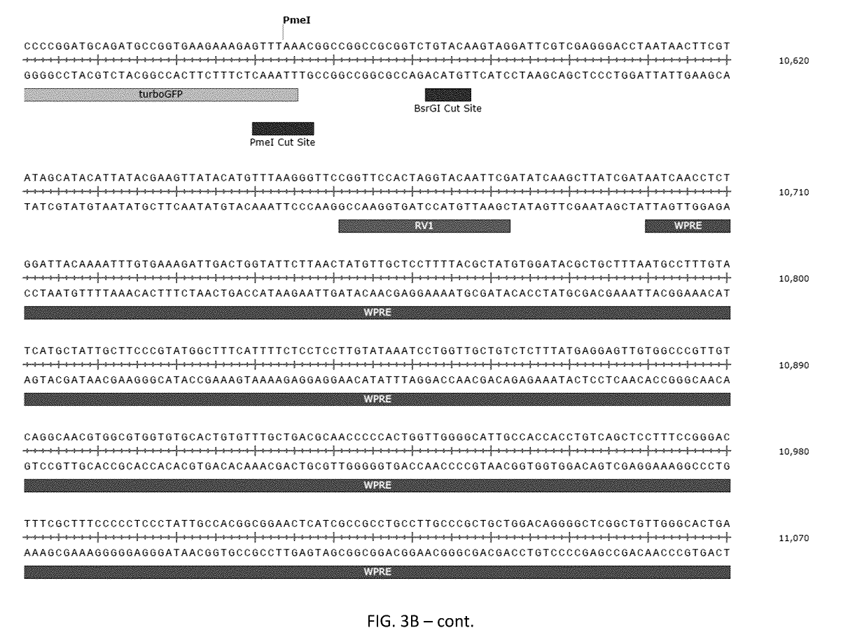

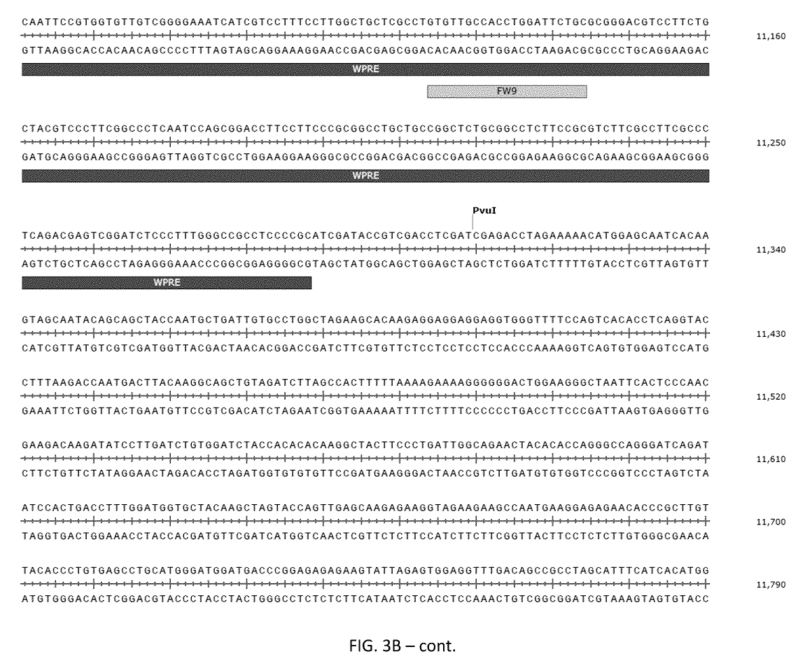

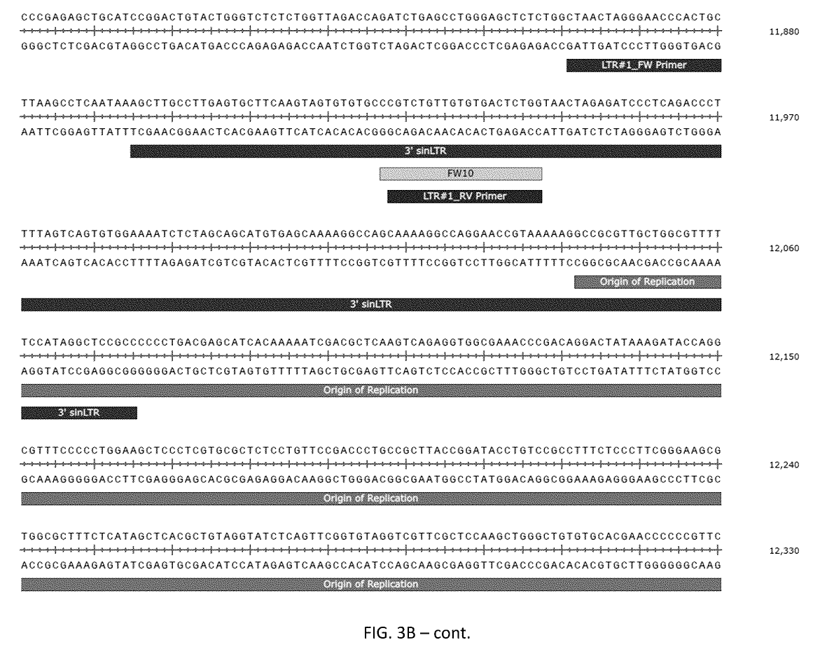

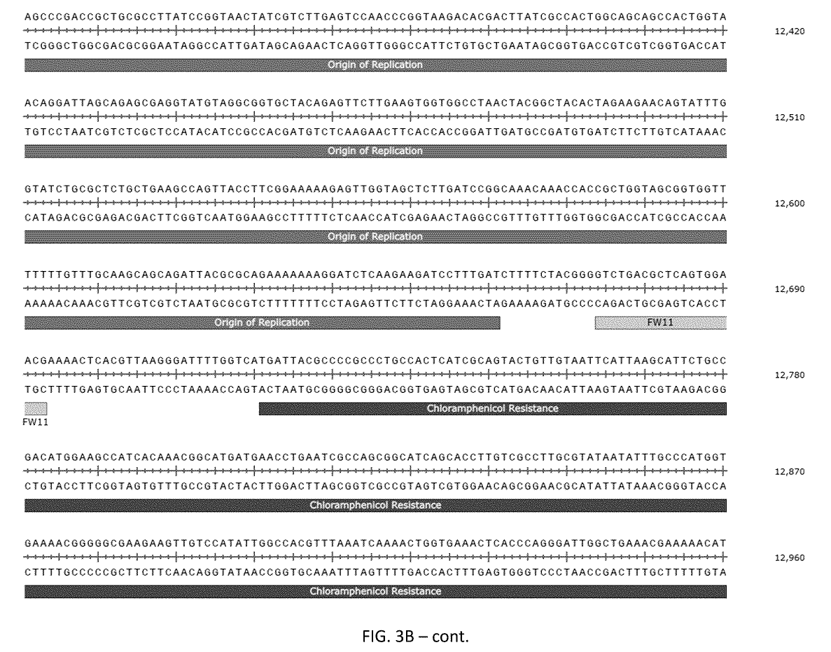

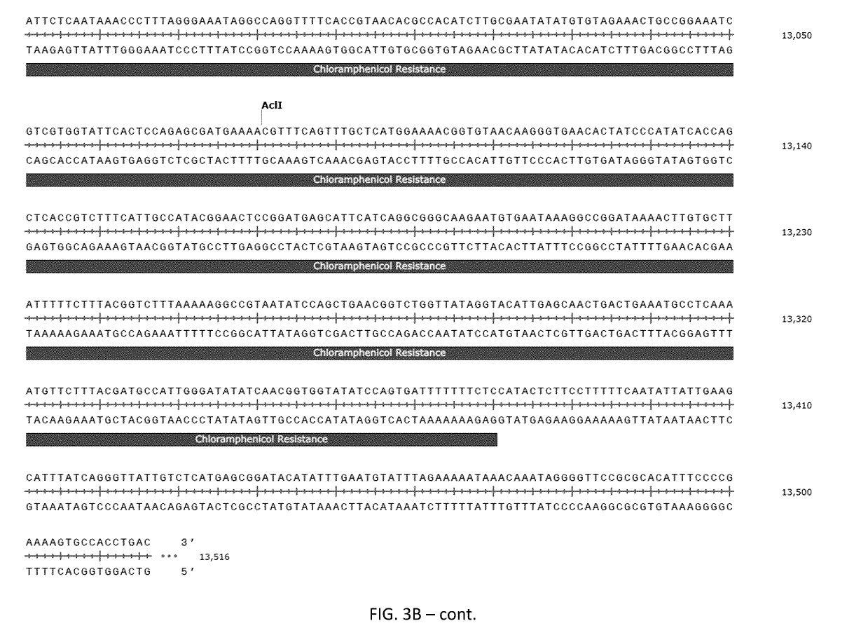

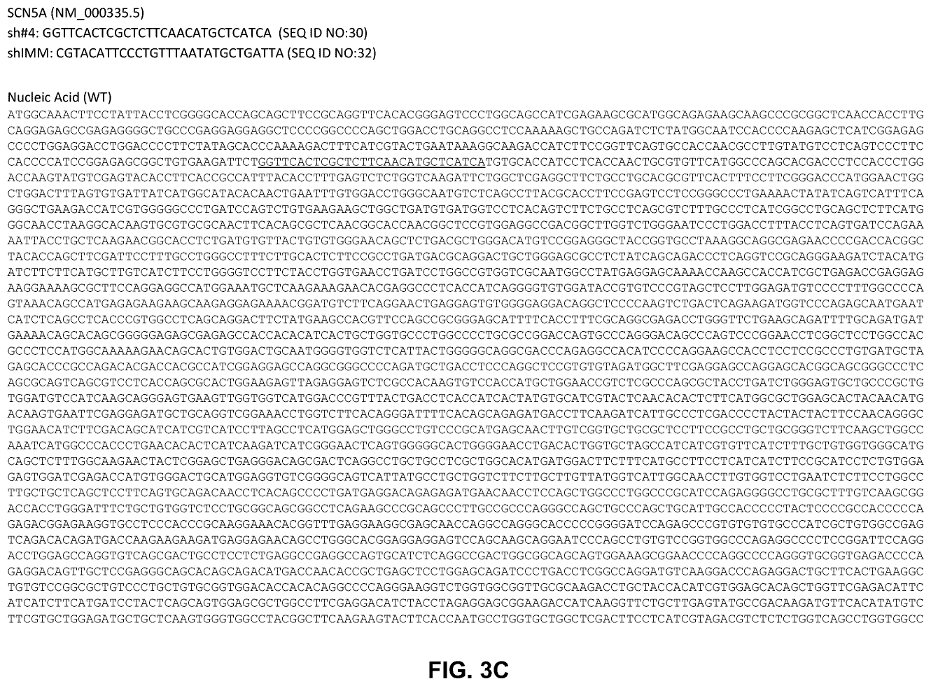

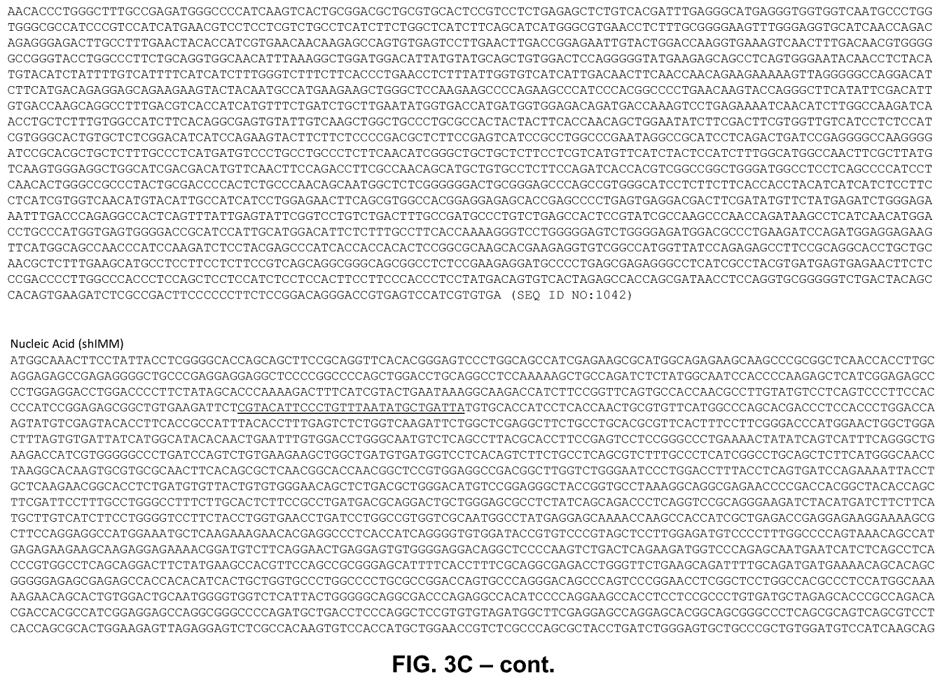

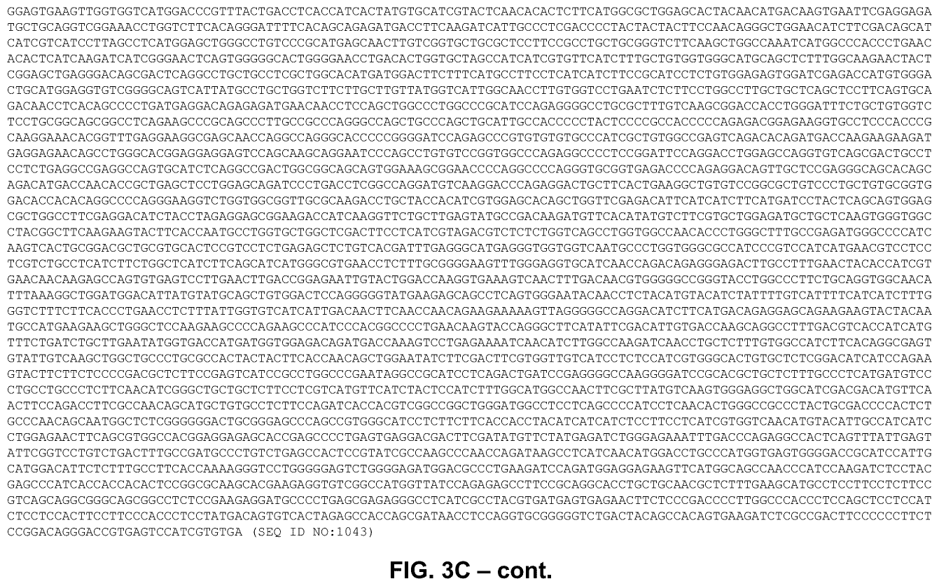

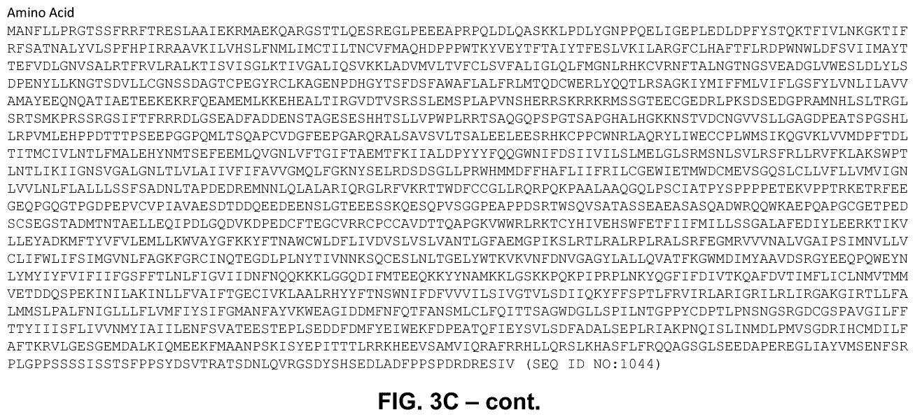

In some cases, the mammal can have LQT3 or BrS, and the gene to be suppressed and replaced can be SCN5A (which encodes sodium channel protein type 5 subunit alpha isoform b). An example of a SCN5A construct is shown in FIGS. 3A and 3B. An exemplary SCN5A sequence is set forth in NCBI RefSeq accession number NM_000335 (e.g., version NM_000335.5; FIG. 3C). A SCN5A polypeptide can, in some cases, have the amino acid sequence set forth in NCBI RefSeq accession number NP_000326 (e.g., version NP_000326.2; FIG. 3C).

Examples of shRNA sequences and corresponding shIMM sequences targeted to SCN5A are set forth in TABLE 1C.

TABLE 1C
Representative SCN5A shRNA
and shIMM sequences

| | SEQ | | SEQ |
|---|---|---|---|
| shRNA Sequence | ID | shIMM Sequence | ID |
| GGCAAACTTCCTATT | 198 | GGCTAATTTTCTTTT | 199 |
| ACCT | | GCCA | |
| GACCATCTTCCGGTT | 200 | AACGATTTTTCGCTT | 201 |
| CAGT | | TAGC | |
| GTTCAGTGCCACCAA | 202 | CTTTAGCGCGACGAA | 203 |
| CGCCTTGTAT | | TGCGTTATAC | |
| GGTTCACTCGCTCTT | 30 | CGTACATTCCCTGTT | 32 |
| CAACATGCTCATCA | | TAATATGCTGATTA | |
| GGTTCACTCGCTCTT | 204 | CGTACATTCCCTGTT | 205 |
| CAACAT | | TAATAT | |
| GTTCACTCGCTCTTC | 206 | GTACATTCCCTGTTT | 207 |
| AACATGCTCATCAT | | AATATGCTGATTAT | |
| GCTCTTCAACATGCT | 208 | CCTGTTTAATATGCT | 209 |
| CATCAT | | GATTAT | |
| GCTCTTCATGGGCAA | 210 | ACTGTTTATGGGGAA | 211 |
| CCTAAGGCACAAGT | | TCTTAGACATAAAT | |
| GGCAACCTAAGGCAC | 212 | GGGAATCTTAGACAT | 213 |
| AAGT | | AAAT | |
| GGAATCCCTGGACCT | 214 | GGAGTCGCTCGATCT | 215 |
| TTACCT | | ATATCT | |
| GGACCTTTACCTCAG | 216 | CGATCTATATCTGAG | 217 |
| TGAT | | CGAC | |
| GGGCCTTTCTTGCAC | 218 | GGGCGTTCCTAGCTC | 219 |
| TCTT | | TGTT | |
| GATCTTCTTCATGCT | 220 | GATTTTTTTTATGCT | 221 |
| TGTCAT | | AGTGAT | |
| GGAGGCCATGGAAAT | 222 | AGAAGCGATGGAGAT | 223 |
| GCTCAAGAAA | | GCTGAAAAAG | |
| GGCCATGGAAATGCT | 224 | AGCGATGGAGATGCT | 225 |
| CAAGAA | | GAAAAA | |
| GCCATGGAAATGCTC | 226 | GCGATGGAGATGCTG | 227 |
| AAGAAA | | AAAAAG | |
| GCCATGGAAATGCTC | 228 | GCGATGGAGATGCTG | 229 |
| AAGA | | AAAA | |
| GCCCCAGTAAACAGC | 230 | GCGCCTGTTAATAGT | 231 |
| CATGAGAGAA | | CACGAAAGGA | |
| GATGGTCCCAGAGCA | 232 | GACGGACCGAGGGCT | 233 |
| ATGAAT | | ATGAAC | |
| GTCCCAGAGCAATGA | 234 | GACCGAGGGCTATGA | 235 |
| ATCA | | ACCA | |
| GGAAGAGTTAGAGGA | 236 | CGAGGAATTGGAAGA | 237 |
| GTCTCGCCACAAGT | | ATCACGGCATAAAT | |
| GGAAGAGTTAGAGGA | 238 | CGAGGAATTGGAAGA | 239 |
| GTCT | | ATCA | |
| GTCCATCAAGCAGGG | 240 | GTCGATTAAACAAGG | 241 |
| AGTGAA | | TGTCAA | |
| GACCTCACCATCACT | 242 | GATCTGACGATTACA | 243 |
| ATGT | | ATGT | |
| GCGCTGGAGCACTAC | 244 | GCCCTCGAACATTAT | 245 |
| AACATGACAA | | AATATGACTA | |
| GCTGGAGCACTACAA | 246 | CCTCGAACATTATAA | 247 |
| CATGACAAGT | | TATGACTAGC | |
| GGAGCACTACAACAT | 248 | CGAACATTATAATAT | 249 |
| GACA | | GACT | |
| GAGCACTACAACATG | 250 | GAACATTATAATATG | 251 |
| ACAAGT | | ACTAGC | |
| GAGCACTACAACATG | 252 | GAACATTATAATATG | 253 |
| ACAA | | ACTA | |
| GCACTACAACATGAC | 254 | ACATTATAATATGAC | 255 |
| AAGT | | TAGC | |
| GACAAGTGAATTCGA | 256 | GACTAGCGAGTTTGA | 257 |
| GGAGAT | | AGAAAT | |
| GTCGGAAACCTGGTC | 258 | GTGGGTAATCTCGTG | 259 |
| TTCACA | | TTTACT | |
| GTCGGAAACCTGGTC | 260 | GTGGGTAATCTCGTG | 261 |
| TTCA | | TTTA | |
| GCTGGCACATGATGG | 262 | GGTGGCATATGATGG | 263 |
| ACTTCTTTCA | | ATTTTTTCCA | |
| GCTGGCACATGATGG | 264 | GGTGGCATATGATGG | 265 |
| ACTTCT | | ATTTTT | |
| GCTGGCACATGATGG | 266 | GGTGGCATATGATGG | 267 |
| ACTT | | ATTT | |
| GGCACATGATGGACT | 268 | GGCATATGATGGATT | 269 |
| TCTT | | TTTT | |
| GCACATGATGGACTT | 270 | GCATATGATGGATTT | 271 |
| CTTTCA | | TTTCCA | |
| GCACATGATGGACTT | 272 | GCATATGATGGATTT | 273 |
| CTTT | | TTTC | |
| GCCTGCTGGTCTTCT | 274 | GTCTCCTCGTGTTTT | 275 |
| TGCTTGTTAT | | TACTAGTAAT | |
| GCTGGTCTTCTTGCT | 276 | CCTCGTGTTTTTACT | 277 |
| TGTTAT | | AGTAAT | |
| GCCCCTGATGAGGAC | 278 | GCGCCAGACGAAGAT | 279 |
| AGAGAGATGAACAA | | AGGGAAATGAATAA | |
| GGAAGACCATCAAGG | 280 | GCAAAACGATTAAAG | 281 |
| TTCT | | TACT | |
| GCCTCATCTTCTGGC | 282 | GTCTGATTTTTTGGC | 283 |
| TCATCT | | TGATTT | |
| GCCAGTGTGAGTCCT | 284 | GTCAATGCGAATCGT | 285 |
| TGAACT | | TAAATT | |
| GCCCTTCTGCAGGTG | 286 | GCGCTACTCCAAGTC | 287 |
| GCAACATTTA | | GCTACTTTCA | |
| GCAGGTGGCAACATT | 288 | CCAAGTCGCTACTTT | 289 |
| TAAA | | CAAG | |
| GAAGAGCAGCCTCAG | 290 | GAGGAACAACCACAA | 291 |
| TGGGAATACA | | TGGGAGTATA | |
| GAGCAGCCTCAGTGG | 292 | GAACAACCACAATGG | 293 |
| GAATACAACCTCTA | | GAGTATAATCTGTA | |
| GCAGCCTCAGTGGGA | 294 | ACAACCACAATGGGA | 295 |
| ATACAACCTCTACA | | GTATAATCTGTATA | |
| GCAGCCTCAGTGGGA | 296 | ACAACCACAATGGGA | 297 |
| ATACAA | | GTATAA | |
| GCCTCAGTGGGAATA | 298 | ACCACAATGGGAGTA | 299 |
| CAACCTCTACATGT | | TAATCTGTATATGT | |
| GTGGGAATACAACCT | 300 | ATGGGAGTATAATCT | 301 |
| CTACAT | | GTATAT | |
| GGGAATACAACCTCT | 302 | GGGAGTATAATCTGT | 303 |
| ACATGT | | ATATGT | |
| GGGAATACAACCTCT | 304 | GGGAGTATAATCTGT | 305 |
| ACAT | | ATAT | |
| AAGTACTACAATGCC | 306 | AAATATTATAACGCG | 307 |
| ATGAAG | | ATGAAA | |
| GTACCAGGGCTTCAT | 308 | ATATCAAGGGTTTAT | 309 |
| ATTCGACATTGTGA | | TTTTGATATAGTCA | |
| GGGCTTCATATTCGA | 310 | AGGGTTTATTTTTGA | 311 |
| CATTGT | | TATAGT | |
| GGCTTCATATTCGAC | 312 | GGGTTTATTTTTGAT | 313 |
| ATTGTGACCA | | ATAGTCACGA | |
| GCTTCATATTCGACA | 314 | GGTTTATTTTTGATA | 315 |
| TTGTGA | | TAGTCA | |
| GCTTCATATTCGACA | 316 | GGTTTATTTTTGATA | 317 |
| TTGT | | TAGT | |
| GCTGCTGCTCTTCCT | 318 | CCTCCTCCTGTTTCT | 319 |
| CGTCATGTTCATCT | | GGTGATGTTTATTT | |
| GCTGCTCTTCCTCGT | 320 | CCTCCTGTTTCTGGT | 321 |
| CATGTTCATCTACT | | GATGTTTATTTATT | |
| GCTGCTCTTCCTCGT | 322 | CCTCCTGTTTCTGGT | 323 |
| CATGTT | | GATGTT | |
| GCTCTTCCTCGTCAT | 324 | CCTGTTTCTGGTGAT | 325 |
| GTTCAT | | GTTTAT | |
| GAGGCTGGCATCGAC | 326 | GAAGCAGGGATTGAT | 327 |
| GACATGTTCAACTT | | GATATGTTTAATTT | |
| GCTGGCATCGACGAC | 328 | GCAGGGATTGATGAT | 329 |
| ATGTTCAACT | | ATGTTTAATT | |
| GGCATCGACGACATG | 330 | GGGATTGATGATATG | 331 |
| TTCA | | TTTA | |
| GCATCGACGACATGT | 332 | GGATTGATGATATGT | 333 |
| TCAACT | | TTAATT | |
| GCATCGACGACATGT | 334 | GGATTGATGATATGT | 335 |
| TCAA | | TTAA | |
| GACGACATGTTCAAC | 336 | GATGATATGTTTAAT | 337 |
| TTCCAGACCT | | TTTCAAACGT | |
| GGGCATCCTCTTCTT | 338 | CGGGATTCTGTTTTT | 339 |
| CACCACCTACATCA | | TACGACGTATATTA | |
| GGCATCCTCTTCTTC | 340 | GGGATTCTGTTTTTT | 341 |
| ACCACCTACATCAT | | ACGACGTATATTAT | |
| GGCATCCTCTTCTTC | 342 | GGGATTCTGTTTTTT | 343 |
| ACCACCTACA | | ACGACGTATA | |
| GCATCCTCTTCTTCA | 344 | GGATTCTGTTTTTTA | 345 |
| CCACCTACAT | | CGACGTATAT | |
| GGTCTGACTACAGCC | 346 | GCTCAGATTATAGTC | 347 |
| ACAGTGAAGA | | ATAGCGAGGA | |
| GTCTGACTACAGCCA | 348 | CTCAGATTATAGTCA | 349 |
| CAGTGA | | TAGCGA | |

In some cases, the mammal can have HCM or DCM, and the gene to be suppressed and replaced can be MYH7 (which encodes myosin heavy chain 7). An exemplary MYH7 sequence is set forth in NCBI RefSeq accession number NM_000257 (e.g., version NM_000257.4). A MYH7 polypeptide can, in some cases, have the amino acid sequence set forth in NCBI RefSeq accession number NP_000248 (e.g., version NP_000248.2).

Examples of shRNA sequences and corresponding shIMM sequences targeted to MYH7 are set forth in TABLE 1D.

TABLE 1D
Representative MYH7 shRNA
and shIMM sequences

| | SEQ | | SEQ |
|---|---|---|---|
| shRNA Sequence | ID | shIMM Sequence | ID |
| GACCTCAAGAAGGAT | 350 | GATCTGAAAAAAGAC | 351 |
| GTCT | | GTGT | |
| GTGTCACCGTCAACC | 352 | GCGTGACGGTGAATC | 353 |
| CTTACA | | CATATA | |
| GTCACCGTCAACCCT | 354 | GTGACGGTGAATCCA | 355 |
| TACA | | TATA | |
| GTCAACACCAAGAGG | 356 | GTGAATACGAAAAGA | 357 |
| GTCATCCAGTACTT | | GTGATTCAATATTT | |
| GAGGGTCATCCAGTA | 358 | AAGAGTGATTCAATA | 359 |
| CTTT | | TTTC | |
| GCTGAAAGCAGAGAG | 33 | ACTCAAGGCTGAAAG | 35 |
| AGATTATCACATTT | | GGACTACCATATAT | |
| GGAGCTCATGGCCAC | 360 | AGAACTGATGGCGAC | 361 |
| TGATAA | | AGACAA | |
| GAGCTCATGGCCACT | 362 | GAACTGATGGCGACA | 363 |
| GATA | | GACA | |
| GGGCTTCACTTCAGA | 364 | CGGGTTTACATCTGA | 365 |
| GGAGAA | | AGAAAA | |
| GGCTTCACTTCAGAG | 366 | GGGTTTACATCTGAA | 367 |
| GAGAAA | | GAAAAG | |
| GGCTTCACTTCAGAG | 368 | GGGTTTACATCTGAA | 369 |
| GAGA | | GAAA | |
| GCTTCACTTCAGAGG | 370 | GGTTTACATCTGAAG | 371 |
| AGAA | | AAAA | |
| GGGCAGAATGTCCAG | 372 | GGCCAAAACGTGCAA | 373 |
| CAGGTGATAT | | CAAGTCATTT | |
| GGCAGAATGTCCAGC | 374 | GCCAAAACGTGCAAC | 375 |
| AGGTGATATA | | AAGTCATTTA | |
| GCAGAATGTCCAGCA | 376 | CCAAAACGTGCAACA | 377 |
| GGTGAT | | AGTCAT | |
| GAATGTCCAGCAGGT | 378 | AAACGTGCAACAAGT | 379 |
| GATATA | | CATTTA | |
| GAATGTCCAGCAGGT | 380 | AAACGTGCAACAAGT | 381 |
| GATA | | CATT | |
| GGCCAAGGCAGTGTA | 382 | CGCGAAAGCTGTCTA | 383 |
| TGAGAGGATGTTCA | | CGAAAGAATGTTTA | |
| GGCTGATGCGCCTAT | 384 | CGCAGACGCCCCAAT | 385 |
| TGAGAA | | AGAAAA | |
| GCTGATGCGCCTATT | 386 | GCAGACGCCCCAATA | 387 |
| GAGA | | GAAA | |
| GAAGGGCAAAGGCAA | 388 | AAAAGGGAAGGGGAA | 389 |
| GGCCAAGAAA | | AGCGAAAAAG | |
| GGCAAAGGCAAGGCC | 390 | GGGAAGGGGAAAGCG | 391 |
| AAGAAA | | AAAAAG | |
| GAGACTCCCTGCTGG | 392 | GGGATTCGCTCCTCG | 393 |
| TAAT | | TTAT | |
| GTCAAGAATTGGCCC | 394 | GTGAAAAACTGGCCG | 395 |
| TGGATGAAGCTCTA | | TGGATGAAACTGTA | |
| GGAGAGCATCATGGA | 396 | AGAAAGTATTATGGA | 397 |
| CCTGGAGAAT | | TCTCGAAAAC | |
| GTCCGTGCAGATCGA | 398 | CTCGGTCCAAATTGA | 399 |
| GATGAA | | AATGAA | |
| GTGCAGATCGAGATG | 400 | GTCCAAATTGAAATG | 401 |
| AACA | | AATA | |
| GCAGATCGAGATGAA | 402 | CCAAATTGAAATGAA | 403 |
| CAAGAA | | TAAAAA | |
| GAGCAGATCATCAAG | 404 | GAACAAATTATTAAA | 405 |
| GCCAAGGCTAACCT | | GCGAAAGCAAATCT | |
| GCCAAGGCTAACCTG | 406 | GCGAAAGCAAATCTC | 407 |
| GAGAAGATGT | | GAAAAAATGT | |
| GCTAACCTGGAGAAG | 408 | GCAAATCTCGAAAAA | 409 |
| ATGT | | ATGT | |
| GTGGAGGCTGTTAAT | 410 | GTCGAAGCAGTAAAC | 411 |
| GCCAAGTGCT | | GCGAAATGTT | |
| GCTGTTAATGCCAAG | 412 | GCAGTAAACGCGAAA | 413 |
| TGCT | | TGTT | |
| GCCCAGAAGCAAGTC | 414 | GCGCAAAAACAGGTG | 415 |
| AAGA | | AAAA | |
| AAGAGCCTCCAGAGC | 416 | AAAAGTCTGCAAAGT | 417 |
| TTGTTG | | TTATTA | |
| GCATCAAGGAGCTCA | 418 | GGATTAAAGAACTGA | 419 |
| CCTA | | CGTA | |
| GCTAAAGGTCAAGGC | 420 | ACTTAAAGTGAAAGC | 421 |
| CTACAA | | GTATAA | |

In some cases, the mammal can have ACM, and the gene to be suppressed and replaced can be DSP (which encodes desmoplakin). An exemplary DSP sequence is set forth in NCBI RefSeq accession number NM_004415 (e.g., version NM_004415.4). A DSP polypeptide can, in some cases, have the amino acid sequence set forth in NCBI RefSeq accession number NP_004406 (e.g., version NP_004406.2).

Examples of shRNA sequences and corresponding shIMM sequences targeted to DSP are set forth in TABLE 1E.

TABLE 1E
Representative DSP shRNA
and shIMM sequences

| | SEQ | | SEQ |
|---|---|---|---|
| shRNA Sequence | ID | shIMM Sequence | ID |
| GGAGATGGAATACAA | 422 | GGTGACGGTATTCAG | 423 |
| CTGACT | | CTCACA | |
| GGAAATCCTCGACAG | 424 | GGAGATTCTGGATAG | 425 |
| CTTGATCAGA | | TTTAATTAGG | |
| GAAATCCTCGACAGC | 426 | GAGATTCTGGATAGT | 427 |
| TTGATCAGAGAGAT | | TTAATTAGGGAAAT | |
| GCAAATGCGAGCCCT | 428 | ACAGATGCGTGCGCT | 429 |
| TTATAA | | ATACAA | |
| GGATGAGTTCACCAA | 430 | GGACGAATTTACGAA | 431 |
| ACATGT | | GCACGT | |
| GGATGAGTTCACCAA | 432 | GGACGAATTTACGAA | 433 |
| ACAT | | GCAC | |
| GTCCTCAATCAGCAT | 434 | GTGCTGAACCAACAC | 435 |
| CCAGCTTCAGACAA | | CCTGCATCTGATAA | |
| GCATCCAGCTTCAGA | 436 | ACACCCTGCATCTGA | 437 |
| CAAA | | TAAG | |
| GCAGACGCAGTGGAG | 438 | CCAAACCCAATGGAG | 439 |
| TTGGATTCTTCAGA | | CTGGATACTACAAA | |
| GCAGTGGAGTTGGAT | 440 | CCAATGGAGCTGGAT | 441 |
| TCTT | | ACTA | |
| GGAACAGATCAAGGA | 442 | CGAGCAAATTAAAGA | 443 |
| GCTGGAGAAA | | ACTCGAAAAG | |
| GGTGCAGAACTTGGT | 444 | AGTCCAAAATTTAGT | 445 |
| AAACAA | | TAATAA | |
| GTGCAGAACTTGGTA | 446 | GTCCAAAATTTAGTT | 447 |
| AACA | | AATA | |
| GAGCTCTCTGTGACT | 448 | GGGCACTGTGCGATT | 449 |
| ACAA | | ATAA | |
| GGCTCTGTGGAACCA | 450 | AGCACTCTGGAATCA | 451 |
| GCTCTACATCAACA | | ACTGTATATTAATA | |
| GCACTACTGCATGAT | 44 | GCATTATTGTATGAT | 452 |
| TGACATAGAGAAGA | | AGATATTGAAAAAA | |
| GCATGATTGACATAG | 453 | GTATGATAGATATTG | 454 |
| AGAAGA | | AAAAAA | |
| GCATGATTGACATAG | 455 | GTATGATAGATATTG | 456 |
| AGAA | | AAAA | |
| GGAACCTGCCAAGAT | 457 | GGTACGTGTCAGGAC | 458 |
| GTCAACCATAATAA | | GTGAATCACAACAA | |
| GACCAGGGATCTTCT | 459 | GATCAAGGTTCATCA | 460 |
| CACCACATCACAGT | | CATCATATTACTGT | |
| GACCAGGGATCTTCT | 461 | GATCAAGGTTCATCA | 462 |
| CACCACATCA | | CATCATATTA | |
| GCTTAAGAGTGTGCA | 463 | ACTAAAAAGCGTCCA | 464 |
| GAATGA | | AAACGA | |
| GCCTGGACCTGGATA | 465 | GTCTCGATCTCGACA | 466 |
| AAGT | | AGGT | |
| GTTGGCCACTATGAA | 467 | ATTAGCGACAATGAA | 468 |
| GACAGA | | AACTGA | |
| GTTGGCCACTATGAA | 469 | ATTAGCGACAATGAA | 470 |
| GACA | | AACT | |
| GGCCACTATGAAGAC | 471 | AGCGACAATGAAAAC | 472 |
| AGAACT | | TGAGCT | |
| GGCCACTATGAAGAC | 473 | AGCGACAATGAAAAC | 474 |
| AGAA | | TGAG | |
| GCCACTATGAAGACA | 475 | GCGACAATGAAAACT | 476 |
| GAACTA | | GAGCTT | |
| GCAGATCCACTCTCA | 477 | ACAAATTCATTCACA | 478 |
| GACT | | AACA | |
| GGCTTTCTGCAAGTG | 479 | AGCATTTTGTAAATG | 480 |
| GCTCTATGAT | | GCTGTACGAC | |
| GCTTTCTGCAAGTGG | 481 | GCATTTTGTAAATGG | 482 |
| CTCTAT | | CTGTAC | |
| GTGGCTCTATGATGC | 483 | ATGGCTGTACGACGC | 484 |
| TAAA | | AAAG | |
| GCTCGGTACATTGAA | 485 | GCACGCTATATAGAG | 486 |
| CTACTT | | CTTCTA | |
| GAACTACTTACAAGA | 487 | GAGCTTCTAACTAGG | 488 |
| TCTGGAGACTATTA | | TCAGGTGATTACTA | |
| GAACTACTTACAAGA | 489 | GAGCTTCTAACTAGG | 490 |
| TCTGGAGACT | | TCAGGTGATT | |
| GGCAGAGTGTTCCCA | 491 | AGCTGAATGCTCGCA | 492 |
| GTTCAA | | ATTTAA | |
| GCAGAGTGTTCCCAG | 493 | GCTGAATGCTCGCAA | 494 |
| TTCAAA | | TTTAAG | |
| GCAGAGTGTTCCCAG | 495 | GCTGAATGCTCGCAA | 496 |
| TTCA | | TTTA | |
| GGCAAAGGTAAGAAA | 497 | CGCTAAAGTTAGGAA | 498 |
| CCACTA | | TCATTA | |
| GCAAAGGTAAGAAAC | 499 | GCTAAAGTTAGGAAT | 500 |
| CACTAT | | CATTAC | |
| GACCACCATCAAGGA | 501 | AACGACGATTAAAGA | 502 |
| GATA | | AATT | |
| GAAGGAAGAGGATAC | 503 | AAAAGAGGAAGACAC | 504 |
| CAGT | | GAGC | |
| GGAGCTTATCTGAAG | 505 | GAAGTTTGTCAGAGG | 506 |
| AAAT | | AGAT | |
| GAGCTTATCTGAAGA | 507 | AAGTTTGTCAGAGGA | 508 |
| AATA | | GATT | |
| GATCGACAAAGAAAC | 509 | CATTGATAAGGAGAC | 510 |
| AAATGA | | TAACGA | |
| GATCGACAAAGAAAC | 511 | CATTGATAAGGAGAC | 512 |
| AAAT | | TAAC | |
| GCAGAAAGCAAACAG | 513 | CCAAAAGGCTAATAG | 514 |
| TAGT | | CAGC | |
| GGAGAGGACTGTGAA | 515 | AGAAAGAACAGTCAA | 516 |
| GGACCAGGATATCA | | AGATCAAGACATTA | |
| GACTGTGAAGGACCA | 517 | AACAGTCAAAGATCA | 518 |
| GGATAT | | AGACAT | |
| GTGAAGGACCAGGAT | 519 | GTCAAAGATCAAGAC | 520 |
| ATCA | | ATTA | |
| GAAGCAGAAGGTGGA | 521 | AAAACAAAAAGTCGA | 522 |
| AGAGGA | | GGAAGA | |
| GGAGCAGGCATCCAT | 523 | CGAACAAGCTTCGAT | 524 |
| TGTT | | AGTA | |
| GGAACAGGAAAGTGT | 525 | AGAGCAAGAGAGCGT | 526 |
| CAAA | | GAAG | |
| GAAATTGAGAGGCTG | 527 | GAGATAGAAAGACTC | 528 |
| CAGTCT | | CAATCA | |
| GAACCTGACCAAGGA | 529 | AAATCTCACGAAAGA | 530 |
| GCACTT | | ACATTT | |
| GGAGCACTTGATGTT | 531 | AGAACATTTAATGTT | 532 |
| AGAA | | GGAG | |
| GAGCACTTGATGTTA | 533 | GAACATTTAATGTTG | 534 |
| GAAGAA | | GAGGAG | |
| GCACTTGATGTTAGA | 535 | ACATTTAATGTTGGA | 536 |
| AGAA | | GGAG | |
| GCAACCATCTTGGAA | 537 | GCTACGATTTTAGAG | 538 |
| CTAA | | CTTA | |
| GAGGAGGCTATTAGG | 539 | GAAGAAGCAATAAGA | 540 |
| AAGATA | | AAAATT | |
| GGAGGCTATTAGGAA | 541 | AGAAGCAATAAGAAA | 542 |
| GATA | | AATT | |
| GGAGTGAGATCGAAA | 543 | GAAGCGAAATTGAGA | 544 |
| GACT | | GGCT | |
| GAGGATTCTACCAGG | 545 | GAAGACTCAACGAGA | 546 |
| GAGACA | | GAAACT | |
| GGATTCTACCAGGGA | 547 | AGACTCAACGAGAGA | 548 |
| GACA | | AACT | |
| GGAGATTGATAAACT | 549 | AGAAATAGACAAGCT | 550 |
| CAGACA | | GAGGCA | |
| GGAGATTGATAAACT | 551 | AGAAATAGACAAGCT | 552 |
| CAGA | | GAGG | |
| GCTGAGGAAGAAGGT | 553 | CCTCAGAAAAAAAGT | 554 |
| GACA | | CACT | |
| GAGGCCAAGAGAAAG | 555 | GAAGCGAAAAGGAAA | 556 |
| AAATTAATCA | | AAGTTGATTA | |
| GAGGCCAAGAGAAAG | 557 | GAAGCGAAAAGGAAA | 558 |
| AAATTA | | AAGTTG | |
| GAGGCCAAGAGAAAG | 559 | GAAGCGAAAAGGAAA | 560 |
| AAAT | | AAGT | |
| GGCCAAGAGAAAGAA | 561 | AGCGAAAAGGAAAAA | 562 |
| ATTAAT | | GTTGAT | |
| GGCCAAGAGAAAGAA | 563 | AGCGAAAAGGAAAAA | 564 |
| ATTA | | GTTG | |
| GCCAAGAGAAAGAAA | 565 | GCGAAAAGGAAAAAG | 566 |
| TTAA | | TTGA | |
| GAAATTAATCAGCCC | 567 | AAAGTTGATTAGTCC | 568 |
| AGAATCCACAGTCA | | TGAGTCGACTGTGA | |
| GCCCAGAATCCACAG | 569 | GTCCTGAGTCGACTG | 570 |
| TCAT | | TGAT | |
| GGTATAATTGATCCC | 571 | GGAATTATAGACCCG | 572 |
| CATCGGAATGAGAA | | CACCGCAACGAAAA | |
| GATCCCCATCGGAAT | 573 | GACCCGCACCGCAAC | 574 |
| GAGA | | GAAA | |
| AAGAAGGTCAGTTAC | 575 | AAAAAAGTGAGCTAT | 576 |
| GTGCAG | | GTCCAA | |
| GGTCTGCTCTTGCTT | 577 | GGACTCCTGTTACTA | 578 |
| TCAGTA | | TCTGTT | |
| GTCTGCTCTTGCTTT | 579 | GACTCCTGTTACTAT | 580 |
| CAGT | | CTGT | |
| GCTTTCAGTACAGAA | 581 | ACTATCTGTTCAAAA | 582 |
| GAGA | | AAGG | |
| GCATAGCAGGCATAT | 583 | GTATTGCTGGGATTT | 584 |
| ACAA | | ATAA | |
| GGCATTTATGAGGCC | 585 | GGGATATACGAAGCG | 586 |
| ATGAAA | | ATGAAG | |
| GCAACTTGAGGTTAC | 587 | GTAATTTAAGATTGC | 588 |
| CAGT | | CTGT | |
| GCAGAACGAGCTGTC | 589 | GCTGAGCGTGCAGTG | 590 |
| ACTGGGTATAATGA | | ACAGGCTACAACGA | |
| GAGCTGTCACTGGGT | 591 | GTGCAGTGACAGGCT | 592 |
| ATAA | | ACAA | |
| GCTGTCACTGGGTAT | 593 | GCAGTGACAGGCTAC | 594 |
| AATGAT | | AACGAC | |
| GGGTATAATGATCCT | 595 | GGCTACAACGACCCA | 596 |
| GAAACA | | GAGACT | |
| GAAACAGGAAACATC | 597 | GAGACTGGTAATATT | 598 |
| ATCTCT | | ATTTCA | |
| GAAACAGGAAACATC | 599 | GAGACTGGTAATATT | 600 |
| ATCT | | ATTT | |
| GGGCCACGGTATTCG | 601 | AGGGCATGGAATACG | 602 |
| CTTATTAGAA | | GTTGTTGGAG | |
| GGGCCACGGTATTCG | 603 | AGGGCATGGAATACG | 604 |
| CTTATT | | GTTGTT | |
| GGCCACGGTATTCGC | 605 | GGGCATGGAATACGG | 606 |
| TTATTA | | TTGTTG | |
| GCCACGGTATTCGCT | 607 | GGCATGGAATACGGT | 608 |
| TATT | | TGTT | |
| GACCCAAAGGAGAGC | 609 | GATCCTAAAGAAAGT | 610 |
| CATCGTTTACCAGT | | CACCGATTGCCTGT | |
| GGAGAGCCATCGTTT | 611 | AGAAAGTCACCGATT | 612 |
| ACCAGT | | GCCTGT | |
| GGAGAGCCATCGTTT | 613 | AGAAAGTCACCGATT | 614 |
| ACCA | | GCCT | |
| GAGCCATCGTTTACC | 615 | AAGTCACCGATTGCC | 616 |
| AGTTGACATA | | TGTAGATATT | |
| GAGCCATCGTTTACC | 617 | AAGTCACCGATTGCC | 618 |
| AGTTGA | | TGTAGA | |
| GAGCCATCGTTTACC | 619 | AAGTCACCGATTGCC | 620 |
| AGTT | | TGTA | |
| GCCATCGTTTACCAG | 621 | GTCACCGATTGCCTG | 622 |
| TTGACA | | TAGATA | |
| GCCATCGTTTACCAG | 623 | GTCACCGATTGCCTG | 624 |
| TTGA | | TAGA | |
| GTTTACCAGTTGACA | 625 | GATTGCCTGTAGATA | 626 |
| TAGCATATAA | | TTGCTTACAA | |
| GTTGACATAGCATAT | 627 | GTAGATATTGCTTAC | 628 |
| AAGA | | AAAA | |
| GATTCTCTCAGATCC | 629 | AATACTGTCTGACCC | 630 |
| AAGTGATGAT | | TAGCGACGAC | |
| GATTCTCTCAGATCC | 631 | AATACTGTCTGACCC | 632 |
| AAGTGA | | TAGCGA | |
| GATCCAAGTGATGAT | 633 | GACCCTAGCGACGAC | 634 |
| ACCA | | ACGA | |
| GCTCTGTCTTCTGCC | 635 | CCTGTGCCTACTCCC | 636 |
| TCTGAA | | ACTCAA | |
| GGAAGCGTAGAGTGG | 637 | GAAAACGAAGGGTCG | 638 |
| TCATAGTTGA | | TGATTGTAGA | |
| GGAAGCGTAGAGTGG | 639 | GAAAACGAAGGGTCG | 640 |
| TCAT | | TGAT | |
| GAAGCGTAGAGTGGT | 641 | AAAACGAAGGGTCGT | 642 |
| CATAGT | | GATTGT | |
| GAAGCGTAGAGTGGT | 643 | AAAACGAAGGGTCGT | 644 |
| CATA | | GATT | |
| GCGTAGAGTGGTCAT | 645 | ACGAAGGGTCGTGAT | 646 |
| AGTTGA | | TGTAGA | |
| GCGTAGAGTGGTCAT | 647 | ACGAAGGGTCGTGAT | 648 |
| AGTT | | TGTA | |
| GTTGACCCAGAAACC | 649 | GTAGATCCTGAGACG | 650 |
| AATAAA | | AACAAG | |
| GACCCAGAAACCAAT | 651 | GATCCTGAGACGAAC | 652 |
| AAAGAAATGT | | AAGGAGATGT | |
| GACCCAGAAACCAAT | 653 | GATCCTGAGACGAAC | 654 |
| AAAGAA | | AAGGAG | |
| GTCTGTTCAGGAGGC | 655 | GTCAGTACAAGAAGC | 656 |
| CTACAA | | GTATAA | |
| GCCTACAAGAAGGGC | 657 | GCGTATAAAAAAGGG | 658 |
| CTAATT | | CTTATA | |
| GCAGGAATGTGAATG | 659 | ACAAGAGTGCGAGTG | 660 |
| GGAAGAAATA | | GGAGGAGATT | |
| GGAATGTGAATGGGA | 661 | AGAGTGCGAGTGGGA | 662 |
| AGAAAT | | GGAGAT | |
| GAATGTGAATGGGAA | 663 | GAGTGCGAGTGGGAG | 664 |
| GAAATA | | GAGATT | |
| GGGAAGAAATAACCA | 665 | GGGAGGAGATTACGA | 666 |
| TCACGGGATCAGAT | | TTACCGGTTCTGAC | |
| GCAGTCAGTATGATA | 667 | GGAGCCAATACGACA | 668 |
| TTCAAGATGCTATT | | TACAGGACGCAATA | |
| GCAGTCAGTATGATA | 669 | GGAGCCAATACGACA | 670 |
| TTCA | | TACA | |
| GCCTCAGCCTCACTC | 671 | GTCTGAGTCTGACAC | 672 |
| AATT | | AGTT | |
| GCTGACATGATCTCC | 673 | GCAGATATGATTTCG | 674 |
| TTGAAA | | TTAAAG | |
| GCTCCCGACATGAAT | 675 | GTTCGCGTCACGAGT | 676 |
| CAGTAA | | CTGTTA | |
| GCTCCCGACATGAAT | 677 | GTTCGCGTCACGAGT | 678 |
| CAGT | | CTGT | |
| GCGTCAGGAATTTAA | 679 | GTGTGAGAAACTTGA | 680 |
| CCATAA | | CGATTA | |
| GTCAGGAATTTAACC | 681 | GTGAGAAACTTGACG | 682 |
| ATAA | | ATTA | |
| GTGTGATTGACCAAG | 683 | GAGTCATAGATCAGG | 684 |
| ACAT | | ATAT | |
| GCAGCAGAGGCAGTG | 685 | GCTGCTGAAGCTGTC | 686 |
| AAAGAA | | AAGGAG | |
| GGAAGTGCATGGGAG | 687 | CGAGGTCCACGGCAG | 688 |
| GATA | | AATT | |
| GAAGTGCATGGGAGG | 689 | GAGGTCCACGGCAGA | 690 |
| ATAA | | ATTA | |
| GCTCCATGGTAGAAG | 691 | GGTCGATGGTTGAGG | 692 |
| ATATCA | | ACATTA | |
| GACGCCACAGGGAAT | 693 | GATGCGACTGGCAAC | 694 |
| TCTT | | TCAT | |
| GAATTCTTCCTACTC | 695 | CAACTCATCGTATTC | 696 |
| TTAT | | ATAC | |

In some cases, the mammal can have HCM, and the gene to be suppressed and replaced can be MYBPC3 (which encodes myosin binding protein C3). An exemplary MYBPC3 sequence is set forth in NCBI RefSeq accession number NM_000256 (e.g., version NM_000256.3). A MYBPC3 polypeptide can, in some cases, have the amino acid sequence set forth in NCBI RefSeq accession number NP_000247 (e.g., version NP_000247.2).

Examples of shRNA sequences and corresponding shIMM sequences targeted to MYBPC3 are set forth in TABLE 1F.

TABLE 1F
Representative MYBPC3
shRNA and shIMM sequences

| | SEQ | | SEQ |
|---|---|---|---|
| shRNA Sequence | ID | shIMM Sequence | ID |
| GCTCCTCCAAGGTCA | 697 | GGTCGTCGAAAGTGA | 698 |
| AGTT | | AATT | |
| GCTCCAACTTCAATC | 699 | GTTCGAATTTTAACC | 700 |
| TCACTGTCCA | | TGACAGTGCA | |
| GCCATGAGGACACTG | 701 | GTCACGAAGATACAG | 702 |
| GGATTCTGGACTT | | GCATACTCGATTT | |
| GAGGACACTGGGATT | 703 | GAAGATACAGGCATA | 704 |
| CTGGACTTCA | | CTCGATTTTA | |
| GGACACTGGGATTCT | 705 | AGATACAGGCATACT | 706 |
| GGACTT | | CGATTT | |
| GAGAAGAAGAGCACA | 707 | GAAAAAAAAAGTACT | 708 |
| GCCTTTCAGA | | GCGTTCCAAA | |
| GAGAAGAAGAGCACA | 709 | GAAAAAAAAAGTACT | 710 |
| GCCTTTCAGAAGA | | GCGTTCCAAAAAA | |
| GGTGAGCAAAGGCCA | 711 | AGTCAGTAAGGGGCA | 712 |
| CAAGAT | | TAAAAT | |
| GAGGTCAAATGGCTC | 713 | GAAGTGAAGTGGCTG | 714 |
| AAGAAT | | AAAAAC | |
| GAGGTCAAATGGCTC | 715 | GAAGTGAAGTGGCTG | 716 |
| AAGA | | AAAA | |
| GGTCAAATGGCTCAA | 717 | AGTGAAGTGGCTGAA | 718 |
| GAAT | | AAAC | |
| GCTCAAGAATGGCCA | 719 | GCTGAAAAACGGGCA | 720 |
| GGAGATCCAGATGA | | AGAAATTCAAATGA | |
| GCTCAAGAATGGCCA | 721 | GCTGAAAAACGGGCA | 722 |
| GGAGAT | | AGAAAT | |
| GGAGGAGACCTTCAA | 46 | CGAAGAAACGTTTAA | 723 |
| ATACCGGTTCAAGA | | GTATCGCTTTAAAA | |
| AAGGACCGCAGCATC | 724 | AAAGATCGGAGTATT | 725 |
| TTCACG | | TTTACC | |
| GGGCAGAGAAGGAAG | 726 | GCGCTGAAAAAGAGG | 727 |
| ATGA | | ACGA | |
| GAGAAGGAAGATGAG | 728 | GAAAAAGAGGACGAA | 729 |
| GGCGTCTACA | | GGGGTGTATA | |
| GAAGATGAGGGCGTC | 730 | GAGGACGAAGGGGTG | 731 |
| TACA | | TATA | |
| GCTACATCCTGGAGC | 732 | GGTATATTCTCGAAC | 733 |
| GCAAGAAGAA | | GGAAAAAAAA | |
| GCGCAAGAAGAAGAA | 734 | ACGGAAAAAAAAAAA | 735 |
| GAGCTA | | AAGTTA | |
| GCAAGAAGAAGAAGA | 736 | GGAAAAAAAAAAAAA | 737 |
| GCTA | | GTTA | |
| GCGCCAGACCATTCA | 738 | CCGGCAAACGATACA | 739 |
| GAAGAA | | AAAAAA | |
| GCCAGACCATTCAGA | 740 | GGCAAACGATACAAA | 741 |
| AGAA | | AAAA | |
| GGCATCACCTATGAG | 742 | GGGATTACGTACGAA | 743 |
| CCACCCAACTATAA | | CCTCCGAATTACAA | |
| GTAGCCCCAAGCCCA | 744 | GAAGTCCGAAACCGA | 745 |
| AGATTT | | AAATAT | |
| GCCCAAGATTTCCTG | 746 | ACCGAAAATATCGTG | 747 |
| GTTCAAGAAT | | GTTTAAAAAC | |
| GCCCAAGATTTCCTG | 748 | ACCGAAAATATCGTG | 749 |
| GTTCAA | | GTTTAA | |
| GATTTCCTGGTTCAA | 750 | AATATCGTGGTTTAA | 751 |
| GAAT | | AAAC | |
| GTTGACTCTGGAGAT | 752 | CTTAACACTCGAAAT | 753 |
| TAGA | | AAGG | |

In some cases, the mammal can have DCM, and the gene to be suppressed and replaced can be RBM20 (which encodes RNA binding motif protein 20). An exemplary RBM20 sequence is set forth in NCBI RefSeq accession number NM_001134363 (e.g., version NM_001134363.3). A RBM20 polypeptide can, in some cases, have the amino acid sequence set forth in NCBI RefSeq accession number NP_001127835 (e.g., version NP_001127835.2).

Examples of shRNA sequences and corresponding shIMM sequences targeted to RBM20 are set forth in TABLE 1G.

TABLE 1G
Representative RBM20 shRNA
and shIMM sequences

| | SEQ | | SEQ |
|---|---|---|---|
| shRNA Sequence | ID | shIMM Sequence | ID |
| GCCATGTCCCAGCCT | 754 | GCGATGTCGCAACCA | 755 |
| CTCTTCAATCAACT | | CTGTTTAACCAGCT | |
| GTCCCAGCCTCTCTT | 756 | GTCGCAACCACTGTT | 757 |
| CAATCA | | TAACCA | |
| GCCCTGAAACAGATG | 758 | GGCCAGAGACTGACG | 759 |
| GTCA | | GACA | |
| GCCAAAGCAAGCCTG | 760 | GGCAGAGTAAACCAG | 761 |
| ATCTCA | | ACCTGA | |
| GCCAAAGCAAGCCTG | 762 | GGCAGAGTAAACCAG | 763 |
| ATCT | | ACCT | |
| GCCGCATATCTGTAG | 764 | ACCCCACATTTGCAG | 765 |
| CATCTGTGACAAGA | | TATTTGCGATAAAA | |
| GCATATCTGTAGCAT | 766 | CCACATTTGCAGTAT | 767 |
| CTGTGA | | TTGCGA | |
| GCATATCTGTAGCAT | 768 | CCACATTTGCAGTAT | 769 |
| CTGT | | TTGC | |
| GTAGCATCTGTGACA | 770 | GCAGTATTTGCGATA | 771 |
| AGAA | | AAAA | |
| GCATCTGTGACAAGA | 772 | GTATTTGCGATAAAA | 773 |
| AGGTGT | | AAGTCT | |
| GAAAGGGAAGCTGCA | 774 | CAAGGGCAAACTCCA | 775 |
| CGCTCAGAAA | | TGCACAAAAG | |
| GGAAGCTGCACGCTC | 776 | GCAAACTCCATGCAC | 777 |
| AGAAAT | | AAAAGT | |
| GAAGCTGCACGCTCA | 778 | CAAACTCCATGCACA | 779 |
| GAAA | | AAAG | |
| GCTCAGAAATGCCTG | 780 | GCACAAAAGTGTCTC | 781 |
| GTCT | | GTGT | |
| GCTGGCATCCGGTGT | 782 | GCAGGGATTCGCTGC | 783 |
| ATACTT | | ATTCTA | |
| GCTGTTTATAACCCT | 784 | GCAGTATACAATCCA | 785 |
| GCTGGGAATGAAGA | | GCAGGCAACGAGGA | |
| GCCCATTCCAGCAAG | 786 | CCCGATACCTGCTAG | 787 |
| GTCATTCACTCAGT | | ATCTTTTACACAAT | |
| GCCCATTCCAGCAAG | 788 | CCCGATACCTGCTAG | 789 |
| GTCATT | | ATCTTT | |
| GCAAGGTCATTCACT | 790 | GCTAGATCTTTTACA | 791 |
| CAGTCA | | CAATCT | |
| GCAAGGTCATTCACT | 792 | GCTAGATCTITTACA | 793 |
| CAGT | | CAAT | |
| GGTCATTCACTCAGT | 48 | GATCTTTTACACAAT | 794 |
| CAAGCCCCACATTT | | CTAGTCCGACTTTC | |
| GAAGGAAGCTGCACT | 795 | GAGGGTAGTTGTACA | 796 |
| GAGAAT | | GAAAAC | |
| GAAGGAAGCTGCACT | 797 | GAGGGTAGTTGTACA | 798 |
| GAGA | | GAAA | |
| GAAGCTGCACAGGCC | 799 | GAGGCAGCTCAAGCG | 800 |
| ATGGTCCAGTATTA | | ATGGTGCAATACTA | |
| GCTGCACAGGCCATG | 801 | GCAGCTCAAGCGATG | 802 |
| GTCCAGTATTATCA | | GTGCAATACTACCA | |
| GCACAGGCCATGGTC | 803 | GCTCAAGCGATGGTG | 804 |
| CAGTATTATCAAGA | | CAATACTACCAGGA | |
| GGCCATGGTCCAGTA | 805 | AGCGATGGTGCAATA | 806 |
| TTATCAAGAA | | CTACCAGGAG | |
| GGCCATGGTCCAGTA | 807 | AGCGATGGTGCAATA | 808 |
| TTATCA | | CTACCA | |
| GGCCATGGTCCAGTA | 809 | AGCGATGGTGCAATA | 810 |
| TTAT | | CTAC | |
| GCCATGGTCCAGTAT | 811 | GCGATGGTGCAATAC | 812 |
| TATCAAGAAA | | TACCAGGAGA | |
| GGTCCAGTATTATCA | 813 | GGTGCAATACTACCA | 814 |
| AGAA | | GGAG | |
| GTCCAGTATTATCAA | 815 | GTGCAATACTACCAG | 816 |
| GAAA | | GAGA | |
| GCTGTGATCAATGGT | 817 | GCAGTCATTAACGGA | 818 |
| GAGA | | GAAA | |
| GAAGTTGCTCATTCG | 819 | AAAATTACTGATACG | 820 |
| GATGTCCAAGAGAT | | CATGTCGAAAAGGT | |
| GTTGCTCATTCGGAT | 821 | ATTACTGATACGCAT | 822 |
| GTCCAAGAGATACA | | GTCGAAAAGGTATA | |
| GTTGCTCATTCGGAT | 823 | ATTACTGATACGCAT | 824 |
| GTCCAAGAGA | | GTCGAAAAGG | |
| GGATGTCCAAGAGAT | 825 | GCATGTCGAAAAGGT | 826 |
| ACAA | | ATAA | |
| GGAATTGCAGCTCAA | 827 | AGAGTTACAACTGAA | 828 |
| GAAA | | AAAG | |
| GTGGGCAGACAGGAG | 829 | GTCGGGAGGCAAGAA | 830 |
| AAAGAAGCAGAGTT | | AAGGAGGCTGAATT | |
| GTGGGCAGACAGGAG | 831 | GTCGGGAGGCAAGAA | 832 |
| AAAGAA | | AAGGAG | |
| GGGCAGACAGGAGAA | 833 | CGGGAGGCAAGAAAA | 834 |
| AGAA | | GGAG | |
| GCAGACAGGAGAAAG | 835 | GGAGGCAAGAAAAGG | 836 |
| AAGCAGAGTT | | AGGCTGAATT | |
| AAGAAGCAGAGTTCT | 837 | AGGAGGCTGAATTTT | 838 |
| CTGATC | | CAGACC | |
| GAGCTGGAAGAAATT | 839 | GAACTCGAGGAGATA | 840 |
| GTGCCCATTGACCA | | GTCCCGATAGATCA | |
| GCCCATTGACCAGAA | 841 | CCCGATAGATCAAAA | 842 |
| AGACAA | | GGATAA | |
| GTGTGACAACCACCT | 843 | GCGTCACTACGACGT | 844 |
| TAGACT | | TGGATT | |
| GTGTGACAACCACCT | 845 | GCGTCACTACGACGT | 846 |
| TAGA | | TGGA | |
| GTGACAACCACCTTA | 847 | GTCACTACGACGTTG | 848 |
| GACTTA | | GATTTG | |
| GTGACAACCACCTTA | 849 | GTCACTACGACGTTG | 850 |
| GACT | | GATT | |
| GACAACCACCTTAGA | 851 | CACTACGACGTTGGA | 852 |
| CTTA | | TTTG | |
| GCAGAAATCAGCCTC | 853 | GCTGAGATTAGTCTG | 854 |
| AAGTCA | | AAATCT | |
| GCAGAAATCAGCCTC | 855 | GCTGAGATTAGTCTG | 856 |
| AAGT | | AAAT | |
| GAAATCAGCCTCAAG | 857 | GAGATTAGTCTGAAA | 858 |
| TCACCCAGAGAACT | | TCTCCGAGGGAGCT | |
| GCAAAGGGAGTGGAG | 859 | GCTAAAGGTGTCGAA | 860 |
| AGCTCAGATGTTCA | | AGTTCTGACGTACA | |
| GGAGTGGAGAGCTCA | 861 | GGTGTCGAAAGTTCT | 862 |
| GATGTT | | GACGTA | |
| GAGTGGAGAGCTCAG | 863 | GTGTCGAAAGTTCTG | 864 |
| ATGT | | ACGT | |
| GTGGATGATTGCAAG | 865 | GTCGACGACTGTAAA | 866 |
| ACCA | | ACGA | |
| GAGGTCACCAGAGTA | 867 | CAGATCTCCTGAATA | 868 |
| CACT | | TACA | |

In some cases, the mammal can have LQTS or Timothy syndrome, and the gene to be suppressed and replaced can be CACNA1C (which encodes calcium voltage-gated channel subunit alpha1 C). An exemplary CACNA1C sequence is set forth in NCBI RefSeq accession number NM_000719 (e.g., version NM_000719.7). A CACNA1C polypeptide can, in some cases, have the amino acid sequence set forth in NCBI RefSeq accession number NP_000710 (e.g., version NP_000710.5).

Examples of shRNA sequences and corresponding shIMM sequences targeted to CACNA1C are set forth in TABLE 111.

TABLE 1H
Representative CACNA1C shRNA
and shIMM sequences

| | SEQ | | SEQ |
|---|---|---|---|
| shRNA Sequence | ID | shIMM Sequence | ID |
| GTCAATGAGAATACG | 869 | GTGAACGAAAACACC | 870 |
| AGGATGTACA | | AGAATGTATA | |
| GGAACGAGTGGAATA | 50 | CGAGCGTGTCGAGTA | 871 |
| TCTCTTTCTCATAA | | CCTGTTCCTGATTA | |
| GGAGAAGCAGCAGCT | 872 | CGAAAAACAACAACT | 873 |
| AGAAGA | | TGAGGA | |
| GGAGAAGCAGCAGCT | 874 | CGAAAAACAACAACT | 875 |
| AGAA | | TGAG | |
| GCAGCAGCTAGAAGA | 876 | ACAACAACTTGAGGA | 877 |
| GGATCTCAAA | | AGACCTGAAG | |
| GCAGCAGCTAGAAGA | 878 | ACAACAACTTGAGGA | 879 |
| GGATCT | | AGACCT | |
| GCAGCTAGAAGAGGA | 880 | ACAACTTGAGGAAGA | 881 |
| TCTCAA | | CCTGAA | |
| GCTAGAAGAGGATCT | 882 | ACTTGAGGAAGACCT | 883 |
| CAAA | | GAAG | |
| GATTGGATCACTCAG | 884 | GACTGGATTACACAA | 885 |
| GCCGAAGACA | | GCGGAGGATA | |
| GCTCCTTCTCCTCTT | 886 | CCTGCTACTGCTGTT | 887 |
| CCTCTTCATCATCA | | TCTGTTTATTATTA | |
| AAGTTCAACTTTGAT | 888 | AAATTTAATTTCGAC | 889 |
| GAGATG | | GAAATG | |
| GGACTGGAATTCGGT | 890 | AGATTGGAACTCCGT | 891 |
| GATGTA | | CATGTA | |
| GACTGGAATTCGGTG | 892 | GATTGGAACTCCGTC | 893 |
| ATGTAT | | ATGTAC | |
| GACTGGAATTCGGTG | 894 | GATTGGAACTCCGTC | 895 |
| ATGT | | ATGT | |
| GGAATTCGGTGATGT | 896 | GGAACTCCGTCATGT | 897 |
| ATGA | | ACGA | |
| GGAGGAGGAAGAGGA | 898 | AGAAGAAGAGGAAGA | 899 |
| GAAGGAGAGAAAGA | | AAAAGAAAGGAAAA | |
| GCCGGAACTACTTCA | 900 | GTCGCAATTATTTTA | 901 |
| ACAT | | ATAT | |
| GTCCAGTGCAATCAA | 902 | ATCGAGCGCTATTAA | 903 |
| TGTCGTGAAGATCT | | CGTGGTCAAAATTT | |
| GCTCTTCAAGGGAAA | 904 | ACTGTTTAAAGGTAA | 905 |
| GCTGTACACCTGTT | | ACTCTATACGTGCT | |
| GGGAGCAGGAGTACA | 906 | GCGAACAAGAATATA | 907 |
| AGAACTGTGA | | AAAATTGCGA | |
| GGGAGCAGGAGTACA | 908 | GCGAACAAGAATATA | 909 |
| AGAACT | | AAAATT | |
| GAGCAGGAGTACAAG | 910 | GAACAAGAATATAAA | 911 |
| AACTGT | | AATTGC | |
| GCAGGAGTACAAGAA | 912 | ACAAGAATATAAAAA | 913 |
| CTGTGA | | TTGCGA | |
| GGAACAACAACTTTC | 914 | GCAATAATAATTTCC | 915 |
| AGACCT | | AAACGT | |
| GAAGCCAAGGGTCGT | 916 | GAGGCGAAAGGACGA | 917 |
| ATCAAA | | ATTAAG | |
| GAAGCCAAGGGTCGT | 918 | GAGGCGAAAGGACGA | 919 |
| ATCA | | ATTA | |

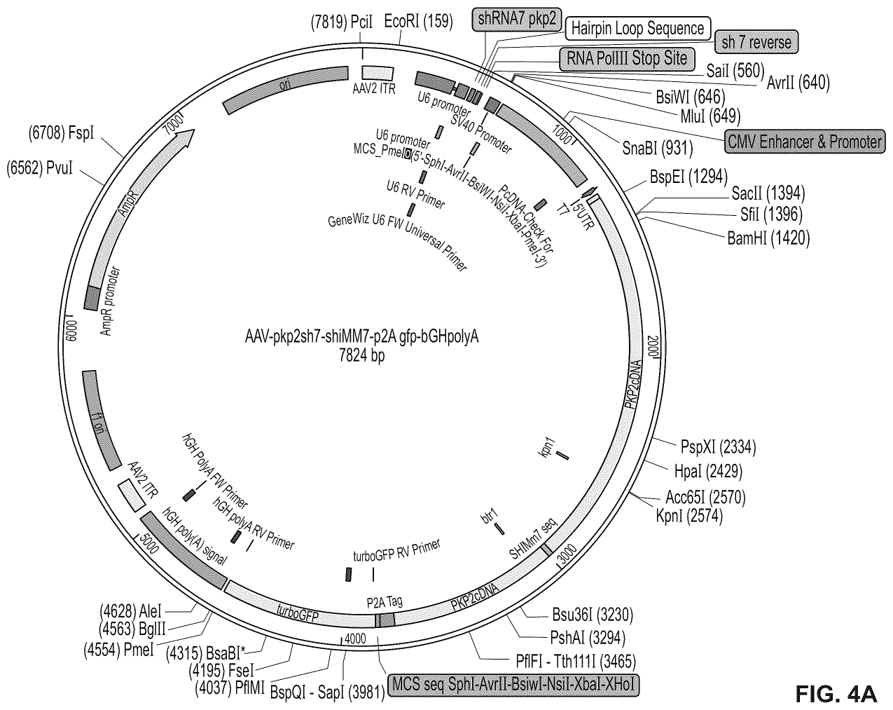

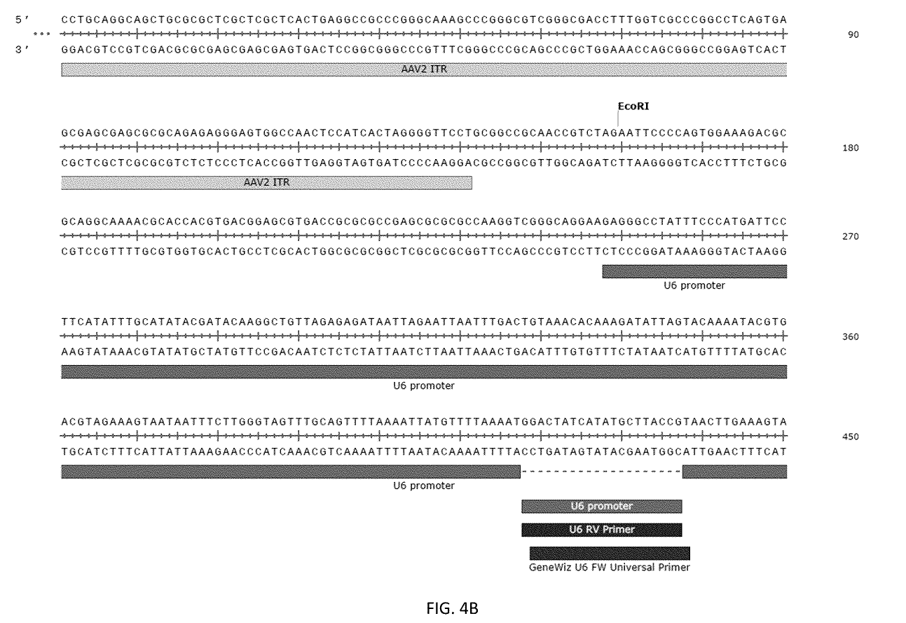

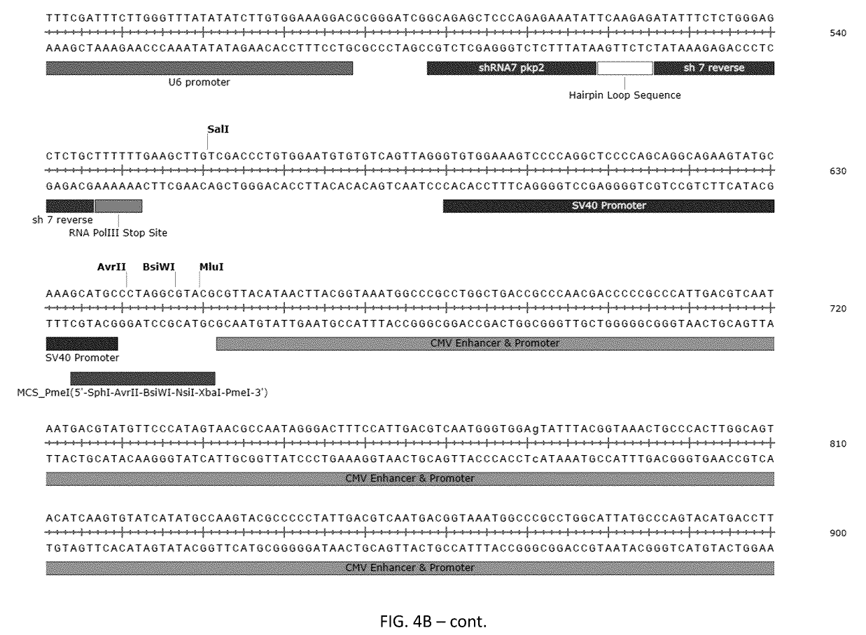

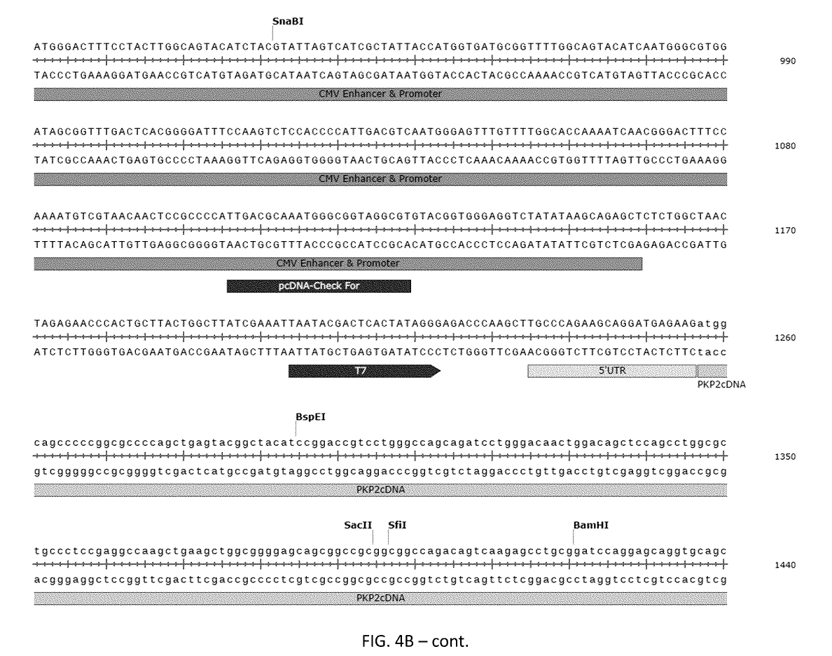

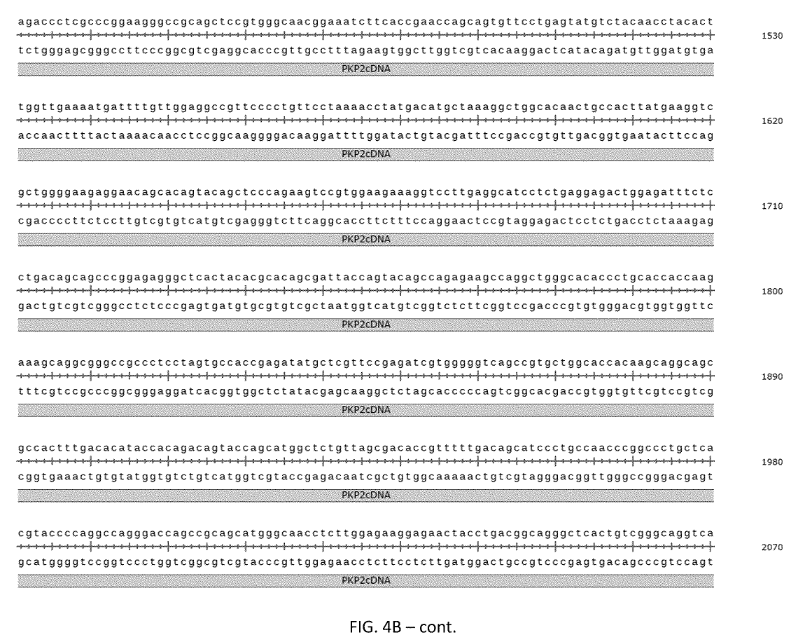

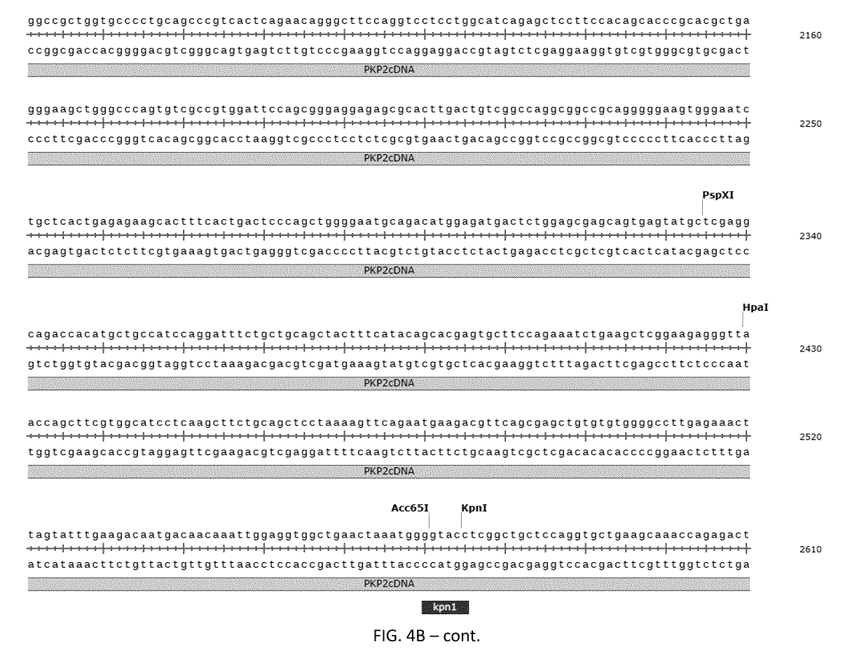

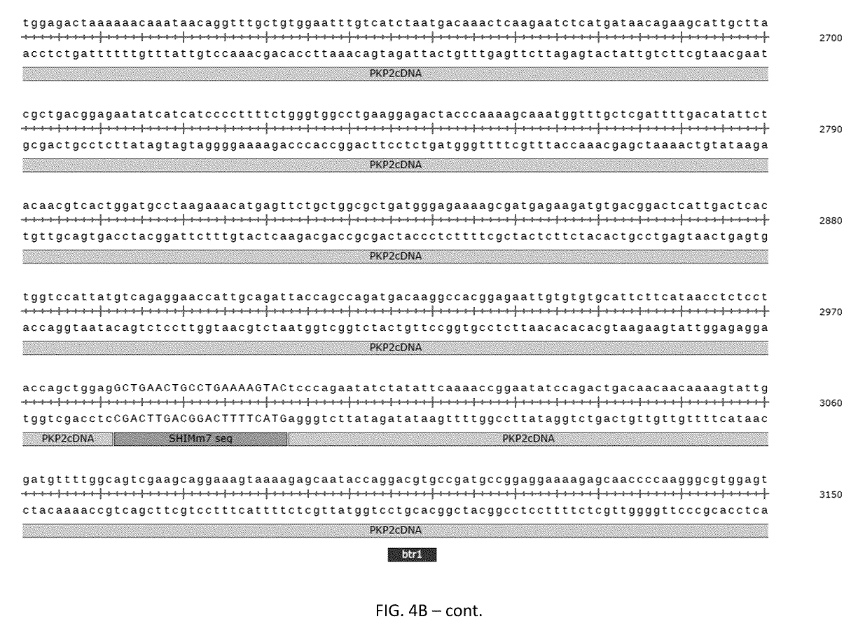

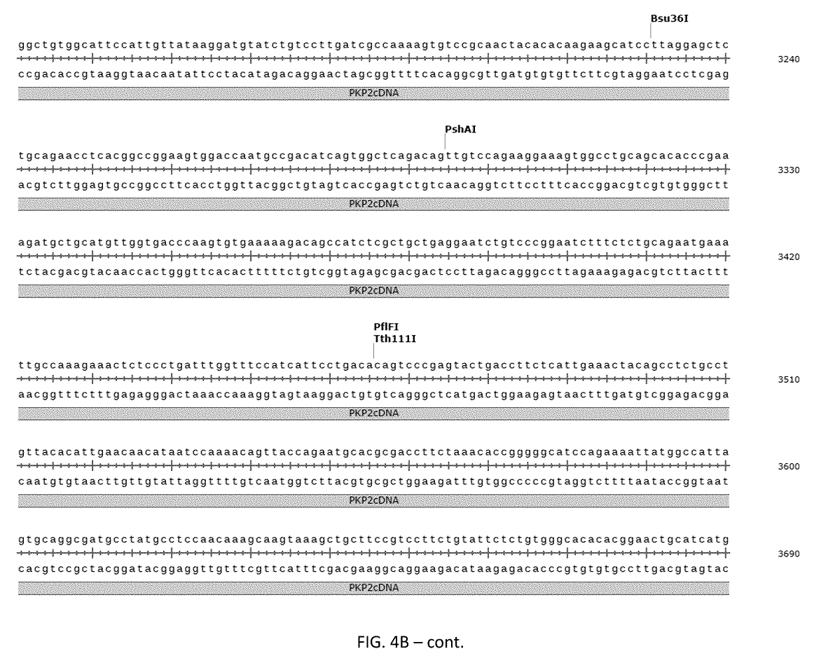

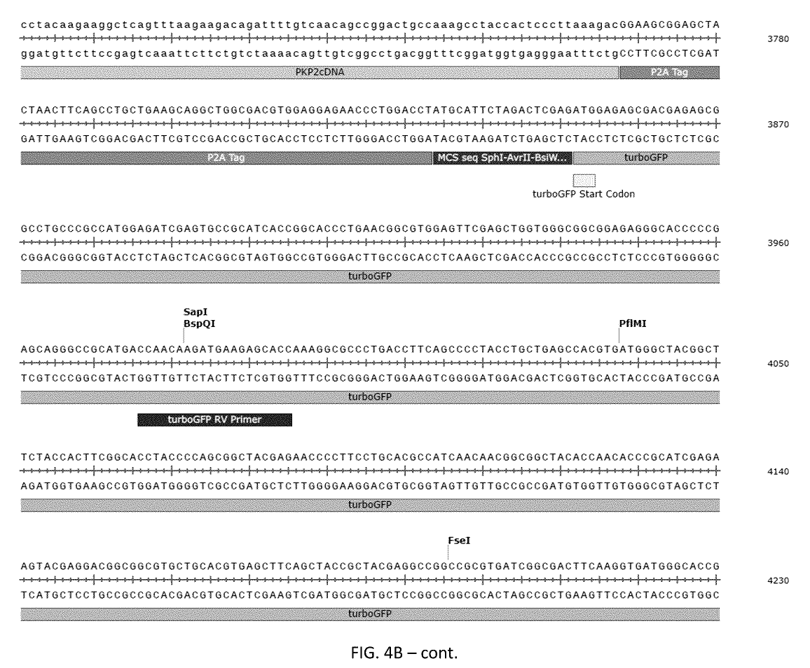

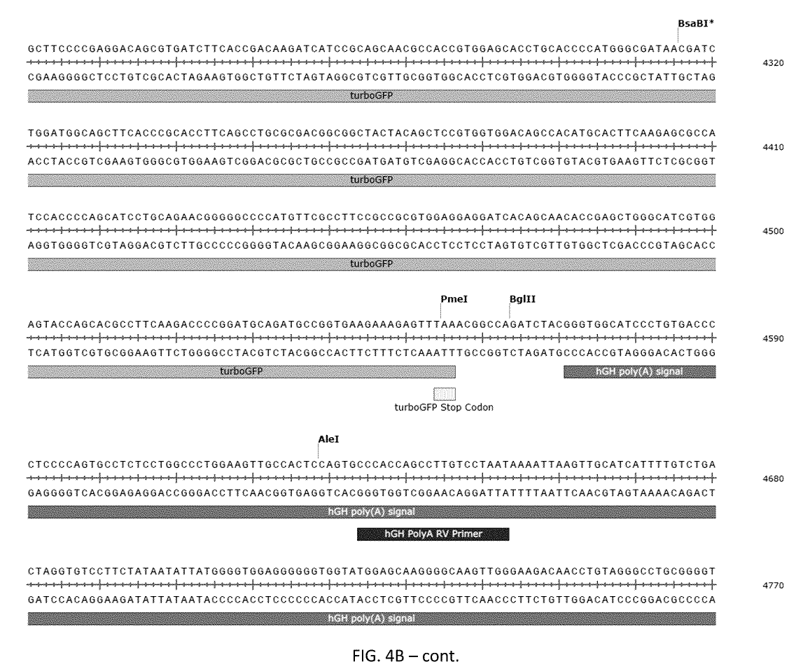

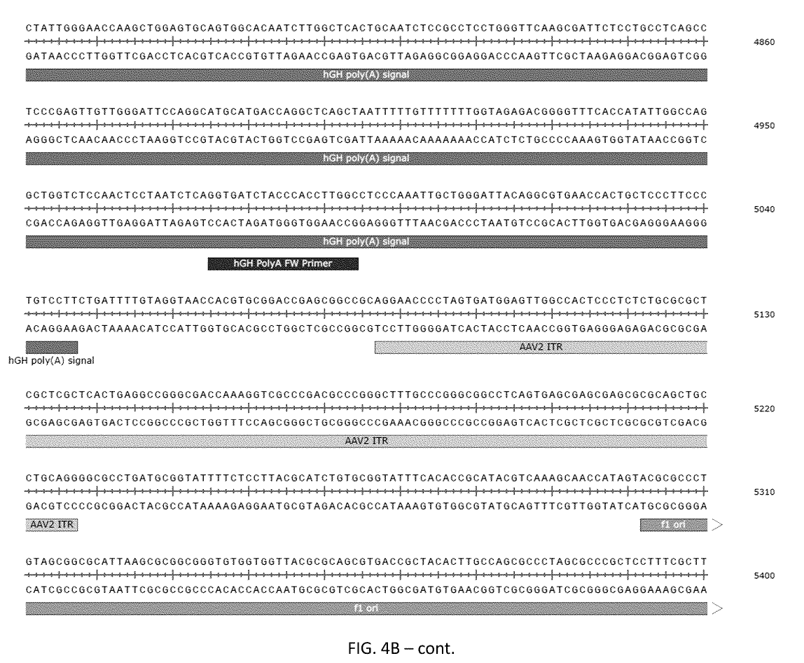

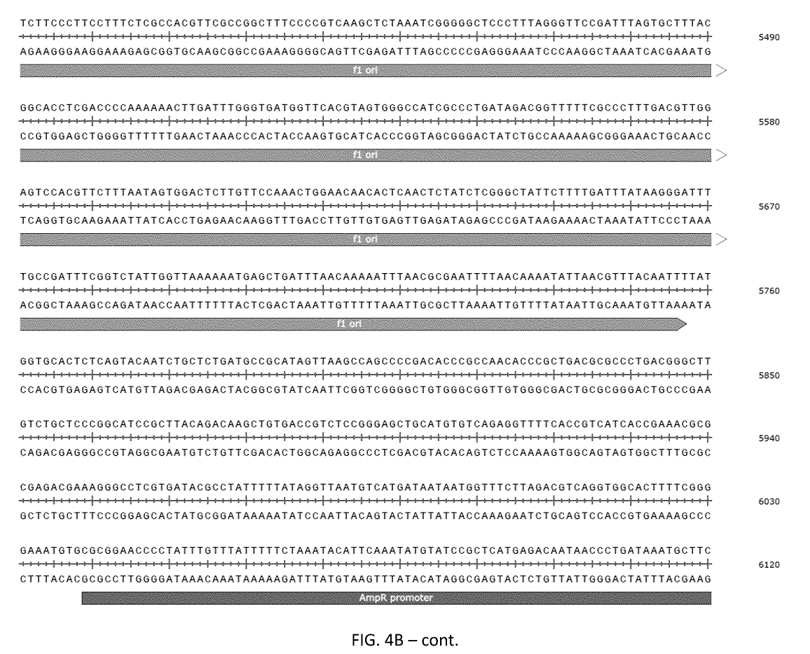

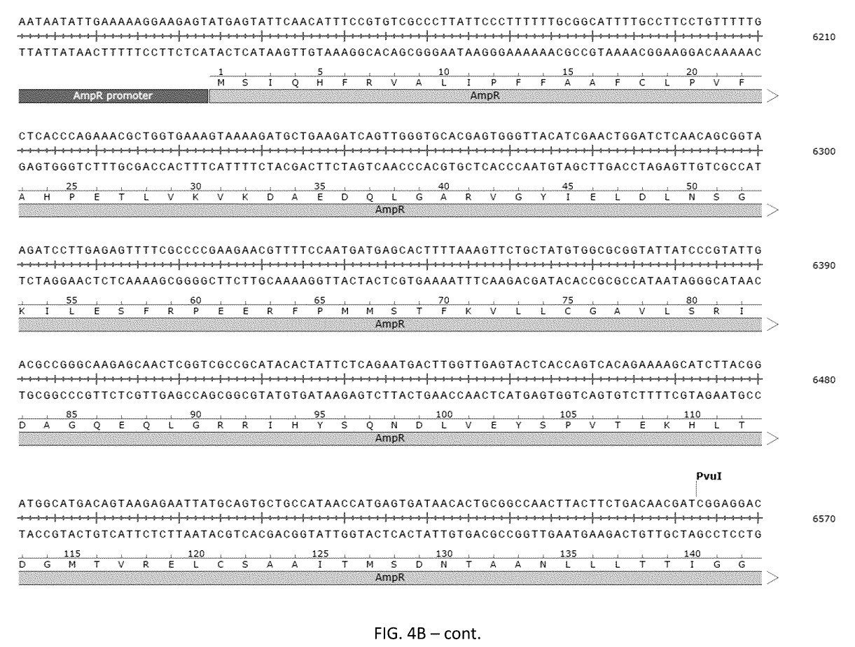

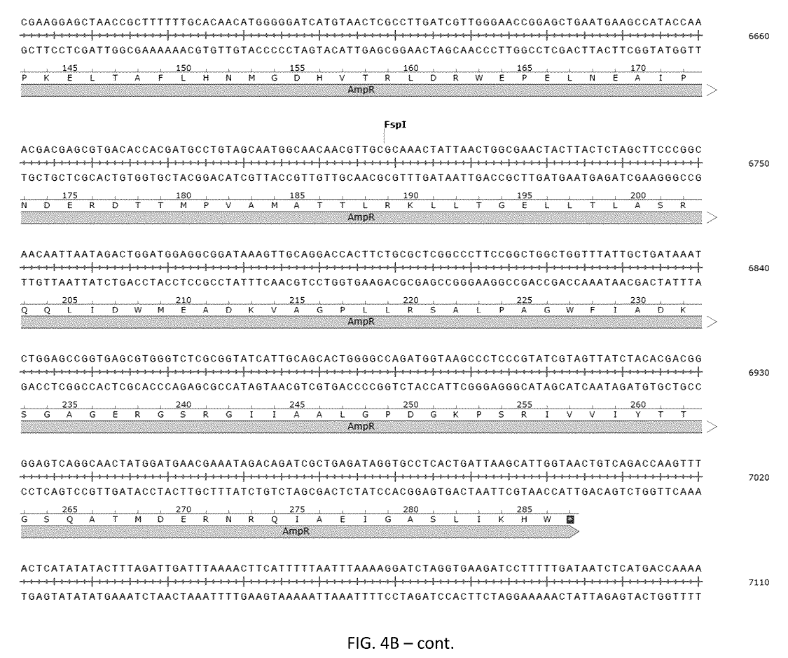

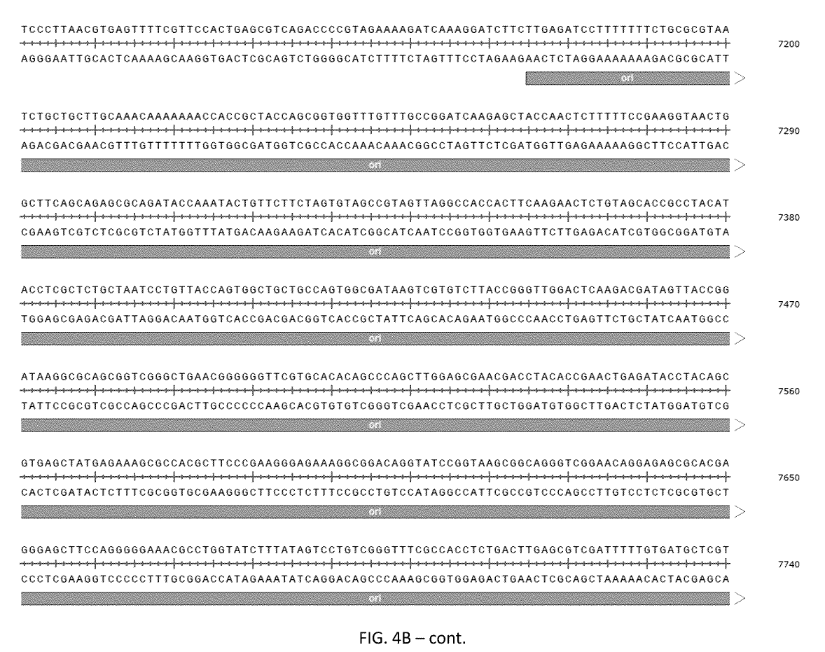

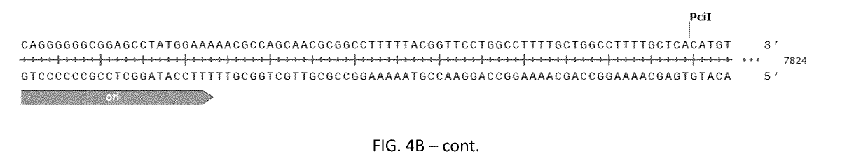

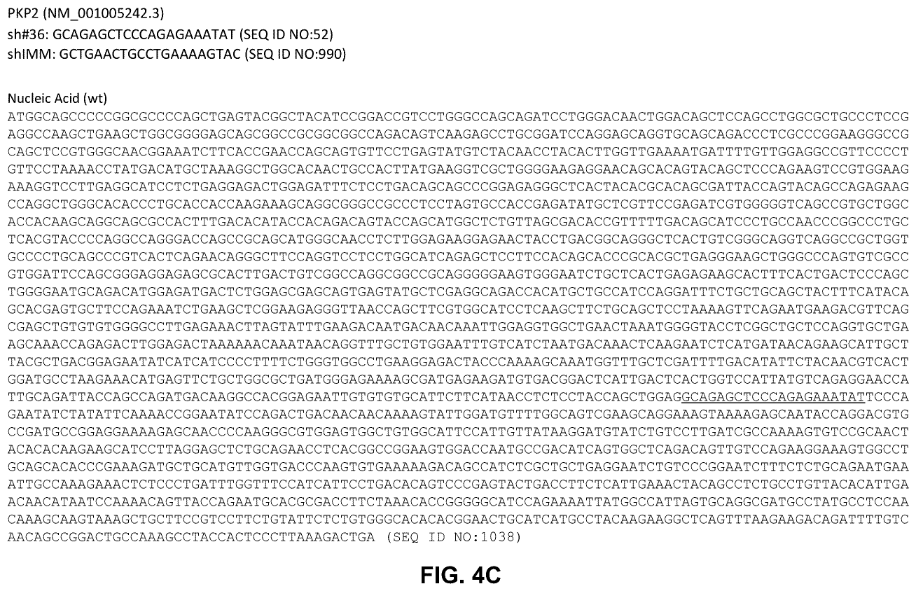

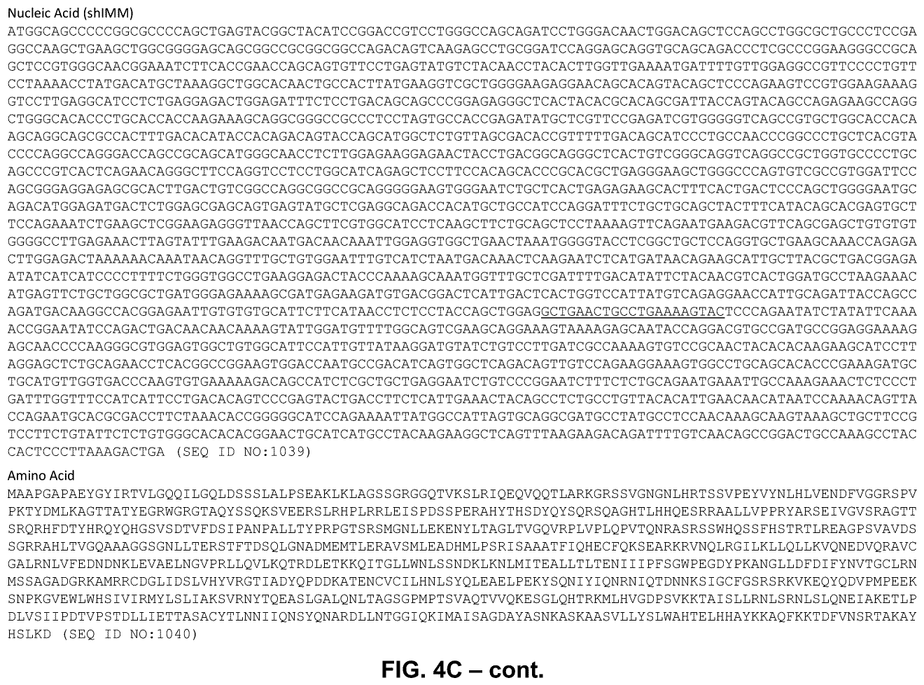

In some cases, the mammal can have ACM, and the gene to be suppressed and replaced can be PKP2 (which encodes plakophilin 2). An example of a PKP2 construct is shown in FIGS. 4A and 4B. An exemplary PKP2 sequence is set forth in NCBI RefSeq accession number NM_001005242 (e.g., version NM_001005242.3; FIG. 4C). A PKP2 polypeptide can, in some cases, have the amino acid sequence set forth in NCBI RefSeq accession number NP_001005242 (e.g., version NP_001005242.2; FIG. 4C).

Examples of shRNA sequences and corresponding shIMM sequences targeted to PKP2 are set forth in TABLE 1I.

TABLE 1I
Representative PKP2 shRNA
and shIMM sequences

| | SEQ | | SEQ |
|---|---|---|---|
| shRNA Sequence | ID | shIMM Sequence | ID |
| GCAGTGTTCCTGAGT | 920 | GTAGCGTACCAGAAT | 921 |
| ATGTCTACAA | | ACGTGTATAA | |
| GCAGTGTTCCTGAGT | 922 | GTAGCGTACCAGAAT | 923 |
| ATGT | | ACGT | |
| GTGTTCCTGAGTATG | 924 | GCGTACCAGAATACG | 925 |
| TCTACA | | TGTATA | |
| GTTCCTGAGTATGTC | 926 | GTACCAGAATACGTG | 927 |
| TACAACCTACACTT | | TATAATCTTCATTT | |
| GTTCCTGAGTATGTC | 928 | GTACCAGAATACGTG | 929 |
| TACA | | TATA | |
| GCTAAAGGCTGGCAC | 930 | GCTTAAAGCAGGGAC | 931 |
| AACT | | TACA | |
| GCACAACTGCCACTT | 932 | GGACTACAGCGACAT | 933 |
| ATGA | | ACGA | |
| GGGAAGAGGAACAGC | 934 | GGGTAGGGGTACTGC | 935 |
| ACAGTA | | TCAATA | |
| GAAGAGGAACAGCAC | 936 | GTAGGGGTACTGCTC | 937 |
| AGTACA | | AATATA | |
| GAAGAGGAACAGCAC | 938 | GTAGGGGTACTGCTC | 939 |
| AGTA | | AATA | |
| CTCTGAGGAGACTGG | 940 | CACTCAGAAGGCTCG | 941 |
| AGATTT | | AAATAT | |
| GAGGAGACTGGAGAT | 942 | CAGAAGGCTCGAAAT | 943 |
| TTCT | | ATCA | |
| GCTCACTACACGCAC | 944 | GCACATTATACCCAT | 945 |
| AGCGATTACCAGTA | | AGTGACTATCAATA | |
| GTACCAGCATGGCTC | 946 | ATATCAACACGGGTC | 947 |
| TGTT | | AGTA | |
| GGCAACCTCTTGGAG | 948 | GGGAATCTGTTAGAA | 949 |
| AAGGAGAACTACCT | | AAAGAAAATTATCT | |
| GGAATGCAGACATGG | 950 | GCAACGCTGATATGG | 951 |
| AGATGACTCT | | AAATGACACT | |
| GGAATGCAGACATGG | 952 | GCAACGCTGATATGG | 953 |
| AGATGA | | AAATGA | |
| GGGCCTTGAGAAACT | 954 | GCGCGTTAAGGAATT | 955 |
| TAGT | | TGGT | |
| GGCCTTGAGAAACTT | 956 | CGCGTTAAGGAATTT | 957 |
| AGTA | | GGTT | |
| GACAACAAATTGGAG | 958 | GATAATAAGTTAGAA | 959 |
| GTGGCTGAACTAAA | | GTCGCAGAGCTTAA | |
| GCTGAAGCAAACCAG | 960 | CCTCAAACAGACGAG | 961 |
| AGACTTGGAGACTA | | GGATTTAGAAACAA | |
| GCTGAAGCAAACCAG | 962 | CCTCAAACAGACGAG | 963 |
| AGACTT | | GGATTT | |
| GAAGCAAACCAGAGA | 964 | CAAACAGACGAGGGA | 965 |
| CTTGGAGACT | | TTTAGAAACA | |
| GCAAACCAGAGACTT | 966 | ACAGACGAGGGATTT | 967 |
| GGAGACTAAA | | AGAAACAAAG | |
| GGATGCCTAAGAAAC | 968 | GGTTGTCTTAGGAAT | 969 |
| ATGAGT | | ATGAGC | |
| GGATGCCTAAGAAAC | 970 | GGTTGTCTTAGGAAT | 971 |
| ATGA | | ATGA | |
| GATGCCTAAGAAACA | 972 | GTTGTCTTAGGAATA | 973 |
| TGAGTT | | TGAGCT | |
| GAGAAGATGTGACGG | 974 | GAGGAGGTGCGATGG | 975 |
| ACTCAT | | TCTGAT | |
| GAAGATGTGACGGAC | 976 | GGAGGTGCGATGGTC | 977 |
| TCAT | | TGAT | |
| GAGGAACCATTGCAG | 978 | GGGGTACGATAGCTG | 979 |
| ATTA | | ACTA | |
| GGAACCATTGCAGAT | 980 | GGTACGATAGCTGAC | 981 |
| TACCAGCCAGATGA | | TATCAACCTGACGA | |
| GAACCATTGCAGATT | 982 | GTACGATAGCTGACT | 983 |
| ACCA | | ATCA | |
| GATGACAAGGCCACG | 984 | GACGATAAAGCGACC | 985 |
| GAGAAT | | GAAAAC | |
| GCATTCTTCATAACC | 986 | GTATACTACACAATC | 987 |
| TCTCCTACCA | | TGTCGTATCA | |
| GCATTCTTCATAACC | 988 | GTATACTACACAATC | 989 |
| TCTCCT | | TGTCGT | |
| GCAGAGCTCCCAGAG | 52 | GCTGAACTGCCTGAA | 990 |
| AAATAT | | AAGTAC | |
| GGCAGTCGAAGCAGG | 991 | GGGAGCCGTAGTAGA | 992 |
| AAAGTA | | AAGGTT | |
| GCAGTCGAAGCAGGA | 993 | GGAGCCGTAGTAGAA | 994 |
| AAGTAA | | AGGTTA | |
| GTCGAAGCAGGAAAG | 995 | GCCGTAGTAGAAAGG | 996 |
| TAAA | | TTAA | |
| GTGGCTGTGGCATTC | 997 | ATGGCTCTGGCACTC | 998 |
| CATTGTTATA | | GATAGTAATT | |
| GGCTGTGGCATTCCA | 999 | GGCTCTGGCACTCGA | 1000 |
| TTGTTA | | TAGTAA | |
| GCTGTGGCATTCCAT | 1001 | GCTCTGGCACTCGAT | 1002 |
| TGTTAT | | AGTAAT | |
| GCTGTGGCATTCCAT | 1003 | GCTCTGGCACTCGAT | 1004 |
| TGTT | | AGTA | |
| GTGGCATTCCATTGT | 1005 | CTGGCACTCGATAGT | 1006 |
| TATA | | AATT | |
| AAGACAGCCATCTCG | 1007 | AAAACTGCGATTTCC | 1008 |
| CTGCTG | | CTCCTC | |
| GCTGAGGAATCTGTC | 1009 | CCTCAGAAACCTCTC | 1010 |
| CCGGAATCTT | | GCGCAACCTA | |
| GAGGAATCTGTCCCG | 1011 | CAGAAACCTCTCGCG | 1012 |
| GAATCTTTCT | | CAACCTATCA | |
| GGAATCTGTCCCGGA | 1013 | GAAACCTCTCGCGCA | 1014 |
| ATCTTT | | ACCTAT | |
| GAATCTGTCCCGGAA | 1015 | AAACCTCTCGCGCAA | 1016 |
| TCTT | | CCTA | |
| GAAGGCTCAGTTTAA | 1017 | AAAAGCACAATTCAA | 1018 |
| GAAGACAGAT | | AAAAACTGAC | |
| AAGGCTCAGTTTAAG | 1019 | AAAGCACAATTCAAA | 1020 |
| AAGACA | | AAAACT | |
| GGCTCAGTTTAAGAA | 1021 | AGCACAATTCAAAAA | 1022 |
| GACAGA | | AACTGA | |
| GGCTCAGTTTAAGAA | 1023 | AGCACAATTCAAAAA | 1024 |
| GACA | | AACT | |
| GCTCAGTTTAAGAAG | 1025 | GCACAATTCAAAAAA | 1026 |
| ACAGAT | | ACTGAC | |
| GGACTGCCAAAGCCT | 1027 | GCACAGCGAAGGCGT | 1028 |
| ACCACTCCCTTAAA | | ATCATTCGCTAAAG | |

In some cases, the mammal can have ACM, and the gene to be suppressed and replaced can be DSG2 (which encodes desmoglein 2). An exemplary DSG2 sequence is set forth in NCBI RefSeq accession number NM_001943 (e.g., version NM_001943.5). A DSG2 polypeptide can, in some cases, have the amino acid sequence set forth in NCBI RefSeq accession number NP_001934 (e.g., version NP_001934.2).

Examples of shRNA sequences and corresponding shIMM sequences targeted to DSG2 are set forth in TABLE 1J.

TABLE 1J
Representative DSG2 shRNA
and shIMM sequences

| | SEQ | | SEQ |
|---|---|---|---|
| shRNA Sequence | ID | shIMM Sequence | ID |
| GTGGACTTCACTTAC | 1048 | GCGGTCTACATTTGC | 1049 |
| AGGT | | AAGT | |
| GGGAGGGAGAGGATC | 1050 | GCGAAGGTGAAGACC | 1051 |
| TGTCCAAGAAGAAT | | TCTCGAAAAAAAAC | |
| GAGGGAGAGGATCTG | 1052 | GAAGGTGAAGACCTC | 1053 |
| TCCAAGAAGA | | TCGAAAAAAA | |
| GGGAGAGGATCTGTC | 1054 | AGGTGAAGACCTCTC | 1055 |
| CAAGAAGAAT | | GAAAAAAAAC | |
| GAGGATCTGTCCAAG | 1056 | GAAGACCTCTCGAAA | 1057 |
| AAGAAT | | AAAAAC | |
| GAGGATCTGTCCAAG | 1058 | GAAGACCTCTCGAAA | 1059 |
| AAGA | | AAAA | |
| GGATCTGTCCAAGAA | 1060 | AGACCTCTCGAAAAA | 1061 |
| GAAT | | AAAC | |
| GTCCAAGAAGAATCC | 1062 | CTCGAAAAAAAACCC | 1063 |
| AATTGCCAAGATA | | TATAGCGAAAATT | |
| GAATCCAATTGCCAA | 1064 | AAACCCTATAGCGAA | 1065 |
| GATA | | AATT | |
| GCAGAAGAAAGAGGA | 1066 | GCTGAGGAGAGGGGT | 1067 |
| CTCAAA | | CTGAAG | |
| GCAGAAGAAAGAGGA | 1068 | GCTGAGGAGAGGGGT | 1069 |
| CTCA | | CTGA | |
| GGAGAACTGAATGTT | 1070 | GGTGAGCTCAACGTA | 1071 |
| ACCA | | ACGA | |
| GGATGCAAGAGGAAA | 1072 | AGACGCTAGGGGTAA | 1073 |
| CAATGT | | TAACGT | |
| GGATGCAAGAGGAAA | 1074 | AGACGCTAGGGGTAA | 1075 |
| CAAT | | TAAC | |
| GATGCAAGAGGAAAC | 1076 | GACGCTAGGGGTAAT | 1077 |
| AATGTAGAGA | | AACGTTGAAA | |
| GATGCAAGAGGAAAC | 1078 | GACGCTAGGGGTAAT | 1079 |
| AATGTA | | AACGTT | |
| GAGGAAACAATGTAG | 1080 | GGGGTAATAACGTTG | 1081 |
| AGAA | | AAAA | |
| GTTCTACCTAAATAA | 1082 | CTTTTATCTTAACAA | 1083 |
| AGATACAGGAGAGA | | GGACACTGGTGAAA | |
| GTGTTACCTTGGACA | 1084 | GCGTAACGTTAGATA | 1085 |
| GAGA | | GGGA | |
| GATGCAGATGAAATA | 1086 | GACGCTGACGAGATT | 1087 |
| GGTTCT | | GGATCA | |
| GATGCAGATGAAATA | 1088 | GACGCTGACGAGATT | 1089 |
| GGTT | | GGAT | |
| GATGAAATAGGTTCT | 1090 | GACGAGATTGGATCA | 1091 |
| GATA | | GACA | |
| GGAGGTTATTTCCAC | 1092 | GGTGGATACTTTCAT | 1093 |
| ATAGAA | | ATTGAG | |
| GAGGTTATTTCCACA | 1094 | GTGGATACTTTCATA | 1095 |
| TAGA | | TTGA | |
| GAAACAGATGCTCAA | 1096 | GAGACTGACGCACAG | 1097 |
| ACTA | | ACAA | |
| GTTAGCGAGAGCATG | 1098 | GTAAGTGAAAGTATG | 1099 |
| GATAGA | | GACAGG | |
| GATCAAGCAAAGGCC | 1100 | GGTCTAGTAAGGGGC | 1101 |
| AAATAA | | AGATTA | |
| GTGGCCATATCAGAA | 1102 | GTCGCGATTTCTGAG | 1103 |
| GATTATCCTAGAAA | | GACTACCCAAGGAA | |
| GTGGCCATATCAGAA | 1104 | GTCGCGATTTCTGAG | 1105 |
| GATTAT | | GACTAC | |
| GTGGCCATATCAGAA | 1106 | GTCGCGATTTCTGAG | 1107 |
| GATT | | GACT | |
| GGCCATATCAGAAGA | 1108 | CGCGATTTCTGAGGA | 1109 |
| TTATCCTAGA | | CTACCCAAGG | |
| GGCCATATCAGAAGA | 1110 | CGCGATTTCTGAGGA | 1111 |
| TTAT | | CTAC | |
| GCCATATCAGAAGAT | 1112 | GCGATTTCTGAGGAC | 1113 |
| TATCCTAGAA | | TACCCAAGGA | |
| GCCATATCAGAAGAT | 1114 | GCGATTTCTGAGGAC | 1115 |
| TATCCT | | TACCCA | |
| GGCACAGTCCTTATC | 1116 | GGGACTGTGCTAATT | 1117 |
| AATGTT | | AACGTA | |
| GCACAGTCCTTATCA | 1118 | GGACTGTGCTAATTA | 1119 |
| ATGT | | ACGT | |
| GGATGGACACCCAAA | 1120 | CGACGGTCATCCTAA | 1121 |
| CAGT | | TAGC | |
| GCTGCTGCAACAAAG | 1122 | CCTCCTCCAGCAGAG | 1123 |
| TGAGAA | | CGAAAA | |
| GCTGCAACAAAGTGA | 1124 | CCTCCAGCAGAGCGA | 1125 |
| GAAA | | AAAG | |
| GGGAAGCACAGCATG | 1126 | GAGAGGCTCAACACG | 1127 |
| ACTCCTATGT | | ATTCGTACGT | |
| GGAAGCACAGCATGA | 1128 | AGAGGCTCAACACGA | 1129 |
| CTCCTA | | TTCGTA | |
| GAAGCACAGCATGAC | 1130 | GAGGCTCAACACGAT | 1131 |
| TCCTAT | | TCGTAC | |
| GAAGCACAGCATGAC | 1132 | GAGGCTCAACACGAT | 1133 |
| TCCT | | TCGT | |
| GCTGCATCCTTGGAA | 1134 | GCTCCACCCATGGAA | 1135 |
| TAATGA | | CAACGA | |
| GCTGCATCCTTGGAA | 1136 | GCTCCACCCATGGAA | 1137 |
| TAAT | | CAAC | |
| GCATCCTTGGAATAA | 1138 | CCACCCATGGAACAA | 1139 |
| TGAA | | CGAG | |
| GAGCACCACCTGAAG | 1140 | GTGCTCCTCCAGAGG | 1141 |
| ACAA | | ATAA | |
| GCCATCATTTCTGCC | 1142 | CCCTTCTTTCCTCCC | 1143 |
| AGTGGATCAA | | TGTCGACCAG | |
| GCCATCATTTCTGCC | 1144 | CCCTTCTTTCCTCCC | 1145 |
| AGTGGA | | TGTCGA | |
| GGGCAGTCTAGTAGG | 1146 | CGGGAGCCTTGTTGG | 1147 |
| AAGAAA | | TAGGAA | |
| GGCAGTCTAGTAGGA | 1148 | GGGAGCCTTGTTGGT | 1149 |
| AGAAAT | | AGGAAC | |
| GCAGTCTAGTAGGAA | 1150 | GGAGCCTTGTTGGTA | 1151 |
| GAAATGGAGTAGGA | | GGAACGGTGTTGGT | |
| GCAGTCTAGTAGGAA | 1152 | GGAGCCTTGTTGGTA | 1153 |
| GAAA | | GGAA | |
| GAAATGGAGTAGGAG | 1154 | GGAACGGTGTTGGTG | 1155 |
| GTAT | | GAAT | |
| GCCAAGGAAGCCACG | 1156 | GCGAAAGAGGCGACC | 1157 |
| ATGAAA | | ATGAAG | |
| GAAGCCACGATGAAA | 1158 | GAGGCGACCATGAAG | 1159 |
| GGAAGTAGCT | | GGTAGCAGTT | |
| GCTGCTGTTGCACTG | 1160 | GCAGCAGTAGCTCTC | 1161 |
| AACGAAGAAT | | AATGAGGAGT | |
| GTTGCACTGAACGAA | 1162 | GTAGCTCTCAATGAG | 1163 |
| GAAT | | GAGT | |
| GAATCGCTGAATGCT | 1164 | GAGTCCCTCAACGCA | 1165 |
| TCTATT | | TCAATA | |
| GGAAATAGTCACTGA | 1166 | AGAGATTGTGACAGA | 1167 |
| AAGA | | GAGG | |
| GAAATAGTCACTGAA | 1168 | GAGATTGTGACAGAG | 1169 |
| AGATCT | | AGGTCA | |
| GAAATAGTCACTGAA | 1170 | GAGATTGTGACAGAG | 1171 |
| AGAT | | AGGT | |
| GAAATGTGATAGCAA | 1172 | GGAACGTCATTGCTA | 1173 |
| CAGA | | CTGA | |
| GATCGAATCCTCTGG | 1174 | GTTCCAACCCACTCG | 1175 |
| AAGGCACTCA | | AGGGGACACA | |
| GAATCCTCTGGAAGG | 1176 | CAACCCACTCGAGGG | 1177 |
| CACTCA | | GACACA | |
| AAGGCACTCAGCATC | 1178 | AGGGGACACAACACC | 1179 |
| TTCAAG | | TACAGG | |

In some cases, the mammal can have ACM, DCM, left ventricular non-compaction cardiomyopathy (LVNC), or skeletal myopathy, and the gene to be suppressed and replaced can be DES (which encodes desmin). An exemplary DES sequence is set forth in NCBI RefSeq accession number NM_001927 (e.g., version NM_001927.4). A DES polypeptide can, in some cases, have the amino acid sequence set forth in NCBI RefSeq accession number NP_001918 (e.g., version NP_001918.3).

Examples of shRNA sequences and corresponding shIMM sequences targeted to DES are set forth in TABLE 1K.

TABLE 1K
Representative DES shRNA and
shIMM sequences

| shRNA | SEQ | shIMM | SEQ |
|---|---|---|---|
| Sequence | ID | Sequence | ID |
| GTGAACCAGG | 1180 | GTCAATCAAG | 1181 |
| AGTTTCTGA | | AATTCCTCA | |
| GAGCTCAATG | 1182 | GAACTGAACG | 1183 |
| ACCGCTTCGC | | ATCGGTTTGC | |
| CAACTACAT | | GAATTATAT | |
| GTTGAAGGAA | 1184 | ATTAAAAGAG | 1185 |
| GAAGCAGAGA | | GAGGCTGAAA | |
| ACAAT | | ATAAC | |
| GTTGAAGGAA | 1186 | ATTAAAAGAG | 1187 |
| GAAGCAGAGA | | GAGGCTGAAA | |
| A | | A | |
| GAAGGAAGAA | 1188 | AAAAGAGGAG | 1189 |
| GCAGAGAACA | | GCTGAAAATA | |
| A | | A | |
| GGAAGAAGCA | 1190 | AGAGGAGGCT | 1191 |
| GAGAACAATT | | GAAAATAACT | |
| T | | T | |
| GGAAGAAGCA | 1192 | AGAGGAGGCT | 1193 |
| GAGAACAAT | | GAAAATAAC | |
| GGAGCGCAGA | 1194 | CGAACGGAGG | 1195 |
| ATTGAATCTC | | ATAGAGTCAC | |
| T | | T | |
| GGAGCGCAGA | 1196 | CGAACGGAGG | 1197 |
| ATTGAATCT | | ATAGAGTCA | |
| GAAAGTGCAT | 1198 | AAAGGTCCAC | 1199 |
| GAAGAGGAGA | | GAGGAAGAAA | |
| T | | T | |
| GAACATTTCT | 1200 | AAATATATCA | 1201 |
| GAAGCTGAGG | | GAGGCAGAAG | |
| AGTGGTACA | | AATGGTATA | |
| GAAGCTGAGG | 1202 | GAGGCAGAAG | 1203 |
| AGTGGTACA | | AATGGTATA | |
| GCTGAGGAGT | 1204 | GCAGAAGAAT | 1205 |
| GGTACAAGT | | GGTATAAAT | |
| GGAATACCGA | 1206 | GGAGTATCGT | 1207 |
| CACCAGATCC | | CATCAAATTC | |
| AGTCCTACA | | AATCGTATA | |
| GACACCAGAT | 1208 | GTCATCAAAT | 1209 |
| CCAGTCCTAC | | TCAATCGTAT | |
| A | | A | |
| GTCCTACACC | 1210 | ATCGTATACG | 1211 |
| TGCGAGATTG | | TGTGAAATAG | |
| A | | A | |
| GCACTAACGA | 1212 | GGACAAATGA | 1213 |
| TTCCCTGATG | | CTCGCTCATG | |
| A | | A | |
| GTGGCTACCA | 1214 | GCGGGTATCA | 1215 |
| GGACAACAT | | AGATAATAT | |
| GACCTACTCT | 1216 | AACGTATTCA | 1217 |
| GCCCTCAACT | | GCGCTGAATT | |
| T | | T | |
| GGTTCTGAGG | 1218 | GGATCAGAAG | 1219 |
| TCCATACCA | | TGCACACGA | |
| GAGGTCCATA | 1220 | GAAGTGCACA | 1221 |
| CCAAGAAGAC | | CGAAAAAAAC | |
| GGTGATGAT | | CGTCATGAT | |
| GAGGTCCATA | 1222 | GAAGTGCACA | 1223 |
| CCAAGAAGA | | CGAAAAAAA | |
| GTCCATACCA | 1224 | GTGCACACGA | 1225 |
| AGAAGACGGT | | AAAAAACCGT | |
| GATGA | | CATGA | |

In some cases, the mammal can have Andersen-Tawil syndrome (ATS) or CPVT, and the gene to be suppressed and replaced can be KCNJ2 (which encodes potassium inwardly rectifying channel subfamily J member 2). An exemplary KCNJ2 sequence is set forth in NCBI RefSeq accession number NM_000891 (e.g., version NM_000891.3). A KCNJ2 polypeptide can, in some cases, have the amino acid sequence set forth in NCBI RefSeq accession number NP_000882 (e.g., version NP_000882.1).

Examples of shRNA sequences and corresponding shIMM sequences targeted to KCNJ2 are set forth in TABLE 1L.

TABLE 1L
Representative KCNJ2 shRNA
and shIMM sequences

| | SEQ | | SEQ |
|---|---|---|---|
| shRNA Sequence | ID | shIMM Sequence | ID |
| GAACCAACCG | 1226 | GTACGAATCG | 1227 |
| CTACAGCATC | | GTATAGTATT | |
| GTCTCTTCA | | GTGTCATCT | |
| GGGAAGAGTA | 1228 | GGCAAAAGCA | 1229 |
| AAGTCCACAC | | AGGTGCATAC | |
| CCGACAACA | | GCGTCAGCA | |
| GAAGAGTAAA | 1230 | CAAAAGCAAG | 1231 |
| GTCCACACCC | | GTGCATACGC | |
| GACAACAGT | | GTCAGCAAT | |
| GCACAGCTCC | 1232 | GCTCAACTGC | 1233 |
| TCAAATCCAG | | TGAAGTCGAG | |
| AATTACTT | | GATAACAT | |
| GGATCAAATA | 1234 | CGACCAGATT | 1235 |
| GACATCAAT | | GATATTAAC | |
| GGTGTCCCCA | 1236 | CGTCTCGCCT | 1237 |
| ATCACTATA | | ATTACAATT | |
| GAAATAGATG | 1238 | GAGATTGACG | 1239 |
| AAGACAGTCC | | AGGATAGCCC | |
| TTTATATGA | | ATTGTACGA | |
| GATGAAGACA | 1240 | GACGAGGATA | 1241 |
| GTCCTTTATA | | GCCCATTGTA | |
| T | | C | |
| GAAGACAGTC | 1242 | GAGGATAGCC | 1243 |
| CTTTATATGA | | CATTGTACGA | |
| T | | C | |
| GTGCCGTAGC | 1244 | ATGTCGAAGT | 1245 |
| TCTTATCTAG | | TCATACCTTG | |
| CAAATGAAA | | CTAACGAGA | |
| GAAGAGAAGC | 1246 | GAGGAAAAAC | 1247 |
| ACTACTACA | | ATTATTATA | |
| AAGCACTACT | 1248 | AAACATTATT | 1249 |
| ACAAAGTGGA | | ATAAGGTCGA | |
| C | | T | |
| GAGGAAGACG | 1250 | GAAGAGGATG | 1251 |
| ACAGTGAAA | | ATAGCGAGA | |
| GCGAGAGTCG | 1252 | CCGTGAATCC | 1253 |
| GAGATATGA | | GAAATTTGA | |

In some cases, the mammal can have CPVT, and the gene to be suppressed and replaced can be CASQ2 (which encodes calsequestrin 2). An exemplary CASQ2 sequence is set forth in NCBI RefSeq accession number NM_001232 (e.g., version NM_001232). A CASQ2 polypeptide can, in some cases, have the amino acid sequence set forth in NCBI RefSeq accession number NP_001223.2 (e.g., version NP_001223.2).

Examples of shRNA sequences and corresponding shIMM sequences targeted to CASQ2 are set forth in TABLE 1M.

TABLE 1M
Representative CASQ2 shRNA
and shIMM sequences

| | SEQ | | SEQ |
|---|---|---|---|
| shRNA Sequence | ID | shIMM Sequence | ID |
| GGGCTTAATT | 1254 | GGCCTAAACT | 1255 |
| TCCCCACATA | | TTCCGACTTA | |
| T | | C | |
| GCTTAATTTC | 1256 | CCTAAACTTT | 1257 |
| CCCACATATG | | CCGACTTACG | |
| A | | A | |
| GCTTAATTTC | 1258 | CCTAAACTTT | 1259 |
| CCCACATAT | | CCGACTTAC | |
| GATGGGAAGG | 1260 | GACGGCAAAG | 1261 |
| ACCGAGTGGT | | ATCGTGTCGT | |
| AAGTCTTT | | TAGCCTAT | |
| GGGAAGGACC | 1262 | GGCAAAGATC | 1263 |
| GAGTGGTAAG | | GTGTCGTTAG | |
| TCTTT | | CCTAT | |
| GAAGGACCGA | 1264 | CAAAGATCGT | 1265 |
| GTGGTAAGTC | | GTCGTTAGCC | |
| T | | T | |
| GAAGGACCGA | 1266 | CAAAGATCGT | 1267 |
| GTGGTAAGT | | GTCGTTAGC | |
| GGACCGAGTG | 1268 | AGATCGTGTC | 1269 |
| GTAAGTCTT | | GTTAGCCTA | |
| GACCGAGTGG | 1270 | GATCGTGTCG | 1271 |
| TAAGTCTTT | | TTAGCCTAT | |
| GGCCCAGGTC | 1272 | CGCGCAAGTG | 1273 |
| CTTGAACATA | | CTAGAGCACA | |
| A | | A | |
| GCCCAGGTCC | 1274 | GCGCAAGTGC | 1275 |
| TTGAACATA | | TAGAGCACA | |
| GTCCTTGAAC | 1276 | GTGCTAGAGC | 1277 |
| ATAAAGCTAT | | ACAAGGCAAT | |
| A | | T | |
| GGTGGATGCC | 1278 | GGTCGACGCG | 1279 |
| AAGAAAGAA | | AAAAAGGAG | |
| GATGAAGAAG | 1280 | GACGAGGAGG | 1281 |
| GAAGCCTGTA | | GTAGTCTCTA | |
| T | | C | |
| GAAGAAGGAA | 1282 | GAGGAGGGTA | 1283 |
| GCCTGTATAT | | GTCTCTACAT | |
| T | | A | |
| GAAGAAGGAA | 1284 | GAGGAGGGTA | 1285 |
| GCCTGTATA | | GTCTCTACA | |
| AAGGAAGCCT | 1286 | AGGGTAGTCT | 1287 |
| GTATATTCTT | | CTACATACTA | |
| A | | A | |
| GGAAGCCTGT | 1288 | GGTAGTCTCT | 1289 |
| ATATTCTTA | | ACATACTAA | |
| GAAGCCTGTA | 1290 | GTAGTCTCTA | 1291 |
| TATTCTTAA | | CATACTAAA | |
| GGTGATCGCA | 1292 | GGAGACCGGA | 1293 |
| CAATAGAGT | | CTATTGAAT | |
| GGTGGAGTTC | 1294 | AGTCGAATTT | 1295 |
| CTCTTGGATC | | CTGTTAGACC | |
| TAATT | | TTATA | |
| GTGGAGTTCC | 1296 | GTCGAATTTC | 1297 |
| TCTTGGATCT | | TGTTAGACCT | |
| A | | T | |
| GGAGTTCCTC | 1298 | CGAATTTCTG | 1299 |
| TTGGATCTAA | | TTAGACCTTA | |
| T | | T | |
| GGAGTTCCTC | 1300 | CGAATTTCTG | 1301 |
| TTGGATCTA | | TTAGACCTT | |
| GAGTTCCTCT | 1302 | GAATTTCTGT | 1303 |
| TGGATCTAAT | | TAGACCTTAT | |
| T | | A | |
| GAGTTCCTCT | 1304 | GAATTTCTGT | 1305 |
| TGGATCTAA | | TAGACCTTA | |
| GTTCCTCTTG | 1306 | ATTTCTGTTA | 1307 |
| GATCTAATTG | | GACCTTATAG | |
| A | | A | |
| GTTCCTCTTG | 1308 | ATTTCTGTTA | 1309 |
| GATCTAATT | | GACCTTATA | |
| GAAGACCCAG | 1310 | GAGGATCCTG | 1311 |
| TGGAGATCA | | TCGAAATTA | |
| GACCCAGTGG | 1312 | GATCCTGTCG | 1313 |
| AGATCATCA | | AAATTATTA | |
| GCCTTACATC | 1314 | ACCATATATT | 1315 |
| AAATTCTTT | | AAGTTTTTC | |
| GGTTGCAAAG | 1316 | CGTAGCTAAA | 1317 |
| AAATTATCT | | AAGTTGTCA | |
| GAAGATGAAT | 1318 | AAAAATGAAC | 1319 |
| GAGGTTGACT | | GAAGTAGATT | |
| T | | T | |
| GATGAATGAG | 1320 | AATGAACGAA | 1321 |
| GTTGACTTCT | | GTAGATTTTT | |
| A | | A | |
| GAATGAGGTT | 1322 | GAACGAAGTA | 1323 |
| GACTTCTAT | | GATTTTTAC | |
| GAGCCCATTG | 1324 | GAACCGATAG | 1325 |
| CCATCCCCAA | | CGATTCCGAA | |
| CAAACCTTA | | TAAGCCATA | |
| GCCCATTGCC | 1326 | ACCGATAGCG | 1327 |
| ATCCCCAACA | | ATTCCGAATA | |
| AACCTTACA | | AGCCATATA | |
| GCAGAGAAGA | 1328 | GCTGAAAAAA | 1329 |
| GTGATCCAGA | | GCGACCCTGA | |
| TGGCTACGA | | CGGGTATGA | |
| GACAATACTG | 1330 | GATAACACAG | 1331 |
| ACAACCCCGA | | ATAATCCGGA | |
| TCTGA | | CCTCA | |
| GTTGCCTACT | 1332 | GTAGCGTATT | 1333 |
| GGGAGAAGAC | | GGGAAAAAAC | |
| TTTCAAGAT | | ATTTAAAAT | |
| GTTGCCTACT | 1334 | GTAGCGTATT | 1335 |
| GGGAGAAGAC | | GGGAAAAAAC | |
| T | | A | |
| GTTGCCTACT | 1336 | GTAGCGTATT | 1337 |
| GGGAGAAGA | | GGGAAAAAA | |
| GCCTACTGGG | 1338 | GCGTATTGGG | 1339 |
| AGAAGACTTT | | AAAAAACATT | |
| CAAGA | | TAAAA | |
| GCCTACTGGG | 1340 | GCGTATTGGG | 1341 |
| AGAAGACTT | | AAAAAACAT | |
| GGGAGAAGAC | 1342 | GGGAAAAAAC | 1343 |
| TTTCAAGAT | | ATTTAAAAT | |
| GGAGAAGACT | 1344 | GGAAAAAACA | 1345 |
| TTCAAGATT | | TTTAAAATA | |
| GGAAAGATAA | 1346 | GGTAAAATTA | 1347 |
| ACACTGAAGA | | ATACAGAGGA | |
| T | | C | |
| GGAAAGATAA | 1348 | GGTAAAATTA | 1349 |
| ACACTGAAGA | | ATACAGAGGA | |
| TGATGATGA | | CGACGACGA | |
| GAAAGATAAA | 1350 | GTAAAATTAA | 1351 |
| CACTGAAGAT | | TACAGAGGAC | |
| GATGA | | GACGA | |
| GAAAGATAAA | 1352 | GTAAAATTAA | 1353 |
| CACTGAAGA | | TACAGAGGA | |
| GAAGAGGATA | 1354 | GAGGAAGACA | 1355 |
| ATGATGACAG | | ACGACGATAG | |
| T | | C | |
| GAGGATAATG | 1356 | GAAGACAACG | 1357 |
| ATGACAGTGA | | ACGATAGCGA | |
| T | | C | |
| GGATAATGAT | 1358 | AGACAACGAC | 1359 |
| GACAGTGAT | | GATAGCGAC | |

In some cases, the mammal can have DCM, and the gene to be suppressed and replaced can be LMNA (which encodes lamin A/C). An exemplary LMNA sequence is set forth in NCBI RefSeq accession number NM_170707 (e.g., version NM_170707.4). A LMNA polypeptide can, in some cases, have the amino acid sequence set forth in NCBI RefSeq accession number NP_733821 (e.g., version NP_733821.1).

Examples of shRNA sequences and corresponding shIMM sequences targeted to LMNA are set forth in TABLE 1N.

TABLE 1N
Representative LMNA shRNA and
shIMM sequences

| | SEQ | | SEQ |
|---|---|---|---|
| shRNA Sequence | ID | shIMM Sequence | ID |
| GAGCTCAATG | 1360 | GAACTGAACG | 1361 |
| ATCGCTTGGC | | ACCGGTTAGC | |
| GGTCTACAT | | CGTGTATAT | |
| GGAGCTGAGC | 1362 | CGAACTCAGT | 1363 |
| AAAGTGCGTG | | AAGGTCCGAG | |
| AGGAGTTTA | | AAGAATTCA | |
| GAGCTGAGCA | 1364 | GAACTCAGTA | 1365 |
| AAGTGCGTGA | | AGGTCCGAGA | |
| GGAGTTTAA | | AGAATTCAA | |
| GCTGAGCAAA | 1366 | ACTCAGTAAG | 1367 |
| GTGCGTGAGG | | GTCCGAGAAG | |
| AGTTT | | AATTC | |
| GAGCAAAGTG | 1368 | CAGTAAGGTC | 1369 |
| CGTGAGGAGT | | CGAGAAGAAT | |
| T | | T | |
| GCAAAGTGCG | 1370 | GTAAGGTCCG | 1371 |
| TGAGGAGTTT | | AGAAGAATTC | |
| A | | A | |
| GCAAAGTGCG | 1372 | GTAAGGTCCG | 1373 |
| TGAGGAGTT | | AGAAGAATT | |
| GCAATACCAA | 1374 | GGAACACGAA | 1375 |
| GAAGGAGGGT | | AAAAGAAGGA | |
| GACCT | | GATCT | |
| GCATGAGGAC | 1376 | ACACGAAGAT | 1377 |
| CAGGTGGAGC | | CAAGTCGAAC | |
| AGTATAAGA | | AATACAAAA | |
| GAGGACCAGG | 1378 | GAAGATCAAG | 1379 |
| TGGAGCAGTA | | TCGAACAATA | |
| TAAGA | | CAAAA | |
| GGACCAGGTG | 1380 | AGATCAAGTC | 1381 |
| GAGCAGTATA | | GAACAATACA | |
| A | | A | |
| GACCAGGTGG | 1382 | GATCAAGTCG | 1383 |
| AGCAGTATA | | AACAATACA | |
| AAGCTGGACA | 1384 | AAACTCGATA | 1385 |
| ATGCCAGGCA | | ACGCGAGACA | |
| G | | A | |
| GACCAGTCCA | 1386 | GATCAATCGA | 1387 |
| TGGGCAATTG | | TGGGGAACTG | |
| GCAGATCAA | | GCAAATTAA | |
| GGCAGATCAA | 1388 | GGCAAATTAA | 1389 |
| GCGCCAGAAT | | ACGGCAAAAC | |
| GGAGATGA | | GGTGACGA | |
| GCGCCAGAAT | 1390 | ACGGCAAAAC | 1391 |
| GGAGATGAT | | GGTGACGAC | |
| GATGATCCCT | 1392 | GACGACCCGT | 1393 |
| TGCTGACTT | | TACTCACAT | |
| GGATGAGGAT | 1394 | AGACGAAGAC | 1395 |
| GGAGATGACC | | GGTGACGATC | |
| T | | T | |

In some cases, the mammal can have DCM, and the gene to be suppressed and replaced can be TPM1 (which encodes tropomyosin 1). An exemplary TPM1 sequence is set forth in NCBI RefSeq accession number NM_001018005 (e.g., version NM_001018005.2). A TPM1 polypeptide can, in some cases, have the amino acid sequence set forth in NCBI RefSeq accession number NP_001018005 (e.g., version NP_001018005.1).

Examples of shRNA sequences and corresponding shIMM sequences targeted to TPM1 are set forth in TABLE 10.

TABLE 10
Representative TPM1 shRNA and
shIMM sequences

| | SEQ | | SEQ |
|---|---|---|---|
| shRNA Sequence | ID | shIMM Sequence | ID |
| GAAGACAGGA | 1396 | GAGGATAGAA | 1397 |
| GCAAGCAGCT | | GTAAACAACT | |
| GGAAGATGA | | CGAGGACGA | |
| AAGCTGAGAA | 1398 | AGGCAGAAAA | 1399 |
| GGCAGCAGAT | | AGCTGCTGAC | |
| G | | G | |
| GAGAAGGCAG | 1400 | GAAAAAGCTG | 1401 |
| CAGATGAGAG | | CTGACGAAAG | |
| TGAGA | | CGAAA | |
| GAGAAGGCAG | 1402 | GAAAAAGCTG | 1403 |
| CAGATGAGA | | CTGACGAAA | |
| GAAGGCAGCA | 1404 | AAAAGCTGCT | 1405 |
| GATGAGAGTG | | GACGAAAGCG | |
| A | | A | |
| GCAGCAGATG | 1406 | GCTGCTGACG | 1407 |
| AGAGTGAGA | | AAAGCGAAA | |
| GCAGATGAGA | 1408 | GCTGACGAAA | 1409 |
| GTGAGAGAGG | | GCGAAAGGGG | |
| CATGAAAGT | | GATGAAGGT | |
| GCAAATGTGC | 1410 | GGAAGTGCGC | 1411 |
| CGAGCTTGAA | | GGAACTAGAG | |
| GAAGAAT | | GAGGAGT | |
| GAAGGAAGAC | 1412 | AAAAGAGGAT | 1413 |
| AGATATGAGG | | AGGTACGAAG | |
| AAGAGATCA | | AGGAAATTA | |
| AAGACGAGCT | 1414 | AGGATGAACT | 1415 |
| GTACGCTCAG | | CTATGCACAA | |
| A | | A | |

In some cases, the mammal can have DCM or ACM, and the gene to be suppressed and replaced can be PLN (which encodes phospholamban). An exemplary PLN sequence is set forth in NCBI RefSeq accession number NM_002667 (e.g., version NM_002667.5). A PLN polypeptide can, in some cases, have the amino acid sequence set forth in NCBI RefSeq accession number NP_002658 (e.g., version NP_002658.1).

Examples of shRNA sequences and corresponding shIMM sequences targeted to PLN are set forth in TABLE 1P.

TABLE 1P
Representative PLN shRNA
and shIMM sequences

| | SEQ | | SEQ |
|---|---|---|---|
| shRNA Sequence | ID | shIMM Sequence | ID |
| AAGAGCCTCA | 1416 | GAGGGCGTCT | 1417 |
| ACCATTGAAA | | ACGATAGAGA | |
| T | | T | |
| AACCATTGAA | 1418 | TACGATAGAG | 1419 |
| ATGCCTCAAC | | ATGCCACAGC | |
| A | | A | |
| AATGCCTCAA | 1420 | GATGCCACAG | 1421 |
| CAAGCACGTC | | CAGGCTCGAC | |
| A | | A | |
| TCAATTTCTG | 1422 | TTAACTTTTG | 1423 |
| TCTCATCTTA | | CCTGATTTTG | |
| A | | A | |
| TGTCTCTTGC | 1424 | TGCCTGTTAC | 1425 |
| TGATCTGTAT | | TCATTTGCAT | |
| C | | T | |
| GTCTCTTGCT | 1426 | GCCTGTTACT | 1427 |
| GATCTGTAT | | CATTTGCAT | |

In some cases, the mammal can have familial hypercholesterolemia (FH), and the gene to be suppressed and replaced can be LDLR (which encodes the low density lipoprotein receptor). An exemplary LDLR sequence is set forth in NCBI RefSeq accession number NM_000527 (e.g., version NM_000527.5). A LDLR polypeptide can, in some cases, have the amino acid sequence set forth in NCBI RefSeq accession number NP_000518 (e.g., version NP_000518.1).

Examples of shRNA sequences and corresponding shIMM sequences targeted to LDLR are set forth in TABLE 1Q.

TABLE 1Q
Representative LDLR shRNA and
shIMM sequences

| | SEQ | | SEQ |
|---|---|---|---|
| shRNA Sequence | ID | shIMM Sequence | ID |
| GTGCCAAGAC | 1428 | ATGTCAGGAT | 1429 |
| GGGAAATGCA | | GGCAAGTGTA | |
| TCTCCTACA | | TTTCGTATA | |
| GCCAAGACGG | 1430 | GTCAGGATGG | 1431 |
| GAAATGCATC | | CAAGTGTATT | |
| TCCTA | | TCGTA | |
| GACGGGAAAT | 1432 | GATGGCAAGT | 1433 |
| GCATCTCCTA | | GTATTTCGTA | |
| CAAGT | | TAAAT | |
| GACGGGAAAT | 1434 | GATGGCAAGT | 1435 |
| GCATCTCCT | | GTATTTCGT | |
| GGGAAATGCA | 1436 | GGCAAGTGTA | 1437 |
| TCTCCTACA | | TTTCGTATA | |
| GGAAATGCAT | 1438 | GCAAGTGTAT | 1439 |
| CTCCTACAAG | | TTCGTATAAA | |
| T | | T | |
| GGAAATGCAT | 1440 | GCAAGTGTAT | 1441 |
| CTCCTACAA | | TTCGTATAA | |
| GTCAACCGCT | 1442 | GTGAATCGGT | 1443 |
| GCATTCCTCA | | GTATACCACA | |
| GTTCT | | ATTTT | |
| GCAGTTCGTC | 1444 | CCAATTTGTG | 1445 |
| TGTGACTCA | | TGCGATTCT | |
| GAAGATGGCT | 1446 | GAGGACGGGT | 1447 |
| CGGATGAGT | | CCGACGAAT | |
| GACGAATTCC | 1448 | GATGAGTTTC | 1449 |
| AGTGCTCTGA | | AATGTTCAGA | |
| T | | C | |
| GGACATGAGC | 1450 | AGATATGAGT | 1451 |
| GATGAAGTT | | GACGAGGTA | |
| GCGAATGCAT | 1452 | GGGAGTGTAT | 1453 |
| CACCCTGGAC | | TACGCTCGAT | |
| AAAGT | | AAGGT | |
| GCATCACCCT | 1454 | GTATTACGCT | 1455 |
| GGACAAAGT | | CGATAAGGT | |
| GCTACAAGTG | 1456 | GGTATAAATG | 1457 |
| CCAGTGTGA | | TCAATGCGA | |
| GACCTGTCCC | 1458 | GATCTCTCGC | 1459 |
| AGAGAATGA | | AAAGGATGA | |
| GAGAATGATC | 1460 | AAGGATGATT | 1461 |
| TGCAGCACCC | | TGTAGTACGC | |
| AGCTTGACA | | AACTAGATA | |
| GGATCCACAG | 1462 | GGATTCATAG | 1463 |
| CAACATCTAC | | TAATATTTAT | |
| T | | T | |
| GGATCCACAG | 1464 | GGATTCATAG | 1465 |
| CAACATCTA | | TAATATTTA | |
| GCAACATCTA | 1466 | GTAATATTTA | 1467 |
| CTGGACCGAC | | TTGGACGGAT | |
| TCTGT | | TCAGT | |
| GCTTCATGTA | 1468 | GGTTTATGTA | 1469 |
| CTGGACTGAC | | TTGGACAGAT | |
| T | | T | |
| GCTTCATGTA | 1470 | GGTTTATGTA | 1471 |
| CTGGACTGA | | TTGGACAGA | |
| GGACATCTAC | 1472 | CGATATTTAT | 1473 |
| TCGCTGGTGA | | TCCCTCGTCA | |
| CTGAA | | CAGAG | |
| GCATCACCCT | 1474 | GGATTACGCT | 1475 |
| AGATCTCCT | | TGACCTGCT | |
| GACGTTGCTG | 1476 | GATGTAGCAG | 1477 |
| GCAGAGGAAA | | GGAGGGGTAA | |
| TGAGAAGAA | | CGAAAAAAA | |
| GACGTTGCTG | 1478 | GATGTAGCAG | 1479 |
| GCAGAGGAAA | | GGAGGGGTAA | |
| TGAGA | | CGAAA | |
| GTTGCTGGCA | 1480 | GTAGCAGGGA | 1481 |
| GAGGAAATGA | | GGGGTAACGA | |
| GAAGA | | AAAAA | |
| GAACATCAAC | 1482 | AAATATTAAT | 1483 |
| AGCATCAACT | | AGTATTAATT | |
| T | | T | |
| GAGGATGAGG | 1484 | GAAGACGAAG | 1485 |
| TCCACATTT | | TGCATATAT | |

In some cases, the mammal can have FH, and the gene to be suppressed and replaced can be PCSK9 (which encodes proprotein convertase subtilisin/kexin type 9). An exemplary PCSK9 sequence is set forth in NCBI RefSeq accession number NM_174936 (e.g., version NM_174936.4). A PCSK9 polypeptide can, in some cases, have the amino acid sequence set forth in NCBI RefSeq accession number NP_777596 (e.g., version NP_777596.2).

Examples of shRNA sequences and corresponding shIMM sequences targeted to PCSK9 are set forth in TABLE 1R.

TABLE 1R
Representative PCSK9 shRNA and
shIMM sequences

| | SEQ | | SEQ |
|---|---|---|---|
| shRNA Sequence | ID | shIMM Sequence | ID |
| GAGGTGTATC | 1486 | GAAGTCTACC | 1487 |
| TCCTAGACA | | TGCTTGATA | |
| GGTCTGGAAT | 1488 | GGACTCGAGT | 1489 |
| GCAAAGTCA | | GTAAGGTGA | |
| AATGCAAAGT | 1490 | AGTGTAAGGT | 1491 |
| CAAGGAGCAT | | GAAAGAACAC | |
| G | | G | |
| AAAGTCAAGG | 1492 | AAGGTGAAAG | 1493 |
| AGCATGGAAT | | AACACGGTAT | |
| C | | T | |
| AAGGATCCGT | 2757 | AAAGACCCCT | 2758 |
| GGAGGTTGCC | | GGAGATTACC | |
| T | | A | |
| AAGATCCTGC | 2759 | AAAATTCTCC | 2760 |
| ATGTCTTCCA | | ACGTGTTTCA | |
| T | | C | |
| GGTCACCGAC | 2761 | GGTGACGGAT | 2762 |
| TTCGAGAATG | | TTTGAAAACG | |
| T | | T | |
| GCACCCTCAT | 2763 | GGACGCTGAT | 2764 |
| AGGCCTGGAG | | TGGGCTCGAA | |
| TTTAT | | TTCAT | |
| GAGTTGAGGC | 2765 | GAATTAAGAC | 2766 |
| AGAGACTGA | | AAAGGCTCA | |
| GAGGCAGAGA | 2767 | AAGACAAAGG | 2768 |
| CTGATCCACT | | CTCATTCATT | |
| T | | T | |
| GGCAGAGACT | 2769 | GACAAAGGCT | 2770 |
| GATCCACTTC | | CATTCATTTT | |
| T | | T | |
| GGCAGAGACT | 2771 | GACAAAGGCT | 2772 |
| GATCCACTT | | CATTCATTT | |
| AACTGCAGCG | 2773 | AATTGTAGTG | 2774 |
| TCCACACAGC | | TGCATACTGC | |
| T | | A | |

In some cases, the mammal can have HCM or DCM, and the gene to be suppressed and replaced can be TNNT2 (which encodes cardiac type troponin T2). An exemplary TNNT2 sequence is set forth in NCBI RefSeq accession number NM_001276345 (e.g., version NM_001276345.2). A TNNT2 polypeptide can, in some cases, have the amino acid sequence set forth in NCBI RefSeq accession number NP_001263274 (e.g., version NP_001263274.1).

Examples of shRNA sequences and corresponding shIMM sequences targeted to TNNT2 are set forth in TABLE 1S.

TABLE 1S
Representative TNNT2 shRNA and
shIMM sequences

| | SEQ | | SEQ |
|---|---|---|---|
| shRNA Sequence | ID | shIMM Sequence | ID |
| GGAGCAGGAA | 1494 | AGAACAAGAG | 1495 |
| GAAGCAGCTG | | GAGGCTGCAG | |
| TTGAA | | TAGAG | |
| GAGCAGGAAG | 1496 | GAACAAGAGG | 1497 |
| AAGCAGCTGT | | AGGCTGCAGT | |
| TGAAGAAGA | | AGAGGAGGA | |
| GCAGGAAGAA | 1498 | ACAAGAGGAG | 1499 |
| GCAGCTGTTG | | GCTGCAGTAG | |
| A | | A | |
| GGAAGAAGCA | 1500 | AGAGGAGGCT | 1501 |
| GCTGTTGAA | | GCAGTAGAG | |
| AAGAGGAGGA | 1502 | AGGAAGAAGA | 1503 |
| CTGGAGAGAG | | TTGGAGGGAA | |
| G | | G | |
| GGAGACCAGG | 1504 | AGAAACGAGA | 1505 |
| GCAGAAGAAG | | GCTGAGGAGG | |
| ATGAA | | ACGAG | |
| GACCAGGGCA | 1506 | AACGAGAGCT | 1507 |
| GAAGAAGATG | | GAGGAGGACG | |
| AAGAA | | AGGAG | |
| GACCAGGGCA | 1508 | AACGAGAGCT | 1509 |
| GAAGAAGATG | | GAGGAGGACG | |
| AAGAAGAA | | AGGAGGAG | |
| GGGCAGAAGA | 1510 | GAGCTGAGGA | 1511 |
| AGATGAAGAA | | GGACGAGGAG | |
| GAAGA | | GAGGA | |
| GGCAGAAGAA | 1512 | AGCTGAGGAG | 1513 |
| GATGAAGAAG | | GACGAGGAGG | |
| A | | A | |
| GGCAGAAGAA | 1514 | AGCTGAGGAG | 1515 |
| GATGAAGAA | | GACGAGGAG | |
| GCAGAAGAAG | 1516 | GCTGAGGAGG | 1517 |
| ATGAAGAAGA | | ACGAGGAGGA | |
| A | | G | |
| AAGATGAAGA | 1518 | AGGACGAGGA | 1519 |
| AGAAGAGGAA | | GGAGGAAGAG | |
| G | | G | |
| GAAGAAGAAG | 1520 | GAGGAGGAGG | 1521 |
| AGGAAGCAAA | | AAGAGGCTAA | |
| G | | A | |
| AAGCAAAGGA | 1522 | AGGCTAAAGA | 1523 |
| GGCTGAAGAT | | AGCAGAGGAC | |
| G | | G | |
| GAAGCGCATG | 1524 | CAAACGGATG | 1525 |
| GAGAAGGACC | | GAAAAAGATC | |
| TGAATGAGT | | TCAACGAAT | |
| AGCTGTGGCA | 1526 | AACTCTGGCA | 1527 |
| GAGCATCTAT | | AAGTATTTAC | |
| A | | A | |

In some cases, the mammal can have LQTS or CPVT, and the gene to be suppressed and replaced can be CALM1 (which encodes calmodulin 1). An exemplary CALM1 sequence is set forth in NCBI RefSeq accession number NM_006888 (e.g., version NM_006888.6). A CALM1 polypeptide can, in some cases, have the amino acid sequence set forth in NCBI RefSeq accession number NP_008819 (e.g., version NP_008819.1).

Examples of shRNA sequences and corresponding shIMM sequences targeted to CALM1 are set forth in TABLE 1T.

TABLE 1T
Representative CALMI shRNA and
shIMM sequences

| | SEQ | | SEQ |
|---|---|---|---|
| shRNA Sequence | ID | shIMM Sequence | ID |
| AAGAACAGAT | 1528 | AGGAGCAAAT | 1529 |
| TGCTGAATTC | | AGCAGAGTTT | |
| A | | A | |
| GAAAGATACA | 1530 | GAAGGACACT | 1531 |
| GATAGTGAAG | | GACAGCGAGG | |
| AAGAA | | AGGAG | |
| AAGATACAGA | 1532 | AGGACACTGA | 1533 |
| TAGTGAAGAA | | CAGCGAGGAG | |
| G | | G | |
| GATGAAGAAG | 1534 | GACGAGGAGG | 1535 |
| TAGATGAAAT | | TTGACGAGAT | |
| GATCAGAGA | | GATTAGGGA | |
| GATGAAGAAG | 1536 | GACGAGGAGG | 1537 |
| TAGATGAAAT | | TTGACGAGAT | |
| GATCA | | GATTA | |
| AAGTAGATGA | 1538 | AGGTTGACGA | 1539 |
| AATGATCAGA | | GATGATTAGG | |
| G | | G | |

In some cases, the mammal can have LQTS or CPVT, and the gene to be suppressed and replaced can be CALM2 (which encodes calmodulin 2). An exemplary CALM2 sequence is set forth in NCBI RefSeq accession number NM_001743 (e.g., version NM_001743.6). A CALM2 polypeptide can, in some cases, have the amino acid sequence set forth in NCBI RefSeq accession number NP_001734 (e.g., version NP_001734.1).

Examples of shRNA sequences and corresponding shIMM sequences targeted to CALM2 are set forth in TABLE 1U.

TABLE 1U
Representative CALM2 shRNA and
shIMM sequences

| | SEQ | | SEQ |
|---|---|---|---|
| shRNA Sequence | ID | shIMM Sequence | ID |
| GGCTGACCAA | 1540 | GGCAGATCAG | 1541 |
| CTGACTGAA | | CTCACAGAG | |
| AAGAGCAGAT | 1542 | AGGAACAAAT | 1543 |
| TGCAGAATTC | | AGCTGAGTTT | |
| A | | A | |
| GAGCAGATTG | 1544 | GAACAAATAG | 1545 |
| CAGAATTCAA | | CTGAGTTTAA | |
| A | | G | |
| GAGCAGATTG | 1546 | GAACAAATAG | 1547 |
| CAGAATTCA | | CTGAGTTTA | |
| GCAGATTGCA | 1548 | ACAAATAGCT | 1549 |
| GAATTCAAAG | | GAGTTTAAGG | |
| A | | A | |
| GCAGATTGCA | 1550 | ACAAATAGCT | 1551 |
| GAATTCAAA | | GAGTTTAAG | |
| GACAAAGATG | 1552 | GATAAGGACG | 1553 |
| GTGATGGAAC | | GAGACGGTAC | |
| TATAA | | AATTA | |
| AAAGATGGTG | 1554 | AAGGACGGAG | 1555 |
| ATGGAACTAT | | ACGGTACAAT | |
| A | | T | |
| AAGATGGTGA | 1556 | AGGACGGAGA | 1557 |
| TGGAACTATA | | CGGTACAATT | |
| A | | A | |
| GGCAGAATCC | 1558 | GCCAAAACCC | 1559 |
| CACAGAAGCA | | GACTGAGGCT | |
| GAGTT | | GAATT | |
| GCAGAATCCC | 1560 | CCAAAACCCG | 1561 |
| ACAGAAGCAG | | ACTGAGGCTG | |
| AGTTA | | AATTG | |
| GAATCCCACA | 1562 | AAACCCGACT | 1563 |
| GAAGCAGAGT | | GAGGCTGAAT | |
| T | | T | |
| GAAGTAGATG | 1564 | GAGGTTGACG | 1565 |
| CTGATGGTAA | | CAGACGGAAA | |
| T | | C | |
| AAGTAGATGC | 1566 | AGGTTGACGC | 1567 |
| TGATGGTAAT | | AGACGGAAAC | |
| G | | G | |
| GCTGATGGTA | 1568 | GCAGACGGAA | 1569 |
| ATGGCACAAT | | ACGGGACTAT | |
| TGACT | | AGATT | |
| GCTGATGGTA | 1570 | GCAGACGGAA | 1571 |
| ATGGCACAAT | | ACGGGACTAT | |
| T | | A | |
| GGTAATGGCA | 1572 | GGAAACGGGA | 1573 |
| CAATTGACT | | CTATAGATT | |
| GGAGAGAAGT | 1574 | GGTGAAAAAT | 1575 |
| TAACAGATGA | | TGACTGACGA | |
| AGAAGTTGA | | GGAGGTAGA | |
| GAGAGAAGTT | 1576 | GTGAAAAATT | 1577 |
| AACAGATGAA | | GACTGACGAG | |
| GAAGT | | GAGGT | |
| GAGAAGTTAA | 1578 | GAAAAATTGA | 1579 |
| CAGATGAAGA | | CTGACGAGGA | |
| AGTTGATGA | | GGTAGACGA | |

In some cases, the mammal can have LQTS or CPVT, and the gene to be suppressed and replaced can be CALM3 (which encodes calmodulin 3). An exemplary CALM3 sequence is set forth in NCBI RefSeq accession number NM_005184 (e.g., version NM_005184.4). A CALM3 polypeptide can, in some cases, have the amino acid sequence set forth in NCBI RefSeq accession number NP_005175.2 (e.g., version NP_005175.2).

Examples of shRNA sequences and corresponding shIMM sequences targeted to CALM3 are set forth in TABLE 1V.

TABLE 1V
Representative CALM3 shRNA and
shIMM sequences

| | SEQ | | SEQ |
|---|---|---|---|
| shRNA Sequence | ID | shIMM Sequence | ID |
| GCTGACTGAG | 1580 | ACTCACAGAA | 1581 |
| GAGCAGATTG | | GAACAAATAG | |
| CAGAGTTCA | | CTGAATTTA | |
| GAGCAGATTG | 1582 | GAACAAATAG | 1583 |
| CAGAGTTCA | | CTGAATTTA | |
| GACAAGGATG | 1584 | GATAAAGACG | 1585 |
| GAGATGGCAC | | GTGACGGGAC | |
| TATCA | | AATTA | |
| AAGGATGGAG | 1586 | AAAGACGGTG | 1587 |
| ATGGCACTAT | | ACGGGACAAT | |
| C | | T | |
| GATGGAGATG | 1588 | GACGGTGACG | 1589 |
| GCACTATCA | | GGACAATTA | |
| GGAGATGGCA | 1590 | GGTGACGGGA | 1591 |
| CTATCACCAC | | CAATTACGAC | |
| CAAGGAGTT | | GAAAGAATT | |
| AAGCAGAGCT | 1592 | AGGCTGAACT | 1593 |
| GCAGGATATG | | CCAAGACATG | |
| A | | A | |
| GCTGCAGGAT | 1594 | ACTCCAAGAC | 1595 |
| ATGATCAATG | | ATGATTAACG | |
| A | | A | |
| AAAGATGAAG | 1596 | GAAAATGAAA | 1597 |
| GACACAGACA | | GATACTGATA | |
| G | | G | |
| AAGATGAAGG | 1598 | AAAATGAAAG | 1599 |
| ACACAGACAG | | ATACTGATAG | |
| T | | C | |
| AAGGACACAG | 1600 | AAAGATACTG | 1601 |
| ACAGTGAGGA | | ATAGCGAAGA | |
| G | | A | |
| AAGCTGACCG | 1602 | AAACTCACGG | 1603 |
| ATGAGGAGGT | | ACGAAGAAGT | |
| G | | C | |
| GACCGATGAG | 1604 | CACGGACGAA | 1605 |
| GAGGTGGATG | | GAAGTCGACG | |
| AGATGATCA | | AAATGATTA | |
| GATGAGGAGG | 1606 | GACGAAGAAG | 1607 |
| TGGATGAGAT | | TCGACGAAAT | |
| GATCA | | GATTA | |
| GAGGAGGTGG | 1608 | GAAGAAGTCG | 1609 |
| ATGAGATGA | | ACGAAATGA | |
| GAGGTGGATG | 1610 | GAAGTCGACG | 1611 |
| AGATGATCA | | AAATGATTA | |

In some cases, the mammal can have Triadin Knockout Syndrome (TKOS), and the gene to be suppressed and replaced can be TRDN (which encodes triadin). An exemplary TRDN sequence is set forth in NCBI RefSeq accession number NM_006073 (e.g., version NM_006073.4). A TRDN polypeptide can, in some cases, have the amino acid sequence set forth in NCBI RefSeq accession number NP_006064 (e.g., version NP_006064.2).

Examples of shRNA sequences and corresponding shIMM sequences targeted to CALM3 are set forth in TABLE 1W.

TABLE 1W
Representative TRDN shRNA and
shIMM sequences

| | SEQ | | SEQ |
|---|---|---|---|
| shRNA Sequence | ID | shIMM Sequence | ID |
| GTGCTGAAGA | 1612 | GTCCTCAAAA | 1613 |
| GGACAGTCAC | | GAACTGTGAC | |
| AGAAGACAT | | TGAGGATAT | |
| GTGCTGAAGA | 1614 | GTCCTCAAAA | 1615 |
| GGACAGTCAC | | GAACTGTGAC | |
| A | | T | |
| GTGCTGAAGA | 1616 | GTCCTCAAAA | 1617 |
| GGACAGTCA | | GAACTGTGA | |
| GCTGAAGAGG | 1618 | CCTCAAAAGA | 1619 |
| ACAGTCACA | | ACTGTGACT | |
| GAAGAGGACA | 1620 | CAAAAGAACT | 1621 |
| GTCACAGAAG | | GTGACTGAGG | |
| ACATA | | ATATT | |
| GAAGAGGACA | 1622 | CAAAAGAACT | 1623 |
| GTCACAGAAG | | GTGACTGAGG | |
| A | | A | |
| GAGGACAGTC | 1624 | AAGAACTGTG | 1625 |
| ACAGAAGACA | | ACTGAGGATA | |
| T | | T | |
| GGACAGTCAC | 1626 | GAACTGTGAC | 1627 |
| AGAAGACAT | | TGAGGATAT | |
| GACAGTCACA | 1628 | AACTGTGACT | 1629 |
| GAAGACATAG | | GAGGATATTG | |
| T | | T | |
| GACAGTCACA | 1630 | AACTGTGACT | 1631 |
| GAAGACATA | | GAGGATATT | |
| GCCTGGCTTC | 1632 | GCGTGGCTAC | 1633 |
| TGGTCATTGC | | TCGTGATAGC | |
| CCTGATAAT | | GCTCATTAT | |
| GGCTTCTGGT | 1634 | GGCTACTCGT | 1635 |
| CATTGCCCTG | | GATAGCGCTC | |
| ATAAT | | ATTAT | |
| GATTGGCTCA | 1636 | AATAGGGTCT | 1637 |
| GATCCTTTAA | | GACCCATTGA | |
| A | | A | |
| GCTATGGAGG | 1638 | GCAATGGAAG | 1639 |
| AAACCACGGA | | AGACGACCGA | |
| CTGGATCTA | | TTGGATTTA | |
| GGAGGAAACC | 1640 | GGAAGAGACG | 1641 |
| ACGGACTGGA | | ACCGATTGGA | |
| TCTAT | | TTTAC | |
| GGAAACCACG | 1642 | AGAGACGACC | 1643 |
| GACTGGATCT | | GATTGGATTT | |
| A | | A | |
| GAAACCACGG | 1644 | GAGACGACCG | 1645 |
| ACTGGATCTA | | ATTGGATTTA | |
| T | | C | |
| GGCAAGAAGC | 1646 | GGGAAAAAAC | 1647 |
| ACATGCAGTG | | ATATGCAATG | |
| A | | A | |

In some cases, the mammal can have CPVT, and the gene to be suppressed and replaced can be RYR2 (which encodes ryanodine receptor 2). An exemplary RYR2 sequence is set forth in NCBI RefSeq accession number NM_001035 (e.g., version NM_001035.3). A RYR2 polypeptide can, in some cases, have the amino acid sequence set forth in NCBI RefSeq accession number NP_001026 (e.g., version NP_001026.2).

Examples of shRNA sequences and corresponding shIMM sequences targeted to RYR2 are set forth in TABLE 1X.

TABLE 1X
Representative RYR2 shRNA and
shIMM sequences

| | SEQ | | SEQ |
|---|---|---|---|
| shRNA Sequence | ID | shIMM Sequence | ID |
| GAGAACATGGTGAAG | 1648 | GTGAGCACGGAGAGG | 1649 |
| AGCAGCGGAGAACT | | AACAACGCAGGACA | |
| GAACATGGTGAAGAG | 1650 | GAGCACGGAGAGGAA | 1651 |
| CAGCGGAGAACTGT | | CAACGCAGGACAGT | |
| GAGCATTTAGAGCAT | 1652 | GAACACTTGGAACAC | 1653 |
| GAAGACAAACAGAA | | GAGGATAAGCAAAA | |
| GCATTTAGAGCATGA | 1654 | ACACTTGGAACACGA | 1655 |
| AGACAAACAGAACA | | GGATAAGCAAAATA | |
| GTTGCAGTCCGTTCT | 1656 | GTAGCTGTGCGATCA | 1657 |
| AACCAGCATCTCAT | | AATCAACACCTGAT | |
| GTCCGTTCTAACCAG | 1658 | GTGCGATCAAATCAA | 1659 |
| CATCTCATCTGTGA | | CACCTGATTTGCGA | |
| GGGCGTCAGTGAAGG | 1660 | CGGGGTGAGCGAGGG | 1661 |
| TTCTGCTCAGTATA | | ATCAGCACAATACA | |
| GGCGTCAGTGAAGGT | 1662 | GGGGTGAGCGAGGGA | 1663 |
| TCTGCTCAGTATAA | | TCAGCACAATACAA | |
| GATGGCCTCTTCTTT | 1664 | GACGGGCTGTTTTTC | 1665 |
| CCAGTCGTTAGTTT | | CCTGTGGTAAGCTT | |
| GCCTCTTCTTTCCAG | 1666 | GGCTGTTTTTCCCTG | 1667 |
| TCGTTAGTTTCTCT | | TGGTAAGCTTTTCA | |
| GTCCGGTTAGAGATG | 1668 | GACCCGTAAGGGACG | 1669 |
| ACAACAAGAGACAA | | ATAATAAAAGGCAG | |
| GAAGAAATCCTCGCC | 1670 | GGAGGAACCCACGGC | 1671 |
| TTGTTCCCTACACT | | TAGTACCGTATACA | |
| GAAATCCTCGCCTTG | 1672 | GGAACCCACGGCTAG | 1673 |
| TTCCCTACACTCTT | | TACCGTATACACTA | |
| GCGGGATTATTCAAG | 1674 | GCCGGTTTGTTTAAA | 1675 |
| AGTGAGCACAAGAA | | AGCGAACATAAAAA | |
| GGATCCTCTGCAGTT | 1676 | AGACCCACTCCAATT | 1677 |
| CATGTCTCTTCATA | | TATGTCACTACACA | |
| GATCCTCTGCAGTTC | 1678 | GACCCACTCCAATTT | 1679 |
| ATGTCTCTTCATAT | | ATGTCACTACACAT | |
| GCCATGTGGATGAAC | 1680 | GTCACGTCGACGAGC | 1681 |
| CTCAGCTCCTCTAT | | CACAACTGCTGTAC | |
| GCTCCTCTATGCCAT | 1682 | ACTGCTGTACGCGAT | 1683 |
| TGAGAACAAGTACA | | AGAAAATAAATATA | |
| GCTGGCTACTATGAC | 1684 | GCAGGGTATTACGAT | 1685 |
| CTGCTGATTGACAT | | CTCCTCATAGATAT | |
| GAGGACTTGAAGCAC | 1686 | GAAGATTTAAAACAT | 1687 |
| ATCTTGCAGTTGAT | | ATTTTACAATTAAT | |
| GCAAGCCTTAAACAT | 1688 | GCAGGCGTTGAATAT | 1689 |
| GTCAGCTGCACTCA | | GTCTGCAGCTCTGA | |
| GATGCCTCTTAAACT | 1690 | AATGCCACTAAAGCT | 1691 |
| GCTGACAAATCATT | | CCTCACTAACCACT | |
| GCCCTATGATACACT | 1692 | CCCGTACGACACTCT | 1693 |
| GACAGCCAAAGAGA | | CACTGCGAAGGAAA | |
| GACCTGGAACTGGAC | 1694 | GATCTCGAGCTCGAT | 1695 |
| ACGCCTTCTATTGA | | ACCCCATCAATAGA | |
| GGTGGCAGCAGAGGC | 1696 | GGAGGGAGTAGGGGG | 1697 |
| AAAGGAGAACATTT | | AAGGGTGAGCACTT | |
| GGAGGACATGCTTCC | 1698 | GGTGGTCACGCATCG | 1699 |
| AACAAAGAGAAAGA | | AATAAGGAAAAGGA | |
| GAGGACATGCTTCCA | 1700 | GTGGTCACGCATCGA | 1701 |
| ACAAAGAGAAAGAA | | ATAAGGAAAAGGAG | |
| GGACATGCTTCCAAC | 1702 | GGTCACGCATCGAAT | 1703 |
| AAAGAGAAAGAAAT | | AAGGAAAAGGAGAT | |
| GGAGTTCTTGTCAGG | 1704 | GGTGTACTAGTGAGA | 1705 |
| CATAGGATTTCACT | | CACAGAATATCTCT | |
| GAGTTCTTGTCAGGC | 1706 | GTGTACTAGTGAGAC | 1707 |
| ATAGGATTTCACTA | | ACAGAATATCTCTT | |
| GGCCAGCATCAGTTC | 1708 | GGGCAACACCAATTT | 1709 |
| GGAGAAGACCTAAT | | GGTGAGGATCTTAT | |
| GCCAGCATCAGTTCG | 1710 | GGCAACACCAATTTG | 1711 |
| GAGAAGACCTAATA | | GTGAGGATCTTATT | |
| GTGGAGAGGCAACGT | 1712 | GTCGAAAGACAGCGA | 1713 |
| TCTGCATTAGGAGA | | TCAGCTTTGGGTGA | |
| GCTATTAGATGGCAA | 1714 | GCAATAAGGTGGCAG | 1715 |
| ATGGCTCTTTACAA | | ATGGCACTATATAA | |
| GCTGTCAATCTCTTT | 1716 | GCAGTGAACCTGTTC | 1717 |
| CTTCAGGGATATGA | | CTACAAGGTTACGA | |
| GGCCTATGCAGATAT | 1718 | GGCGTACGCTGACAT | 1719 |
| TATGGCAAAGAGTT | | AATGGCTAAAAGCT | |
| GCAGATATTATGGCA | 1720 | GCTGACATAATGGCT | 1721 |
| AAGAGTTGTCATGA | | AAAAGCTGCCACGA | |
| GGATGGTGACAGAGG | 1722 | GCATGGTCACTGAAG | 1723 |
| AAGGATCAGGAGAA | | AGGGTTCTGGTGAG | |
| GATGGTGACAGAGGA | 1724 | CATGGTCACTGAAGA | 1725 |
| AGGATCAGGAGAAA | | GGGTTCTGGTGAGA | |
| GAGAATGAAACCCTC | 1726 | GAAAACGAGACGCTG | 1727 |
| GACTACGAAGAGTT | | GATTATGAGGAATT | |
| GGATCTGAAGAGAGA | 1728 | CGACCTCAAAAGGGA | 1729 |
| AGGAGGACAGTACA | | GGGTGGTCAATATA | |
| GAAAGCCAAGGAAGA | 1730 | AAAGGCGAAAGAGGA | 1731 |
| CAAGGGCAAACAAA | | TAAAGGGAAGCAGA | |
| GCTACATGGAGCCCA | 1732 | GGTATATGGAACCGA | 1733 |
| CGTTGCGTATCTTA | | CCTTACGAATTTTG | |
| GATGATATTAAAGGC | 1734 | GACGACATAAAGGGG | 1735 |
| CAGTGGGATAGACT | | CAATGGGACAGGCT | |
| GAAGACCCAGCAGGA | 1736 | GAGGATCCTGCTGGT | 1737 |
| GATGAATATGAGAT | | GACGAGTACGAAAT | |

In some cases, the mammal can have FH, and the gene to be suppressed and replaced can be APOB (which encodes apolipoprotein B). An exemplary APOB sequence is set forth in NCBI RefSeq accession number NM_000384 (e.g., version NM_000384.3). An APOB polypeptide can, in some cases, have the amino acid sequence set forth in NCBI RefSeq accession number NP_000375 (e.g., version NP_000375.3).

Examples of shRNA sequences and corresponding shIMM sequences targeted to APOB are set forth in TABLE 1Y.

TABLE 1Y
Representative APOB shRNA and shIMM sequences

| | SEQ | | SEQ |
|---|---|---|---|
| shRNA Sequence | ID | shIMM Sequence | ID |
| GGGACTGCTGATTCA | 1738 | GGCACAGCAGACTCT | 1739 |
| AGAAGT | | AGGAGC | |
| GGGACTGCTGATTCA | 1740 | GGCACAGCAGACTCT | 1741 |
| AGAA | | AGGA | |
| GACTGCTGATTCAAG | 1742 | CACAGCAGACTCTAG | 1743 |
| AAGT | | GAGC | |
| GATTCAAGAAGTGCC | 1744 | GACTCTAGGAGCGCG | 1745 |
| ACCAGGATCA | | ACGAGAATTA | |
| GAAAGATGAACCTAC | 1746 | AAAGGACGAGCCAAC | 1747 |
| TTACAT | | ATATAT | |
| GAACCTACTTACATC | 1748 | GAGCCAACATATATT | 1749 |
| CTGAACATCA | | CTCAATATTA | |
| GGAAACTGCTCCACT | 1750 | GGTAATTGTTCGACA | 1751 |
| CACTTT | | CATTTC | |
| GGAAACTGCTCCACT | 1752 | GGTAATTGTTCGACA | 1753 |
| CACT | | CATT | |
| GAAACTGCTCCACTC | 1754 | GTAATTGTTCGACAC | 1755 |
| ACTT | | ATTT | |
| GAAGCCATCTGCAAG | 1756 | GAGGCGATTTGTAAA | 1757 |
| GAGCAACACCTCTT | | GAACAGCATCTGTT | |
| GCAAGGAGCAACACC | 1758 | GTAAAGAACAGCATC | 1759 |
| TCTT | | TGTT | |
| GAGCAACACCTCTTC | 1760 | GAACAGCATCTGTTT | 1761 |
| CTGCCTTTCTCCTA | | CTCCCATTTTCGTA | |
| GGGATGGTAGCACAA | 1762 | GGCATGGTTGCTCAG | 1763 |
| GTGACA | | GTCACT | |
| GGGATGGTAGCACAA | 1764 | GGCATGGTTGCTCAG | 1765 |
| GTGA | | GTCA | |
| GATGGTAGCACAAGT | 1766 | CATGGTTGCTCAGGT | 1767 |
| GACA | | CACT | |
| GAGCACCAAATCCAC | 1768 | AAGTACGAAGTCGAC | 1769 |
| ATCA | | TTCT | |
| GAGCTAATCTCTTCA | 1770 | GGGCAAACCTGTTTA | 1771 |
| ATAA | | ACAA | |
| GCCTCAGTGATGAAG | 1772 | GGCTGAGCGACGAGG | 1773 |
| CAGTCA | | CTGTGA | |
| GAAGCAGTCACATCT | 1774 | GAGGCTGTGACTTCA | 1775 |
| CTCT | | CTGT | |
| GAGCTGCTGGACATT | 1776 | GAACTCCTCGATATA | 1777 |
| GCTAAT | | GCAAAC | |
| GCTGCTGGACATTGC | 1778 | ACTCCTCGATATAGC | 1779 |
| TAATTA | | AAACTA | |
| GCTGCTGGACATTGC | 1780 | ACTCCTCGATATAGC | 1781 |
| TAAT | | AAAC | |
| GCTGGACATTGCTAA | 1782 | CCTCGATATAGCAAA | 1783 |
| TTACCT | | CTATCT | |
| GGACATTGCTAATTA | 1784 | CGATATAGCAAACTA | 1785 |
| CCTGATGGAACAGA | | TCTCATGGAGCAAA | |
| GCTAATTACCTGATG | 1786 | GCAAACTATCTCATG | 1787 |
| GAACAGATTCAAGA | | GAGCAAATACAGGA | |
| GATGGAACAGATTCA | 1788 | CATGGAGCAAATACA | 1789 |
| AGATGA | | GGACGA | |
| GATGGAACAGATTCA | 1790 | CATGGAGCAAATACA | 1791 |
| AGAT | | GGAC | |
| GAACAGATTCAAGAT | 1792 | GAGCAAATACAGGAC | 1793 |
| GACT | | GATT | |
| GGGATGAAGATTACA | 1794 | GCGACGAGGACTATA | 1795 |
| CCTATT | | CGTACT | |
| GGATGAAGATTACAC | 1796 | CGACGAGGACTATAC | 1797 |
| CTATTT | | GTACTT | |
| GGATGAAGATTACAC | 1798 | CGACGAGGACTATAC | 1799 |
| CTAT | | GTAC | |
| GATGAAGATTACACC | 1800 | GACGAGGACTATACG | 1801 |
| TATT | | TACT | |
| GGCCAAACCATGGAG | 1802 | GGGCAGACGATGGAA | 1803 |
| CAGTTA | | CAATTG | |
| GCCAAACCATGGAGC | 1804 | GGCAGACGATGGAAC | 1805 |
| AGTTAA | | AATTGA | |
| GTCCAAAGTACAAAG | 1806 | GTGCAGAGCACTAAA | 1807 |
| CCATCACTGA | | CCTTCTCTCA | |
| GAGCCTAAAGACAAG | 1808 | GAACCAAAGGATAAA | 1809 |
| GACCAGGAGGTTCT | | GATCAAGAAGTACT | |
| GGACCAGGAGGTTCT | 1810 | AGATCAAGAAGTACT | 1811 |
| TCTTCA | | ACTACA | |
| GGACCAGGAGGTTCT | 1812 | AGATCAAGAAGTACT | 1813 |
| TCTTCAGACT | | ACTACAAACA | |
| GGACCAGGAGGTTCT | 1814 | AGATCAAGAAGTACT | 1815 |
| TCTT | | ACTA | |
| GACCAGGAGGTTCTT | 1816 | GATCAAGAAGTACTA | 1817 |
| CTTCAGACTT | | CTACAAACAT | |
| GGAGGTTCTTCTTCA | 1818 | AGAAGTACTACTACA | 1819 |
| GACTTT | | AACATT | |
| GGAGGTTCTTCTTCA | 1820 | AGAAGTACTACTACA | 1821 |
| GACT | | AACA | |
| GAGGTTCTTCTTCAG | 1822 | GAAGTACTACTACAA | 1823 |
| ACTT | | ACAT | |
| GGCTGCCTATCTTAT | 1824 | CGCAGCGTACCTAAT | 1825 |
| GTTGAT | | GTTAAT | |
| GCTGCCTATCTTATG | 1826 | GCAGCGTACCTAATG | 1827 |
| TTGA | | TTAA | |
| GCCTATCTTATGTTG | 1828 | GCGTACCTAATGTTA | 1829 |
| ATGA | | ATGA | |
| GGAGTCCTTCACAGG | 1830 | GAAGCCCATCTCAAG | 1831 |
| CAGATATTAA | | CTGACATAAA | |
| GGAGTCCTTCACAGG | 1832 | GAAGCCCATCTCAAG | 1833 |
| CAGATATTAACAAA | | CTGACATAAATAAG | |
| GAGTCCTTCACAGGC | 1834 | AAGCCCATCTCAAGC | 1835 |
| AGATAT | | TGACAT | |
| GTCCTTCACAGGCAG | 1836 | GCCCATCTCAAGCTG | 1837 |
| ATATTA | | ACATAA | |
| GTCCTTCACAGGCAG | 1838 | GCCCATCTCAAGCTG | 1839 |
| ATAT | | ACAT | |
| GTCCAAATTCTACCA | 1840 | GTGCAGATACTTCCT | 1841 |
| TGGGAACAGA | | TGGGAGCAAA | |
| GGGAACAGAATGAGC | 1842 | GGGAGCAAAACGAAC | 1843 |
| AAGTGA | | AGGTCA | |
| GGGAACAGAATGAGC | 1844 | GGGAGCAAAACGAAC | 1845 |
| AAGTGAAGAA | | AGGTCAAAAA | |
| GGAACAGAATGAGCA | 1846 | GGAGCAAAACGAACA | 1847 |
| AGTGAA | | GGTCAA | |
| GAACAGAATGAGCAA | 1848 | GAGCAAAACGAACAG | 1849 |
| GTGAAGAACT | | GTCAAAAATT | |
| GAACAGAATGAGCAA | 1850 | GAGCAAAACGAACAG | 1851 |
| GTGA | | GTCA | |
| GTTGAGAAGCTGATT | 1852 | GTAGAAAAACTCATA | 1853 |
| AAAGAT | | AAGGAC | |
| GAGAAGCTGATTAAA | 1854 | GAAAAACTCATAAAG | 1855 |
| GATT | | GACT | |
| GGAGCTGGATTACAG | 1856 | GGTGCAGGTTTGCAA | 1857 |
| TTGCAAATAT | | TTACAGATTT | |
| GAGCTGGATTACAGT | 1858 | GTGCAGGTTTGCAAT | 1859 |
| TGCAAATATCTTCA | | TACAGATTTCATCT | |
| GCTGGATTACAGTTG | 1860 | GCAGGTTTGCAATTA | 1861 |
| CAAATATCTT | | CAGATTTCAT | |
| GGATTACAGTTGCAA | 1862 | GGTTTGCAATTACAG | 1863 |
| ATATCT | | ATTTCA | |
| GGATTACAGTTGCAA | 1864 | GGTTTGCAATTACAG | 1865 |
| ATAT | | ATTT | |
| GTTGCAAATATCTTC | 1866 | ATTACAGATTTCATC | 1867 |
| ATCT | | TTCA | |
| GCAAATATCTTCATC | 1868 | ACAGATTTCATCTTC | 1869 |
| TGGAGTCATT | | AGGTGTGATA | |
| GACAAATATGGGCAT | 1870 | CACTAACATGGGGAT | 1871 |
| CATCAT | | TATTAT | |
| GCATCATCATTCCGG | 1872 | GGATTATTATACCCG | 1873 |
| ACTTCGCTAGGAGT | | ATTTTGCAAGAAGC | |
| GGGAAGCTGAAGTTT | 1874 | GGCAAACTCAAATTC | 1875 |
| ATCATT | | ATTATA | |
| GAAGCTGAAGTTTAT | 1876 | CAAACTCAAATTCAT | 1877 |
| CATT | | TATA | |
| GAGGCCTACAGGAGA | 1878 | CAGACCAACTGGTGA | 1879 |
| GATT | | AATA | |
| GGTGGATACCCTGAA | 1880 | AGTCGACACGCTCAA | 1881 |
| GTTT | | ATTC | |
| GAAGCAGACTGAGGC | 1882 | CAAACAAACAGAAGC | 1883 |
| TACCAT | | AACGAT | |
| GTTGACCTCGGAACA | 1884 | GTAGATCTGGGTACT | 1885 |
| ATCCTCAGAGTTAA | | ATTCTGAGGGTAAA | |
| GACCTCGGAACAATC | 1886 | GATCTGGGTACTATT | 1887 |
| CTCAGAGTTA | | CTGAGGGTAA | |
| GACCTCGGAACAATC | 1888 | GATCTGGGTACTATT | 1889 |
| CTCAGAGTTAATGA | | CTGAGGGTAAACGA | |
| GGAACAATCCTCAGA | 1890 | GGTACTATTCTGAGG | 1891 |
| GTTA | | GTAA | |
| GAACAATCCTCAGAG | 1892 | GTACTATTCTGAGGG | 1893 |
| TTAA | | TAAA | |
| GGACATTCAGAACAA | 1894 | CGATATACAAAATAA | 1895 |
| GAAA | | AAAG | |
| GCAAGCAGAAGCCAG | 1896 | ACAGGCTGAGGCGAG | 1897 |
| AAGTGA | | GAGCGA | |
| GCAAGCAGAAGCCAG | 1898 | ACAGGCTGAGGCGAG | 1899 |
| AAGT | | GAGC | |
| GCAGAAGCCAGAAGT | 1900 | GCTGAGGCGAGGAGC | 1901 |
| GAGA | | GAAA | |
| GAACATGGGATTGCC | 1902 | AAATATGGGTTTACC | 1903 |
| AGACTT | | TGATTT | |
| GACCTCTCCACGAAT | 1904 | GATCTGTCGACCAAC | 1905 |
| GTCT | | GTGT | |
| GTGCAAGGATCTGGA | 1906 | GTCCAGGGTTCAGGT | 1907 |
| GAAACA | | GAGACT | |
| GTGCAAGGATCTGGA | 1908 | GTCCAGGGTTCAGGT | 1909 |
| GAAACAACAT | | GAGACTACTT | |
| GTGCAAGGATCTGGA | 1910 | GTCCAGGGTTCAGGT | 1911 |
| GAAACAACATATGA | | GAGACTACTTACGA | |
| GTGCAAGGATCTGGA | 1912 | GTCCAGGGTTCAGGT | 1913 |
| GAAA | | GAGA | |
| GCAAGGATCTGGAGA | 1914 | CCAGGGTTCAGGTGA | 1915 |
| AACAACATAT | | GACTACTTAC | |
| GCAAGGATCTGGAGA | 1916 | CCAGGGTTCAGGTGA | 1917 |
| AACA | | GACT | |
| GGATCTGGAGAAACA | 1918 | GGTTCAGGTGAGACT | 1919 |
| ACATAT | | ACTTAC | |
| GGATCTGGAGAAACA | 1920 | GGTTCAGGTGAGACT | 1921 |
| ACAT | | ACTT | |
| GATCTGGAGAAACAA | 1922 | GTTCAGGTGAGACTA | 1923 |
| CATA | | CTTA | |
| GGAGAAACAACATAT | 1924 | GGTGAGACTACTTAC | 1925 |
| GACCACAAGA | | GATCATAAAA | |
| GAAACAACATATGAC | 1926 | GAGACTACTTACGAT | 1927 |
| CACAAGAATA | | CATAAAAACA | |
| GGCACATATGGCCTG | 1928 | GGGACTTACGGGCTC | 1929 |
| TCTTGT | | TCATGC | |
| GGCACATATGGCCTG | 1930 | GGGACTTACGGGCTC | 1931 |
| TCTTGTCAGA | | TCATGCCAAA | |
| GGCACATATGGCCTG | 1932 | GGGACTTACGGGCTC | 1933 |
| TCTT | | TCAT | |
| GCCTGTCTTGTCAGA | 1934 | GGCTCTCATGCCAAA | 1935 |
| GGGATCCTAA | | GAGACCCAAA | |
| GAGAACTACGAGCTG | 1936 | GAAAATTATGAACTC | 1937 |
| ACTTTA | | ACATTG | |
| GAGAACTACGAGCTG | 1938 | GAAAATTATGAACTC | 1939 |
| ACTT | | ACAT | |
| GAACTACGAGCTGAC | 1940 | AAATTATGAACTCAC | 1941 |
| TTTA | | ATTG | |
| GACACCAATGGGAAG | 1942 | GATACGAACGGCAAA | 1943 |
| TATA | | TACA | |
| GATGGATATGACCTT | 1944 | AATGGACATGACGTT | 1945 |
| CTCTAA | | TTCAAA | |
| GATGGATATGACCTT | 1946 | AATGGACATGACGTT | 1947 |
| CTCT | | TTCA | |
| GCTTTCTGGATCACT | 1948 | CCTATCAGGTTCTCT | 1949 |
| AAAT | | TAAC | |
| GGATCACTAAATTCC | 1950 | GGTTCTCTTAACTCG | 1951 |
| CATGGTCTTGAGTT | | CACGGACTAGAATT | |
| GGTCTTGAGTTAAAT | 1952 | GGACTAGAATTGAAC | 1953 |
| GCTGACATCT | | GCAGATATTT | |
| GAGTTAAATGCTGAC | 1954 | GAATTGAACGCAGAT | 1955 |
| ATCTTA | | ATTTTG | |
| GAGTTAAATGCTGAC | 1956 | GAATTGAACGCAGAT | 1957 |
| ATCTTAGGCACTGA | | ATTTTGGGGACAGA | |
| GTTAAATGCTGACAT | 1958 | ATTGAACGCAGATAT | 1959 |
| CTTA | | TTTG | |
| GGGCATCTATGAAAT | 1960 | GCGCTTCAATGAAGT | 1961 |
| TAACAA | | TGACTA | |
| GGGCATCTATGAAAT | 1962 | GCGCTTCAATGAAGT | 1963 |
| TAACAACAAA | | TGACTACTAA | |
| GGCATCTATGAAATT | 1964 | CGCTTCAATGAAGTT | 1965 |
| AACA | | GACT | |
| GAAGGACTTAAGCTC | 1966 | GAGGGTCTAAAACTG | 1967 |
| TCAAAT | | TCTAAC | |
| GAAGGACTTAAGCTC | 1968 | GAGGGTCTAAAACTG | 1969 |
| TCAAATGACA | | TCTAACGATA | |
| GAAGGACTTAAGCTC | 1970 | GAGGGTCTAAAACTG | 1971 |
| TCAAATGACATGAT | | TCTAACGATATGAT | |
| GAAGGACTTAAGCTC | 1972 | GAGGGTCTAAAACTG | 1973 |
| TCAA | | TCTA | |
| GGACTTAAGCTCTCA | 1974 | GGTCTAAAACTGTCT | 1975 |
| AATGACATGA | | AACGATATGA | |
| GCAGGCTTATCACTG | 1976 | GCTGGGTTGTCTCTC | 1977 |
| GACTTCTCTT | | GATTTTTCAT | |
| GCAGGCTTATCACTG | 1978 | GCTGGGTTGTCTCTC | 1979 |
| GACTTCTCTTCAAA | | GATTTTTCATCTAA | |
| GGCTTATCACTGGAC | 1980 | GGGTTGTCTCTCGAT | 1981 |
| TTCTCT | | TTTTCA | |
| GGCTTATCACTGGAC | 1982 | GGGTTGTCTCTCGAT | 1983 |
| TTCTCTTCAA | | TTTTCATCTA | |
| GGCTTATCACTGGAC | 1984 | GGGTTGTCTCTCGAT | 1985 |
| TTCT | | TTTT | |
| GCTTATCACTGGACT | 1986 | GGTTGTCTCTCGATT | 1987 |
| TCTCTT | | TTTCAT | |
| GCTTATCACTGGACT | 1988 | GGTTGTCTCTCGATT | 1989 |
| TCTCTTCAAA | | TTTCATCTAA | |
| GCTACAGCCCTATTC | 1990 | ACTTCAACCGTACTC | 1991 |
| TCTGGTAACT | | ACTCGTTACA | |
| GCCCTATTCTCTGGT | 1992 | ACCGTACTCACTCGT | 1993 |
| AACT | | TACA | |
| GCTCTGGATCTCACC | 1994 | GCACTCGACCTGACG | 1995 |
| AACAAT | | AATAAC | |
| GGATCTCACCAACAA | 1996 | CGACCTGACGAATAA | 1997 |
| TGGGAAACTA | | CGGCAAGCTT | |
| GCCTTATCAGCAAGC | 1998 | GCGTTGTCTGCTAGT | 1999 |
| TATA | | TACA | |
| GCAAGCTATAAAGCA | 2000 | GCTAGTTACAAGGCT | 2001 |
| GACACT | | GATACA | |
| GCTATAAAGCAGACA | 2002 | GTTACAAGGCTGATA | 2003 |
| CTGT | | CAGT | |
| GCAGACACTGTTGCT | 2004 | GCTGATACAGTAGCA | 2005 |
| AAGGTT | | AAAGTA | |
| GCTTCAGCCATTGAC | 2006 | GCATCTGCGATAGAT | 2007 |
| ATGA | | ATGA | |
| GGGCAGCTGTATAGC | 2008 | GGCCAACTCTACAGT | 2009 |
| AAATTCCTGTTGAA | | AAGTTTCTCTTAAA | |
| GGCAGCTGTATAGCA | 2010 | GCCAACTCTACAGTA | 2011 |
| AATTCCTGTTGAAA | | AGTTTCTCTTAAAG | |
| GCTGTATAGCAAATT | 2012 | ACTCTACAGTAAGTT | 2013 |
| CCTGTTGAAA | | TCTCTTAAAG | |
| GAACCTCTGGCATTT | 2014 | GAGCCACTCGCTTTC | 2015 |
| ACTTTCTCTCATGA | | ACATTTTCACACGA | |
| GAACCTCTGGCATTT | 2016 | GAGCCACTCGCTTTC | 2017 |
| ACTT | | ACAT | |
| GGCATTTACTTTCTC | 2018 | CGCTTTCACATTTTC | 2019 |
| TCATGA | | ACACGA | |
| GGCATTTACTTTCTC | 2020 | CGCTTTCACATTTTC | 2021 |
| TCAT | | ACAC | |
| GCATTTACTTTCTCT | 2022 | GCTTTCACATTTTCA | 2023 |
| CATGAT | | CACGAC | |
| GCATTTACTTTCTCT | 2024 | GCTTTCACATTTTCA | 2025 |
| CATGATTACA | | CACGACTATA | |
| GCTCCACAAGTCATC | 2026 | GGTCGACTAGCCACC | 2027 |
| ATCT | | ACCT | |
| GGCACCTGGAAACTC | 2028 | GGGACGTGGAAGCTG | 2029 |
| AAGACCCAATTTAA | | AAAACGCAGTTCAA | |
| GGCACCTGGAAACTC | 2030 | GGGACGTGGAAGCTG | 2031 |
| AAGA | | AAAA | |
| GGAAACTCAAGACCC | 2032 | GGAAGCTGAAAACGC | 2033 |
| AATTTA | | AGTTCA | |
| GGAAACTCAAGACCC | 2034 | GGAAGCTGAAAACGC | 2035 |
| AATTTAACAA | | AGTTCAATAA | |
| GGAAACTCAAGACCC | 2036 | GGAAGCTGAAAACGC | 2037 |
| AATTTAACAACAAT | | AGTTCAATAATAAC | |
| GAAACTCAAGACCCA | 2038 | GAAGCTGAAAACGCA | 2039 |
| ATTTAA | | GTTCAA | |
| GACCCAATTTAACAA | 2040 | AACGCAGTTCAATAA | 2041 |
| CAATGA | | TAACGA | |
| GACCCAATTTAACAA | 2042 | AACGCAGTTCAATAA | 2043 |
| CAAT | | TAAC | |
| GGACGAACTCTGGCT | 2044 | GGTCGTACACTCGCA | 2045 |
| GACCTAACTCTACT | | GATCTTACACTTCT | |
| GACGAACTCTGGCTG | 2046 | GTCGTACACTCGCAG | 2047 |
| ACCTAA | | ATCTTA | |
| GACGAACTCTGGCTG | 2048 | GTCGTACACTCGCAG | 2049 |
| ACCTAACTCT | | ATCTTACACT | |
| GAACTCTGGCTGACC | 2050 | GTACACTCGCAGATC | 2051 |
| TAACTCTACT | | TTACACTTCT | |
| GATGCTTTAGAGATG | 2052 | GACGCATTGGAAATG | 2053 |
| AGAGAT | | AGGGAC | |
| GAAGCCCCAAGAATT | 2054 | AAAACCGCAGGAGTT | 2055 |
| TACAAT | | CACTAT | |
| GAAACCTGAAGCACA | 2056 | GGAATCTCAAACATA | 2057 |
| TCAATA | | TTAACA | |
| GAAACTGACTGCTCT | 2058 | AAAGCTCACAGCACT | 2059 |
| CACA | | GACT | |
| GGGAACTACAATTTC | 2060 | AGGTACAACTATATC | 2061 |
| ATTT | | TTTC | |
| GCCTTCAGAGCCAAA | 2062 | GCGTTTAGGGCGAAG | 2063 |
| GTCCATGAGT | | GTGCACGAAT | |
| GCCTTCAGAGCCAAA | 2064 | GCGTTTAGGGCGAAG | 2065 |
| GTCCATGAGT | | GTGCACGAAT | |
| GCCTTCAGAGCCAAA | 2066 | GCGTTTAGGGCGAAG | 2067 |
| GTCCATGAGTTAAT | | GTGCACGAATTGAT | |
| GAGCCAAAGTCCATG | 2068 | GGGCGAAGGTGCACG | 2069 |
| AGTTAA | | AATTGA | |
| GAGCCAAAGTCCATG | 2070 | GGGCGAAGGTGCACG | 2071 |
| AGTT | | AATT | |
| GCCAAAGTCCATGAG | 2072 | GCGAAGGTGCACGAA | 2073 |
| TTAATCGAGAGGTA | | TTGATTGAAAGATA | |
| GCCAAAGTCCATGAG | 2074 | GCGAAGGTGCACGAA | 2075 |
| TTAA | | TTGA | |
| GTCCATGAGTTAATC | 2076 | GTGCACGAATTGATT | 2077 |
| GAGAGGTATGAAGT | | GAAAGATACGAGGT | |
| GGCCCACCAATACAA | 2078 | AGCGCATCAGTATAA | 2079 |
| GTTGAA | | ATTAAA | |
| GGCCCACCAATACAA | 2080 | AGCGCATCAGTATAA | 2081 |
| GTTGAAGGAGACTA | | ATTAAAAGAAACAA | |
| GCCCACCAATACAAG | 2082 | GCGCATCAGTATAAA | 2083 |
| TTGAAGGAGACTAT | | TTAAAAGAAACAAT | |
| GCCCACCAATACAAG | 2084 | GCGCATCAGTATAAA | 2085 |
| TTGA | | TTAA | |
| GAAGCTAAGCAATGT | 2086 | AAAACTTAGTAACGT | 2087 |
| CCTACA | | GCTTCA | |
| GAAGCTAAGCAATGT | 2088 | AAAACTTAGTAACGT | 2089 |
| CCTA | | GCTT | |
| GCTAAGCAATGTCCT | 2090 | ACTTAGTAACGTGCT | 2091 |
| ACAA | | TCAG | |
| GATTTATTGATGATG | 2092 | GTTTCATAGACGACG | 2093 |
| CTGTCA | | CAGTGA | |
| GATGCTGTCAAGAAG | 2094 | GACGCAGTGAAAAAA | 2095 |
| CTTAAT | | CTAAAC | |
| GATGCTGTCAAGAAG | 2096 | GACGCAGTGAAAAAA | 2097 |
| CTTAATGAAT | | CTAAACGAGT | |
| GCTGTCAAGAAGCTT | 2098 | GCAGTGAAAAAACTA | 2099 |
| AATGAA | | AACGAG | |
| GGTGACTCAGAGACT | 2100 | AGTCACACAAAGGCT | 2101 |
| CAAT | | GAAC | |
| GAGGAAACCAAGGCC | 2102 | GAAGAGACGAAAGCG | 2103 |
| ACAGTT | | ACTGTA | |
| GAGGAAACCAAGGCC | 2104 | GAAGAGACGAAAGCG | 2105 |
| ACAGTTGCAGTGTA | | ACTGTAGCTGTCTA | |
| GGAAACCAAGGCCAC | 2106 | AGAGACGAAAGCGAC | 2107 |
| AGTT | | TGTA | |
| GAAACCAAGGCCACA | 2108 | GAGACGAAAGCGACT | 2109 |
| GTTGCAGTGT | | GTAGCTGTCT | |
| GAAACCAAGGCCACA | 2110 | GAGACGAAAGCGACT | 2111 |
| GTTGCAGTGTATCT | | GTAGCTGTCTACCT | |
| GGCCACAGTTGCAGT | 2112 | AGCGACTGTAGCTGT | 2113 |
| GTATCT | | CTACCT | |
| GGCCACAGTTGCAGT | 2114 | AGCGACTGTAGCTGT | 2115 |
| GTAT | | CTAC | |
| GGTTACAGGAGGCTT | 2116 | GGTTGCAAGAAGCAT | 2117 |
| TAAGTT | | TGAGCT | |
| GGCTTTAAGTTCAGC | 2118 | AGCATTGAGCTCTGC | 2119 |
| ATCTTT | | TTCATT | |
| GGACATTCAGCAGGA | 2120 | GGATATACAACAAGA | 2121 |
| ACTTCA | | GCTACA | |
| GGACATTCAGCAGGA | 2122 | GGATATACAACAAGA | 2123 |
| ACTT | | GCTA | |
| GGTTTATAGCACACT | 2124 | AGTATACAGTACTCT | 2125 |
| TGTCACCTACATTT | | AGTGACGTATATAT | |
| GTTTATAGCACACTT | 2126 | GTATACAGTACTCTA | 2127 |
| GTCACCTACA | | GTGACGTATA | |
| GCACACTTGTCACCT | 2128 | GTACTCTAGTGACGT | 2129 |
| ACATTT | | ATATAT | |
| GCACACTTGTCACCT | 2130 | GTACTCTAGTGACGT | 2131 |
| ACATTTCTGA | | ATATATCAGA | |
| GCACACTTGTCACCT | 2132 | GTACTCTAGTGACGT | 2133 |
| ACAT | | ATAT | |
| GGTAGAGCAAGGGTT | 2134 | AGTTGAACAGGGCTT | 2135 |
| CACTGT | | TACAGT | |
| GGTAGAGCAAGGGTT | 2136 | AGTTGAACAGGGCTT | 2137 |
| CACT | | TACA | |
| GTTCCTGAAATCAAG | 2138 | GTACCAGAGATTAAA | 2139 |
| ACCA | | ACGA | |
| GGCTCTTCAGAAAGC | 2140 | AGCACTACAAAAGGC | 2141 |
| TACCTT | | AACGTT | |
| GCTCTTCAGAAAGCT | 2142 | GCACTACAAAAGGCA | 2143 |
| ACCT | | ACGT | |
| GGATTCCATCAGTTC | 2144 | GAATACCTTCTGTAC | 2145 |
| AGATAA | | AAATTA | |
| GATTCCATCAGTTCA | 2146 | AATACCTTCTGTACA | 2147 |
| GATAAA | | AATTAA | |
| GATTCCATCAGTTCA | 2148 | AATACCTTCTGTACA | 2149 |
| GATA | | AATT | |
| GAATTTACCATCCTT | 2150 | GAGTTCACGATTCTA | 2151 |
| AACA | | AATA | |
| GAAAGTAAAGATCAT | 2152 | GAAGGTTAAAATTAT | 2153 |
| CAGA | | TAGG | |
| GGATCTGAAGGTGGA | 2154 | AGACCTCAAAGTCGA | 2155 |
| GGACAT | | AGATAT | |
| GAGAATCACCCTGCC | 2156 | CAGGATTACGCTCCC | 2157 |
| AGACTT | | TGATTT | |
| GAATCACCCTGCCAG | 2158 | GGATTACGCTCCCTG | 2159 |
| ACTT | | ATTT | |
| GCAAATGCACAACTC | 2160 | GCTAACGCTCAGCTG | 2161 |
| TCAAACCCTAAGAT | | TCTAATCCAAAAAT | |
| GCACAACTCTCAAAC | 2162 | GCTCAGCTGTCTAAT | 2163 |
| CCTAAGATTA | | CCAAAAATAA | |
| GAACGGAGCATGGGA | 2164 | GGACCGAACACGGCA | 2165 |
| GTGAAA | | GCGAGA | |
| GGAGTGATTGTCAAG | 2166 | GGTGTCATAGTGAAA | 2167 |
| ATAA | | ATTA | |
| GAGTGATTGTCAAGA | 2168 | GTGTCATAGTGAAAA | 2169 |
| TAAA | | TTAA | |
| GCTTACCCTGGATAG | 2170 | ACTAACGCTCGACAG | 2171 |
| CAACACTAAA | | TAATACAAAG | |
| GGATAGCAACACTAA | 2172 | CGACAGTAATACAAA | 2173 |
| ATACTT | | GTATTT | |
| GGATAGCAACACTAA | 2174 | CGACAGTAATACAAA | 2175 |
| ATACTTCCACAAAT | | GTATTTTCATAAGT | |
| GCAACACTAAATACT | 2176 | GTAATACAAAGTATT | 2177 |
| TCCACAAATT | | TTCATAAGTT | |
| GAACATCCCCAAACT | 2178 | AAATATTCCGAAGCT | 2179 |
| GGACTTCTCT | | CGATTTTTCA | |
| GACCTGCGCAACGAG | 2180 | GATCTCCGGAATGAA | 2181 |
| ATCAAGACACTGTT | | ATTAAAACTCTCTT | |
| GCGCAACGAGATCAA | 2182 | CCGGAATGAAATTAA | 2183 |
| GACACT | | AACTCT | |
| GCAACGAGATCAAGA | 2184 | GGAATGAAATTAAAA | 2185 |
| CACTGT | | CTCTCT | |
| GCAACGAGATCAAGA | 2186 | GGAATGAAATTAAAA | 2187 |
| CACT | | CTCT | |
| GTTGAAAGCTGGCCA | 2188 | CTTAAAGGCAGGGCA | 2189 |
| CATAGCATGGACTT | | TATTGCTTGGACAT | |
| GAAAGCTGGCCACAT | 2190 | AAAGGCAGGGCATAT | 2191 |
| AGCATGGACTTCTT | | TGCTTGGACATCAT | |
| GCTGGCCACATAGCA | 2192 | GCAGGGCATATTGCT | 2193 |
| TGGACTTCTT | | TGGACATCAT | |
| GGCCACATAGCATGG | 2194 | GGGCATATTGCTTGG | 2195 |
| ACTTCT | | ACATCA | |
| GGCCACATAGCATGG | 2196 | GGGCATATTGCTTGG | 2197 |
| ACTT | | ACAT | |
| GCCACATAGCATGGA | 2198 | GGCATATTGCTTGGA | 2199 |
| CTTCTT | | CATCAT | |
| GCCCCAGATTCTCAG | 2200 | GTCCGAGGTTTTCTG | 2201 |
| ATGA | | ACGA | |
| GATCAATAGCAAACA | 2202 | AATTAACAGTAAGCA | 2203 |
| CCTAAGAGTA | | TCTTAGGGTT | |
| GCTAAAGGCATGGCA | 2204 | GCAAAGGGGATGGCT | 2205 |
| CTGTTT | | CTCTTC | |
| GCTAAAGGCATGGCA | 2206 | GCAAAGGGGATGGCT | 2207 |
| CTGT | | CTCT | |
| GGAGAAGGGAAGGCA | 2208 | GGTGAGGGCAAAGCT | 2209 |
| GAGTTT | | GAATTC | |
| GAGAAGGGAAGGCAG | 2210 | GTGAGGGCAAAGCTG | 2211 |
| AGTTTA | | AATTCA | |
| GAGAAGGGAAGGCAG | 2212 | GTGAGGGCAAAGCTG | 2213 |
| AGTT | | AATT | |
| GAAGGGAAGGCAGAG | 2214 | GAGGGCAAAGCTGAA | 2215 |
| TTTA | | TTCA | |
| GGAAAGGTTATTGGA | 2216 | GGTAAAGTAATAGGT | 2217 |
| ACTT | | ACAT | |
| GCAAGTTGGCAAGTA | 2218 | GCTAGCTGGCAGGTT | 2219 |
| AGTGCTAGGT | | AGCGCAAGAT | |
| GCAAGTTGGCAAGTA | 2220 | GCTAGCTGGCAGGTT | 2221 |
| AGTGCTAGGTTCAA | | AGCGCAAGATTTAA | |
| GTTGGCAAGTAAGTG | 2222 | GCTGGCAGGTTAGCG | 2223 |
| CTAGGT | | CAAGAT | |
| GTTGGCAAGTAAGTG | 2224 | GCTGGCAGGTTAGCG | 2225 |
| CTAGGTTCAA | | CAAGATTTAA | |
| GGCAAGTAAGTGCTA | 2226 | GGCAGGTTAGCGCAA | 2227 |
| GGTTCA | | GATTTA | |
| GGCAAGTAAGTGCTA | 2228 | GGCAGGTTAGCGCAA | 2229 |
| GGTTCAATCA | | GATTTAACCA | |
| GGCAAGTAAGTGCTA | 2230 | GGCAGGTTAGCGCAA | 2231 |
| GGTTCAATCAGTAT | | GATTTAACCAATAC | |
| GGCAAGTAAGTGCTA | 2232 | GGCAGGTTAGCGCAA | 2233 |
| GGTT | | GATT | |
| GCAAGTAAGTGCTAG | 2234 | GCAGGTTAGCGCAAG | 2235 |
| GTTCAA | | ATTTAA | |
| GCAAGTAAGTGCTAG | 2236 | GCAGGTTAGCGCAAG | 2237 |
| GTTCAATCAGTATA | | ATTTAACCAATACA | |
| GTAAGTGCTAGGTTC | 2238 | GTTAGCGCAAGATTT | 2239 |
| AATCAGTATA | | AACCAATACA | |
| GTGCTAGGTTCAATC | 2240 | GCGCAAGATTTAACC | 2241 |
| AGTATA | | AATACA | |
| GTGCTAGGTTCAATC | 2242 | GCGCAAGATTTAACC | 2243 |
| AGTA | | AATA | |
| GCTAGGTTCAATCAG | 2244 | GCAAGATTTAACCAA | 2245 |
| TATA | | TACA | |
| GGAGGCCCATGTAGG | 2246 | GGAAGCGCACGTTGG | 2247 |
| AATAAA | | TATTAA | |
| GAGGCCCATGTAGGA | 2248 | GAAGCGCACGTTGGT | 2249 |
| ATAAAT | | ATTAAC | |
| GGCCCATGTAGGAAT | 2250 | AGCGCACGTTGGTAT | 2251 |
| AAAT | | TAAC | |
| GCTCCCCAGGACCTT | 2252 | ACTGCCGAGAACGTT | 2253 |
| TCAAAT | | CCAGAT | |
| GACCTTTCAAATTCC | 2254 | AACGTTCCAGATACC | 2255 |
| TGGATACACT | | AGGTTATACA | |
| GAGCTGCCAGTCCTT | 2256 | GAACTCCCTGTGCTA | 2257 |
| CATGTCCCTAGAAA | | CACGTGCCAAGGAA | |
| GCCAGTCCTTCATGT | 2258 | CCCTGTGCTACACGT | 2259 |
| CCCTAGAAAT | | GCCAAGGAAC | |
| GTCCTTCATGTCCCT | 2260 | GTGCTACACGTGCCA | 2261 |
| AGAAAT | | AGGAAC | |
| GTCCTTCATGTCCCT | 2262 | GTGCTACACGTGCCA | 2263 |
| AGAA | | AGGA | |
| GCTTTCTCTTCCAGA | 2264 | ACTATCACTACCTGA | 2265 |
| TTTCAA | | CTTTAA | |
| GCCATGGGCAATATT | 2266 | GCGATGGGGAACATA | 2267 |
| ACCTAT | | ACGTAC | |
| GCCATGGGCAATATT | 2268 | GCGATGGGGAACATA | 2269 |
| ACCTATGATT | | ACGTACGACT | |
| GGGCAATATTACCTA | 2270 | GGGGAACATAACGTA | 2271 |
| TGATTT | | CGACTT | |
| GGGCAATATTACCTA | 2272 | GGGGAACATAACGTA | 2273 |
| TGAT | | CGAC | |
| GGCAATATTACCTAT | 2274 | GGGAACATAACGTAC | 2275 |
| GATT | | GACT | |
| GTTGCTCATCTCCTT | 2276 | GTAGCACACCTGCTA | 2277 |
| TCTTCA | | TCATCT | |
| GTTGCTCATCTCCTT | 2278 | GTAGCACACCTGCTA | 2279 |
| TCTT | | TCAT | |
| GCTCATCTCCTTTCT | 2280 | GCACACCTGCTATCA | 2281 |
| TCATCT | | TCTTCA | |
| GCTCATCTCCTTTCT | 2282 | GCACACCTGCTATCA | 2283 |
| TCATCTTCAT | | TCTTCATCTT | |
| GCTCATCTCCTTTCT | 2284 | GCACACCTGCTATCA | 2285 |
| TCATCTTCATCTGT | | TCTTCATCTTCAGT | |
| GCTCATCTCCTTTCT | 2286 | GCACACCTGCTATCA | 2287 |
| TCAT | | TCTT | |
| GAGGGCACCACAAGA | 2288 | GAAGGGACGACTAGG | 2289 |
| TTGACAAGAA | | TTAACTAGGA | |
| GAGGGCACCACAAGA | 2290 | GAAGGGACGACTAGG | 2291 |
| TTGA | | TTAA | |
| GGCACCACAAGATTG | 2292 | GGGACGACTAGGTTA | 2293 |
| ACAAGA | | ACTAGG | |
| GGCACCACAAGATTG | 2294 | GGGACGACTAGGTTA | 2295 |
| ACAA | | ACTA | |
| GCACCACAAGATTGA | 2296 | GGACGACTAGGTTAA | 2297 |
| CAAGAA | | CTAGGA | |
| GTGGAGGGTAGTCAT | 2298 | GTCGAAGGAAGCCAC | 2299 |
| AACAGT | | AATAGC | |
| GGAGGGTAGTCATAA | 2300 | CGAAGGAAGCCACAA | 2301 |
| CAGT | | TAGC | |
| GAGGGTAGTCATAAC | 2302 | GAAGGAAGCCACAAT | 2303 |
| AGTA | | AGCA | |
| GTATGATTTCAATTC | 2304 | ATACGACTTTAACTC | 2305 |
| TTCAATGCTGTACT | | ATCTATGCTCTATT | |
| GATTTCAATTCTTCA | 2306 | GACTTTAACTCATCT | 2307 |
| ATGCTGTACT | | ATGCTCTATT | |
| GGAAAGCCTCACCTC | 2308 | AGAGAGTCTGACGTC | 2309 |
| TTACTT | | ATATTT | |
| GAAAGCCTCACCTCT | 2310 | GAGAGTCTGACGTCA | 2311 |
| TACT | | TATT | |
| GGAGATGTCAAGGGT | 2312 | GGTGACGTGAAAGGA | 2313 |
| TCGGTTCTTT | | TCCGTACTAT | |
| GAGGCCAACACTTAC | 2314 | GAAGCGAATACATAT | 2315 |
| TTGAAT | | TTAAAC | |
| GAGGCCAACACTTAC | 2316 | GAAGCGAATACATAT | 2317 |
| TTGA | | TTAA | |
| GGCCAACACTTACTT | 2318 | AGCGAATACATATTT | 2319 |
| GAAT | | AAAC | |
| GCCAACACTTACTTG | 2320 | GCGAATACATATTTA | 2321 |
| AATT | | AACT | |
| GCAAGTCAGCCCAGT | 2322 | GCTAGCCAACCGAGC | 2323 |
| TCCTTCCATGATTT | | TCGTTTCACGACTT | |
| GCCCAGTTCCTTCCA | 2324 | ACCGAGCTCGTTTCA | 2325 |
| TGATTT | | CGACTT | |
| GTTCCTTCCATGATT | 2326 | GCTCGTTTCACGACT | 2327 |
| TCCCTGACCT | | TTCCAGATCT | |
| GTGGCCCTGAATGCT | 2328 | GTCGCGCTCAACGCA | 2329 |
| AACACT | | AATACA | |
| GTGGCCCTGAATGCT | 2330 | GTCGCGCTCAACGCA | 2331 |
| AACA | | AATA | |
| GGCCCTGAATGCTAA | 2332 | CGCGCTCAACGCAAA | 2333 |
| CACTAA | | TACAAA | |
| GGCCCTGAATGCTAA | 2334 | CGCGCTCAACGCAAA | 2335 |
| CACT | | TACA | |
| GCCCTGAATGCTAAC | 2336 | GCGCTCAACGCAAAT | 2337 |
| ACTA | | ACAA | |
| GGTTCCATCGTGCAA | 2338 | AGTACCTTCCTGTAA | 2339 |
| ACTTGA | | GCTAGA | |
| GGTTCCATCGTGCAA | 2340 | AGTACCTTCCTGTAA | 2341 |
| ACTT | | GCTA | |
| GTTCCATCGTGCAAA | 2342 | GTACCTTCCTGTAAG | 2343 |
| CTTGACTTCA | | CTAGATTTTA | |
| GTTCCATCGTGCAAA | 2344 | GTACCTTCCTGTAAG | 2345 |
| CTTGACTTCAGAGA | | CTAGATTTTAGGGA | |
| GTGCAAACTTGACTT | 2346 | CTGTAAGCTAGATTT | 2347 |
| CAGAGA | | TAGGGA | |
| GTGCAAACTTGACTT | 2348 | CTGTAAGCTAGATTT | 2349 |
| CAGA | | TAGG | |
| GCAAACTTGACTTCA | 2350 | GTAAGCTAGATTITA | 2351 |
| GAGAAA | | GGGAGA | |
| GCAAACTTGACTTCA | 2352 | GTAAGCTAGATTITA | 2353 |
| GAGA | | GGGA | |
| GCTGAGAACTTCATC | 2354 | ACTCAGGACATCTTC | 2355 |
| ATTT | | TTTC | |
| GTACCTGCTGGAATT | 2356 | GTTCCAGCAGGTATA | 2357 |
| GTCA | | GTGA | |
| GTGACTTCAGTGCAG | 2358 | GAGATTTTAGCGCTG | 2359 |
| AATA | | AGTA | |
| GTGCAGAATATGAAG | 2360 | GCGCTGAGTACGAGG | 2361 |
| AAGA | | AGGA | |
| GATGGCAAATATGAA | 2362 | GACGGGAAGTACGAG | 2363 |
| GGACTT | | GGTCTA | |
| GCTTCTGGCTTGCTA | 2364 | GCATCAGGGTTACTT | 2365 |
| ACCTCTCTGA | | ACGTCACTCA | |
| GCTTCTGGCTTGCTA | 2366 | GCATCAGGGTTACTT | 2367 |
| ACCTCTCTGAAAGA | | ACGTCACTCAAGGA | |
| GCTTCTGGCTTGCTA | 2368 | GCATCAGGGTTACTT | 2369 |
| ACCT | | ACGT | |
| GGCTTGCTAACCTCT | 2370 | GGGTTACTTACGTCA | 2371 |
| CTGAAA | | CTCAAG | |
| GGCTTGCTAACCTCT | 2372 | GGGTTACTTACGTCA | 2373 |
| CTGAAAGACA | | CTCAAGGATA | |
| GGCTTGCTAACCTCT | 2374 | GGGTTACTTACGTCA | 2375 |
| CTGA | | CTCA | |
| GCTTGCTAACCTCTC | 2376 | GGTTACTTACGTCAC | 2377 |
| TGAAAGACAA | | TCAAGGATAA | |
| GCTTGCTAACCTCTC | 2378 | GGTTACTTACGTCAC | 2379 |
| TGAA | | TCAA | |
| GCTAACCTCTCTGAA | 2380 | ACTTACGTCACTCAA | 2381 |
| AGACAA | | GGATAA | |
| GGGCCATTAGGCAAA | 2382 | GCGCGATAAGACAGA | 2383 |
| TTGA | | TAGA | |
| GGCCATTAGGCAAAT | 2384 | CGCGATAAGACAGAT | 2385 |
| TGATGA | | AGACGA | |
| GGCCATTAGGCAAAT | 2386 | CGCGATAAGACAGAT | 2387 |
| TGAT | | AGAC | |
| GGACCTACCAAGAGT | 2388 | GCACGTATCAGGAAT | 2389 |
| GGAAGGACAA | | GGAAAGATAA | |

In some cases, the mammal can have DCM or HCM, and the gene to be suppressed and replaced can be TNNI3 (which encodes cardiac type Troponin 13). An exemplary TNNI3 sequence is set forth in NCBI RefSeq accession number NM_000363 (e.g., version NM_000363.5). A TNNI3 polypeptide can, in some cases, have the amino acid sequence set forth in NCBI RefSeq accession number Q59H18 (e.g., version Q59H18.3).

Examples of shRNA sequences and corresponding shIMM sequences targeted to TNNI3 are set forth in TABLE 1Z.

TABLE 1Z
Representative TNNI3 shRNA and
shIMM sequences

| shRNA | SEQ | shIMM | SEQ |
|---|---|---|---|
| Sequence | ID | Sequence | ID |
| GTGGACAAGG | 2390 | CTGGACTAGA | 2391 |
| TGGATGAAGA | | TGGATGAAAA | |
| GAGAT | | GGGAC | |
| GACAAGGTGG | 2392 | GACTAGATGG | 2393 |
| ATGAAGAGA | | ATGAAAAGG | |
| GACCTTCGAG | 2394 | GATCTACGTG | 2395 |
| GCAAGTTTA | | GGAAATTCA | |

In some cases, the mammal can have DCM or HCM, and the gene to be suppressed and replaced can be TNNC1 (which encodes slow skeletal and cardiac type Troponin C1). An exemplary TNNC1 sequence is set forth in NCBI RefSeq accession number NM_003280 (e.g., version NM_003280.3). A TNNC1 polypeptide can, in some cases, have the amino acid sequence set forth in NCBI RefSeq accession number NP_003271 (e.g., version NP_003271.1).

Examples of shRNA sequences and corresponding shIMM sequences targeted to TNNC1 are set forth in TABLE 1AA.

TABLE 1AA
Representative TNNC1 shRNA
and shIMM sequences

| shRNA | SEQ | shIMM | SEQ |
|---|---|---|---|
| Sequence | ID | Sequence | ID |
| GGTAGAGCAG | 2396 | CGTTGAACAA | 2397 |
| CTGACAGAA | | CTCACTGAG | |
| AGGAGCTGCA | 2398 | AAGAACTCCA | 2399 |
| GGAGATGAT | | AGAAATGAT | |
| GATGGTTCGG | 2400 | GATGGTACGC | 2401 |
| TGCATGAAG | | TGTATGAAA | |
| GCATGAAGGA | 2402 | GTATGAAAGA | 2403 |
| CGACAGCAAA | | TGATAGTAAG | |
| GGGAAATCT | | GGCAAGTCA | |
| ATGAAGGACG | 2404 | ATGAAAGATG | 2405 |
| ACAGCAAAGG | | ATAGTAAGGG | |
| GAAATCTGA | | CAAGTCAGA | |
| GAAGGACGAC | 2406 | GAAAGATGAT | 2407 |
| AGCAAAGGGA | | AGTAAGGGCA | |
| AATCT | | AGTCA | |
| AGGACGACAG | 2408 | AAGATGATAG | 2409 |
| CAAAGGGAAA | | TAAGGGCAAG | |
| TCTGA | | TCAGA | |
| GGACGACAGC | 2410 | AGATGATAGT | 2411 |
| AAAGGGAAAT | | AAGGGCAAGT | |
| CTGAG | | CAGAA | |
| GACGACAGCA | 2412 | GATGATAGTA | 2413 |
| AAGGGAAATC | | AGGGCAAGTC | |
| T | | A | |
| ACAGCAAAGG | 2414 | ATAGTAAGGG | 2415 |
| GAAATCTGA | | CAAGTCAGA | |
| GCAAAGGGAA | 2416 | GTAAGGGCAA | 2417 |
| ATCTGAGGAG | | GTCAGAAGAA | |
| GAGCTGTCT | | GAACTCTCA | |
| AAAGGGAAAT | 2418 | AAGGGCAAGT | 2419 |
| CTGAGGAGGA | | CAGAAGAAGA | |
| GCTGTCTGA | | ACTCTCAGA | |
| AGGGAAATCT | 2420 | GGGCAAGTCA | 2421 |
| GAGGAGGAGC | | GAAGAAGAAC | |
| TGTCT | | TCTCA | |
| GGAAATCTGA | 2422 | GCAAGTCAGA | 2423 |
| GGAGGAGCTG | | AGAAGAACTC | |
| TCTGA | | TCAGA | |
| GAAATCTGAG | 2424 | CAAGTCAGAA | 2425 |
| GAGGAGCTGT | | GAAGAACTCT | |
| CTGAC | | CAGAT | |
| AAATCTGAGG | 2426 | AAGTCAGAAG | 2427 |
| AGGAGCTGTC | | AAGAACTCTC | |
| T | | A | |
| ATCTGAGGAG | 2428 | GTCAGAAGAA | 2429 |
| GAGCTGTCT | | GAACTCTCA | |
| AGGAGCTGTC | 2430 | AAGAACTCTC | 2431 |
| TGACCTCTTC | | AGATCTGTTT | |
| CGCATGTTT | | CGGATGTTC | |
| AGGAGCTGTC | 2432 | AAGAACTCTC | 2433 |
| TGACCTCTT | | AGATCTGTT | |
| GCTGTCTGAC | 2434 | ACTCTCAGAT | 2435 |
| CTCTTCCGCA | | CTGTTTCGGA | |
| TGTTT | | TGTTC | |
| ATCGACCTGG | 2436 | ATTGATCTCG | 2437 |
| ATGAGCTGAA | | ACGAACTCAA | |
| GATAA | | AATTA | |
| GACCTGGATG | 2438 | GATCTCGACG | 2439 |
| AGCTGAAGAT | | AACTCAAAAT | |
| A | | T | |
| GACCTGGATG | 2440 | GATCTCGACG | 2441 |
| AGCTGAAGA | | AACTCAAAA | |
| ACCTGGATGA | 2442 | ATCTCGACGA | 2443 |
| GCTGAAGATA | | ACTCAAAATT | |
| A | | A | |
| ACCTGGATGA | 2444 | ATCTCGACGA | 2445 |
| GCTGAAGAT | | ACTCAAAAT | |
| GGATGAGCTG | 2446 | CGACGAACTC | 2447 |
| AAGATAATG | | AAAATTATG | |
| GAGCTGAAGA | 2448 | GAACTCAAAA | 2449 |
| TAATGCTGCA | | TTATGCTCCA | |
| GGCTACAGG | | AGCAACTGG | |
| AGGACGACAT | 2450 | AAGATGATAT | 2451 |
| CGAGGAGCTC | | TGAAGAACTG | |
| ATGAA | | ATGAA | |
| ACGACATCGA | 2452 | ATGATATTGA | 2453 |
| GGAGCTCATG | | AGAACTGATG | |
| A | | A | |
| ATCGAGGAGC | 2454 | ATTGAAGAAC | 2455 |
| TCATGAAGGA | | TGATGAAAGA | |
| CGGAGACAA | | TGGTGATAA | |
| GAGGAGCTCA | 2456 | GAAGAACTGA | 2457 |
| TGAAGGACGG | | TGAAAGATGG | |
| AGACAAGAA | | TGATAAAAA | |
| AGGAGCTCAT | 2458 | AAGAACTGAT | 2459 |
| GAAGGACGGA | | GAAAGATGGT | |
| GACAA | | GATAA | |
| GAGCTCATGA | 2460 | GAACTGATGA | 2461 |
| AGGACGGAGA | | AAGATGGTGA | |
| CAAGAACAA | | TAAAAATAA | |
| GAGCTCATGA | 2462 | GAACTGATGA | 2463 |
| AGGACGGAGA | | AAGATGGTGA | |
| CAAGA | | TAAAA | |
| AGCTCATGAA | 2464 | AACTGATGAA | 2465 |
| GGACGGAGAC | | AGATGGTGAT | |
| AAGAA | | AAAAA | |
| AGCTCATGAA | 2466 | AACTGATGAA | 2467 |
| GGACGGAGAC | | AGATGGTGAT | |
| A | | A | |
| GCTCATGAAG | 2468 | ACTGATGAAA | 2469 |
| GACGGAGACA | | GATGGTGATA | |
| AGAAC | | AAAAT | |
| GCTCATGAAG | 2470 | ACTGATGAAA | 2471 |
| GACGGAGACA | | GATGGTGATA | |
| A | | A | |
| GAAGGACGGA | 2472 | GAAAGATGGT | 2473 |
| GACAAGAACA | | GATAAAAATA | |
| A | | A | |
| GAAGGACGGA | 2474 | GAAAGATGGT | 2475 |
| GACAAGAAC | | GATAAAAAT | |
| AGGACGGAGA | 2476 | AAGATGGTGA | 2477 |
| CAAGAACAA | | TAAAAATAA | |
| GGACGGAGAC | 2478 | AGATGGTGAT | 2479 |
| AAGAACAAC | | AAAAATAAT | |

In some cases, the mammal can have HCM or DCM, and the gene to be suppressed and replaced can be MYL2 (which encodes myosin light chain 2). An exemplary MYL2 sequence is set forth in NCBI RefSeq accession number NM_000432 (e.g., version NM_000432.4). A MYL2 polypeptide can, in some cases, have the amino acid sequence set forth in NCBI RefSeq accession number NP_000423 (e.g., version NP_000423.2).

Examples of shRNA sequences and corresponding shIMM sequences targeted to MYL2 are set forth in TABLE 1BB.

TABLE 1BB
Representative MYL2 shRNA
and shIMM sequences

| shRNA | SEQ | shIMM | SEQ |
|---|---|---|---|
| Sequence | ID | Sequence | ID |
| GGCACCTAAG | 2480 | GGCTCCAAAA | 2481 |
| AAAGCAAAGA | | AAGGCTAAAA | |
| A | | A | |
| GGCACCTAAG | 2482 | GGCTCCAAAA | 2483 |
| AAAGCAAAGA | | AAGGCTAAAA | |
| AGAGA | | AAAGG | |
| GCCAACTCCA | 2484 | GCGAATTCGA | 2485 |
| ACGTGTTCT | | ATGTCTTTT | |
| GGAGGCCTTC | 2486 | AGAAGCGTTT | 2487 |
| ACTATCATGG | | ACAATTATGG | |
| ACCAGAACA | | ATCAAAATA | |
| GCCTTCACTA | 2488 | GCGTTTACAA | 2489 |
| TCATGGACCA | | TTATGGATCA | |
| GAACA | | AAATA | |
| GGACCAGAAC | 2490 | GGATCAAAAT | 2491 |
| AGGGATGGCT | | AGAGACGGGT | |
| TCATT | | TTATA | |
| GGACCAGAAC | 2492 | GGATCAAAAT | 2493 |
| AGGGATGGCT | | AGAGACGGGT | |
| TCATTGACA | | TTATAGATA | |
| GGGATGGCTT | 2494 | GAGACGGGTT | 2495 |
| CATTGACAAG | | TATAGATAAA | |
| A | | A | |
| GGATGGCTTC | 2496 | AGACGGGTTT | 2497 |
| ATTGACAAGA | | ATAGATAAAA | |
| A | | A | |
| GGATGGCTTC | 2498 | AGACGGGTTT | 2499 |
| ATTGACAAGA | | ATAGATAAAA | |
| ACGATCTGA | | ATGACCTCA | |
| GATGGCTTCA | 2500 | GACGGGTTTA | 2501 |
| TTGACAAGA | | TAGATAAAA | |
| GGCTTCATTG | 2502 | GGGTTTATAG | 2503 |
| ACAAGAACGA | | ATAAAAATGA | |
| TCTGA | | CCTCA | |
| GGCTTCATTG | 2504 | GGGTTTATAG | 2505 |
| ACAAGAACGA | | ATAAAAATGA | |
| TCTGAGAGA | | CCTCAGGGA | |
| GAACGATCTG | 2506 | AAATGACCTC | 2507 |
| AGAGACACCT | | AGGGATACGT | |
| T | | T | |
| GAGGCTCCGG | 2508 | GAAGCACCCG | 2509 |
| GTCCAATTAA | | GACCTATAAA | |
| CTTTACTGT | | TTTCACAGT | |
| GGCTCCGGGT | 2510 | AGCACCCGGA | 2511 |
| CCAATTAACT | | CCTATAAATT | |
| T | | T | |
| GGCTCCGGGT | 2512 | AGCACCCGGA | 2513 |
| CCAATTAACT | | CCTATAAATT | |
| TTACT | | TCACA | |
| GCTCCGGGTC | 2514 | GCACCCGGAC | 2515 |
| CAATTAACTT | | CTATAAATTT | |
| T | | C | |
| GGGTCCAATT | 2516 | CGGACCTATA | 2517 |
| AACTTTACT | | AATTTCACA | |
| GGGTCCAATT | 2518 | CGGACCTATA | 2519 |
| AACTTTACTG | | AATTTCACAG | |
| T | | T | |
| GTCCAATTAA | 2520 | GACCTATAAA | 2521 |
| CTTTACTGT | | TTTCACAGT | |
| GAGGAAACCA | 2522 | GAAGAGACGA | 2523 |
| TTCTCAACGC | | TACTGAATGC | |
| ATTCAAAGT | | TTTTAAGGT | |
| GGAAACCATT | 2524 | AGAGACGATA | 2525 |
| CTCAACGCAT | | CTGAATGCTI | |
| TCAAA | | TTAAG | |
| GAAACCATTC | 2526 | GAGACGATAC | 2527 |
| TCAACGCATT | | TGAATGCTTT | |
| CAAAGTGTT | | TAAGGTCTT | |
| GGGTGCTGAA | 2528 | GCGTCCTCAA | 2529 |
| GGCTGATTA | | AGCAGACTA | |
| GGTGCTGAAG | 2530 | CGTCCTCAAA | 2531 |
| GCTGATTACG | | GCAGACTATG | |
| T | | T | |
| GGCTGATTAC | 2532 | AGCAGACTAT | 2533 |
| GTTCGGGAAA | | GTACGCGAGA | |
| TGCTGACCA | | TGCTCACGA | |
| GTTCGGGAAA | 2534 | GTACGCGAGA | 2535 |
| TGCTGACCA | | TGCTCACGA | |
| GGAGGAGGTT | 2536 | AGAAGAAGTA | 2537 |
| GACCAGATGT | | GATCAAATGT | |
| T | | T | |
| GAGGAGGTTG | 2538 | GAAGAAGTAG | 2539 |
| ACCAGATGT | | ATCAAATGT | |
| GACGTGACTG | 2540 | GATGTCACAG | 2541 |
| GCAACTTGGA | | GGAATTTAGA | |
| CTACA | | TTATA | |
| GACTGGCAAC | 2542 | CACAGGGAAT | 2543 |
| TTGGACTACA | | TTAGATTATA | |
| A | | A | |
| GGACTACAAG | 2544 | AGATTATAAA | 2545 |
| AACCTGGTGC | | AATCTCGTCC | |
| ACATCATCA | | ATATTATTA | |
| GTGCACATCA | 2546 | GTCCATATTA | 2547 |
| TCACCCACGG | | TTACGCATGG | |
| AGAAGAGAA | | TGAGGAAAA | |

In some cases, the mammal can have HCM or DCM, and the gene to be suppressed and replaced can be MYL3 (which encodes myosin light chain 3). An exemplary MYL3 sequence is set forth in NCBI RefSeq accession number NM_000258 (e.g., version NM_000258.3). An MYL3 polypeptide can, in some cases, have the amino acid sequence set forth in NCBI RefSeq accession number NP_000249 (e.g., version NP_000249.1).

Examples of shRNA sequences and corresponding shIMM sequences targeted to MYL3 are set forth in TABLE 1CC.

TABLE 1CC
Representative MYL3 shRNA
and shIMM sequences

| shRNA | SEQ | shIMM | SEQ |
|---|---|---|---|
| Sequence | ID | Sequence | ID |
| GATGCTTCCA | 2548 | GACGCATCGA | 2549 |
| AGATCAAGA | | AAATTAAAA | |
| GATGAAGATC | 2550 | AATGAAAATT | 2551 |
| ACCTACGGGC | | ACGTATGGCC | |
| AGTGT | | AATGC | |
| GAAGCCAAGA | 2552 | CAAACCTAGG | 2553 |
| CAGGAAGAGC | | CAAGAGGAAC | |
| T | | T | |
| GAAGCCAAGA | 2554 | CAAACCTAGG | 2555 |
| CAGGAAGAGC | | CAAGAGGAAC | |
| TCAATACCA | | TGAACACGA | |
| GAAGAGCTCA | 2556 | GAGGAACTGA | 2557 |
| ATACCAAGAT | | ACACGAAAAT | |
| GATGGACTT | | GATGGATTT | |
| GAGCTCAATA | 2558 | GAACTGAACA | 2559 |
| CCAAGATGA | | CGAAAATGA | |
| GCTCAATACC | 2560 | ACTGAACACG | 2561 |
| AAGATGATGG | | AAAATGATGG | |
| ACTTT | | ATTTC | |
| GAGGCTGACA | 2562 | AAGACTCACT | 2563 |
| GAAGACGAAG | | GAGGATGAGG | |
| TGGAGAAGT | | TCGAAAAAT | |
| GCTGACAGAA | 2564 | ACTCACTGAG | 2565 |
| GACGAAGTGG | | GATGAGGTCG | |
| A | | A | |
| GCTGACAGAA | 2566 | ACTCACTGAG | 2567 |
| GACGAAGTGG | | GATGAGGTCG | |
| AGAAGTTGA | | AAAAATTAA | |
| GAAGACGAAG | 2568 | GAGGATGAGG | 2569 |
| TGGAGAAGT | | TCGAAAAAT | |
| GACGAAGTGG | 2570 | GATGAGGTCG | 2571 |
| AGAAGTTGA | | AAAAATTAA | |
| GCAAGAGGAC | 2572 | CCAGGAAGAT | 2573 |
| TCCAATGGCT | | TCGAACGGGT | |
| GCATCAACT | | GTATTAATT | |
| GAGGACTCCA | 2574 | GAAGATTCGA | 2575 |
| ATGGCTGCAT | | ACGGGTGTAT | |
| CAACT | | TAATT | |

In some cases, the mammal can have HCM or DCM, and the gene to be suppressed and replaced can be JPH2 (which encodes junctophilin 2). Exemplary JPH2 sequences are set forth in NCBI RefSeq accession number NM_020433 (e.g., version NM_020433.5) and NCBI RefSeq accession number NM_175913 (e.g., version NM_175913.4). A JPH2 polypeptide can, in some cases, have the amino acid sequence set forth in NCBI RefSeq accession number NP_065166 (e.g., version NP_065166.2) or NCBI RefSeq accession number NP_787109 (e.g., version NP_787109.2).

Examples of shRNA sequences and corresponding shIMM sequences targeted to JPH2 are set forth in TABLE 1DD.

TABLE 1DD
Representative JPH2 shRNA
and shIMM sequences

| shRNA | SEQ | shIMM | SEQ |
|---|---|---|---|
| Sequence | ID | Sequence | ID |
| GCGAATACTC | 2576 | GGGAGTATTC | 2577 |
| TGGCTCCTGG | | AGGGTCGTGG | |
| AACTT | | AATTT | |
| GCCGTGTCAG | 2578 | GTCGAGTGAG | 2579 |
| CTTCCTTAAG | | TTTTCTAAAA | |
| A | | A | |
| GCCAACCAGG | 2580 | GCGAATCAAG | 2581 |
| AGTCCAACAT | | AATCGAATAT | |
| T | | A | |
| GTCCAACATT | 2582 | ATCGAATATA | 2583 |
| GCTCGCACTT | | GCACGGACAT | |
| T | | T | |
| GACTTCTACC | 2584 | GATTTTTATC | 2585 |
| AGCCAGGTCC | | AACCTGGACC | |
| GGAATATCA | | CGAGTACCA | |
| GCATGGTGAT | 2586 | GTATGGTCAT | 2587 |
| CCTGCTGAAC | | TCTCCTCAAT | |
| A | | A | |

In some cases, the mammal can have LQTS, HCM, or limb-girdle muscular dystrophy (LGMD), and the gene to be suppressed and replaced can be CAV3 (which encodes caveolin 3). Exemplary CAV3 sequences are set forth in NCBI RefSeq accession number NM_033337 (e.g., version NM_033337.3) and NCBI RefSeq accession number NM_001234 (e.g., version NM_001234.5). A CAV3 polypeptide can, in some cases, have the amino acid sequence set forth in NCBI RefSeq accession number NP_203123 (e.g., version NP_203123.1) or NCBI RefSeq accession number NP_001225 (e.g., version NP_001225.1).

Examples of shRNA sequences and corresponding shIMM sequences targeted to CAV3 are set forth in TABLE 1EE.

TABLE 1EE
Representative CAV3 shRNA
and shIMM sequences

| shRNA | SEQ | shIMM | SEQ |
|---|---|---|---|
| Sequence | ID | Sequence | ID |
| GGCCCAGATC | 2588 | AGCGCAAATT | 2589 |
| GTCAAGGATA | | GTGAAAGACA | |
| T | | T | |
| GGTGAACCGA | 2590 | CGTCAATCGT | 2591 |
| GACCCCAAGA | | GATCCGAAAA | |
| ACATT | | ATATA | |
| GTGAACCGAG | 2592 | GTCAATCGTG | 2593 |
| ACCCCAAGAA | | ATCCGAAAAA | |
| CATTA | | TATAA | |
| GAACCGAGAC | 2594 | CAATCGTGAT | 2595 |
| CCCAAGAACA | | CCGAAAAATA | |
| T | | T | |
| GAGACCCCAA | 2596 | GTGATCCGAA | 2597 |
| GAACATTAAC | | AAATATAAAT | |
| GAGGACATA | | GAAGATATT | |
| GACCCCAAGA | 2598 | GATCCGAAAA | 2599 |
| ACATTAACGA | | ATATAAATGA | |
| GGACA | | AGATA | |
| GACCCCAAGA | 2600 | GATCCGAAAA | 2601 |
| ACATTAACGA | | ATATAAATGA | |
| GGACATAGT | | AGATATTGT | |
| GAACATTAAC | 2602 | AAATATAAAT | 2603 |
| GAGGACATA | | GAAGATATT | |
| GAACATTAAC | 2604 | AAATATAAAT | 2605 |
| GAGGACATAG | | GAAGATATTG | |
| T | | T | |
| GAGCTACACC | 2606 | CAGTTATACG | 2607 |
| ACCTTCACT | | ACGTTTACA | |
| GAGCTACACC | 2608 | CAGTTATACG | 2609 |
| ACCTTCACTG | | ACGTTTACAG | |
| T | | T | |
| GCTACACCAC | 2610 | GTTATACGAC | 2611 |
| CTTCACTGT | | GTTTACAGT | |
| GCTACACCAC | 2612 | GTTATACGAC | 2613 |
| CTTCACTGTC | | GTTTACAGTG | |
| T | | T | |
| GCATCTCCTT | 2614 | GTATTTCGTT | 2615 |
| CTGCCACATC | | TTGTCATATT | |
| T | | T | |
| GCCATGCATT | 2616 | CCCTTGTATA | 2617 |
| AAGAGCTACC | | AAAAGTTATC | |
| T | | T | |

In some cases, the mammal can have LQTS or CPVT, and the gene to be suppressed and replaced can be TECRL (which encodes trans-2,3-enoyl-CoA reductase like protein). Exemplary TECRL sequences are set forth in NCBI RefSeq accession number NM_001010874 (e.g., version NM_001010874.5) and NCBI RefSeq accession number NM_001363796 (e.g., version NM_001363796.1). A TECRL polypeptide can, in some cases, have the amino acid sequence set forth in NCBI RefSeq accession number NP_001010874 (e.g., version NP_001010874.2) or NCBI RefSeq accession number NP_001350725 (e.g., version NP_001350725.1).

Examples of shRNA sequences and corresponding shIMM sequences targeted to TECRL are set forth in TABLE 1FF.

TABLE 1FF
Representative TECRL shRNA
and shIMM sequences

| shRNA | SEQ | shIMM | SEQ |
|---|---|---|---|
| Sequence | ID | Sequence | ID |
| GGAACGCAAG | 2618 | CGAGCGGAAA | 2619 |
| AGAGCATTAC | | AGGGCTTTGC | |
| T | | T | |
| GAACGCAAGA | 2620 | GAGCGGAAAA | 2621 |
| GAGCATTACT | | GGGCTTTGCT | |
| T | | A | |
| GAGCTACACG | 2622 | GGGCAACTCG | 2623 |
| GTTCATACT | | CTTTATTCT | |
| GAGCTACACG | 2624 | GGGCAACTCG | 2625 |
| GTTCATACTG | | CTTTATTCTC | |
| A | | A | |
| GCTACACGGT | 2626 | GCAACTCGCT | 2627 |
| TCATACTGA | | TTATTCTCA | |
| GAAGGATGAT | 2628 | CAAAGACGAC | 2629 |
| ATGAGAAAT | | ATGAGGAAC | |
| GCCCTCTAAG | 2630 | GGCCACTTAG | 2631 |
| ACCAACTCCA | | GCCTACACCT | |
| GCAGTCAAA | | GCTGTGAAG | |
| GACCAACTCC | 2632 | GGCCTACACC | 2633 |
| AGCAGTCAA | | TGCTGTGAA | |
| GATGCTCAAA | 2634 | GACGCACAGA | 2635 |
| CAAGGAAACA | | CTAGAAAGCA | |
| GATAT | | AATTT | |
| GCTCAAACAA | 2636 | GCACAGACTA | 2637 |
| GGAAACAGA | | GAAAGCAAA | |
| GCTCAAACAA | 2638 | GCACAGACTA | 2639 |
| GGAAACAGAT | | GAAAGCAAAT | |
| A | | T | |
| GCTCAAACAA | 2640 | GCACAGACTA | 2641 |
| GGAAACAGAT | | GAAAGCAAAT | |
| ATGTATTCT | | TTGCATACT | |
| GGAAACAGAT | 2642 | GAAAGCAAAT | 2643 |
| ATGTATTCT | | TTGCATACT | |
| GAAGGACTAC | 2644 | AAAAGATTAT | 2645 |
| ATTACCATTC | | ATAACGATAC | |
| AAAGT | | AGAGC | |
| GGACTACATT | 2646 | AGATTATATA | 2647 |
| ACCATTCAA | | ACGATACAG | |
| GGACTACATT | 2648 | AGATTATATA | 2649 |
| ACCATTCAAA | | ACGATACAGA | |
| GTATT | | GCATA | |
| GCAGCTTCCT | 2650 | GCTGCATCGT | 2651 |
| CCATTGTCA | | CGATAGTGA | |
| GCAGCTTCCT | 2652 | GCTGCATCGT | 2653 |
| CCATTGTCAC | | CGATAGTGAC | |
| A | | T | |
| GCAGCTTCCT | 2654 | GCTGCATCGT | 2655 |
| CCATTGTCAC | | CGATAGTGAC | |
| ACTGT | | TCTCT | |
| GTCAGTTGGA | 2656 | GTGAGCTGGA | 2657 |
| CCACAGTGT | | CGACTGTCT | |
| GGACCTCTGC | 2658 | GGTCCACTCC | 2659 |
| TAATATACCT | | TTATTTATCT | |
| CCTCT | | GCTGT | |
| GACCTCTGCT | 2660 | GTCCACTCCT | 2661 |
| AATATACCT | | TATTTATCT | |
| GACCTCTGCT | 2662 | GTCCACTCCT | 2663 |
| AATATACCTC | | TATTTATCTG | |
| CTCTT | | CTGTT | |
| GCTAATATAC | 2664 | CCTTATTTAT | 2665 |
| CTCCTCTTT | | CTGCTGTTC | |
| GAGGATCCCA | 2666 | AAGAATTCCT | 2667 |
| TGTATATAT | | TGCATTTAC | |
| GGCTTGCTTC | 2668 | AGCATGTTTT | 2669 |
| TGTCATTGT | | TGCCACTGC | |
| GGCTTGCTTC | 2670 | AGCATGTTTT | 2671 |
| TGTCATTGTA | | TGCCACTGCA | |
| T | | T | |
| GCTTGCTTCT | 2672 | GCATGTTTTT | 2673 |
| GTCATTGTA | | GCCACTGCA | |
| GCTTGCTTCT | 2674 | GCATGTTTTT | 2675 |
| GTCATTGTAT | | GCCACTGCAT | |
| A | | T | |
| GGGATTTACT | 2676 | GGGTTTCACA | 2677 |
| TCTTGGATTG | | TCATGGATAG | |
| CCTACTACA | | CGTATTATA | |
| GGATTTACTT | 2678 | GGTTTCACAT | 2679 |
| CTTGGATTGC | | CATGGATAGC | |
| CTACTACAT | | GTATTATAT | |
| GATTTACTTC | 2680 | GTTTCACATC | 2681 |
| TTGGATTGCC | | ATGGATAGCG | |
| TACTA | | TATTA | |
| GATTTACTTC | 2682 | GTTTCACATC | 2683 |
| TTGGATTGCC | | ATGGATAGCG | |
| TACTACATT | | TATTATATA | |
| GATTGCCTAC | 2684 | GATAGCGTAT | 2685 |
| TACATTAAT | | TATATAAAC | |
| GCCTACTACA | 2686 | GCGTATTATA | 2687 |
| TTAATCATCC | | TAAACCACCC | |
| ACTAT | | TCTTT | |
| GAAACAGGCA | 2688 | GTAATAGACA | 2689 |
| AATCACAGT | | GATTACTGT | |
| GGCAAATCAC | 2690 | GACAGATTAC | 2691 |
| AGTATCTGCT | | TGTTTCAGCA | |
| ATCAA | | ATTAA | |
| GCAAATCACA | 2692 | ACAGATTACT | 2693 |
| GTATCTGCTA | | GTTTCAGCAA | |
| TCAAT | | TTAAC | |
| GCTGGGAATC | 2694 | GCAGGCAACC | 2695 |
| ATTTCATCA | | ACTTTATTA | |
| GCTGGGAATC | 2696 | GCAGGCAACC | 2697 |
| ATTTCATCAA | | ACTTTATTAA | |
| T | | C | |
| GCCTGTTTCC | 2698 | GCGTGCTTTC | 2699 |
| CAAGTCCAAA | | CTAGCCCTAA | |
| TTATA | | CTACA | |
| GTTTCCCAAG | 2700 | GCTTTCCTAG | 2701 |
| TCCAAATTA | | CCCTAACTA | |
| GTTTCCCAAG | 2702 | GCTTTCCTAG | 2703 |
| TCCAAATTAT | | CCCTAACTAC | |
| A | | A | |
| GGTTTCATGT | 2704 | CGTATCTTGC | 2705 |
| CCTAACTACA | | CCAAATTATA | |
| CCTAT | | CGTAC | |
| GTTTCATGTC | 2706 | GTATCTTGCC | 2707 |
| CTAACTACA | | CAAATTATA | |
| GTCCTAACTA | 2708 | GCCCAAATTA | 2709 |
| CACCTATGA | | TACGTACGA | |
| GTCCTAACTA | 2710 | GCCCAAATTA | 2711 |
| CACCTATGAG | | TACGTACGAA | |
| A | | A | |
| GAGATTGGAT | 2712 | GAAATAGGTT | 2713 |
| CATGGATTAG | | CTTGGATAAG | |
| T | | C | |
| GAGATTGGAT | 2714 | GAAATAGGTT | 2715 |
| CATGGATTAG | | CTTGGATAAG | |
| TTTCACAGT | | CTTTACTGT | |
| GATTGGATCA | 2716 | AATAGGTTCT | 2717 |
| TGGATTAGT | | TGGATAAGC | |
| GATTGGATCA | 2718 | AATAGGTTCT | 2719 |
| TGGATTAGTT | | TGGATAAGCT | |
| T | | T | |
| GATTGGATCA | 2720 | AATAGGTTCT | 2721 |
| TGGATTAGTT | | TGGATAAGCT | |
| TCACA | | TTACT | |
| GATTGGATCA | 2722 | AATAGGTTCT | 2723 |
| TGGATTAGTT | | TGGATAAGCT | |
| TCACAGTCA | | TTACTGTGA | |
| GGATCATGGA | 2724 | GGTTCTTGGA | 2725 |
| TTAGTTTCA | | TAAGCTTTA | |
| GGATCATGGA | 2726 | GGTTCTTGGA | 2727 |
| TTAGTTTCAC | | TAAGCTTTAC | |
| A | | T | |
| GGATCATGGA | 2728 | GGTTCTTGGA | 2729 |
| TTAGTTTCAC | | TAAGCTTTAC | |
| AGTCA | | TGTGA | |
| GATCATGGAT | 2730 | GTTCTTGGAT | 2731 |
| TAGTTTCACA | | AAGCTTTACT | |
| GTCAT | | GTGAT | |
| GGATTAGTTT | 2732 | GGATAAGCTT | 2733 |
| CACAGTCAT | | TACTGTGAT | |
| GGATTAGTTT | 2734 | GGATAAGCTT | 2735 |
| CACAGTCATG | | TACTGTGATG | |
| A | | A | |
| GATGAGTATC | 2736 | CATGAGCATT | 2737 |
| CAGATGTCT | | CAAATGTCA | |

Any appropriate method can be used to deliver a SupRep nucleic acid construct to cells (e.g., cardiac cells) within a living mammal. For example, a SupRep construct containing a suppressive component and a replacement component can be administered to a mammal using one or more vectors, such as viral vectors. In some cases, vectors for administering SupRep nucleic acids can be used for transient expression of the suppressive and corrective components. In some cases, vectors for administering SupRep nucleic acids can be used for stable expression of the suppressive and corrective components. In some cases, where a vector for administering nucleic acid can be used for stable expression, the vector can be engineered to integrate nucleic acid designed to express the suppressive component and/or nucleic acid designed to express the corrective component into the genome of a cell. In such cases, any appropriate method can be used to integrate the nucleic acid(s) into the genome of a cell. For example, gene therapy techniques can be used to integrate nucleic acid designed to express a suppressive component (e.g., a shRNA) and/or nucleic acid designed to express a corrective component (e.g., a wild type polypeptide that is immune to the suppressive component) into the genome of a cell. In some cases, stable expression does not necessarily require integration into the genome. Using AAV9, for example, the SupRep DNA can persist on its own in the cell, without integrating into the human genome. Non-integrated DNA typically is destroyed as genomic DNA replicates, but in non-dividing cells such as cardiomyocytes or neurons, the SupRep DNA can persist indefinitely since the cells do not replicate or divide to remove the SupRep DNA.

Vectors for administering SupRep nucleic acids to cells can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, NJ (2002), and Viral Vectors for Gene Therapy: Methods and Protocols, edited by Curtis A. Machida, Humana Press, Totowa, NJ (2003). Virus-based nucleic acid delivery vectors typically are derived from animal viruses, such as adenoviruses, adeno-associated viruses (AAVs), retroviruses, lentiviruses, vaccinia viruses, herpes viruses, and papilloma viruses. In some cases, a SupRep nucleic acid construct can be delivered to cells using adeno-associated virus vectors (e.g., an AAV serotype 1 viral vector, an AAV serotype 2 viral vector, an AAV serotype 3 viral vector, an AAV serotype 4 viral vector, an AAV serotype 5 viral vector, an AAV serotype 6 viral vector, an AAV serotype 7 viral vector, an AAV serotype 8 viral vector, an AAV serotype 9 viral vector, an AAV serotype 10 viral vector, an AAV serotype 11 viral vector, an AAV serotype 12 viral vector, or a recombinant AAV serotype viral vector such as an AAV serotype 2/9 viral vector in which the AAV2 inverted terminal repeats and genome are contained within the AAV9 capsid, which can result in AAV9 tropism for cardiomyocytes), lentiviral vectors, retroviral vectors, adenoviral vectors, herpes simplex virus vectors, or poxvirus vector. In some cases, an AAV9 vector can be used to deliver one or more SupRep nucleic acids to cells.

In addition to nucleic acid encoding a suppressive component and nucleic acid encoding a corrective component, a viral vector can contain regulatory elements operably linked to the nucleic acid encoding the suppressive component and the corrective component. As used herein, “operably linked” refers to positioning of a regulatory element in a vector relative to a nucleic acid in such a way as to permit or facilitate expression of the encoded RNA and/or polypeptide. Such regulatory elements can include promoter sequences, enhancer sequences, response elements, signal peptides, internal ribosome entry sequences (IRES), P2A self-cleaving peptide sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a suppressive component (e.g., a shRNA) and a corrective component (e.g., a WT polypeptide that is immune to the suppression by the suppressive component). A promoter can be constitutive or inducible (e.g., in the presence of tetracycline or rapamycin), and can affect the expression of a nucleic acid encoding a shRNA or a polypeptide in a general or tissue-specific manner. Examples of promoters that can be used to drive expression of suppressive and corrective components (e.g., in cardiomyocyte cells) include, without limitation, a U6 promoter, a H1 promoter a cytomegalovirus immediate-early (CMV) promoter, an alpha-myosin heavy chain promoter, a myosin light chain 2 promoter, cardiac troponin T, and a cardiac troponin C promoter.

As used herein, the term “AAV particle” refers to packaged capsid forms of the AAV virus that transmits its nucleic acid genome to cells. The term “viral genome” refers to one copy of a virus genome. Each virus particle contains one viral genome, and each AAV vector contains one viral genome. In some cases, a composition containing an AAV particle encoded by an AAV vector as provided herein can be administered at a concentration from about 10¹⁰ AAV particles/mL to about 10¹⁵ AAV particles/mL (e.g., from about 10¹⁰ AAV particles/mL to about 10¹¹ AAV particles/mL, from about 10¹⁰ AAV particles/mL to about 10¹² AAV particles/mL, from about 10¹⁰ AAV particles/mL to about 10¹³ AAV particles/mL, from about 10¹¹ AAV particles/mL to about 10¹² AAV particles/mL, from about 10¹¹ AAV particles/mL to about 10¹³ AAV particles/mL, from about 10¹¹ AAV particles/mL to about 10¹⁴ AAV particles/mL, from about 10¹² AAV particles/mL to about 10¹³ AAV particles/mL, from about 10¹² AAV particles/mL to about 10¹⁴ AAV particles/mL, or from about 10¹³ AAV particles/mL to about 10¹⁴ AAV particles/mL). In some cases, a composition containing an AAV particle encoded by an AAV vector as provided herein can be administered at a concentration from about 10¹⁰ viral genomes per kilogram body weight (vg/kg) to about 10¹⁵ vg/kg (e.g., from about 10¹⁰ to about 10¹¹ vg/kg, from about 10¹⁰ to about 10¹² vg/kg, from about 10¹⁰ to about 10¹³ vg/kg, from about 10¹¹ to about 10¹² vg/kg, from about 10¹¹ to about 10¹³ vg/kg, from about 10¹¹ to about 10¹⁴ vg/kg, from about 10¹² to about 10¹³ vg/kg, from about 10¹² to about 10¹⁴ vg/kg, or from about 10¹³ to about 10¹⁴ vg/kg).

In some cases, a SupRep nucleic acid construct can be administered to a mammal using a non-viral vector. Methods of using non-viral vectors for nucleic acid delivery are described elsewhere. See, for example, Gene Therapy Protocols (Methods in Molecular Medicine), edited by Jeffrey R. Morgan, Humana Press, Totowa, NJ (2002). For example, a SupRep nucleic acid encoding a suppressive component and a corrective component can be administered to a mammal by direct injection of nucleic acid molecules (e.g., plasmids) comprising SupRep nucleic acid, or by administering nucleic acid molecules complexed with lipids, polymers, or nanospheres. In some cases, a SupRep nucleic acid designed to express a suppressive component and a corrective component can be delivered to cells (e.g., cardiomyocytes) via direct injection (e.g., into the myocardium), intraperitoneal administration, intranasal administration, intravenous administration, intrathecal administration, intracerebral administration, intraparenchymal administration, or oral delivery in nanoparticles and/or drug tablets, capsules, or pills.

When KCNQ1-SupRep and/or KCNH2-SupRep and/or SCN5A-SupRep gene therapy is efficiently delivered to the majority of cardiomyocytes, the gene therapy-mediated restoration of repolarization reserve may distribute via gap junctions to partially or completely compensate for neighboring untransduced cardiomyocytes. From the studies described herein, it was noteworthy that during measurement of optical action potentials, no arrhythmic activity was observed in electrically coupled iPSC-CMs that had been transduced with KCNQ1-SupRep—suggesting that efficient transduction of cells may be sufficient to maintain normal rhythm and compensate for untransduced neighboring cells.

Any appropriate amount of a SupRep nucleic acid can be administered to a mammal (e.g., a human) having a congenital disorder. An effective amount of a SupRep nucleic acid can reduce one or more symptoms of the disorder being treated. In some cases, for example, effective suppression-and-replacement of KCNQ1 (e.g., for patients having LQT1, severe cases where multiple pathogenic variants in KCNQ1 are inherited such as autosomal recessive LQT1 and Jervell and Lange-Nielsen syndrome (JLNS), or type 2 SQTS (SQT2)) using KCNQ1-SupRep gene therapy can produce I_{Ks} current density similar to that of a healthy individual (e.g., within about 50%, about 25%, about 20%, about 15%, about 10%, or about 5% of the I_{Ks} current density of a healthy individual). Pathogenic variants in KCNQ1 that lead to a gain-of-function and an abnormal increase in I_{Ks} current density can lead to SQTS. In some cases, a therapeutically effective amount can provide enough I_{Ks} to ameliorate the LQTS phenotype without overcompensating and causing SQTS. In LQT1 and JLNS, disease severity correlates with the degree of lost I_{Ks} (Moss et al., Circulation, 115:2481-2489 (2007)). Heterozygous nonsense or frameshift mutations cause haploinsufficiency and typically result in mild LQT1 with −50% I_{Ks}. Dominant-negative missense mutations reduce I_{Ks} beyond 50% and are more strongly associated with breakthrough cardiac events. In the most severe cases, patients with JLNS inherit two mutant alleles that result in either minimal (<10%) or no I_{Ks} (Bhuiyan et al., Prog. Biophys. Mol. Biol., 98:319-327 (2008)). Conversely, KCNQ1 variants with substantial gain of function cause SQT2, though the exact degree of increased I_{Ks} is not well established (Chen et al., Science, 299:251-254 (2003); Bellocq et al., Circulation, 109:2394-2397 (2004); Hong et al., Cardiovasc. Res., 68:433-440 (2005); and Das et al., Heart Rhythm, 6:1146-1153 (2009)). Thus, the therapeutic window for KCNQ1-SupRep in humans may be relatively wide, allowing flexibility for achieving optimal efficacy. In some cases, KCNQ1-SupRep dosing can be modified by the promoters and/or enhancers driving expression, or by the amount of viral particles delivered to the mammal. In some cases, a therapeutically effective amount of a KCNQ1-SupRep construct can increase I_{Ks} by at least 25% (e.g., at least 50%, at least 60%, at least 70%, at least 75%, at least 80%, at least 90%, at least 100%, or at least 200%), as compared to the I_{Ks} prior to treatment.

In some cases, effective suppression-and-replacement of KCNH2 (e.g., for patients having LQT2 or type 1 short QT syndrome (SQT1)) using KCNH2-SupRep gene therapy can produce I_{Kr} current density similar to that of a healthy individual (e.g., within about 50%, about 25%, about 20%, about 15%, about 10%, or about 5% of the I_{Kr} current density of a healthy individual). In some cases, a therapeutically effective amount can provide enough I_{Kr} to ameliorate the LQTS phenotype without overcompensating and causing SQTS. Like LQT1, in LQT2, disease severity correlates with the degree of lost I_{Kr} (Moss et al., Circulation, 105:794-799 (2002)). Heterozygous nonsense or frameshift mutations cause haploinsufficiency and typically result in LQT2 with ˜50% I_{Kr}. Dominant-negative missense mutations reduce I_{Kr} beyond 50% and are more strongly associated with cardiac events, especially when localized to the pore region of the channel (Moss et al., supra). Conversely, KCNH2 variants with substantial gain of function can cause SQT1 (Brugada et al., Circulation, 109:30-35 (2004); and Sun et al., JMCC, 50:433-441 (2011)). Thus, the therapeutic window for KCNH2-SupRep in humans may be relatively wide, allowing flexibility for achieving optimal efficacy. In some cases, KCNH2-SupRep dosing can be modified by the promoters and/or enhancers driving expression, or by the amount of viral particles delivered to the mammal. In some cases, a therapeutically effective amount of a KCNH2-SupRep construct can increase I_{Kr} by at least 25% (e.g., at least 50%, at least 60%, at least 70%, at least 75%, at least 80%, at least 90%, at least 100%, or at least 200%), as compared to the I_{Kr} prior to treatment.

In some cases, effective suppression-and-replacement of SCN5A (e.g., for patients having LQT3, multifocal ectopic premature Purkinje-related contraction (MEPPC) syndrome, SCN5A-mediated dilated cardiomyopathy, recessive sick sinus syndrome, or BrS) using SCN5A-SupRep gene therapy can produce I_{Na} current density and sodium channel kinetics similar to that of a healthy individual (e.g., within about 50%, about 25%, about 20%, about 15%, about 10%, or about 5% of the I_{Na} current density of a healthy individual). In some cases, SCN5A-SupRep dosing can be modified by the promoters and/or enhancers driving expression, or by the amount of viral particles delivered to the mammal. In some cases, a therapeutically effective amount of a SCN5A-SupRep construct can suppress the amount of pathological increase in I_{Na} late current or window current by at least 25% (e.g., at least 50%, at least 60%, at least 70%, at least 75%, at least 80%, at least 90%, at least 100%, or at least 200%), as compared to the I_{Na} late current prior to treatment.

The typical QT range is about 350-450 ms for men and about 350-460 ms for women, but QT above about 430-440 generally is considered to be borderline high. The QT for males having LQTS is typically greater than 450 ms, and the QT for women having LQTS is typically greater than 460 ms. Most LQTS patients top out at less than 520 ms. In some cases, an effective amount of a KCNQ1-SupRep construct and/or a KCNH2-SupRep construct and/or a SCN5A-SupRep construct administered to a mammal (e.g., a human) having LQT1 and/or LQT2 and/or LQT3 can shorten the APD to a length similar to that of a healthy individual, such that the APD is within the normal range. In some cases, an effective amount of a KCNQ1-SupRep construct and/or a KCNH2-SupRep construct and/or a SCN5A-SupRep construct administered to a mammal (e.g., a human) having LQT1 and/or LQT2 and/or LQT3 can shorten the APD to a length that is within about 10% (e.g., within about 8%, about 5%, or about 3%, of the APD of a healthy individual). In some cases, a therapeutically effective amount of a KCNQ1-SupRep construct and/or a KCNH2-SupRep construct and/or a SCN5A-SupRep construct to a mammal (e.g., a human) having LQT1 and/or LQT2 and/or LQT3 can shorten the APD by at least 10% (e.g., at least 15%, at least 20%, at least 25%, at least 30%, at least 40%, or at least 50%), as compared to the APD prior to treatment.

In some cases, symptoms can be assessed on the day of treatment, 1 day post treatment, 3 months post treatment, 6 months post treatment, 1 year post treatment and every year thereafter post treatment. In some cases, symptoms can be assessed between 1 day post treatment and 7 days post treatment (e.g., between 1 day and 2 days post treatment, between 1 day and 3 days post treatment, between 1 day and 4 days post treatment, between 2 days and 3 days post treatment, between 2 days and 4 days post treatment, between 2 days and 5 days post treatment, between 3 days and 4 days post treatment, between 3 days and 5 days post treatment, 3 days and 6 days post treatment, between 4 days and 5 days post treatment, between 4 days and 6 days post treatment, between 4 days and 7 days post treatment, between 5 days and 6 days post treatment, between 5 days and 7 days post treatment, or between 6 days and 7 days post treatment). In some cases, symptoms can be assessed between 1 week post treatment and 4 weeks post treatment (e.g., between 1 week and 2 weeks post treatment, between 1 week and 3 weeks post treatment, between 1 week and 4 weeks post treatment, between 2 weeks and 3 weeks post treatment, between 2 weeks and 4 weeks post treatment, or between 3 weeks and 4 weeks post treatment). In some cases, symptoms can be assessed between 1 month post treatment and 12 months post treatment (e.g., between 1 month and 2 months post treatment, between 1 month and 3 months post treatment, between 1 month and 4 months post treatment, between 2 months and 3 months post treatment, between 2 months and 4 months post treatment, between 2 months and 5 months post treatment, between 3 months and 4 months post treatment, between 3 months and 5 months post treatment, between 3 months and 6 months post treatment, between 4 months and 5 months post treatment, between 4 and 6 months post treatment, between 4 months and 7 months post treatment, between 5 months and 6 months post treatment, between 5 months and 7 months post treatment, between 5 months and 8 months post treatment, between 6 months and 7 months post treatment, between 6 months and 8 months post treatment, between 6 months and 9 months post treatment, between 7 months and 8 months post treatment, between 7 months and 9 months post treatment, between 7 months and 10 months post treatment, between 8 months and 9 months post treatment, between 8 months and 10 months post treatment, between 8 months and 11 months post treatment, between 9 months and 10 months post treatment, between 9 months and 11 months post treatment, between 9 months and 12 months post treatment, between 10 months and 11 months post treatment, between 10 months and 12 months post treatment, or between 11 months and 12 months post treatment). In some cases, symptoms can be assessed between 1 year post treatment and about 20 years post treatment (e.g., between 1 year and 5 years post treatment, between 1 year and 10 years post treatment, between 1 year and 15 years post treatment, between 5 years and 10 years post treatment, between 5 years and 15 years post treatment, between 5 years and 20 years post treatment, between 10 years and 15 years post treatment, between 10 years and 20 years post treatment, or between 15 years and 20 years post treatment).

In some cases, a treatment as provided herein can be administered to a mammal (e.g., a human) having a congenital disease (e.g., a congenital heart disease such as LQTS, or more specifically, LQT1 or LQT2 or LQT3) in a single dose, without further administration.

In some cases, a treatment as provided herein can be administered to a mammal (e.g., a human) having a congenital disease (e.g., a congenital heart disease such as LQTS, or more specifically, LQT1) at least once daily, or at least once weekly for at least two consecutive days or weeks. In some cases, a treatment as provided herein is administered to a mammal (e.g., a human) having a congenital disease at least 3, 4, 5, 6, 7, 8, 9, 10, 11, 12, 13, 14, or 15 consecutive days or weeks. In some cases, a treatment as provided herein is administered to a mammal (e.g., a human) having a congenital disease at least once daily or at least once weekly for at least 1, 2, 3, 4, 5, 6, 7, 8, 9, 10, 11, or 12 consecutive weeks. In some cases, a treatment as provided herein is administered to a mammal (e.g., a human) having a congenital disease at least once daily or at least once weekly for at most 4, 5, 6, 7, 8, 9, 10, 11, 12, 13, 14, 15, 16, 17, 18, 19, or 20 consecutive days or weeks. In some cases, a treatment as provided herein is administered to a mammal (e.g., a human) having a congenital disease at least once weekly for at most 1, 2, 3, 4, 5, 6, 7, 8, 9, 10, 11, or 12 consecutive weeks or months. In some cases, a treatment as provided herein is administered to a mammal (e.g., a human) having a congenital disease at least 1, 2, 3, 4, 5, 6, 7, 8, 9, 10, 11, or 12 consecutive months or years, chronically for a subject's entire life span, or an indefinite period of time.

In one embodiment, a mammal having LQT1 or SQTS associated with a pathogenic mutation in the KCNQ1 gene can be identified by, for example, analyzing a biological sample (e.g., analyzing a blood sample using PCR and/or DNA sequencing methods) obtained from the mammal to determine whether DNA in the sample includes a pathogenic mutation in the KCNQ1 gene. Pathogenic mutations in or encoded by or encoded by the KCNQ1 gene include, without limitation, c.421G>A (p.V141M), c.919G>C (p.V307L), c.513C>A (p.Y171X), c.760G>A (p.V254M), c.1700T>G (p.I567S), c.1377C>T (p.D459D), c.1380C>A (p.G460G), c.1383T>C (p.Y461Y), c.1386C>T (p.D462D), c.1389T>C (p.S463S), c.1392T>C (p.S464S), c.1395A>C (p.V465V), c.1398G>A (p.R466R), c.1401G>A (p.K467K), and c.1404C>T (p.S468S). See, also, Wu et al., J Arrhythm. 2016, 32(5):381-388; Hedley et al., Hum Mutat. 2009, 30:1486-1551; and Morita et al., Lancet 2008, 372:750-763. SupRep constructs targeted to mutant KCNQ1 alleles can be designed to suppress the mutant KCNQ1 alleles and replace them with a wild type KCNQ1 allele. SupRep constructs targeted to mutant KCNQ1 alleles can be designed and prepared using methods described, for example, in the Examples herein. For example, a SupRep construct can be generated to target a KCNQ1 allele containing a pathogenic mutation, either by targeting a region of a disease-associated KCNQ1 allele that contains a pathogenic mutation, or by targeting a region of a disease-associated KCNQ1 allele that does not contain a pathogenic mutation. The SupRep constructs can be tested for their ability to suppress a mutant KCNQ1 allele and replace it with a wild type KCNQ1 allele. For example, constructs can be tested in an in vitro model system by co-transfecting cultured cells with a wild type KCNQ1 construct and a shKCNQ1 construct, and measuring KCNQ1 expression with qRT-PCR and/or western blotting. A construct having a relatively high ability to knock down KCNQ1 expression (e.g., the ability to knock down at least 70 percent, at least 75 percent, at least 80 percent, at least 85 percent, at least 90 percent, at least 95 percent, at least 98 percent, or at least 99 percent of KCNQ1 expression at the mRNA and/or protein level) can be selected. The selected construct can be packaged in a virus particle (e.g., an AAV particle) and delivered to a mammal identified as having a pathogenic mutation in the KCNQ1 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to KCNQ1 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQT1 or SQTS, to determine whether one or more symptoms of the disorder are diminished. For example, effective SupRep treatment of a mammal having LQT1 or SQTS associated with a pathogenic mutation in KCNQ1 can result in a reduction in symptoms such as rapid heartbeat, fainting, and/or seizures. In some cases, effective SupRep treatment of a mammal having LQT1 or SQTS associated with a pathogenic KCNQ1 mutation can result in an I_{Ks} current density and/or cardiac APD that is similar to the I_{Ks} current density and/or cardiac APD of a healthy individual.

In another embodiment, a mammal having LQT2 or SQTS associated with a pathogenic mutation in the KCNH2 gene can be identified by, for example, analyzing a biological sample (e.g., analyzing a blood sample using PCR and/or DNA sequencing methods) obtained from the mammal to determine whether DNA in the sample includes a pathogenic mutation in the KCNH2 gene. Pathogenic mutations in or encoded by the KCNH2 gene include, without limitation, c.1764C>G (p.N588K), c.82A>G (p.K28E), c.2893G>T (p.G965X), c.3036_3048del (p.R1014fs), and c.3107_3111dup (p.V1038fs). See, also, Hedley et al., Hum Mutat. 2009, 30:1486-1551; Curran et al., Cell 1995, 80:795-803; and Smith et al., J Arrhythm. 2016, 32(5):373-380. SupRep constructs targeted to mutant KCNH2 alleles can be designed to suppress the mutant KCNH2 alleles and replace them with a wild type KCNH2 allele. SupRep constructs targeted to mutant KCNH2 alleles can be designed and prepared using methods described, for example, in the Examples herein. For example, a SupRep construct can be generated to target a KCNH2 allele containing a pathogenic mutation, either by targeting a region of a disease-associated KCNH2 allele that contains a pathogenic mutation, or by targeting a region of a disease-associated KCNH2 allele that does not contain a pathogenic mutation. The SupRep constructs can be tested for their ability to suppress a mutant KCNH2 allele and replace it with a wild type KCNH2 allele. For example, constructs can be tested in an in vitro model system by co-transfecting cultured cells with a wild type KCNH2 construct and a shKCNH2 construct, and measuring KCNH2 expression with qRT-PCR and/or western blotting. A construct having a relatively high ability to knock down KCNH2 expression (e.g., the ability to knock down at least 70 percent, at least 75 percent, at least 80 percent, at least 85 percent, at least 90 percent, at least 95 percent, at least 98 percent, or at least 99 percent of KCNH2 expression at the mRNA and/or protein level) can be selected. The selected construct can be packaged in a virus particle (e.g., an AAV particle) and delivered to a mammal identified as having a pathogenic mutation in the KCNH2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to KCNH2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQT2 or SQTS, to determine whether one or more symptoms of the disorder are diminished. For example, effective SupRep treatment of a mammal having LQT2 or SQTS associated with a pathogenic mutation in KCNH2 can result in a reduction in symptoms such as rapid heartbeat, fainting (e.g., during periods of strenuous exercise or emotional distress), and/or seizures. In some cases, effective SupRep treatment of a mammal having LQT2 or SQTS associated with a pathogenic KCNH2 mutation can result in shortening of the APD to a length similar to that of a healthy individual, such that the APD is within the normal range.

In another embodiment, a mammal having LQT3 or BrS associated with a pathogenic mutation in the SCN5A gene can be identified by, for example, analyzing a biological sample (e.g., analyzing a blood sample using PCR and/or DNA sequencing methods) obtained from the mammal to determine whether DNA in the sample includes a pathogenic mutation in the SCN5A gene. Pathogenic mutations in or encoded by the SCN5A gene include, without limitation, c.100C>T (p.R34C), c.1571C>A (p.S524Y), c.1673A>G (p.H558R), c.3308C>A (p.S1103Y), c.3578G>A (p.R1193Q), c.3908C>T (p.T1304M), c.4509_4516del (p.1505-1507del), c.4865G>A (p.R1623Q), and c.5851G>T (p.V1951L). See, also, Kapa et al., Circulation 2009, 120:1752-1760; and Hedley et al., Hum Mutat. 2009, 30:1486-1551. SupRep constructs targeted to mutant SCN5A alleles can be designed to suppress the mutant SCN5A alleles and replace them with a wild type SCN5A allele. SupRep constructs targeted to mutant SCN5A alleles can be designed and prepared using methods described, for example, in the Examples herein. For example, a SupRep construct can be generated to target a SCN5A allele containing a pathogenic mutation, either by targeting a region of a disease-associated SCN5A allele that contains a pathogenic mutation, or by targeting a region of a disease-associated SCN5A allele that does not contain a pathogenic mutation. The SupRep constructs can be tested for their ability to suppress a mutant SCN5A allele and replace it with a wild type gene allele. For example, constructs can be tested in an in vitro model system by co-transfecting cultured cells with a wild type SCN5A construct and a shSCN5A construct, and measuring SCN5A expression with qRT-PCR and/or western blotting. A construct having a relatively high ability to knock down SCN5A expression (e.g., the ability to knock down at least 70 percent, at least 75 percent, at least 80 percent, at least 85 percent, at least 90 percent, at least 95 percent, at least 98 percent, or at least 99 percent of SCN5A expression at the mRNA and/or protein level) can be selected. The selected construct can be packaged in a virus particle (e.g., an AAV particle) and delivered to a mammal identified as having a pathogenic mutation in the SCN5A gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to SCN5A can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQT3 or BrS, to determine whether one or more symptoms of the disorder are diminished. For example, effective SupRep treatment of a mammal having LQT3 or BrS associated with a pathogenic mutation in SCN5A can result in a reduction in symptoms such as fainting and/or seizures. In some cases, effective SupRep treatment of a mammal having LQT3 or BrS associated with a pathogenic SCN5A mutation can result in shortening of the APD to a length similar to that of a healthy individual, such that the APD is within the normal range.

In another embodiment, a mammal having HCM or DCM associated with a pathogenic mutation in the MYH7 gene can be identified by, for example, analyzing a biological sample (e.g., analyzing a blood sample using PCR and/or DNA sequencing methods) obtained from the mammal to determine whether DNA in the sample includes a pathogenic mutation in the MYH7 gene. Pathogenic mutations in or encoded by the MYH7 gene include, without limitation, c.1156T>C (p.Y386H), c.1680T>C (p.S532P), c.1816G>A (p.V606M), c.2602G>C (p.A868P), c.2945T>C (p.M982T), c.4258A>T (p.R1420W), and c.5779A>T (p.I1927F). See, also, Millat et al., Eur J Med Genet. 2010, 53:261-267; Van Driest et al., Mayo Clin Proc 2005, 80(4):463-469; references. SupRep constructs targeted to mutant MYH7 alleles can be designed to suppress the mutant MYH7 alleles and replace them with a wild type MYH7 allele. SupRep constructs targeted to mutant MYH7 alleles can be designed and prepared using methods described, for example, in the Examples herein. For example, a SupRep construct can be generated to target a MYH7 allele containing a pathogenic mutation, either by targeting a region of a disease-associated MYH7 allele that contains a pathogenic mutation, or by targeting a region of a disease-associated MYH7 allele that does not contain a pathogenic mutation. The SupRep constructs can be tested for their ability to suppress a mutant MYH7 allele and replace it with a wild type gene allele. For example, constructs can be tested in an in vitro model system by co-transfecting cultured cells with a wild type MYH7 construct and a shMYH7 construct, and measuring MYH7 expression with qRT-PCR and/or western blotting. A construct having a relatively high ability to knock down MYH7 expression (e.g., the ability to knock down at least 70 percent, at least 75 percent, at least 80 percent, at least 85 percent, at least 90 percent, at least 95 percent, at least 98 percent, or at least 99 percent of MYH7 expression at the mRNA and/or protein level) can be selected. The selected construct can be packaged in a virus particle (e.g., an AAV particle) and delivered to a mammal identified as having a pathogenic mutation in the MYH7 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to MYH7 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of HCM or DCM, to determine whether one or more symptoms of the disorder are diminished. For example, effective SupRep treatment of a mammal having HCM or DCM associated with a pathogenic mutation in MYH7 can result in a reduction in symptoms such as dyspnea, fatigue, edema of the legs and/or ankles, chest pain, arrhythmia, fainting, lightheadedness, and/or heart palpitations. In some cases, effective SupRep treatment of a mammal having HCM or DCM associated with a pathogenic MYH7 mutation can result in reduced cardiac hypertrophy and cardiomyocyte size, and/or decreased interstitial fibrosis and myocardial disarray.

In another embodiment, a mammal having ACM associated with a pathogenic mutation in the DSP gene can be identified by, for example, analyzing a biological sample (e.g., analyzing a blood sample using PCR and/or DNA sequencing methods) obtained from the mammal to determine whether DNA in the sample includes a pathogenic mutation in the DSP gene. Pathogenic mutations in or encoded by the DSP gene include, without limitation, c.151C>T (p.N51X), c.478C>T (p. R160X), c.897C>G (p.S299R), c.1264G>A (p.E422K), c.1333A>G (p.I445V), c. 3160_3169delAAGAACAA (p.K1052fsX26), c.3337C>T (p. R1113X), c.4775A>G (p.K1592R), c.5212C>T (p. R1738X), c.6478C>T (p.R2160X), and c.6496C>T (p.R2166X). See, also, Bhonsale et al., Eur Heart J. 2015, 36(14):847-855; Sen-Chowdhry et al., Circulation 2007, 115:1710-1720; and Norman et al., Circulation 2005, 112:636-642. SupRep constructs targeted to mutant DSP alleles can be designed to suppress the mutant DSP alleles and replace them with a wild type DSP allele. SupRep constructs targeted to mutant DSP alleles can be designed and prepared using methods described, for example, in the Examples herein. For example, a SupRep construct can be generated to target a DSP allele containing a pathogenic mutation, either by targeting a region of a disease-associated DSP allele that contains a pathogenic mutation, or by targeting a region of a disease-associated DSP allele that does not contain a pathogenic mutation. The SupRep constructs can be tested for their ability to suppress a mutant DSP allele and replace it with a wild type DSP allele. For example, constructs can be tested in an in vitro model system by co-transfecting cultured cells with a wild type DSP construct and a shDSP construct, and measuring DSP expression with qRT-PCR and/or western blotting. A construct having a relatively high ability to knock down DSP expression (e.g., the ability to knock down at least 70 percent, at least 75 percent, at least 80 percent, at least 85 percent, at least 90 percent, at least 95 percent, at least 98 percent, or at least 99 percent of DSP expression at the mRNA and/or protein level) can be selected. The selected construct can be packaged in a virus particle (e.g., an AAV particle) and delivered to a mammal identified as having a pathogenic mutation in the DSP gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to DSP can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of ACM, to determine whether one or more symptoms of the disorder are diminished. For example, effective SupRep treatment of a mammal having ACM associated with a pathogenic mutation in DSP can result in a reduction in symptoms such as fibrofatty replacement of the myocardium, ventricular arrhythmias, syncope, sustained ventricular tachycardia (VT) or fibrillation (VF), and/or heart failure. In some cases, effective SupRep treatment of a mammal having ACM associated with a pathogenic DSP mutation can result a reduction in LV inflammation, fibrosis, and/or systolic dysfunction.

In another embodiment, a mammal having HCM associated with a pathogenic mutation in the MYBPC3 gene can be identified by, for example, analyzing a biological sample (e.g., analyzing a blood sample using PCR and/or DNA sequencing methods) obtained from the mammal to determine whether DNA in the sample includes a pathogenic mutation in the MYBPC3 gene. Pathogenic mutations in or encoded by the MYBPC3 gene include, without limitation, c.3535G>A (p.E1179K), c.3413G>A (p.R1138H), c.3392T>C (p.I1131T), c.3106C>T (p.R1036C), c.3004C>T (p.R1002W), c.2992C>G (p.Q998E), c.2870C>G (p.T957S), c.2686G>A (p.V896M), c.2498C>T (p.A833V), c.2497G>A (p.A833T), c.1144C>T (p.R382TW), c.977G>A (p.R326Q), c.706A>G (p.S236G), and c.472G>A (p.V158M). See, also, Helms et al., Circ: Gen Precision Med. 2020, 13:396-405; Carrier et al., Gene. 2015, 573(2):188-197; Millat et al., supra; and Page et al., Circ Cardiovasc Genet. 2012, 5:156-166. SupRep constructs targeted to mutant MYBPC3 alleles can be designed to suppress the mutant MYBPC3 alleles and replace them with a wild type MYBPC3 allele. SupRep constructs targeted to mutant MYBPC3 alleles can be designed and prepared using methods described, for example, in the Examples herein. For example, a SupRep construct can be generated to target a MYBPC3 allele containing a pathogenic mutation, either by targeting a region of a disease-associated MYBPC3 allele that contains a pathogenic mutation, or by targeting a region of a disease-associated MYBPC3 allele that does not contain a pathogenic mutation. The SupRep constructs can be tested for their ability to suppress a mutant MYBPC3 allele and replace it with a wild type MYBPC3 allele. For example, constructs can be tested in an in vitro model system by co-transfecting cultured cells with a wild type MYBPC3 construct and a shMYBPC3 construct, and measuring MYBPC3 expression with qRT-PCR and/or western blotting. A construct having a relatively high ability to knock down MYBPC3 expression (e.g., the ability to knock down at least 70 percent, at least 75 percent, at least 80 percent, at least 85 percent, at least 90 percent, at least 95 percent, at least 98 percent, or at least 99 percent of MYBPC3 expression at the mRNA and/or protein level) can be selected. The selected construct can be packaged in a virus particle (e.g., an AAV particle) and delivered to a mammal identified as having a pathogenic mutation in the MYBPC3 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to MYBPC3 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of HCM, to determine whether one or more symptoms of the disorder are diminished. For example, effective SupRep treatment of a mammal having HCM associated with a pathogenic mutation in MYBPC3 can result in a reduction in symptoms such as dyspnea, rapid heartbeat, chest pain, fainting, dizziness, and/or fatigue. In some cases, effective SupRep treatment of a mammal having HCM associated with a pathogenic MYBPC3 mutation can result in reduced contractility, improved relaxation, and/or reduced energy consumption.

In another embodiment, a mammal having DCM associated with a pathogenic mutation in the RBM20 gene can be identified by, for example, analyzing a biological sample (e.g., analyzing a blood sample using PCR and/or DNA sequencing methods) obtained from the mammal to determine whether DNA in the sample includes a pathogenic mutation in the RBM20 gene. Pathogenic mutations in or encoded by the RBM20 gene include, without limitation, c.1913C>T (p.P638L), c.1901G>A (p.R634Q), c.1906C>A (p.R636S), c.1907G>A (p.R636H), c.1909A>G (p.S637G), c.1661G>A (p.V535I), c.1958C>T (p.R634W), c.1964C>T (p.R636C), and c.2205G>A (p.R716Q). See, also, Brauch et al., J Am Coll Cardiol. 2009, 54:930-941; Li et al., Clin Transl Sci. 2010, 3:90-97; and Refaat et al., Heart Rhythm. 2012, 9:390-396. SupRep constructs targeted to mutant RBM20 alleles can be designed to suppress the mutant RBM20 alleles and replace them with a wild type RBM20 allele. SupRep constructs targeted to mutant RBM20 alleles can be designed and prepared using methods described, for example, in the Examples herein. For example, a SupRep construct can be generated to target a RBM20 allele containing a pathogenic mutation, either by targeting a region of a disease-associated RBM20 allele that contains a pathogenic mutation, or by targeting a region of a disease-associated RBM20 allele that does not contain a pathogenic mutation. The SupRep constructs can be tested for their ability to suppress a mutant RBM20 allele and replace it with a wild type RBM20 allele. For example, constructs can be tested in an in vitro model system by co-transfecting cultured cells with a wild type RBM20 construct and a shRBM20 construct, and measuring RBM20 expression with qRT-PCR and/or western blotting. A construct having a relatively high ability to knock down RBM20 expression (e.g., the ability to knock down at least 70 percent, at least 75 percent, at least 80 percent, at least 85 percent, at least 90 percent, at least 95 percent, at least 98 percent, or at least 99 percent of RBM20 expression at the mRNA and/or protein level) can be selected. The selected construct can be packaged in a virus particle (e.g., an AAV particle) and delivered to a mammal identified as having a pathogenic mutation in the RBM20 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to RBM20 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM, to determine whether one or more symptoms of the disorder are diminished. For example, effective SupRep treatment of a mammal having DCM associated with a pathogenic mutation in RBM20 can result in a reduction in symptoms such as dyspnea, fatigue, edema of the legs and/or ankles, chest pain, arrhythmia, fainting, lightheadedness, and/or heart palpitations. In some cases, effective SupRep treatment of a mammal having DCM associated with a pathogenic RBM20 mutation can result in normalization of LV size and/or strengthening of the LV.

In another embodiment, a mammal having LQTS or Timothy syndrome associated with a pathogenic mutation in the CACNA1C gene can be identified by, for example, analyzing a biological sample (e.g., analyzing a blood sample using PCR and/or DNA sequencing methods) obtained from the mammal to determine whether DNA in the sample includes a pathogenic mutation in the CACNA1C gene. Pathogenic mutations in or encoded by the CACNA1C gene include, without limitation, c.2570C>G (p.P857R), c.2500A>G (p.K834Q), c.2570C>T (p.P857L), c.5717G>A (p.R1906Q), c.82G>A (p.A28T), c.2578C>G (p.R860G), c.3497T>C (p.I166T), c.3496A>G (p.I1166V), c.4425C>G (p.I1475M), and c.4486G>A (p.E1496K). See, also, Boczek et al., Circ Cardiovasc Genet. 2013, 6(3):279-289; Wemhoner et al., J Mol Cell Cardiol. 2015, 80:186-195; references. SupRep constructs targeted to mutant CACNA1C alleles can be designed to suppress the mutant CACNA1C alleles and replace them with a wild type CACNA1C allele. SupRep constructs targeted to mutant CACNA1C alleles can be designed and prepared using methods described, for example, in the Examples herein. For example, a SupRep construct can be generated to target a CACNA1C allele containing a pathogenic mutation, either by targeting a region of a disease-associated CACNA1C allele that contains a pathogenic mutation, or by targeting a region of a disease-associated CACNA1C allele that does not contain a pathogenic mutation. The SupRep constructs can be tested for their ability to suppress a mutant CACNA1C allele and replace it with a wild type CACNA1C allele. For example, constructs can be tested in an in vitro model system by co-transfecting cultured cells with a wild type CACNA1C construct and a shCACNA1C construct, and measuring CACNA1C expression with qRT-PCR and/or western blotting. A construct having a relatively high ability to knock down CACNA1C expression (e.g., the ability to knock down at least 70 percent, at least 75 percent, at least 80 percent, at least 85 percent, at least 90 percent, at least 95 percent, at least 98 percent, or at least 99 percent of CACNA1C expression at the mRNA and/or protein level) can be selected. The selected construct can be packaged in a virus particle (e.g., an AAV particle) and delivered to a mammal identified as having a pathogenic mutation in the CACNA1C gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CACNA1C can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS or Timothy syndrome, to determine whether one or more symptoms of the disorder are diminished. For example, effective SupRep treatment of a mammal having LQTS or Timothy syndrome associated with a pathogenic mutation in CACNA1C can result in a reduction in symptoms such as rapid heartbeat, fainting, seizures, episodes of hypoglycemia, and/or episodes of hypothermia. In some cases, effective SupRep treatment of a mammal having LQTS or Timothy syndrome associated with a pathogenic CACNA1C mutation can result in an I_{Ks} current density and/or cardiac APD that is similar to the I_{Ks} current density and/or cardiac APD of a healthy individual.

In another embodiment, a mammal having ACM associated with a pathogenic mutation in the PKP2 gene can be identified by, for example, analyzing a biological sample (e.g., analyzing a blood sample using PCR and/or DNA sequencing methods) obtained from the mammal to determine whether DNA in the sample includes a pathogenic mutation in the PKP2 gene. Pathogenic mutations in or encoded by the PKP2 gene include, without limitation, c.235C>T (p.R79X), c.397C>T (p.Q133X), c.2386T>C (p.C796R), c.2011delC (p.P671fsX683), c.1368delA (p.N456fsX458), c.145-148delCAGA (p.S50fsX110), c.2509delA (p.V837fsX930), c.2489+1G>A (p.mutant splice product), c.1171-2A>G (p.mutant splice product), c.2146-1G>C (p.mutant splice product), c.2197-2202insGdelCACACC (p.A733fsX740), c.1613G>A (p.W538X), c.1271T>C (p.F424S), c.1642delG (p.V548fsX562), and c.419C>T (p.S140F). See, also, Dalal et al., Circulation. 2006, 113:1641-1649; van Tintelen et al., Circulation. 2006, 113(13):1650-1658; and Fressart et al., Europace. 2010, 12(6):861-868. SupRep constructs targeted to mutant PKP2 alleles can be designed to suppress the mutant PKP2 alleles and replace them with a wild type PKP2 allele. SupRep constructs targeted to mutant PKP2 alleles can be designed and prepared using methods described, for example, in the Examples herein. For example, a SupRep construct can be generated to target a PKP2 allele containing a pathogenic mutation, either by targeting a region of a disease-associated PKP2 allele that contains a pathogenic mutation, or by targeting a region of a disease-associated PKP2 allele that does not contain a pathogenic mutation. The SupRep constructs can be tested for their ability to suppress a mutant PKP2 allele and replace it with a wild type PKP2 allele. For example, constructs can be tested in an in vitro model system by co-transfecting cultured cells with a wild type PKP2 construct and a shPKP2 construct, and measuring PKP2 expression with qRT-PCR and/or western blotting. A construct having a relatively high ability to knock down PKP2 expression (e.g., the ability to knock down at least 70 percent, at least 75 percent, at least 80 percent, at least 85 percent, at least 90 percent, at least 95 percent, at least 98 percent, or at least 99 percent of PKP2 expression at the mRNA and/or protein level) can be selected. The selected construct can be packaged in a virus particle (e.g., an AAV particle) and delivered to a mammal identified as having a pathogenic mutation in the PKP2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to PKP2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of ACM, to determine whether one or more symptoms of the disorder are diminished. For example, effective SupRep treatment of a mammal having ACM associated with a pathogenic mutation in PKP2 can result in a reduction in symptoms such as fibrofatty replacement of the myocardium, ventricular arrhythmias, syncope, sustained VT or VF, and/or heart failure. In some cases, effective SupRep treatment of a mammal having ACM associated with a pathogenic PKP2 mutation can result in reduction in LV inflammation, fibrosis, and/or systolic dysfunction.

In another embodiment, a mammal having ACM associated with a pathogenic mutation in the DSG2 gene can be identified by, for example, analyzing a biological sample (e.g., analyzing a blood sample using PCR and/or DNA sequencing methods) obtained from the mammal to determine whether DNA in the sample includes a pathogenic mutation in the DSG2 gene. Pathogenic mutations in or encoded by the DSG2gene include, without limitation, c.378+1G>T (p.mutant splice product), c.560A>G (p.D187G), c.146 G>A (p.R49H), c.560 A>G (p.D187G), c.1520 G>A (p.C507Y), c.1003A>G (p.T335A), and c.961 T>A (p.F3211), as well as mutations resulting in p.K294E, p.D154E, p.V3921, p.L772X, and p.R773K. See, also, Brodehl et al., Int J Mol Sci. 2021, 22(7):3786; Debus et al., J Mol Cell Cardiol. 2019, 129:303-313; and Xu et al., J Am Coll Cardiol. 2010, 55(6):587-597. SupRep constructs targeted to mutant DSG2 alleles can be designed to suppress the mutant DSG2 alleles and replace them with a wild type DSG2 allele. SupRep constructs targeted to mutant DSG2 alleles can be designed and prepared using methods described, for example, in the Examples herein. For example, a SupRep construct can be generated to target a DSG2 allele containing a pathogenic mutation, either by targeting a region of a disease-associated DSG2 allele that contains a pathogenic mutation, or by targeting a region of a disease-associated DSG2 allele that does not contain a pathogenic mutation. The SupRep constructs can be tested for their ability to suppress a mutant DSG2 allele and replace it with a wild type DSG2 allele. For example, constructs can be tested in an in vitro model system by co-transfecting cultured cells with a wild type DSG2 construct and a shDSG2 construct, and measuring DSG2 expression with qRT-PCR and/or western blotting. A construct having a relatively high ability to knock down DSG2 expression (e.g., the ability to knock down at least 70 percent, at least 75 percent, at least 80 percent, at least 85 percent, at least 90 percent, at least 95 percent, at least 98 percent, or at least 99 percent of DSG2 expression at the mRNA and/or protein level) can be selected. The selected construct can be packaged in a virus particle (e.g., an AAV particle) and delivered to a mammal identified as having a pathogenic mutation in the DSG2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to DSG2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of ACM, to determine whether one or more symptoms of the disorder are diminished. For example, effective SupRep treatment of a mammal having ACM associated with a pathogenic mutation in DSG2 can result in a reduction in symptoms such as fibrofatty replacement of the myocardium, ventricular arrhythmias, syncope, sustained VT or VF, and/or heart failure. In some cases, effective SupRep treatment of a mammal having ACM associated with a pathogenic DSG2 mutation can result in reduction in LV inflammation, fibrosis, and/or systolic dysfunction.

In another embodiment, a mammal having ACM, DCM, LVNC, or skeletal myopathy associated with a pathogenic mutation in the DES gene can be identified by, for example, analyzing a biological sample (e.g., analyzing a blood sample using PCR and/or DNA sequencing methods) obtained from the mammal to determine whether DNA in the sample includes a pathogenic mutation in the DES gene. Pathogenic mutations in or encoded by the DES gene include, without limitation, c.407C>T (p.L136P), c.1009G>C (p.A337P), c.1013T>G (p.L338R), c.1195G>T (p.D399Y), and c.1201G>A (p.E401K). See, also, Brodehl et al., J Mol Cell Cardiol. 2016, 91:207-214; Goudeau et al., Hum Mutat. 2006, 27(9):906-913; references. SupRep constructs targeted to mutant DES alleles can be designed to suppress the mutant DES alleles and replace them with a wild type DES allele. SupRep constructs targeted to mutant DES alleles can be designed and prepared using methods described, for example, in the Examples herein. For example, a SupRep construct can be generated to target a DES allele containing a pathogenic mutation, either by targeting a region of a disease-associated DES allele that contains a pathogenic mutation, or by targeting a region of a disease-associated DES allele that does not contain a pathogenic mutation. The SupRep constructs can be tested for their ability to suppress a mutant DES allele and replace it with a wild type DES allele. For example, constructs can be tested in an in vitro model system by co-transfecting cultured cells with a wild type DES construct and a shDES construct, and measuring DES expression with qRT-PCR and/or western blotting. A construct having a relatively high ability to knock down DES expression (e.g., the ability to knock down at least 70 percent, at least 75 percent, at least 80 percent, at least 85 percent, at least 90 percent, at least 95 percent, at least 98 percent, or at least 99 percent of DES expression at the mRNA and/or protein level) can be selected. The selected construct can be packaged in a virus particle (e.g., an AAV particle) and delivered to a mammal identified as having a pathogenic mutation in the DES gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to DES can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of ACM, DCM, LVNC, or skeletal myopathy, to determine whether one or more symptoms of the disorder are diminished. For example, effective SupRep treatment of a mammal having ACM, DCM, LVNC, or skeletal myopathy associated with a pathogenic mutation in DES can result in a reduction in symptoms such as fibrofatty replacement of the myocardium, ventricular arrhythmias, fainting, sustained VT or VF, dyspnea, fatigue, edema of the legs and/or ankles, chest pain, lightheadedness, heart palpitations, and/or heart failure. In some cases, effective SupRep treatment of a mammal having ACM, DCM, LVNC, or skeletal myopathy associated with a pathogenic DES mutation can result in reduction in LV inflammation, fibrosis, systolic dysfunction, and/or endomyocardial trabeculations, as well as normalization of LV size, and/or strengthening of the LV.

In another embodiment, a mammal having ATS (also referred to as LQT7) or CPVT associated with a pathogenic mutation in the KCNJ2 gene can be identified by, for example, analyzing a biological sample (e.g., analyzing a blood sample using PCR and/or DNA sequencing methods) obtained from the mammal to determine whether DNA in the sample includes a pathogenic mutation in the KCNJ2 gene. Pathogenic mutations in or encoded by the KCNJ2 gene include, without limitation, c.199C>T (p.R67W), c.271_282del12 (p.A91 L94del), c.653G>A (p.R218Q), c.953A>G (p.N318S), c.966G>C (p.W322C), and c.1244C>T (p.P415L). See, also, Limberg et al., Basic Res Cardiol. 2013, 108:353; Andelfinger et al., Am J Hum Genet. 2002, 71(3):663-668; and Tristani-Firouzi et al., J Clin Invest. 2002, 110(3):381-388. SupRep constructs targeted to mutant KCNJ2 alleles can be designed to suppress the mutant KCNJ2 alleles and replace them with a wild type KCNJ2 allele. SupRep constructs targeted to mutant KCNJ2 alleles can be designed and prepared using methods described, for example, in the Examples herein. For example, a SupRep construct can be generated to target a KCNJ2 allele containing a pathogenic mutation, either by targeting a region of a disease-associated KCNJ2 allele that contains a pathogenic mutation, or by targeting a region of a disease-associated KCNJ2 allele that does not contain a pathogenic mutation. The SupRep constructs can be tested for their ability to suppress a mutant KCNJ2 allele and replace it with a wild type KCNJ2 allele. For example, constructs can be tested in an in vitro model system by co-transfecting cultured cells with a wild type KCNJ2 construct and a shKCNJ2 construct, and measuring KCNJ2 expression with qRT-PCR and/or western blotting. A construct having a relatively high ability to knock down KCNJ2 expression (e.g., the ability to knock down at least 70 percent, at least 75 percent, at least 80 percent, at least 85 percent, at least 90 percent, at least 95 percent, at least 98 percent, or at least 99 percent of KCNJ2 expression at the mRNA and/or protein level) can be selected. The selected construct can be packaged in a virus particle (e.g., an AAV particle) and delivered to a mammal identified as having a pathogenic mutation in the KCNJ2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to KCNJ2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of ATS or CPVT, to determine whether one or more symptoms of the disorder are diminished. For example, effective SupRep treatment of a mammal having ATS or CPVT associated with a pathogenic mutation in KCNJ2 can result in a reduction in symptoms such as muscle weakness, fainting, lightheadedness, dizziness, periodic paralysis, and/or arrhythmia (e.g., VT). In some cases, effective SupRep treatment of a mammal having ATS or CPVT associated with a pathogenic KCNJ2 mutation can result in normalization and/or regulation of the heart rhythm.

In another embodiment, a mammal having CPVT associated with a pathogenic mutation in the CASQ2 gene can be identified by, for example, analyzing a biological sample (e.g., analyzing a blood sample using PCR and/or DNA sequencing methods) obtained from the mammal to determine whether DNA in the sample includes a pathogenic mutation in the CASQ2 gene. Pathogenic mutations in or encoded by the CASQ2 gene include, without limitation, c.62delA (p.E21Gfs*15), c.97C>T (p.R33*), c.98G>A (p.R33Q), c.115G>T (p.E39*), c.115G>A (p.E39K), c.158G>T (p.C53F), c.164A>G (p.Y55C), c.199C>T (p.Q67*), c.204delA (p.K68Nfs*5), c.213delA (p.Q71Hfs*2), c.230T>C (p.L77P), c.234+2T>C (p.mutant splice site), c.259A>T (p.K87*), c.339-354del (p.S113Rfs*6), c.500T>A (p.L167H), c.518G>T (p.S173I), c.532+1G>A (p. mutant splice site), c.539A>G (p.K180R), c.545T>C (p.F182S), c.546delT (p.F182Lfs*28), c.572C>T (p.P191L), c.603delA (p.V203Lfs*7), c.618A>C (p.K206N), and c.691C>T (p.P231S). See, also, Ng et al., Circulation. 2020, 142(10):932-947; and Gray et al., Heart Rhythm. 2016, 13(8):1652-1660. SupRep constructs targeted to mutant CASQ2 alleles can be designed to suppress the mutant CASQ2 alleles and replace them with a wild type CASQ2 allele. SupRep constructs targeted to mutant CASQ2 alleles can be designed and prepared using methods described, for example, in the Examples herein. For example, a SupRep construct can be generated to target a CASQ2 allele containing a pathogenic mutation, either by targeting a region of a disease-associated CASQ2 allele that contains a pathogenic mutation, or by targeting a region of a disease-associated CASQ2 allele that does not contain a pathogenic mutation. The SupRep constructs can be tested for their ability to suppress a mutant CASQ2 allele and replace it with a wild type CASQ2 allele. For example, constructs can be tested in an in vitro model system by co-transfecting cultured cells with a wild type CASQ2 construct and a shCASQ2 construct, and measuring CASQ2 expression with qRT-PCR and/or western blotting. A construct having a relatively high ability to knock down CASQ2 expression (e.g., the ability to knock down at least 70 percent, at least 75 percent, at least 80 percent, at least 85 percent, at least 90 percent, at least 95 percent, at least 98 percent, or at least 99 percent of CASQ2 expression at the mRNA and/or protein level) can be selected. The selected construct can be packaged in a virus particle (e.g., an AAV particle) and delivered to a mammal identified as having a pathogenic mutation in the CASQ2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CASQ2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of CPVT, to determine whether one or more symptoms of the disorder are diminished. For example, effective SupRep treatment of a mammal having CPVT associated with a pathogenic mutation in CASQ2 can result in a reduction in symptoms such as dizziness, lightheadedness, fainting, and/or VT. In some cases, effective SupRep treatment of a mammal having CPVT associated with a pathogenic CASQ2 mutation can result in normalization and/or regulation of the heart rhythm.

In another embodiment, a mammal having DCM associated with a pathogenic mutation in the LMNA gene can be identified by, for example, analyzing a biological sample (e.g., analyzing a blood sample using PCR and/or DNA sequencing methods) obtained from the mammal to determine whether DNA in the sample includes a pathogenic mutation in the LMNA gene. Pathogenic mutations in or encoded by the LMNA gene include, without limitation, c.481G>A (p.E161K), c.1130G>A (p.R377H), c.1621C>T (p.R541C), c.1621C>G (p.R541G), c.266G>T (p.R89L), c.736C>T (p.Q246*), c.1197_1240del44 (p.G400Rfs*11), c.1292C>G (p.S431*), 1526_1527insC (p.T510Yfs*42), c.1443C>G (p.Y481*), and c.767 T>G (p.V256G). See, also, Saj et al., BMC Med Genet. 2013, 14:55; Sebillon et al., J Med Genet. 2003, 40:560-567; and Parks et al., Am Heart J. 2008, 156(1):161-169. SupRep constructs targeted to mutant LMNA alleles can be designed to suppress the mutant LMNA alleles and replace them with a wild type LMNA allele. SupRep constructs targeted to mutant LMNA alleles can be designed and prepared using methods described, for example, in the Examples herein. For example, a SupRep construct can be generated to target a LMNA allele containing a pathogenic mutation, either by targeting a region of a disease-associated LMNA allele that contains a pathogenic mutation, or by targeting a region of a disease-associated LMNA allele that does not contain a pathogenic mutation. The SupRep constructs can be tested for their ability to suppress a mutant LMNA allele and replace it with a wild type LMNA allele. For example, constructs can be tested in an in vitro model system by co-transfecting cultured cells with a wild type LMNA construct and a shLMNA construct, and measuring LMNA expression with qRT-PCR and/or western blotting. A construct having a relatively high ability to knock down LMNA expression (e.g., the ability to knock down at least 70 percent, at least 75 percent, at least 80 percent, at least 85 percent, at least 90 percent, at least 95 percent, at least 98 percent, or at least 99 percent of LMNA expression at the mRNA and/or protein level) can be selected. The selected construct can be packaged in a virus particle (e.g., an AAV particle) and delivered to a mammal identified as having a pathogenic mutation in the LMNA gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to LMNA can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM, to determine whether one or more symptoms of the disorder are diminished. For example, effective SupRep treatment of a mammal having DCM associated with a pathogenic mutation in LMNA can result in a reduction in symptoms such as dyspnea, fatigue, edema of the legs and/or ankles, chest pain, arrhythmia, fainting, lightheadedness, and/or heart palpitations. In some cases, effective SupRep treatment of a mammal having DCM associated with a pathogenic LMNA mutation can result in normalization of LV size and/or strengthening of the LV.

In another embodiment, a mammal having DCM associated with a pathogenic mutation in the TPM1 gene can be identified by, for example, analyzing a biological sample (e.g., analyzing a blood sample using PCR and/or DNA sequencing methods) obtained from the mammal to determine whether DNA in the sample includes a pathogenic mutation in the TPM1 gene. Pathogenic mutations in or encoded by the TPM1 gene include, without limitation, c.688G>A (p. D230N), c.688G>A (p.D230N), c.23T>G (p.M8R), c.632C>G (p.A211G), c.725C>T (p.A242V), c.163G>A (p.D55N), c.337C>G (p.L113V), c.341A>G (p.E114G), c.275T>C (p.I92T), c.423G>C (p.M141I), and c.416A>T (p.E139V). See, also, Pugh et al., Genet Med. 2014, 16:601-608; and McNally and Mestroni, Circ Res. 2017, 121:731-748. SupRep constructs targeted to mutant TPM1 alleles can be designed to suppress the mutant TPM1 alleles and replace them with a wild type TPM1 allele. SupRep constructs targeted to mutant TPM1 alleles can be designed and prepared using methods described, for example, in the Examples herein. For example, a SupRep construct can be generated to target a TPM1 allele containing a pathogenic mutation, either by targeting a region of a disease-associated TPM1 allele that contains a pathogenic mutation, or by targeting a region of a disease-associated TPM1 allele that does not contain a pathogenic mutation. The SupRep constructs can be tested for their ability to suppress a mutant TPM1 allele and replace it with a wild type TPM1 allele. For example, constructs can be tested in an in vitro model system by co-transfecting cultured cells with a wild type TPM1 construct and a shTPM1 construct, and measuring TPM1 expression with qRT-PCR and/or western blotting. A construct having a relatively high ability to knock down TPM1 expression (e.g., the ability to knock down at least 70 percent, at least 75 percent, at least 80 percent, at least 85 percent, at least 90 percent, at least 95 percent, at least 98 percent, or at least 99 percent of TPM1 expression at the mRNA and/or protein level) can be selected. The selected construct can be packaged in a virus particle (e.g., an AAV particle) and delivered to a mammal identified as having a pathogenic mutation in the TPM1 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TPM1 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM, to determine whether one or more symptoms of the disorder are diminished. For example, effective SupRep treatment of a mammal having DCM associated with a pathogenic mutation in TPM1 can result in a reduction in symptoms such as dyspnea, fatigue, edema of the legs and/or ankles, chest pain, arrhythmia, fainting, lightheadedness, and/or heart palpitations. In some cases, effective SupRep treatment of a mammal having DCM associated with a pathogenic TPM1 mutation can result in normalization of LV size and/or strengthening of the LV.

In another embodiment, a mammal having DCM or ACM associated with a pathogenic mutation in the PLN gene can be identified by, for example, analyzing a biological sample (e.g., analyzing a blood sample using PCR and/or DNA sequencing methods) obtained from the mammal to determine whether DNA in the sample includes a pathogenic mutation in the PLN gene. Pathogenic mutations in or encoded by the PLN gene include, without limitation, c.40_42delAGA (p.R14del), c.116T>G (p.L39X), and c.25C>T (p.R9C). See, also, to Rijdt et al., Cardiovasc Pathol. 2019, 40:2-6; Groeneweg et al., Am J Cardiol. 2013, 112:1197-1206; Fish et al., Sci Rep. 2016, 22235; and Haghighi et al., J Clin Invest. 2003, 111(6):869-876. SupRep constructs targeted to mutant PLN alleles can be designed to suppress the mutant PLN alleles and replace them with a wild type PLN allele. SupRep constructs targeted to mutant PLN alleles can be designed and prepared using methods described, for example, in the Examples herein. For example, a SupRep construct can be generated to target a PLN allele containing a pathogenic mutation, either by targeting a region of a disease-associated PLN allele that contains a pathogenic mutation, or by targeting a region of a disease-associated PLN allele that does not contain a pathogenic mutation. The SupRep constructs can be tested for their ability to suppress a mutant PLN allele and replace it with a wild type PLN allele. For example, constructs can be tested in an in vitro model system by co-transfecting cultured cells with a wild type PLN construct and a shPLN construct, and measuring PLN expression with qRT-PCR and/or western blotting. A construct having a relatively high ability to knock down PLN expression (e.g., the ability to knock down at least 70 percent, at least 75 percent, at least 80 percent, at least 85 percent, at least 90 percent, at least 95 percent, at least 98 percent, or at least 99 percent of PLN expression at the mRNA and/or protein level) can be selected. The selected construct can be packaged in a virus particle (e.g., an AAV particle) and delivered to a mammal identified as having a pathogenic mutation in the PLN gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to PLN can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or ACM, to determine whether one or more symptoms of the disorder are diminished. For example, effective SupRep treatment of a mammal having DCM or ACM associated with a pathogenic mutation in PLN can result in a reduction in symptoms such as dyspnea, fatigue, edema of the legs and/or ankles, chest pain, arrhythmia, fainting, lightheadedness, heart palpitations, fibrofatty replacement of the myocardium, sustained VT or VF, and/or heart failure. In some cases, effective SupRep treatment of a mammal having DCM or ACM associated with a pathogenic PLN mutation can result in normalization of LV size, strengthening of the LV, reduction in LV inflammation, reduction in fibrosis, and/or reduction in systolic dysfunction.

In another embodiment, a mammal having FH associated with a pathogenic mutation in the LDLR gene can be identified by, for example, analyzing a biological sample (e.g., analyzing a blood sample using PCR and/or DNA sequencing methods) obtained from the mammal to determine whether DNA in the sample includes a pathogenic mutation in the LDLR gene. Pathogenic mutations in or encoded by the LDLR gene include, without limitation, c.1845+2T>C, c.1012T>A (p.C338S), c.1297G>C (p.D433H), c.1702C>G (p.L568V), and c.2431A>T (p.K811*), c.97C>T (p.Q33X), c.357delG (p.K120fs), c.428G>A (p.C143Y), c.517T>C (p.C173R), c.1448G>A (p.W483X), c.1744C>T (p.L582F), c.1757C>A (p.S586X), and c.1879G>A (p.A627T). See, also, Tada et al., J Clin Lipidol. 2020, 14(3):346-351; Wang et al., J Geriatr Cardiol. 2018, 15(6):434-440; Hori et al., Atherosclerosis. 2019, 289:101-108; and Galicia-Garcia et al., Sci Rep. 2020, 10:1727. SupRep constructs targeted to mutant LDLR alleles can be designed to suppress the mutant LDLR alleles and replace them with a wild type LDLR allele. SupRep constructs targeted to mutant LDLR alleles can be designed and prepared using methods described, for example, in the Examples herein. For example, a SupRep construct can be generated to target a LDLR allele containing a pathogenic mutation, either by targeting a region of a disease-associated LDLR allele that contains a pathogenic mutation, or by targeting a region of a disease-associated LDLR allele that does not contain a pathogenic mutation. The SupRep constructs can be tested for their ability to suppress a mutant LDLR allele and replace it with a wild type LDLR allele. For example, constructs can be tested in an in vitro model system by co-transfecting cultured cells with a wild type LDLR construct and a shLDLR construct, and measuring LDLR expression with qRT-PCR and/or western blotting. A construct having a relatively high ability to knock down LDLR expression (e.g., the ability to knock down at least 70 percent, at least 75 percent, at least 80 percent, at least 85 percent, at least 90 percent, at least 95 percent, at least 98 percent, or at least 99 percent of LDLR expression at the mRNA and/or protein level) can be selected. The selected construct can be packaged in a virus particle (e.g., an AAV particle) and delivered to a mammal identified as having a pathogenic mutation in the LDLR gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to LDLR can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of FH, to determine whether one or more symptoms of the disorder are diminished. For example, effective SupRep treatment of a mammal having FH associated with a pathogenic mutation in LDLR can result in a reduction in symptoms such as elevated total and LDL cholesterol levels, angina, and/or xanthomas. In some cases, effective SupRep treatment of a mammal having FH associated with a pathogenic LDLR mutation can alleviate cerebrovascular disease and/or peripheral vascular disease associated with the FH.

In another embodiment, a mammal having FH associated with a pathogenic mutation in the PCSK9 gene can be identified by, for example, analyzing a biological sample (e.g., analyzing a blood sample using PCR and/or DNA sequencing methods) obtained from the mammal to determine whether DNA in the sample includes a pathogenic mutation in the PCSK9 gene. Pathogenic mutations in or encoded by the PCSK9 gene include, without limitation, c.381T>A (p.S127R), c.644G>A (p.R215H), c.646T>C (p.F216L), c.1120G>T (p.D374Y), and c.1486C>T (p.R496W), as well as p.N157K, p.R218S, p.R237W, p.E670G, p.R218S, p.R357H, p.R469W, p.A443T, p.R496W, p.N425S, p.D374H, p.D129G, p.A168E, p.G236S, p.N354I, p.A245T, p.R272Q, p.R272Q, and p.A245T. See, also, Hori et al., supra; Youngblom et al., “Familial Hypercholesterolemia,” 2014 Jan. 2 (Updated 2016 Dec 8), In: Adam et al., eds., GENEREVIEWS® University of Washington, Seattle; and Guo et al., Front Genet. 2020, 11:1020. SupRep constructs targeted to mutant PCSK9 alleles can be designed to suppress the mutant PCSK9 alleles and replace them with a wild type PCSK9 allele. SupRep constructs targeted to mutant PCSK9 alleles can be designed and prepared using methods described, for example, in the Examples herein. For example, a SupRep construct can be generated to target a PCSK9 allele containing a pathogenic mutation, either by targeting a region of a disease-associated PCSK9 allele that contains a pathogenic mutation, or by targeting a region of a disease-associated PCSK9 allele that does not contain a pathogenic mutation. The SupRep constructs can be tested for their ability to suppress a mutant PCSK9 allele and replace it with a wild type PCSK9 allele. For example, constructs can be tested in an in vitro model system by co-transfecting cultured cells with a wild type PCSK9 construct and a shPCSK9 construct, and measuring PCSK9 expression with qRT-PCR and/or western blotting. A construct having a relatively high ability to knock down PCSK9 expression (e.g., the ability to knock down at least 70 percent, at least 75 percent, at least 80 percent, at least 85 percent, at least 90 percent, at least 95 percent, at least 98 percent, or at least 99 percent of PCSK9 expression at the mRNA and/or protein level) can be selected. The selected construct can be packaged in a virus particle (e.g., an AAV particle) and delivered to a mammal identified as having a pathogenic mutation in the PCSK9 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to PCSK9 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of FH, to determine whether one or more symptoms of the disorder are diminished. For example, effective SupRep treatment of a mammal having FH associated with a pathogenic mutation in PCSK9 can result in a reduction in symptoms such as elevated total and LDL cholesterol levels, angina, and/or xanthomas. In some cases, effective SupRep treatment of a mammal having FH associated with a pathogenic PCSK9 mutation can alleviate cerebrovascular disease and/or peripheral vascular disease associated with the FH.

In another embodiment, a mammal having HCM or DCM associated with a pathogenic mutation in the TNNT2 gene can be identified by, for example, analyzing a biological sample (e.g., analyzing a blood sample using PCR and/or DNA sequencing methods) obtained from the mammal to determine whether DNA in the sample includes a pathogenic mutation in the TNNT2 gene. Pathogenic mutations in or encoded by the TNNT2 gene include, without limitation, c.421C>T (p.R141W), and c.835C>T (p.Q279X), as well as p.P80S, p.D86A, p.R92L, p.K97N, p.K124N, p.R130C, p.R134G, and p.R144W. See, also, Long et al., J Am Heart Assoc. 2015, 4(12):e002443; Gao et al., Medicine. 2020, 99(34):e21843; Millat et al., supra; and Hershberger et al., Circ Cardiovasc Genet. 2009, 2:306-313. SupRep constructs targeted to mutant TNNT2 alleles can be designed to suppress the mutant TNNT2 alleles and replace them with a wild type TNNT2 allele. SupRep constructs targeted to mutant TNNT2 alleles can be designed and prepared using methods described, for example, in the Examples herein. For example, a SupRep construct can be generated to target a TNNT2 allele containing a pathogenic mutation, either by targeting a region of a disease-associated TNNT2 allele that contains a pathogenic mutation, or by targeting a region of a disease-associated TNNT2 allele that does not contain a pathogenic mutation. The SupRep constructs can be tested for their ability to suppress a mutant TNNT2 allele and replace it with a wild type TNNT2 allele. For example, constructs can be tested in an in vitro model system by co-transfecting cultured cells with a wild type TNNT2 construct and a shTNNT2 construct, and measuring TNNT2 expression with qRT-PCR and/or western blotting. A construct having a relatively high ability to knock down TNNT2 expression (e.g., the ability to knock down at least 70 percent, at least 75 percent, at least 80 percent, at least 85 percent, at least 90 percent, at least 95 percent, at least 98 percent, or at least 99 percent of TNNT2 expression at the mRNA and/or protein level) can be selected. The selected construct can be packaged in a virus particle (e.g., an AAV particle) and delivered to a mammal identified as having a pathogenic mutation in the TNNT2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TNNT2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of HCM or DCM, to determine whether one or more symptoms of the disorder are diminished. For example, effective SupRep treatment of a mammal having HCM or DCM associated with a pathogenic mutation in TNNT2 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having HCM or DCM associated with a pathogenic TNNT2 mutation can result in reduced contractility, improved relaxation, reduced energy consumption, normalization of LV size, and/or strengthening of the LV.

In another embodiment, a mammal having LQTS or CPVT associated with a pathogenic mutation in the CALM1 gene can be identified by, for example, analyzing a biological sample (e.g., analyzing a blood sample using PCR and/or DNA sequencing methods) obtained from the mammal to determine whether DNA in the sample includes a pathogenic mutation in the CALM1 gene. Pathogenic mutations in or encoded by the CALM1 gene include, without limitation, p.N541, p.F90L, p.N98S, p.E105A, p.D130G, p.D132V, p.E141G, and p.F142L. See, also, Jensen et al., Front Mol Neurosci. 2018, 11:396; and Boczek et al., Circ Cardiovasc Genet. 2016, 9:136-146. SupRep constructs targeted to mutant CALM1 alleles can be designed to suppress the mutant CALM1 alleles and replace them with a wild type CALM1 allele. SupRep constructs targeted to mutant CALM1 alleles can be designed and prepared using methods described, for example, in the Examples herein. For example, a SupRep construct can be generated to target a CALM1 allele containing a pathogenic mutation, either by targeting a region of a disease-associated CALM1 allele that contains a pathogenic mutation, or by targeting a region of a disease-associated CALM1 allele that does not contain a pathogenic mutation. The SupRep constructs can be tested for their ability to suppress a mutant CALM1 allele and replace it with a wild type CALM1 allele. For example, constructs can be tested in an in vitro model system by co-transfecting cultured cells with a wild type CALM1 construct and a shgene construct, and measuring CALM1 expression with qRT-PCR and/or western blotting. A construct having a relatively high ability to knock down CALM1 expression (e.g., the ability to knock down at least 70 percent, at least 75 percent, at least 80 percent, at least 85 percent, at least 90 percent, at least 95 percent, at least 98 percent, or at least 99 percent of CALM1 expression at the mRNA and/or protein level) can be selected. The selected construct can be packaged in a virus particle (e.g., an AAV particle) and delivered to a mammal identified as having a pathogenic mutation in the CALM1 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CALM1 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS or CPVT, to determine whether one or more symptoms of the disorder are diminished. For example, effective SupRep treatment of a mammal having LQTS or CPVT associated with a pathogenic mutation in CALM1 can result in a reduction in symptoms such as rapid heartbeat, fainting, seizures, dizziness, lightheadedness, and/or VT. In some cases, effective SupRep treatment of a mammal having LQTS or CPVT associated with a pathogenic CALM1 mutation can result in an normalization of I_{Ks} current density, normalization of cardiac APD, and/or regulation of heart rhythm.

In another embodiment, a mammal having LQTS or CPVT associated with a pathogenic mutation in the CALM2 gene can be identified by, for example, analyzing a biological sample (e.g., analyzing a blood sample using PCR and/or DNA sequencing methods) obtained from the mammal to determine whether DNA in the sample includes a pathogenic mutation in the CALM2 gene. Pathogenic mutations in or encoded by the CALM2 gene include, without limitation, p.D96V, p.N98I, p.N98S, p.D130G, p.D130V, p.E132E, p.D132H, p.D134H, and p.Q136P. See, also, Jensen et al., supra; and Boczek et al. supra. SupRep constructs targeted to mutant CALM2 alleles can be designed to suppress the mutant CALM2 alleles and replace them with a wild type CALM2 allele. SupRep constructs targeted to mutant CALM2 alleles can be designed and prepared using methods described, for example, in the Examples herein. For example, a SupRep construct can be generated to target a CALM2 allele containing a pathogenic mutation, either by targeting a region of a disease-associated CALM2 allele that contains a pathogenic mutation, or by targeting a region of a disease-associated CALM2 allele that does not contain a pathogenic mutation. The SupRep constructs can be tested for their ability to suppress a mutant CALM2 allele and replace it with a wild type CALM2 allele. For example, constructs can be tested in an in vitro model system by co-transfecting cultured cells with a wild type CALM2 construct and a shgene construct, and measuring CALM2 expression with qRT-PCR and/or western blotting. A construct having a relatively high ability to knock down CALM2 expression (e.g., the ability to knock down at least 70 percent, at least 75 percent, at least 80 percent, at least 85 percent, at least 90 percent, at least 95 percent, at least 98 percent, or at least 99 percent of CALM2 expression at the mRNA and/or protein level) can be selected. The selected construct can be packaged in a virus particle (e.g., an AAV particle) and delivered to a mammal identified as having a pathogenic mutation in the CALM2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CALM2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS or CPVT, to determine whether one or more symptoms of the disorder are diminished. For example, effective SupRep treatment of a mammal having LQTS or CPVT associated with a pathogenic mutation in CALM2 can result in a reduction in symptoms such as rapid heartbeat, fainting, seizures, dizziness, lightheadedness, and/or VT. In some cases, effective SupRep treatment of a mammal having LQTS or CPVT associated with a pathogenic CALM2 mutation can result in an normalization of I_{Ks} current density, normalization of cardiac APD, and/or regulation of heart rhythm.

In another embodiment, a mammal having LQTS or CPVT associated with a pathogenic mutation in the CALM3 gene can be identified by, for example, analyzing a biological sample (e.g., analyzing a blood sample using PCR and/or DNA sequencing methods) obtained from the mammal to determine whether DNA in the sample includes a pathogenic mutation in the CALM3 gene. Pathogenic mutations in or encoded by the CALM3 gene include, without limitation, p.D96H, p.A103V, p.D130G, and p.F142L. See, also, Jensen et al., supra; and Boczek et al. supra. SupRep constructs targeted to mutant CALM3 alleles can be designed to suppress the mutant CALM3 alleles and replace them with a wild type CALM3 allele. SupRep constructs targeted to mutant CALM3 alleles can be designed and prepared using methods described, for example, in the Examples herein. For example, a SupRep construct can be generated to target a CALM3 allele containing a pathogenic mutation, either by targeting a region of a disease-associated CALM3 allele that contains a pathogenic mutation, or by targeting a region of a disease-associated CALM3 allele that does not contain a pathogenic mutation. The SupRep constructs can be tested for their ability to suppress a mutant CALM3 allele and replace it with a wild type CALM3 allele. For example, constructs can be tested in an in vitro model system by co-transfecting cultured cells with a wild type CALM3 construct and a shgene construct, and measuring CALM3 expression with qRT-PCR and/or western blotting. A construct having a relatively high ability to knock down CALM3 expression (e.g., the ability to knock down at least 70 percent, at least 75 percent, at least 80 percent, at least 85 percent, at least 90 percent, at least 95 percent, at least 98 percent, or at least 99 percent of CALM3 expression at the mRNA and/or protein level) can be selected. The selected construct can be packaged in a virus particle (e.g., an AAV particle) and delivered to a mammal identified as having a pathogenic mutation in the CALM3 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CALM3 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS or CPVT, to determine whether one or more symptoms of the disorder are diminished. For example, effective SupRep treatment of a mammal having LQTS or CPVT associated with a pathogenic mutation in CALM3 can result in a reduction in symptoms such as rapid heartbeat, fainting, seizures, dizziness, lightheadedness, and/or VT. In some cases, effective SupRep treatment of a mammal having LQTS or CPVT associated with a pathogenic CALM3 mutation can result in an normalization of I_{Ks} current density, normalization of cardiac APD, and/or regulation of heart rhythm.

In another embodiment, a mammal having TKOS associated with a pathogenic mutation in the TRDN gene can be identified by, for example, analyzing a biological sample (e.g., analyzing a blood sample using PCR and/or DNA sequencing methods) obtained from the mammal to determine whether DNA in the sample includes a pathogenic mutation in the TRDN gene. Pathogenic mutations in or encoded by the TRDN gene include, without limitation, c.613C>T (p.Q205X), c.22+29A>G (p.N9fs*5), c.438_442delTAAGA (p.K147fs*0), c.53_56delACAG (p.D18fs*13), c.423delA (p.E142fs*33), c.502G>T (p.E168X), c.503G>T (p.E168X), c.545_546insA (p.K182fs*10), c.420delA (p.K140fs*34), c.176C>G (p.T59R), c.613C>T (p.Q205X), c.53_56delACAG (p.D18fs*13), c.618delG (p.A208fs*15), and c.232+2T>A. See, also, Clemens et al., Circulation: Gen Precision Med. 12(2): e002419; and Altmann et al., Circulation. 2015, 131(23):2051-2060. SupRep constructs targeted to mutant TRDN alleles can be designed to suppress the mutant TRDN alleles and replace them with a wild type TRDN allele. SupRep constructs targeted to mutant TRDN alleles can be designed and prepared using methods described, for example, in the Examples herein. For example, a SupRep construct can be generated to target a TRDN allele containing a pathogenic mutation, either by targeting a region of a disease-associated TRDN allele that contains a pathogenic mutation, or by targeting a region of a disease-associated TRDN allele that does not contain a pathogenic mutation. The SupRep constructs can be tested for their ability to suppress a mutant TRDN allele and replace it with a wild type TRDN allele. For example, constructs can be tested in an in vitro model system by co-transfecting cultured cells with a wild type TRDN construct and a shTRDN construct, and measuring TRDN expression with qRT-PCR and/or western blotting. A construct having a relatively high ability to knock down TRDN expression (e.g., the ability to knock down at least 70 percent, at least 75 percent, at least 80 percent, at least 85 percent, at least 90 percent, at least 95 percent, at least 98 percent, or at least 99 percent of TRDN expression at the mRNA and/or protein level) can be selected. The selected construct can be packaged in a virus particle (e.g., an AAV particle) and delivered to a mammal identified as having a pathogenic mutation in the TRDN gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TRDN can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of TKOS, to determine whether one or more symptoms of the disorder are diminished. For example, effective SupRep treatment of a mammal having TKOS associated with a pathogenic mutation in TRDN can result in a reduction in symptoms such as fainting, skeletal myopathy, and/or proximal muscle weakness. In some cases, effective SupRep treatment of a mammal having TKOS associated with a pathogenic TRDN mutation can result in correction of T-wave inversions and/or QT prolongation.

In another embodiment, a mammal having CPVT associated with a pathogenic mutation in the RYR2 gene can be identified by, for example, analyzing a biological sample (e.g., analyzing a blood sample using PCR and/or DNA sequencing methods) obtained from the mammal to determine whether DNA in the sample includes a pathogenic mutation in the RYR2 gene. Pathogenic mutations in or encoded by the RYR2 gene include, without limitation, c.1258C>T (p.R420W), c.1259G>A (p.R420Q), c.1519G>A (p.V507I), c.3407C>T (p.A1136V), c.5170G>A (p.E1724K), c.5654G>A (p.G1885E), c.5656G>A (p.G1886S), c.6504C>G (p.H2168Q), c.7158G>A (p.A2387T), c.8874A>G (p.Q2958R), c.12533A>G (p.N4178S), c.13528G>A (p.A4510T), c.14311G>A (p.V4771I), c.14542G>A (p.I4848V), and c.14876G>A (p.R4959Q). See, also, Medeiros-Domingo et al., J Am Coll Cardiol. 2009, 54(22):2065-2074; and Jiang et al., Proc Natl Acad Sci USA. 2004, 101(35): 13062-13067. SupRep constructs targeted to mutant RYR2 alleles can be designed to suppress the mutant RYR2 alleles and replace them with a wild type RYR2 allele. SupRep constructs targeted to mutant RYR2 alleles can be designed and prepared using methods described, for example, in the Examples herein. For example, a SupRep construct can be generated to target a RYR2 allele containing a pathogenic mutation, either by targeting a region of a disease-associated RYR2 allele that contains a pathogenic mutation, or by targeting a region of a disease-associated RYR2 allele that does not contain a pathogenic mutation. The SupRep constructs can be tested for their ability to suppress a mutant RYR2 allele and replace it with a wild type RYR2 allele. For example, constructs can be tested in an in vitro model system by co-transfecting cultured cells with a wild type RYR2 construct and a shRYR2 construct, and measuring RYR2 expression with qRT-PCR and/or western blotting. A construct having a relatively high ability to knock down RYR2 expression (e.g., the ability to knock down at least 70 percent, at least 75 percent, at least 80 percent, at least 85 percent, at least 90 percent, at least 95 percent, at least 98 percent, or at least 99 percent of RYR2 expression at the mRNA and/or protein level) can be selected. The selected construct can be packaged in a virus particle (e.g., an AAV particle) and delivered to a mammal identified as having a pathogenic mutation in the RYR2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to RYR2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of CPVT, to determine whether one or more symptoms of the disorder are diminished. For example, effective SupRep treatment of a mammal having CPVT associated with a pathogenic mutation in RYR2 can result in a reduction in symptoms such as dizziness, lightheadedness, fainting, and/or VT. In some cases, effective SupRep treatment of a mammal having CPVT associated with a pathogenic RYR2 mutation can result in normalization and/or regulation of the heart rhythm.

In another embodiment, a mammal having FH associated with a pathogenic mutation in the APOB gene can be identified by, for example, analyzing a biological sample (e.g., analyzing a blood sample using PCR and/or DNA sequencing methods) obtained from the mammal to determine whether DNA in the sample includes a pathogenic mutation in the APOB gene. Pathogenic mutations in or encoded by the APOB gene include, without limitation, c.10093C>G (p.H3365D), c.4163G>A (p.R1388H), c.10579C>T (p.R3527W), p.P994L, and p.T3826M. See, also, Alves et al., Atherosclerosis. 2018, 277:P448-456; Sun et al., Lipids Health Dis. 2018, 17:252; and Cui et al., Clin Cardiol. 2019, 42:385-390. SupRep constructs targeted to mutant APOB alleles can be designed to suppress the mutant APOB alleles and replace them with a wild type APOB allele. SupRep constructs targeted to mutant APOB alleles can be designed and prepared using methods described, for example, in the Examples herein. For example, a SupRep construct can be generated to target a APOB allele containing a pathogenic mutation, either by targeting a region of a disease-associated APOB allele that contains a pathogenic mutation, or by targeting a region of a disease-associated APOB allele that does not contain a pathogenic mutation. The SupRep constructs can be tested for their ability to suppress a mutant APOB allele and replace it with a wild type APOB allele. For example, constructs can be tested in an in vitro model system by co-transfecting cultured cells with a wild type APOB construct and a shAPOB construct, and measuring APOB expression with qRT-PCR and/or western blotting. A construct having a relatively high ability to knock down APOB expression (e.g., the ability to knock down at least 70 percent, at least 75 percent, at least 80 percent, at least 85 percent, at least 90 percent, at least 95 percent, at least 98 percent, or at least 99 percent of APOB expression at the mRNA and/or protein level) can be selected. The selected construct can be packaged in a virus particle (e.g., an AAV particle) and delivered to a mammal identified as having a pathogenic mutation in the APOB gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to APOB can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of FH, to determine whether one or more symptoms of the disorder are diminished. For example, effective SupRep treatment of a mammal having FH associated with a pathogenic mutation in APOB can result in a reduction in symptoms such as elevated total and LDL cholesterol levels, angina, and/or xanthomas. In some cases, effective SupRep treatment of a mammal having FH associated with a pathogenic APOB mutation can alleviate cerebrovascular disease and/or peripheral vascular disease associated with the FH.

In another embodiment, a mammal having DCM or HCM associated with a pathogenic mutation in the TNNI3 gene can be identified by, for example, analyzing a biological sample (e.g., analyzing a blood sample using PCR and/or DNA sequencing methods) obtained from the mammal to determine whether DNA in the sample includes a pathogenic mutation in the TNNI3 gene. Pathogenic mutations in or encoded by the TNNI3 gene include, without limitation, p.K36Q, p.N185K, and p.98_{truncation}, c.407G>A (p.R136Q), c.433C>T (p.R145W), c.448A>T (p.S150C), c.549G>T (p.K183N), and c.557G>A (p.R186Q). See, also, Bollen et al., J Physiol. 2017, 595(14):4677-4693; and Millat et al., supra. SupRep constructs targeted to mutant TNNI3 alleles can be designed to suppress the mutant TNNI3 alleles and replace them with a wild type TNNI3 allele. SupRep constructs targeted to mutant TNNI3 alleles can be designed and prepared using methods described, for example, in the Examples herein. For example, a SupRep construct can be generated to target a TNNI3 allele containing a pathogenic mutation, either by targeting a region of a disease-associated TNNI3 allele that contains a pathogenic mutation, or by targeting a region of a disease-associated TNNI3 allele that does not contain a pathogenic mutation. The SupRep constructs can be tested for their ability to suppress a mutant TNNI3 allele and replace it with a wild type TNNI3 allele. For example, constructs can be tested in an in vitro model system by co-transfecting cultured cells with a wild type TNNI3 construct and a shTNNI3 construct, and measuring TNNI3 expression with qRT-PCR and/or western blotting. A construct having a relatively high ability to knock down TNNI3 expression (e.g., the ability to knock down at least 50 percent, at least 75 percent, at least 80 percent, at least 85 percent, at least 90 percent, at least 95 percent, at least 98 percent, or at least 99 percent of TNNI3 expression at the mRNA and/or protein level) can be selected. The selected construct can be packaged in a virus particle (e.g., an AAV particle) and delivered to a mammal identified as having a pathogenic mutation in the TNNI3 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TNNI3 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or HCM, to determine whether one or more symptoms of the disorder are diminished. For example, effective SupRep treatment of a mammal having DCM or HCM associated with a pathogenic mutation in TNNI3 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having DCM or HCM associated with a pathogenic TNNI3 mutation can result in reduced contractility, improved relaxation, reduced energy consumption, normalization of LV size, and/or strengthening of the LV.

In another embodiment, a mammal having DCM or HCM associated with a pathogenic mutation in the TNNC1 gene can be identified by, for example, analyzing a biological sample (e.g., analyzing a blood sample using PCR and/or DNA sequencing methods) obtained from the mammal to determine whether DNA in the sample includes a pathogenic mutation in the TNNC1 gene. Pathogenic mutations in or encoded by the TNNC1 gene include, without limitation, c.91G>T (p.A31S), p.Y5H, p.M103I, p.I148V, p.A8V, p.L29Q, p.C84Y, p.E134D, p.D145E, and p.Q122AfsX30. See, also, Parvatiyar et al., J Blot Chem. 2012, 287(38):31845-31855; and Veltri et al., Front Physiol. 2017, 8:221. SupRep constructs targeted to mutant TNNC1 alleles can be designed to suppress the mutant TNNC1 alleles and replace them with a wild type TNNC1 allele. SupRep constructs targeted to mutant TNNC1 alleles can be designed and prepared using methods described, for example, in the Examples herein. For example, a SupRep construct can be generated to target a TNNC1 allele containing a pathogenic mutation, either by targeting a region of a disease-associated TNNC1 allele that contains a pathogenic mutation, or by targeting a region of a disease-associated TNNC1 allele that does not contain a pathogenic mutation. The SupRep constructs can be tested for their ability to suppress a mutant TNNC1 allele and replace it with a wild type TNNC1 allele. For example, constructs can be tested in an in vitro model system by co-transfecting cultured cells with a wild type TNNC1 construct and a shTNNC1 construct, and measuring TNNC1 expression with qRT-PCR and/or western blotting. A construct having a relatively high ability to knock down TNNC1 expression (e.g., the ability to knock down at least 70 percent, at least 75 percent, at least 80 percent, at least 85 percent, at least 90 percent, at least 95 percent, at least 98 percent, or at least 99 percent of TNNC1 expression at the mRNA and/or protein level) can be selected. The selected construct can be packaged in a virus particle (e.g., an AAV particle) and delivered to a mammal identified as having a pathogenic mutation in the TNNC1 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TNNC1 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or HCM, to determine whether one or more symptoms of the disorder are diminished. For example, effective SupRep treatment of a mammal having DCM or HCM associated with a pathogenic mutation in TNNC1 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having DCM or HCM associated with a pathogenic TNNC1 mutation can result in reduced contractility, improved relaxation, reduced energy consumption, normalization of LV size, and/or strengthening of the LV.

In another embodiment, a mammal having DCM or HCM associated with a pathogenic mutation in the MYL2 gene can be identified by, for example, analyzing a biological sample (e.g., analyzing a blood sample using PCR and/or DNA sequencing methods) obtained from the mammal to determine whether DNA in the sample includes a pathogenic mutation in the MYL2 gene. Pathogenic mutations in or encoded by the MYL2 gene include, without limitation, p.D94A, p.D166A, p.P95A, and p.I158L. See, also, Huang et al., FEBS J. 2015, 282(12):2379-2393; Alvarez-Acosta et al., J Cardiovasc Dis. 2014, 2; and Poetter et al., Nat Genet. 1996, 13:63-69. SupRep constructs targeted to mutant MYL2 alleles can be designed to suppress the mutant MYL2 alleles and replace them with a wild type MYL2 allele. SupRep constructs targeted to mutant MYL2 alleles can be designed and prepared using methods described, for example, in the Examples herein. For example, a SupRep construct can be generated to target a MYL2 allele containing a pathogenic mutation, either by targeting a region of a disease-associated MYL2 allele that contains a pathogenic mutation, or by targeting a region of a disease-associated MYL2 allele that does not contain a pathogenic mutation. The SupRep constructs can be tested for their ability to suppress a mutant MYL2 allele and replace it with a wild type MYL2 allele. For example, constructs can be tested in an in vitro model system by co-transfecting cultured cells with a wild type MYL2 construct and a shMYL2 construct, and measuring MYL2 expression with qRT-PCR and/or western blotting. A construct having a relatively high ability to knock down MYL2 expression (e.g., the ability to knock down at least 70 percent, at least 75 percent, at least 80 percent, at least 85 percent, at least 90 percent, at least 95 percent, at least 98 percent, or at least 99 percent of MYL2 expression at the mRNA and/or protein level) can be selected. The selected construct can be packaged in a virus particle (e.g., an AAV particle) and delivered to a mammal identified as having a pathogenic mutation in the MYL2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to MYL2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or HCM, to determine whether one or more symptoms of the disorder are diminished. For example, effective SupRep treatment of a mammal having DCM or HCM associated with a pathogenic mutation in MYL2 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having DCM or HCM associated with a pathogenic MYL2 mutation can result in reduced contractility, improved relaxation, reduced energy consumption, normalization of LV size, and/or strengthening of the LV.

In another embodiment, a mammal having DCM or HCM associated with a pathogenic mutation in the MYL3 gene can be identified by, for example, analyzing a biological sample (e.g., analyzing a blood sample using PCR and/or DNA sequencing methods) obtained from the mammal to determine whether DNA in the sample includes a pathogenic mutation in the MYL3 gene. Pathogenic mutations in or encoded by the MYL3 gene include, without limitation, c.170C>G (p.A57G), c.530 A>G, c.2155C>T (p. R719W), c.77C>T (p.A26V), c.2654A>C (p.N885T), and c.1987C>T (p.R663C). See, also, Poetter et al., supra; and Zhao et al., Int J Mol Med. 2016, 37:1511-1520. SupRep constructs targeted to mutant MYL3 alleles can be designed to suppress the mutant MYL3 alleles and replace them with a wild type MYL3 allele. SupRep constructs targeted to mutant MYL3 alleles can be designed and prepared using methods described, for example, in the Examples herein. For example, a SupRep construct can be generated to target a MYL3 allele containing a pathogenic mutation, either by targeting a region of a disease-associated MYL3 allele that contains a pathogenic mutation, or by targeting a region of a disease-associated MYL3 allele that does not contain a pathogenic mutation. The SupRep constructs can be tested for their ability to suppress a mutant MYL3 allele and replace it with a wild type MYL3 allele. For example, constructs can be tested in an in vitro model system by co-transfecting cultured cells with a wild type MYL3 construct and a shMYL3 construct, and measuring MYL3 expression with qRT-PCR and/or western blotting. A construct having a relatively high ability to knock down MYL3 expression (e.g., the ability to knock down at least 70 percent, at least 75 percent, at least 80 percent, at least 85 percent, at least 90 percent, at least 95 percent, at least 98 percent, or at least 99 percent of MYL3 expression at the mRNA and/or protein level) can be selected. The selected construct can be packaged in a virus particle (e.g., an AAV particle) and delivered to a mammal identified as having a pathogenic mutation in the MYL3 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to MYL3 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or HCM, to determine whether one or more symptoms of the disorder are diminished. For example, effective SupRep treatment of a mammal having DCM or HCM associated with a pathogenic mutation in MYL3 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having DCM or HCM associated with a pathogenic MYL3 mutation can result in reduced contractility, improved relaxation, reduced energy consumption, normalization of LV size, and/or strengthening of the LV.

In another embodiment, a mammal having DCM or HCM associated with a pathogenic mutation in the JPH2 gene can be identified by, for example, analyzing a biological sample (e.g., analyzing a blood sample using PCR and/or DNA sequencing methods) obtained from the mammal to determine whether DNA in the sample includes a pathogenic mutation in the JPH2 gene. Pathogenic mutations in or encoded by the JPH2 gene include, without limitation, p.S101R, p.Y141H, p.S165F, p.T161K, and p.E641X. See, also, Landstrom et al., J Mol Cell Cardiol. 2007, 42:1026-1035; and Jones et al., Sci Rep. 2019, 9:9038. SupRep constructs targeted to mutant JPH2 alleles can be designed to suppress the mutant JPH2 alleles and replace them with a wild type JPH2 allele. SupRep constructs targeted to mutant JPH2 alleles can be designed and prepared using methods described, for example, in the Examples herein. For example, a SupRep construct can be generated to target a JPH2 allele containing a pathogenic mutation, either by targeting a region of a disease-associated JPH2 allele that contains a pathogenic mutation, or by targeting a region of a disease-associated JPH2 allele that does not contain a pathogenic mutation. The SupRep constructs can be tested for their ability to suppress a mutant JPH2 allele and replace it with a wild type JPH2 allele. For example, constructs can be tested in an in vitro model system by co-transfecting cultured cells with a wild type JPH2 construct and a shJPH2 construct, and measuring JPH2 expression with qRT-PCR and/or western blotting. A construct having a relatively high ability to knock down JPH2 expression (e.g., the ability to knock down at least 70 percent, at least 75 percent, at least 80 percent, at least 85 percent, at least 90 percent, at least 95 percent, at least 98 percent, or at least 99 percent of JPH2 expression at the mRNA and/or protein level) can be selected. The selected construct can be packaged in a virus particle (e.g., an AAV particle) and delivered to a mammal identified as having a pathogenic mutation in the JPH2 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to JPH2 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of DCM or HCM, to determine whether one or more symptoms of the disorder are diminished. For example, effective SupRep treatment of a mammal having DCM or HCM associated with a pathogenic mutation in JPH2 can result in a reduction in symptoms such as dyspnea, rapid heartbeat, chest pain, fainting, dizziness, fatigue, edema of the legs and/or ankles, arrhythmia, lightheadedness, and/or heart palpitations. In some cases, effective SupRep treatment of a mammal having DCM or HCM associated with a pathogenic JPH2 mutation can result in reduced contractility, improved relaxation, reduced energy consumption, normalization of LV size, and/or strengthening of the LV.

In another embodiment, a mammal having LQTS, HCM, or LGMD associated with a pathogenic mutation in the CAV3 gene can be identified by, for example, analyzing a biological sample (e.g., analyzing a blood sample using PCR and/or DNA sequencing methods) obtained from the mammal to determine whether DNA in the sample includes a pathogenic mutation in the CAV3 gene. Pathogenic mutations in or encoded by the CAV3 gene include, without limitation, c.233 C>T (p.T78M), c.253 G>A (p.A85T), c.290 T>G (p.F97C), c.423 C>G (p.S141R), p.P104L, and p.R27Q. See, also, Shah et al., J Cachexia Sarcopenia Muscle 2020, 11(3):838-858; and Vatta et al., Circulation. 2006, 114:2104-2112. SupRep constructs targeted to mutant CAV3 alleles can be designed to suppress the mutant CAV3 alleles and replace them with a wild type CAV3 allele. SupRep constructs targeted to mutant CAV3 alleles can be designed and prepared using methods described, for example, in the Examples herein. For example, a SupRep construct can be generated to target a CAV3 allele containing a pathogenic mutation, either by targeting a region of a disease-associated CAV3 allele that contains a pathogenic mutation, or by targeting a region of a disease-associated CAV3 allele that does not contain a pathogenic mutation. The SupRep constructs can be tested for their ability to suppress a mutant CAV3 allele and replace it with a wild type CAV3 allele. For example, constructs can be tested in an in vitro model system by co-transfecting cultured cells with a wild type CAV3 construct and a shCAV3 construct, and measuring CAV3 expression with qRT-PCR and/or western blotting. A construct having a relatively high ability to knock down CAV3 expression (e.g., the ability to knock down at least 70 percent, at least 75 percent, at least 80 percent, at least 85 percent, at least 90 percent, at least 95 percent, at least 98 percent, or at least 99 percent of CAV3 expression at the mRNA and/or protein level) can be selected. The selected construct can be packaged in a virus particle (e.g., an AAV particle) and delivered to a mammal identified as having a pathogenic mutation in the CAV3 gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to CAV3 can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS, HCM, or LGMD, to determine whether one or more symptoms of the disorder are diminished. For example, effective SupRep treatment of a mammal having LQTS, HCM, or LGMD associated with a pathogenic mutation in CAV3 can result in a reduction in symptoms such as dyspnea, rapid heartbeat, arrhythmia, chest pain, fainting, dizziness, seizures, fatigue, atrophy and/or weakness of muscles in the hip and shoulder areas, cardiomyopathy. In some cases, effective SupRep treatment of a mammal having LQTS, HCM, or LGMD associated with a pathogenic CAV3 mutation can result in reduced contractility, improved relaxation, and/or reduced energy consumption.

In another embodiment, a mammal having LQTS or CPVT associated with a pathogenic mutation in the TECRL gene can be identified by, for example, analyzing a biological sample (e.g., analyzing a blood sample using PCR and/or DNA sequencing methods) obtained from the mammal to determine whether DNA in the sample includes a pathogenic mutation in the TECRL gene. Pathogenic mutations in or encoded by the TECRL gene include, without limitation, p.R196Q, c.331+1G>A, p.Q139X, p.P290H, p.S309X, and p.V298A. See, also, Devalla et al., EMBO Mol Med. 2016, 8(12):1390-1408; and Moscu-Gregor et al., J Cardiovasc Electrophysiol. 2020, 31(6):1527-1535. SupRep constructs targeted to mutant TECRL alleles can be designed to suppress the mutant TECRL alleles and replace them with a wild type TECRL allele. SupRep constructs targeted to mutant TECRL alleles can be designed and prepared using methods described, for example, in the Examples herein. For example, a SupRep construct can be generated to target a TECRL allele containing a pathogenic mutation, either by targeting a region of a disease-associated TECRL allele that contains a pathogenic mutation, or by targeting a region of a disease-associated TECRL allele that does not contain a pathogenic mutation. The SupRep constructs can be tested for their ability to suppress a mutant TECRL allele and replace it with a wild type TECRL allele. For example, constructs can be tested in an in vitro model system by co-transfecting cultured cells with a wild type TECRL construct and a shTECRL construct, and measuring TECRL expression with qRT-PCR and/or western blotting. A construct having a relatively high ability to knock down TECRL expression (e.g., the ability to knock down at least 70 percent, at least 75 percent, at least 80 percent, at least 85 percent, at least 90 percent, at least 95 percent, at least 98 percent, or at least 99 percent of TECRL expression at the mRNA and/or protein level) can be selected. The selected construct can be packaged in a virus particle (e.g., an AAV particle) and delivered to a mammal identified as having a pathogenic mutation in the TECRL gene at a dose of, for example, about 10¹⁰ vg/kg to about 10¹⁵ vg/kg, or about 10¹⁰ AAV particles/mL to about 10¹⁵ AAV particles/mL, using any appropriate route of administration (e.g., via direct injection into a tissue such as the myocardium, or via intraperitoneal administration, intranasal administration, intravenous administration, intrathecal administration, intracerebral administration, intraparenchymal administration, or oral delivery in nanoparticles and/or drug tablets, capsules, or pills). In some cases, a SupRep construct targeted to TECRL can be administered to a mammal in a non-viral vector (e.g., in a plasmid or in a nucleic acid molecule complexed with lipids, polymers, or nanospheres), and can be delivered by direct injection to a tissue (e.g., the myocardium), or by intraperitoneal, intranasal, intravenous, intrathecal, intracerebral, or intraparenchymal administration, or by oral delivery. After administration, the mammal can be monitored for symptoms of LQTS or CPVT, to determine whether one or more symptoms of the disorder are diminished. For example, effective SupRep treatment of a mammal having LQTS or CPVT associated with a pathogenic mutation in TECRL can result in a reduction in symptoms such as rapid heartbeat, fainting, seizures, dizziness, lightheadedness, and/or VT. In some cases, effective SupRep treatment of a mammal having LQTS or CPVT associated with a pathogenic TECRL mutation can result in an normalization of IKs current density, normalization of cardiac APD, and/or regulation of heart rhythm.

The invention will be further described in the following examples, which do not limit the scope of the invention described in the claims.

### EXAMPLES

### Example 1—Materials and Methods

Samples: Human samples were obtained from patients with LQT1 and from an unrelated healthy control (TABLE 2).

Plasmids and Cloning of KCNQ1-SupRep: WT KCNQ1 cDNA (NM_000218.2) was subcloned into pIRES2-EGFP (Clontech; Mountain View, CA) using NheI and BamHI restriction sites. The QuikChange II XL site-directed mutagenesis kit (Agilent; Santa Clara, CA) was used to introduce two missense variants (p. T66S and p. Y67W) into the chromophore domain of EGFP, converting it to a cyan fluorescent protein and creating pIRES2-CFP-KCNQ1-WT. A second round of site-directed mutagenesis was completed using pIRES2-CFP-KCNQ1-WT to introduce the KCNQ1 variants p.Y171X, p.V254M, and p.I5675 (c.513C>A, c.760G>A, and c.1700T>G, respectively). Four pre-designed KCNQ1 shRNAs (sh#1-4) were purchased from OriGene (Rockville, MD) in the pGFP-C-shLenti backbone along with a non-targeting scramble shRNA control (shCT). The shRNA sequences are listed in TABLE 3A. KCNQ1 sh#4 was selected for the final KCNQ1-SupRep gene therapy vector and is referred to throughout this document as shKCNQ1. A DNA fragment containing ten synonymous variants within the KCNQ1 sh#4 (shKCNQ1) target sequence of the KCNQ1-WT cDNA: c.1377C>T, c.1380C>A, c.1383T>C, c.1386C>T, c.1389T>C, c.1392T>C, c.1395A>C, c.1398G>A, c.1401G>A, and c.1404C>T (KCNQ1: p.D459D, p.G460G, p.Y461Y, p.D462D, p.S463S, p.S464S, p.V465V, p.R466R, p.K467K, and p.S468S, respectively) was synthesized and cloned into pIRES2-CFP-KCNQ1-WT using BglII and PvuI restriction sites to create KCNQ1-shIMM (pIRES2-CFP-KCNQ1-shIMM) (GenScript; Piscataway, NJ). KCNQ1-shIMM and the CFP reporter were then PCR subcloned into the pGFP-C-shLenti backbone containing shKCNQ1 using 5′MluI and 3′ BsrGI+reverseBsaI restriction sites, excising the original GFP in the process to create the final KCNQ1-SupRep (pCFP-C-shLenti-shKCNQ1-KCNQ1-shIMM). Primers used for PCR cloning were:

(forward primer; SEQ ID NO: 1)
5′-GGCACGCGTTTATGGCCGCGGCCTCCTC-3′.
and
(reverse primer; SEQ ID NO: 2)
5′-GCCGGTCTCTGTACACCGCTTTACTTGTA
CAGCTCGTCC-3′.

LQT1 and Unrelated Control Patient Selection for iPSC Generation: Patients were evaluated by a genetic cardiologist and LQTS specialist. Dermal fibroblasts or peripheral blood mononuclear cells (PBMCs) were collected by 4 mm skin punch biopsy or blood sample, respectively. Samples were obtained from nearly 1200 patients diagnosed with a variety of inherited cardiac channelopathies and their affected or unaffected family members, including 236 patients with LQT1. Four LQT1 patients were selected to span a variety of variant types (one nonsense, two missense, one synonymous splice) and phenotypes. These four patients included a lifelong asymptomatic patient and three patients with strong LQT1 phenotypes, defined as having at least one ECG with QTc greater than 500 ms, a positive history of LQTS-related symptoms (syncope, seizure, near drowning, sudden cardiac arrest), and a positive family history of LQTS-related symptoms. A presumably healthy, unaffected father of a patient hosting a de novo variant was selected as an unrelated control.

Fibroblast/PBMCs Reprogramming into iPSCs and Quality Control: Fibroblasts or PBMCs were reprogrammed by Sendai virus transduction using the CytoTune-iPS 2.0 reprogramming kit (Thermo; Waltham, MA) or electroporation with four episomal DNA plasmids containing the Yamanaka factors: pCXLE-hUL, pCXLE-hSK, pCXLE-hOCT3/4-shp53-F, and pCXWB-EBNA1 (Addgene; Watertown, MA). At least two colonies were picked within 21 days post-induction and clonally expanded. All iPSCs were cultured in mTeSR™1 (STEMCELL®; Vancouver, Canada) supplemented with 1% penicillin/streptomycin on MATRIGEL®-coated (Corning; Corning, NY) 6 cm culture dishes in a 5% CO₂ incubator at 37° C. At 85% confluence, iPSCs were passaged using ReLeSR (STEMCELL®). Each clone was then karyotyped.

All lines had normal karyotype except the patient with KCNQ1-V254M (and subsequent isogenic control), which had a reprogramming-induced balanced translocation between chromosomes 13 and 22. No genes encoding ion channels critical to the cardiac action potential are located on chromosomes 13 or 22, so these cells were still included in the study. KCNQ1 variant confirmation was conducted by Sanger sequencing of PCR-amplicons from genomic DNA. Expression of pluripotent markers in all iPSC clones was confirmed by confocal immunofluorescence microscopy using primary antibodies against Oct4 (Thermo, PAS-27438), Nanog (Thermo, PA1-097), Tra-1-60 (Santa Cruz; Dallas, TX; sc-21705), and SSEA-4 (Thermo, MA1-021) at a 1:250 dilution. Secondary antibodies were ALEXA FLUOR® 488 goat-anti-mouse (Thermo, A-11001) and ALEXA FLUOR® 594 goat-anti-rabbit (Thermo, A-11037). Counterstaining with DAPI (Thermo) was used at a 1:2000 dilution from a 5 mg/mL stock. Images were acquired on a Zeiss LSM 780 confocal microscope.

iPSC-CM Culture, Differentiation, and Dissociation: When iPSCs were 85% confluent, differentiation into cardiomyocytes (CMs) was induced as described elsewhere (Schwartz 2009, supra; and Schwartz 2013, supra). Differentiation was initiated (day 0) by changing the culture medium to RPMI 1640 GlutaMAX™ plus 25 mM HEPES ((4-(2-hydroxyethyl)-1-piperazineethanesulfonic acid)) supplemented with B27-minus insulin (RPMI/B27-ins) (Thermo) containing 51.1M CHIR99021 (MilliporeSigma; St. Louis, MO). On day 2, the medium was changed to RPMI/B27-ins containing 51.1M IWP-2 (MilliporeSigma). On day 4, the medium was changed back to the maintenance medium RPMI/B27-ins. Spontaneous beating typically began on days 6-7, with expansion to remaining cells by days 10-12. The iPSC-CMs were allowed to mature until at least day 30, changing the media twice per week. After day 30, iPSC-CMs were dissociated enzymatically using STEMdiff™ cardiomyocyte dissociation kit (STEMCELL®). Briefly, cells were rinsed with PBS (without Ca²⁺/Mg²⁺) and placed in dissociation medium for 10 minutes at 37° C., and then deactivated by addition of STEMdiff™ Cardiomyocyte Support Medium (STEMCELL®). Cells were triturated, transferred to a 15 mL conical tube, and pelleted by centrifugation at 300 rcf for 3 minutes. The supernatant was aspirated and the cells suspended in Cardiomyocyte Support Medium before transfer to appropriate MATRIGEL®-coated culture ware. After 24 hours, the medium was changed back to RPMI/B27-ins. Dissociation resulted in a mixture of single cells and small-to-medium sized iPSC-CM clusters, depending on cell density before and after plating. Spontaneous beating generally returned 24 hours after dissociation, with strong electrical coupling and syncytia formation between days 3-7.

CRISPR-Cas9 Corrected Isogenic Control iPSC: Genome editing of iPSC cell lines was contracted through Applied Stem Cell (Milpitas, CA). Isogenic “variant corrected” control iPSC cell lines were created for the two patient-specific LQT1 cells lines harboring KCNQ1-V254M (c.760G>A) and KCNQ1-A344A/spl (c.1032G>A). Guide RNAs (gRNAs) were designed using the gRNA design tool by Applied Stem Cell. Based on proximity to the target site and off-target profile, two gRNAs were selected for assessment of gRNA activity by next generation sequencing. Based on these results, the gRNAs 5′-CTGGCGGTGGATGAAGACCA-3′ (KCNQ1-V254M; SEQ ID NO:3) and 5′-CCCAGCAGTAGGTGCCCCGT-3′ (KCNQ1-A344A/spl; SEQ ID NO:4) were selected. Single-stranded oligodeoxynucleotide donors (ssODNs) were designed to be used as the repair template at the gRNA cut sites during homology directed repair. The isogenic control ssODNs were:

(KCNQ1-V254M; SEQ ID NO: 5)
5′-CAGATCCTGAGGATGCTACACGTCGACCGCC
AGGGAGGCACCTGGAGGCTGCTGGGCTCGGTGGT
CTTCATCCACCGCCAGgtgggtggcccgggttag
gggtgcggggcccag-3′
and
(KCNQ1-A344A/spl; SEQ ID NO: 6)
5′-gtgcagccaccccaggaccccagctgtcca
aggagccagggaaaacgcacacacggggcaccta
cCGCTGGGAGCGCAAAGAAGGAGATGGCAAAGAC
AGAGAAGCAGGAGGCGAT-3′,

where uppercase=exon, lowercase=intron, underline=synonymous variant to prevent re-cutting after successful editing, and underline+bold+italic=WT nucleotide to replace target variant.

The gRNA was cloned into the expression vector pBT-U6-Cas9-2A-GFP, and the resulting plasmid was transfected into iPSCs along with the ssODN. Parental iPSCs (5×10⁵) were plated on six-well plates and transfected by electroporation using 1100V, 30 ms, 1P in the Neon Transfection System (Thermo). The iPSC population was subjected to limiting dilution for cloning and genotype analysis. Genomic DNA was extracted from each iPSC clone and analyzed by Sanger sequencing for the absence of the KCNQ1-V254M and KCNQ1-A344A/spl variants, respectively.

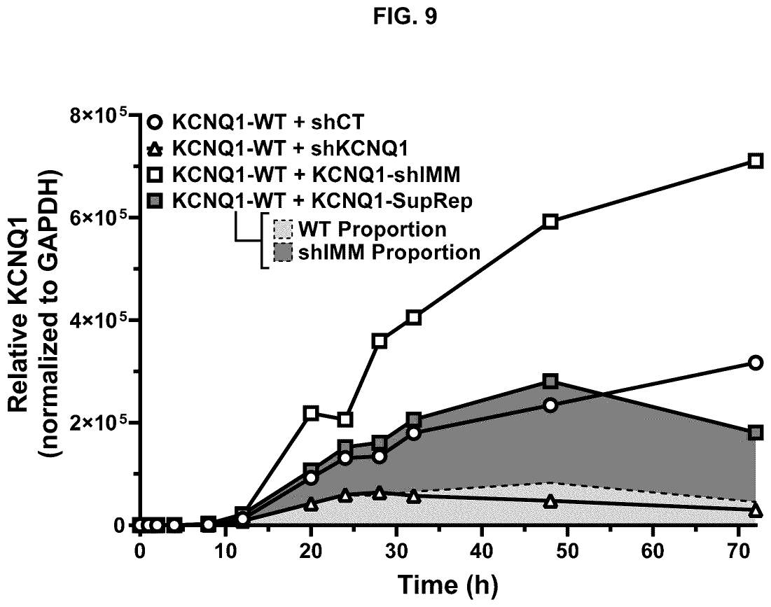

TSA201 Cell Culture and Transfection: TSA201 cells (passage 20 or lower) were maintained in Dulbecco's Modified Eagle Medium (Corning) supplemented with 10% fetal bovine serum, 1% L-glutamine, and 1% penicillin/streptomycin in a 5% CO₂ incubator at 37° C. For patch clamp, cells were split into T25 flasks. After 24 hours, heterologous expression of the Kv7.1 channel (KCNQ1 α-subunit plus KCNE1 (3-subunit) was achieved using 5 μL LIPOFECTAMINE® 2000 (Thermo) to co-transfect 1 μg of pIRES2-CFP-KCNQ1-WT, -shIMM, -Y171X, -V254M, or 4567S and 1 μg of pIRES2-dsRED2-KCNE1-WT in OPTI-MEM® (Thermo). After 4-6 hours, the medium was replaced with the maintenance medium for 48 hours before patch clamp electrophysiology experiments. For allele-specific qRT-PCR, western blot, and trafficking immunofluorescence microscopy, 5×10⁵ cells (or 1.5×10⁶ cells for the activation kinetics time course in FIG. 9) were plated per well in 6-well plates. After 24 hours, cells were co-transfected in maintenance medium using 10 μL EFFECTENE® (Qiagen; Hilden, Germany) with 100 fmol (between 0.3-0.7 μg) equimolar amounts (or as otherwise indicated) of each plasmid pIRES2-CFP-KCNQ1-WT or -variant, pGFP-C-shLenti-shKCNQ1(#1-#4) or -shCT, pCFP-C-shLenti-KCNQ1-SupRep, or pIRES2-dsRED2-KCNE1-WT, as indicated by each figure. Endpoint assays were conducted as described in the appropriate methods sections.

Cell Membrane Trafficking Immunofluorescence Microscopy: TSA201 cells were co-transfected with KCNQ1-WT, -shIMM, or -variants and KCNE1-WT as above. After 24 hours, cells were dissociated using TrypLE™ Express (Thermo) and plated into 8-chamber culture slides (CELLTREAT®; Pepperell, MA). After another 24 hours, cells were fixed with 4% paraformaldehyde for 10 minutes and washed 3 times with PBS. Cells were blocked with 0.2% Tween-20/5% goat serum in PBS for 1 hour and incubated at 4° C. overnight using a primary antibody against KCNQ1 (Santa Cruz, sc-365186) at a 1:100 dilution. Cells were washed 3 times for 15 minutes each with PBS-0.2% TWEEN®-20 and incubated in secondary ALEXA FLUOR® 488 goat-anti-mouse (Thermo) at a dilution of 1:250 for 1 hour before washing again 3 times for 15 minutes each. DAPI (4′,6-diamidino-2-phenylindole) counterstain was added during the first wash at a concentration of 1:2000 as before. VECTASHIELD® mounting media (Vector Labs; Burlingame, CA) was diluted 1:10 in PBS and used as mounting solution, and images were acquired on a Zeiss LSM 780 confocal microscope. Results shown in the figures herein are representative of three independent experiments (defined throughout the study as “three identical repeats of each experiment conducted from start to finish on separate weeks with one biological replicate per treatment group per run”).

Western Blotting: TSA201 cells were co-transfected with KCNQ1-WT, -shIMM, or -variants and shKCNQ1(#1-4), -shCT, or KCNQ1-SupRep as described above. After 48 hours, cells were lysed in 1× RIPA buffer with protease and phosphatase inhibitors and chilled on ice for 10 minutes. Lysates were sonicated for 10 seconds at 50% amplitude and the cell debris was pelleted at 21,000 rcf for 15 minutes at 4° C. The supernatant was collected and the protein concentration quantified by BCA assay (Thermo) before mixing 1:1 with loading buffer (2X Laemmli buffer with 1:20 (3-mercaptoethanol). Importantly, the lysates were NOT denatured at 95° C., which would have caused irreversible SDS-resistant high molecular weight aggregates of the KCNQ1 proteins (Sagné et al., Biochem. J., 316(Pt 3):825-831 (1996); and Little, “Amplification-refractory mutation system (ARMS) analysis of point mutations,” Curr. Protoc. Hum. Genet., Chapter 9:Unit 9.8 (2001)). Proteins (10 μg/lane) were run on a 4-15% TGX gel (Bio-Rad; Hercules, CA) and transferred to a PVDF membrane using a Trans-Blot Turbo Transfer System (Bio-Rad). Membranes were blocked for 1 hour in tris-buffered saline (TB S) with 0.1% TWEEN®-20/3% bovine serum albumin and incubated at 4° C. overnight with primary antibodies against KCNQ1 (Santa Cruz, sc-365186) and Cofilin (Santa Cruz, sc-376476) as a housekeeping control at a 1:1000 dilution in blocking solution. The membrane was washed 3 times for 15 minutes each with TBS-0.1% TWEEN®-20 prior to addition of secondary antibody HRP-conjugated goat-anti-mouse (R&D Systems; Minneapolis, MN; HAF007) at a dilution of 1:5000 in blocking solution. The membrane was washed 3 times for 15 minutes each with TB S and incubated in SuperSignal™ West Pico PLUS chemiluminescent ECL substrate (Thermo) for 3 minutes and exposed using autoradiography film. Pixel density was quantified using freely available ImageJ software. All western blots presented herein are representative images of three independent experiments.

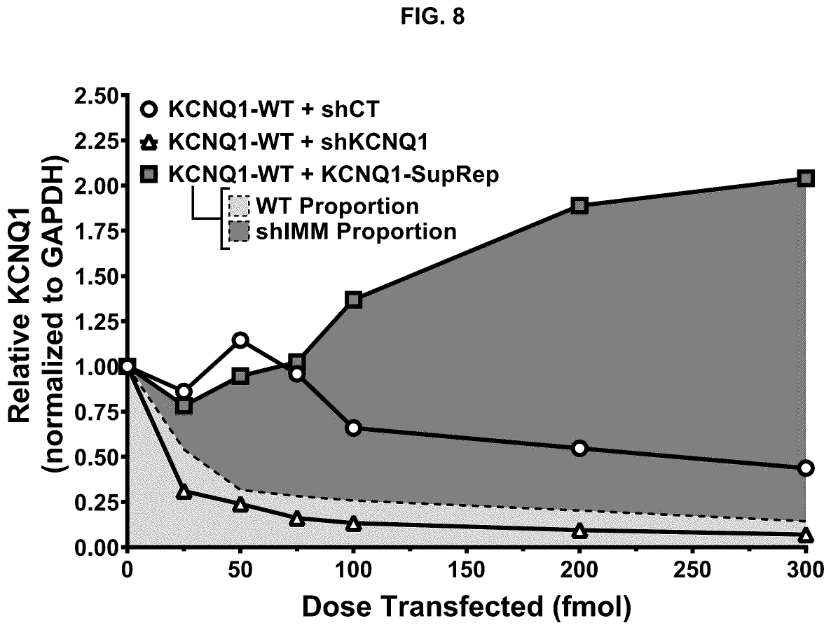

Allele-Specific qRT-PCR: Allele-specific primers were developed for qRT-PCR to specifically amplify (1) total KCNQ1, (2) endogenous KCNQ1 (includes KCNQ1-WT and -variants, but excludes KCNQ1-shIMM), and (3) KCNQ1-shIMM, by adapting allele-specific genotyping methods described elsewhere (TABLE 4) (Rohatgi et al., supra; and Priori et al., supra). For total KCNQ1, primers were purchased from IDT (Coralville, IA; PRIMETIME qPCR Primer Assay, Hs.PT.58.41042304). Allele-specific primers were created by designing two forward primers spanning the shKCNQ1 target site, with one complementary to endogenous KCNQ1 (allele-specific for KCNQ1-WT and -variants) and the other complementary to KCNQ1-shIMM (allele-specific for KCNQ1-shIMM). A common reverse primer was used with both allele-specific forward primers. GAPDH primers were purchased from IDT (PRIMETIME™ qPCR Primer Assay, Hs.PT.39a.22214836) as a housekeeping control. A standard curve was used to correct for PCR amplification bias. TSA201 cells were co-transfected with KCNQ1-WT, -shIMM, or -variants and shKCNQ1(#1-4), -shCT, or KCNQ1-SupRep as above. After 48 hours (or at the indicated time for the activation kinetics time-course in FIG. 9), RNA was harvested using an RNeasy kit (Qiagen) and quantified using a NanoDrop ND-1000 spectrophotometer (Thermo). Complementary DNA (cDNA) was generated by loading 500 ng RNA in the SuperScript™ IV VILO™ Master Mix reverse transcription kit (Thermo). For each sample, four qRT-PCR reactions were run using the SYBR Green Master Mix kit (Qiagen) with the four sets of primers as described. Data was analyzed using the ΔΔCT method by first normalizing KCNQ1 to GAPDH and then comparing the relative fold change to the KCNQ1-WT and shCT treatment group. All qRT-PCR experiments (except the dose-response curve in FIG. 8 and the time-course in FIG. 9) are the results of three independent experiments.

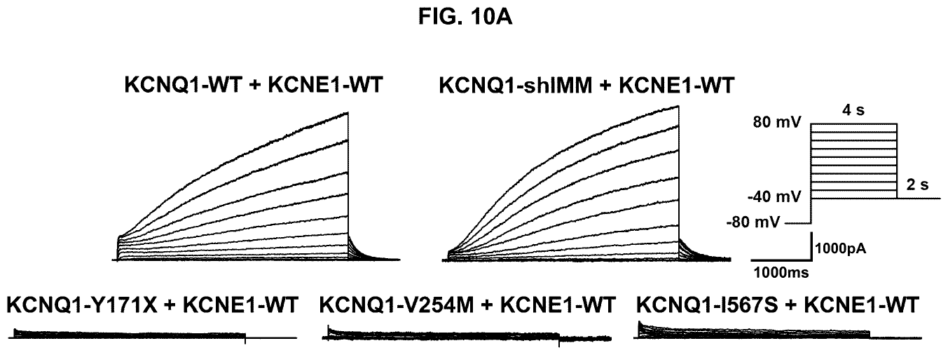

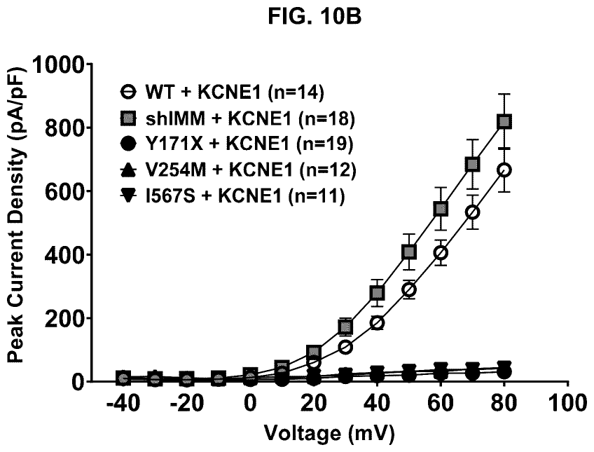

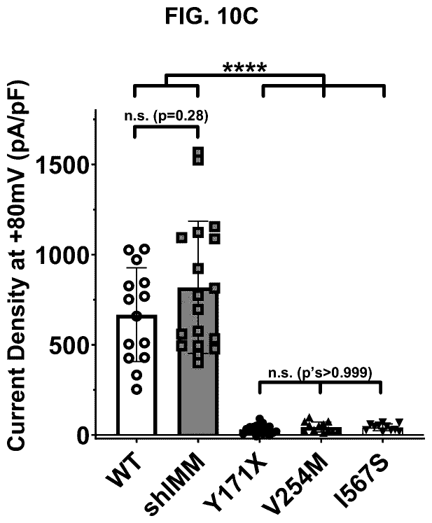

I_{Ks} Whole Cell Patch Clamp Electrophysiology: A standard whole-cell patch clamp technique was used to measure the slow delayed rectifier current, I_{Ks}, produced by KCNQ1-WT, -shIMM, and -variants at room temperature (22-24° C.) with the use of Axopatch 200B amplifier, Digidata 1440A system, and pCLAMP version 10.7 software (Axon Instruments; Sunnyvale, CA). The extracellular (bath) solution contained the following (mmol/L): 150 NaCl, 5.4 KCl, 1.8 CaCl₂, 1.0 MgCl₂, 1 Na-pyruvate, and 15 HEPES. The pH was adjusted to 7.4 with NaOH. The intracellular (pipette) solution contained the following (mmol/L): 20 KCl, 125 K-aspartate, 1 MgCl₂, 10 EGTA, 5 Mg-ATP, 5 HEPES, 2 Nae-phosphocreatine, and 2 Nae-GTP. The pH was adjusted to 7.2 with KOH (Al-Khatib et al., supra). Microelectrodes were pulled on a P-97 puller (Sutter Instruments; Novato, CA) and fire polished to a final resistance of 2-3MS2. The series resistance was compensated by 80-85%. Currents were filtered at 1 kHz and digitized at 5 kHz with an 8-pole Bessel filter. The voltage dependence of activation was determined using voltage-clamp protocols described in the description of FIGS. 10A-10C. Data were analyzed using Clampfit (Axon Instruments) and Excel (Microsoft; Redmond, WA) and fitted with GraphPad Prism 8 software (GraphPad; San Diego, CA).

Lentivirus Generation and Transduction of iPSC-CMs: For application of KCNQ1-SupRep to iPSC-CMs (or shCT as a treatment control), lentivirus was used. Lentiviral particles were generated from pCFP-C-shLenti-shKCNQ1-shIMM (KCNQ1-SupRep) and pGFP-C-shLenti-shCT (shCT), using the pPACKH1 HIV Lentivector Packaging kit (SBI System Biosciences; Palo Alto, CA). Lentiviral titers were quantified by two methods, including qRT-PCR (˜1×10¹¹ viral genomes/mL) to determine the total number of viral particles, and by transducing TSA201 cells in serial dilution to define the number of functional infectious particles (˜5×10⁸ infectious units/mL). Lentivirus was applied to iPSC-CMs at a multiplicity of infection (MOI) of 20-25 infectious units/cell (4,000-5,000 viral genomes/cell). After reaching at least day 30 post-induction of differentiation, iPSC-CMs derived from the healthy unrelated control, the four patients with LQT1, or two isogenic controls, were dissociated and plated into MATRIGEL®-coated 35 mm dishes with glass-bottom insets for FluoVolt™ (MatTek; Ashland, MA) or 8-chamber culture slides for immunofluorescence (CELLTREAT) as described above. After 24-48 hours of recovery, iPSC-CMs were left untreated or were transduced with lentiviral particles containing KCNQ1-SupRep or shCT treatment control at an MOI of 20-25. To increase transduction efficiency, Polybrene infection reagent (MilliporeSigma) was added during transduction to a final concentration of 8 μg/mL and the iPSC-CMs were centrifuged at 250 rcf for 1.5 hours at room temperature in the 35 mm dishes. After 24 hours post-transduction, the medium was exchanged for fresh maintenance medium, RPMI/B27-ins.

Immunofluorescence in iPSC-CMs: Immunofluorescence was conducted 7 days post-transduction of iPSC-CMs with lentiviral particles containing either KCNQ1-SupRep or shCT. Cells were fixed with 4% paraformaldehyde for 10 minutes and washed 3 times with PBS. Cells were blocked with 0.1% Triton X-100/5% donkey serum in PBS for 1 hour and incubated at 4° C. overnight using primary antibodies against cTnT (abcam; Cambridge, UK, ab45932), turboGFP for treatment with shCT (OriGene, TA150041) or eCFP for treatment with KCNQ1-SupRep (MyBioSource; San Diego, CA, MBS9401609), and KCNQ1 (Santa Cruz, sc-10646) at a 1:100 dilution each in blocking solution. Cells were washed 3 times for 15 minutes each with PBS-0.1% Triton X-100 and incubated in secondary ALEXA FLUOR PLUS® 488 donkey-anti-goat (Thermo, A32814), ALEXA FLUOR PLUS® 594 donkey-anti-mouse (Thermo, A32744), and ALEXA FLUOR PLUS® 647 donkey-anti-rabbit (Thermo, A32795) at a dilution of 1:250 each in blocking solution for 1 hour before washing again 3 times for 15 minutes each. DAPI counterstain was added during the first wash at a concentration of 1:2000 as before. VECTASHIELD® mounting media (Vector Labs) was diluted 1:10 in PBS and used as mounting solution, and images were acquired on a Zeiss LSM 780 confocal microscope using identical settings between images.

Voltage Dye Optical Action Potentials in iPSC-CMs: Voltage dye experiments were conducted between 3-7 days post-transduction of iPSC-CMs with lentiviral particles containing either KCNQ1-SupRep or shCT. Unrelated control cells and isogenic controls were not transduced with lentivirus, but rather were left untreated to provide an ideal normal baseline representing a “healthy” APD. On the day of imaging, iPSC-CMs were rinsed with pre-warmed (37° C.) HEPES-buffered Tyrode's solution (Alfa Aesar; Haverhill, MA). Using the FluoVolt™ Membrane Potential kit (Thermo), 0.125 μL FluoVolt™ dye and 1.25 μL PowerLoad were added to 0.5 mL Tyrode's solution for each 35 mm glass-bottom dish and incubated at 37° C. for 20 minutes. Excess dye was removed in three rinses with pre-warmed Tyrode's solution, and a final 2 mL Tyrode's solution was added to the iPSC-CMs for imaging. During imaging, the dishes were kept in a heated 37° C. stage-top chamber (Live Cell Instrument; Seoul, South Korea) with 5% CO₂. Using a Nikon Eclipse Ti light microscope (Nikon; Tokyo, Japan) under 40X-water objective magnification, optical action potentials were recorded in 20s fast time-lapse videos at a rate of 50 frames/sec (fps, 20 ms exposure time) with LED illumination at 5% power. iPSC-CMs were paced at 1 Hz (9 ms pulse duration, 25V) using a MyoPacer field stimulator (Ion Optix; Westwood, MA) to eliminate beat-rate dependent effects on the APD. Videos were focused on electrically-coupled syncytial areas of iPSC-CMs (clusters and monolayers) since these areas of cells best follow the pacing stimulus and produce the greatest signal-to-noise represented by large changes in fluorescence intensity (often ˜8-12%). For analysis, rectangular regions of interest were drawn over flashing areas of cells, and NIS-Elements software (Nikon) was used to quantify the fluorescence intensity over time within each region of interest, resulting in optical action potential traces. Using a custom in-house Excel-based program, traces were corrected for photobleaching and the amplitude was normalized as change in fluorescence divided by the baseline minimum florescence (ΔF/Fₘᵢₙ). In a semi-automated manner, common action potential parameters including APD₉₀, APD₅₀, amplitude, rise time, upstroke velocity, etc. were detected for each individual optical action potential and averaged across all beats within a second trace. The average of all beats within a 20 second trace represents a single data point. For representative traces, the maximum amplitude was further normalized to 1.0 to allow for accurate visualization of APD differences.

3D iPSC-CM Organoid Culture, Immunofluorescence, and Optical Action Potentials: 3D-organoids were generated based on a protocol described elsewhere (Zimmerman et al., Circ. Res., 90:223-230 (2002)). Briefly, a spontaneously beating syncytial monolayer of iPSC-CMs from a patient with KCNQ1-Y171X was dissociated as described above. The pelleted iPSC-CMs were resuspended in a mixture of 80% ice cold undiluted MATRIGEL® (Corning) with 20% fetal bovine serum with 1 million iPSC-CMs per 15 μL. Aliquots of 15 μL (containing 1 million iPSC-CMs each) were transferred to an organoid embedding sheet (STEMCELL®) at 37° C. in a 5% CO₂ incubator for 30 minutes to solidify in a spherical shape. The organoids were then transferred to individual wells of a 24-well plate in RPMI/B27-ins. Organoids were allowed to mature for a minimum of 7 days before transducing with lentiviral shCT or KCNQ1-SupRep. After seven days post-transduction, organoids were fixed for immunofluorescence or live-imaged for electrophysiology using FluoVolt™ voltage dye. For immunofluorescence, organoids were rinsed with PBS, fixed in 4% paraformaldehyde for 10 minutes on ice, and washed three times with PBS. Organoids were suspended in Tissue-Plus™ optimal cutting temperature (O.C.T.) compound (Thermo), transferred to disposable base molds (Thermo), and frozen quickly on dry ice. Frozen organoids were cryosectioned and mounted on slides for imaging. Immunofluorescence was conducted as described above using 0.1% Triton X-100/5% goat serum in PBS as blocking solution, primary antibodies against cTnT (abcam, ab45932) and turboGFP for treatment with shCT (OriGene, TA150041) or eCFP for treatment with KCNQ1-SupRep (MyBioSource, MBS9401609) at a 1:100 dilution each. Secondary antibodies were ALEXA FLUOR PLUS® 488 goat-anti-mouse (Thermo, A32723) and ALEXA FLUOR PLUS® 594 goat-anti-rabbit (Thermo, A32740) at a dilution of 1:250 each. For FluoVolt™, the experiment was conducted as above using whole organoids instead of syncytial monolayers.

Statistical Analysis: GraphPad Prism 8 was used for all statistical analysis and to fit all data for figures. Individual data points are shown wherever practical along with the mean. Error bars represent standard deviation (S.D.) unless otherwise indicated in the figure legend. Specific statistical methods are indicated in each figure legend. Briefly, one-way ANOVA with post-hoc Tukey's or Dunnett's test for multiple comparisons was performed for comparisons among three or more groups as appropriate. An unpaired two-tailed student's t-test was performed to determine statistical significance between two groups when indicated. A p<0.05 was considered to be significant.

### Example 2—Generation of a KCNQ1-SupRep Gene Therapy Construct

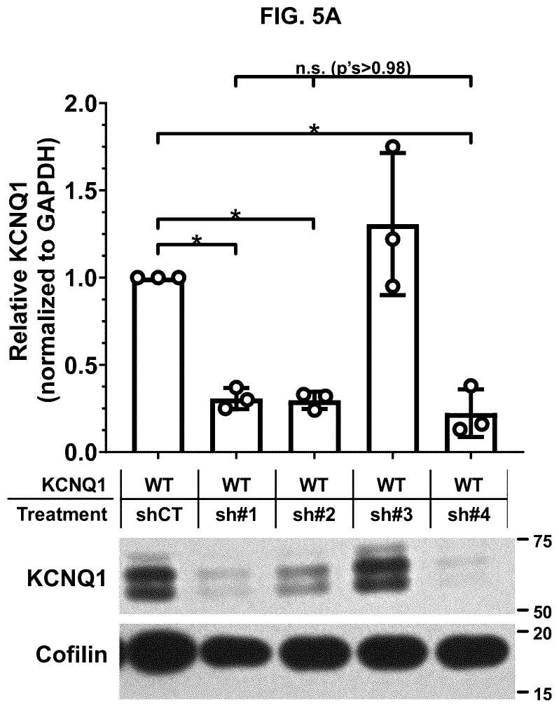

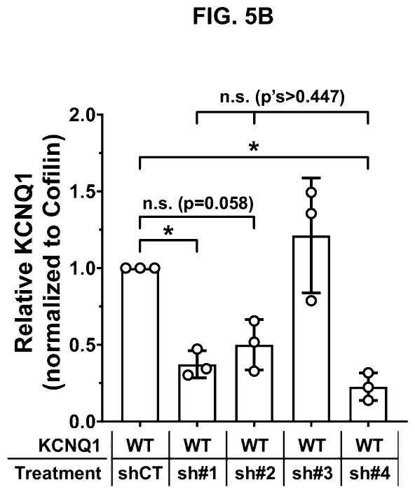

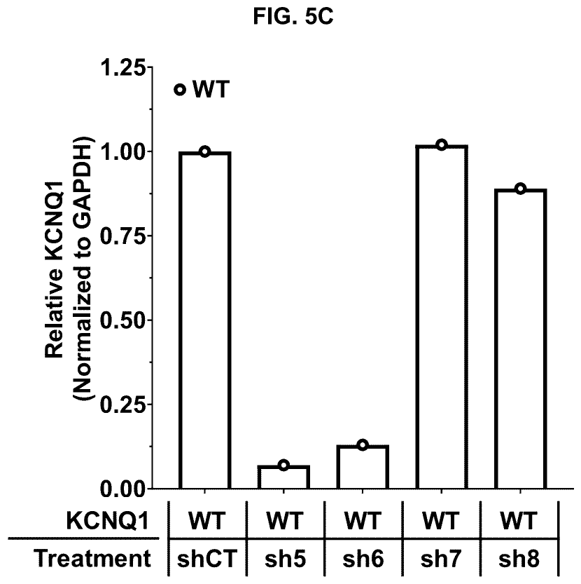

To make KCNQ1-SupRep, four candidate KCNQ1 shRNAs (sh#1-4) in the pGFP-C-shLenti lentiviral backbone were purchased from OriGene, along with a non-targeting scrambled control shRNA (shCT, TABLE 3A). The KD efficiency of each KCNQ1 shRNA was determined by co-transfecting TSA201 cells with KCNQ1-WT and sh#1-4. Expression of KCNQ1 was measured by quantitative reverse transcription PCR (qRT-PCR, FIG. 5A) and confirmed by western blot (FIGS. 5A and 5B). Of the four shRNAs tested, sh#1, sh#2, and sh#4 all resulted in significant KD of KCNQ1 (mRNA: 69-78% KD, protein: 50-77% KD) with no statistically significant differences between the three shRNAs. Any of these shRNAs could in theory have been used as part of the final KCNQ1-SupRep gene therapy vector. To select a final shRNA from the three potential candidates, by raw average KD, KCNQ1 sh#4 provided the strongest KD of KCNQ1 on both the mRNA (78%, p=0.004) and protein (77%, p<0.004) levels. Further, at the time of selection, the KCNQ1 sh#4 target sequence (nucleotides c.1376-1404, exon 10-11 boundary) was assessed using the Genome Aggregation Database (gnomAD) and ClinVar, and was found to be devoid of both common genetic polymorphisms and all known pathogenic LQT1-causative mutations that may interfere with KD efficiency. KCNQ1 sh#4 therefore was selected for the final KCNQ1-SupRep and is referred to herein as “shKCNQ1.”

Four additional, custom-made shRNAs were subsequently tested (sh#5-sh#8; sequences in TABLE 3B). TSA201 cells were co-transfected with KCNQ1-WT and sh#5-sh#8) or non-targeting scrambled shRNA control (shCT). KCNQ1 expression normalized to GAPDH was measured by qRT-PCR. sh#5 had the strongest knockdown (95%) by raw value (FIG. 5C).

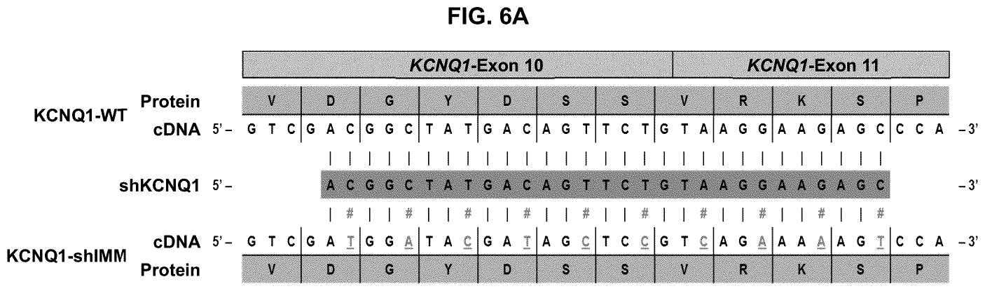

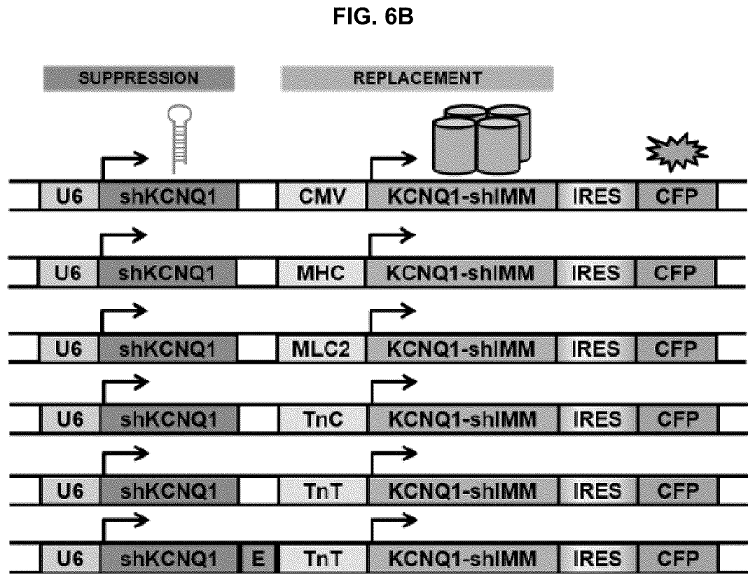

To create the replacement shRNA-immune version of KCNQ1, called KCNQ1-shIMM, ten synonymous variants were introduced into the WT KCNQ1 cDNA at the wobble base of each codon within shKCNQ1's target site, nucleotides c.1376-1404 (FIG. 6A). KCNQ1-shIMM was then cloned into the shKCNQ1-containing vector, pGFP-C-shLenti, downstream of the CMV promoter. In this step, the original GFP reporter (which remained the reporter for shCT) was exchanged for an internal ribosome entry site (IRES) with CFP. The final KCNQ1-SupRep gene therapy vector used in this in vitro study is illustrated in FIG. 6B.

### Example 3—KCNQ1-SupRep Gene Therapy Both Suppresses and Replaces KCNQ1-WT

To confirm that KCNQ1-shIMM is indeed immune to KD by shKCNQ1, TSA201 cells were co-transfected with KCNQ1-WT or KCNQ1-shIMM and shKCNQ1. The expression of KCNQ1-WT versus KCNQ1-shIMM was quantified using allele-specific qRT-PCR. Each sample was run in four separate reactions, using a unique set of allele-specific primers (TABLE 4), to quantify (1) total KCNQ1, (2) endogenous KCNQ1, which includes WT or variant-containing alleles, but excludes KCNQ1-shIMM, (3) KCNQ1-shIMM, and (4) GAPDH as a housekeeping control. Commercial primers were used to amplify total KCNQ1. For exclusive amplification of endogenous KCNQ1 or KCNQ1-shIMM, two forward primers were designed within the shKCNQ1 target site, one complementary to the WT sequence and the other complementary to the unique, modified sequence engineered to create KCNQ1-shIMM. A common reverse primer was used for both reactions, and a standard curve was used to correct for PCR amplification bias.

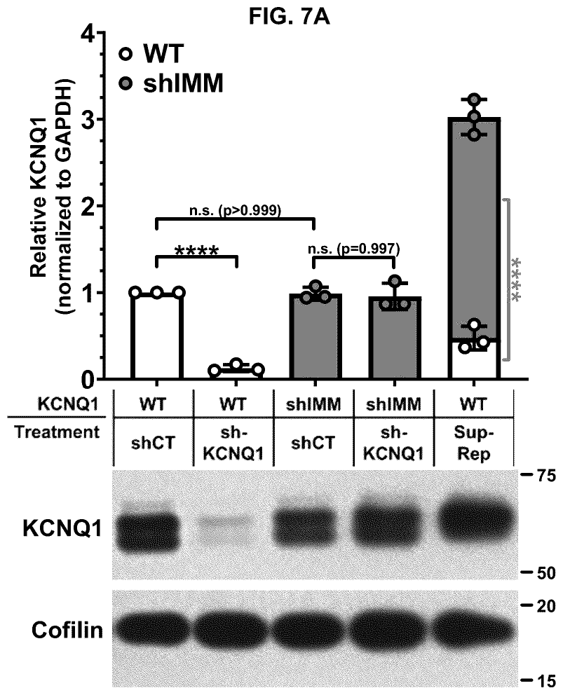

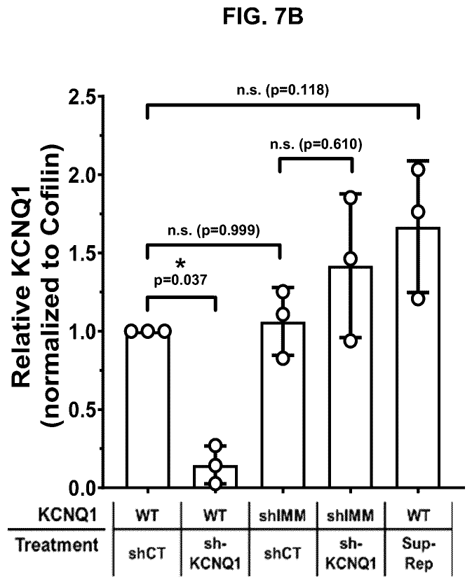

Compared to shCT, shKCNQ1 caused significant (87%) suppression of KCNQ1-WT (p<0.0001), but was unable to suppress KCNQ1-shIMM (p=0.997, FIG. 7A). Notably, there was no difference in the expression of KCNQ1-WT compared to KCNQ1-shIMM (p>0.9999), indicating that introduction of the synonymous variants in KCNQ1-shIMM did not disturb its expressivity as a result of uneven bias in the use of human codons. Next, KCNQ1-SupRep was co-transfected with KCNQ1-WT, which resulted in 52% suppression of KCNQ1-WT with 255% replacement of KCNQ1-shIMM (p<0.0001, FIG. 7A). The dual component KCNQ1-SupRep vector had less potent suppression compared to shKCNQ1 alone, but exhibited stronger expression of KCNQ1-shIMM than KCNQ1-shIMM alone. While the reason for this is unclear, varying amounts of KCNQ1-SupRep were transfected and shown to cause dose-dependent suppression and replacement, suggesting that KCNQ1-SupRep expression can be adjusted as needed (FIG. 8). Results obtained by qRT-PCR were confirmed by western blotting, which demonstrated that shKCNQ1 was able to significantly KD KCNQ1-WT (p=0.037) but not KCNQ1-shIMM (p=0.61, FIGS. 7A and 7B). As a safety metric for onset of the gene therapy, allele-specific qRT-PCR was used to measure the activation kinetics of KCNQ1-SupRep in a three day time course of TSA201 cells co-transfected with WT-KCNQ1 and shCT, shKCNQ1, KCNQ1-shIMM, or KCNQ1-SupRep. Compared to treatment with shCT, KCNQ1-SupRep caused reduction of KCNQ1-WT that was replaced with KCNQ1-shIMM, but the total KCNQ1 was not altered at any time during the three day onset, avoiding over- or under-expression (FIG. 9).

### Example 4—Selection of Patients with LQT1-Causative Variants in KCNQ1

Four patients with LQT1 hosting unique variants, KCNQ1-Y171X, KCNQ1-V254M, KCNQ1-I567S, and KCNQ1-A344A/spl were selected for this study. All four KCNQ1 variants were classified as pathogenic (LQT1-causative) by current American College of Medical Genetics guidelines (Richards et al., Genet. Med., 17:405-424 (2015)). This gene therapy pilot study therefore included a nonsense, premature truncation variant (KCNQ1-Y171X) producing haploinsufficiency in a patient with a mild phenotype, as well as two dominant-negative missense variants (KCNQ1-V254M and KCNQ1-I567S) and a synonymous splice variant (KCNQ1-A344A/spl) that causes skipping of exon 7 (Tsuji et al., J. Mol. Cell Cardiol., 24:662-669 (2007)), in three patients with a strong LQT1 phenotype including documented QTc greater than 500 ms, a positive history of LQTS-related symptoms (syncope, seizure, near drowning, sudden cardiac arrest), and a positive family history of LQTS-related symptoms (TABLE 2).

All four variants have been described elsewhere, though only KCNQ1-V254M and KCNQ1-A344A/spl have been characterized functionally as dominant-negative mutations (Tsjui et al., supra; Piippo et al., J. Am. Coll. Cardiol., 37:562-568 (2001); Wang et al., J. Cardiovasc. Electrophysiol., 10:817-826 (1999); and Choi et al., Circulation, 110:2119-2124 (2004)). Site-directed mutagenesis was used to introduce three of the four LQT1 patient variants (KCNQ1-Y171X, -V254M, and -I567S) into KCNQ1-WT to evaluate the ability of KCNQ1-SupRep to suppress and replace KCNQ1 variants in a mutation-independent manner. KCNQ1-A344A/spl was not included for heterologous expression studies in TSA201 cells since the KCNQ1-WT is a full length cDNA and does not contain the introns necessary to evaluate a splicing variant like KCNQ1-A344A/spl.

### Example 5—Validation of Function for KCNQ1-shIMM and KCNQ1 Pathogenic Variants

KCNQ1-WT and -shIMM, and LQT1-causative variants KCNQ1-Y171X, -V254M, and -I567S were co-transfected into TSA201 cells with the Kv7.1 channel (3-subunit, KCNE1. The resulting I_{Ks} current was measured by standard whole cell patch clamp. Representative traces are shown in FIG. 10A. Importantly, KCNQ1-shIMM produced robust I_{Ks} current with no significant difference from KCNQ1-WT (p=0.28, FIGS. 10B and 10C). All three LQT1 variants (KCNQ1-Y171X, -V254M, and -I567S) resulted in no functional I_{Ks} current beyond the minimal background ion channel activity of TSA201 cells, consistent with complete loss of function (FIGS. 10A-10C). Null current was expected for a nonsense variant like KCNQ1-Y171X and additionally for KCNQ1-V254M, whose null status was in concordance with data described elsewhere (Wang et al., supra). Total lack of current from KCNQ1-I567S was a novel finding, but was consistent with the patient's clinically definitive LQT1 and the fact that most LQT1-causative variants are missense variants.

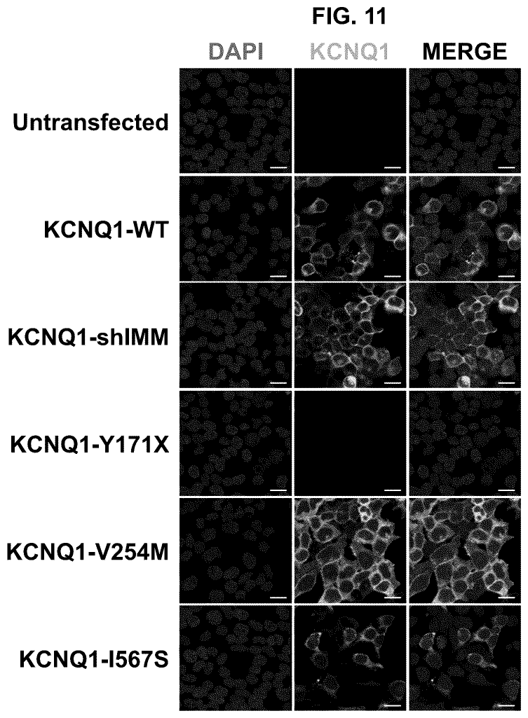

To evaluate trafficking of KCNQ1 to the cell membrane, transfected TSA201 cells were assessed by immunofluorescence microscopy using a KCNQ1 antibody. Both KCNQ1-WT and KCNQ1-shIMM produced bright staining along the cell membrane, indicating that the synonymous variants in KCNQ1-shIMM did not interfere with correct trafficking (FIG. 11). Of the LQT1 variants, KCNQ1-Y171X produced no detectable protein as a result of premature truncation, while KCNQ1-V254M and KCNQ1-I567S exhibited normal cell membrane trafficking, though the overall expression of KCNQ1-I567S appeared to be decreased. Taken together, these results indicated that KCNQ1-shIMM has WT function and that KCNQ1-Y171X, -V254M, and -I567S are LQT1-causative variants with total loss of function.

### Example 6—KCNQ1-SupRep Gene Therapy Both Suppresses and Replaces KCNQ1 Variants in a Mutation-Independent Manner

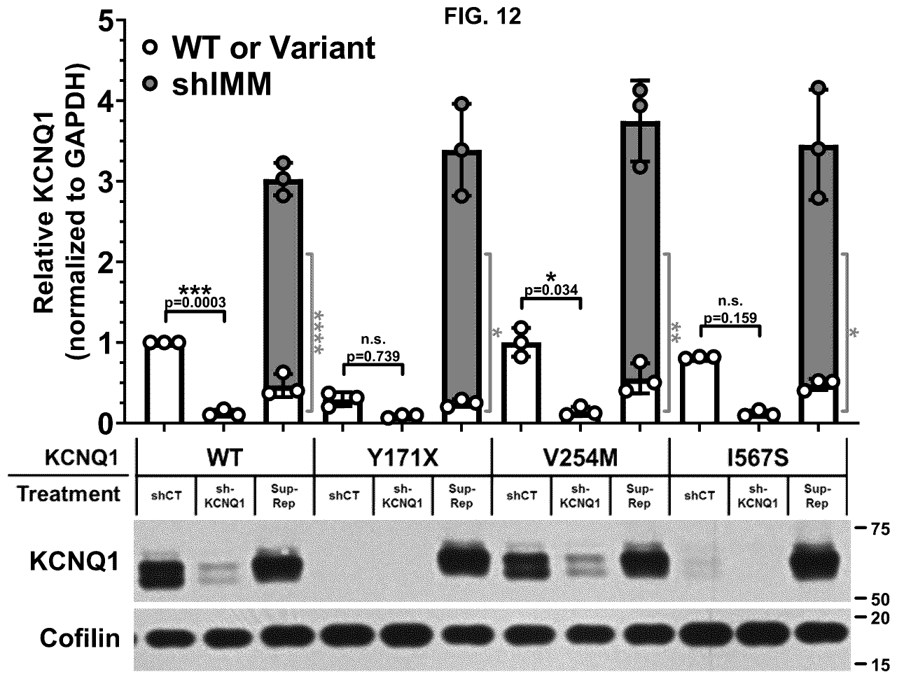

To confirm that treatment with KCNQ1-SupRep gene therapy can suppress and replace LQT1-causative variants in a mutation-independent manner, TSA201 cells were co-transfected with the three KCNQ1 variants and shKCNQ1, KCNQ1-SupRep, or shCT control. All three LQT1-causative variants were suppressed by shKCNQ1, ranging from 87% to 93% KD relative to KCNQ1-WT as measured by allele-specific qRT-PCR (FIG. 12, top). While the suppression was visibly marked for each of the three variants, suppression by shKCNQ1 did not reach statistical significance for KCNQ1-Y171X and KCNQ1-I567S, presumably due to lower baseline expression of these variants. Despite not reaching statistical significance, it is noteworthy that very few mRNA transcripts were detectable in any sample (WT or variant) that was treated with shKCNQ1. Notably, KCNQ1-Y171X had substantially decreased expression at baseline, likely due to its premature stop codon and predicted subsequent nonsense-mediated decay of mRNA transcripts (Hug et al., Nucleic Acids Res., 44:1483-1495 (2016)).

Results obtained by qRT-PCR were confirmed by western blotting. KCNQ1-Y171X produced no detectable protein as a result of its premature truncation, while KCNQ1-V254M was suppressed by shKCNQ1, and KCNQ1-I567S had faint baseline expression that also was suppressed by shKCNQ1 (FIG. 12, bottom). Overall, KCNQ1-SupRep caused suppression and replacement of three LQT1-causative KCNQ1 variants, validating its ability to suppress and replace KCNQ1 in a mutation-independent manner.

### Example 7—Generation of iPSC-CMs from Four Patients with LQT1

From the 236 patients with LQT1 in the iPSC biorepository, four patients with distinct LQT1 mutations were selected to have their iPSCs differentiated into iPSC-CMs, in order to test the APD-shortening potential of this KCNQ1-SupRep gene therapy. A healthy unrelated individual was included as a control, and two isogenic controls were created by CRISPR-Cas9 correction of KCNQ1-V254M and KCNQ1-I567S, respectively. These isogenic controls served as the gold standard for a possible therapeutic cure, thereby providing a marker for the “ideal” rescue/normalization of the prolonged APD and indicating how close to this ideal did treatment with KCNQ1-SupRep gene therapy reach.

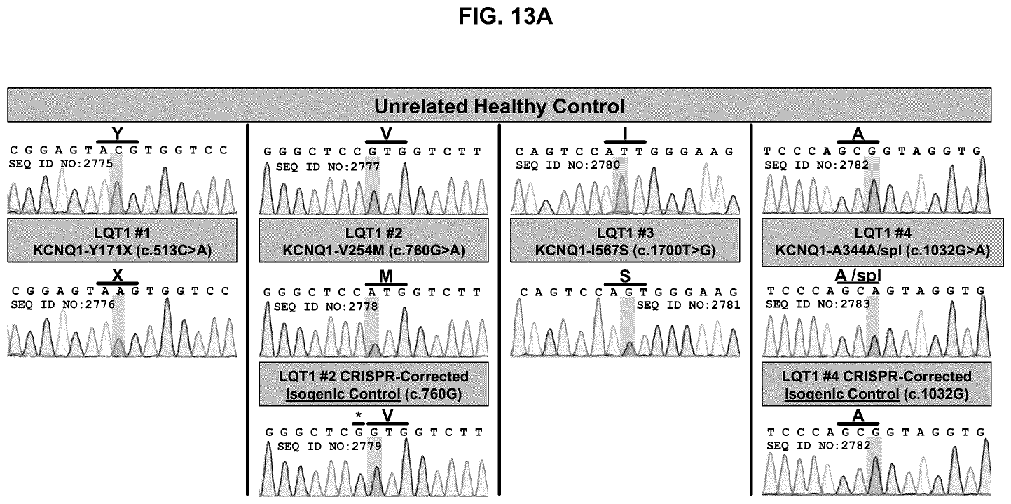

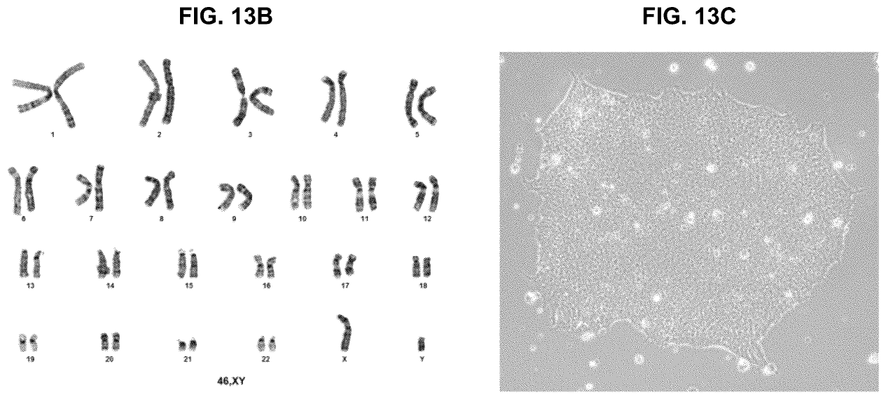

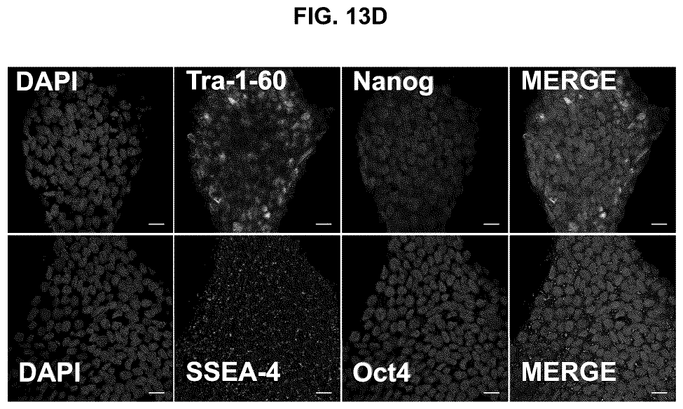

Dermal fibroblasts or peripheral blood mononuclear cells (PBMCs) were collected from each patient and were used to generate iPSCs. Standard quality control assays were performed on each iPSC line, including Sanger sequencing of the LQT1-causative variant, karyotyping, bright field morphology, and immunofluorescence microscopy for pluripotent markers including Tra-1-60, Nanog, SSEA-4, and Oct4 (FIGS. 13A-13D). Differentiation of iPSCs was induced by methods described elsewhere to generate spontaneously beating iPSC-CMs (Burridge et al., Nat. Methods, 11:855-860 (2014); and Mummery et al., Circ. Res., 111:344-358 (2012)). Since the cardiac APD is known to shorten as iPSC-CMs mature over time, all experiments were conducted at least 30 days after the induction of differentiation (Shaheen et al., Stem Cell Reports, 10:1879-1894 (2018)).

### Example 8—KCNQ1-SupRep Gene Therapy Increases KCNQ1 in LQT1 iPSC-CMs

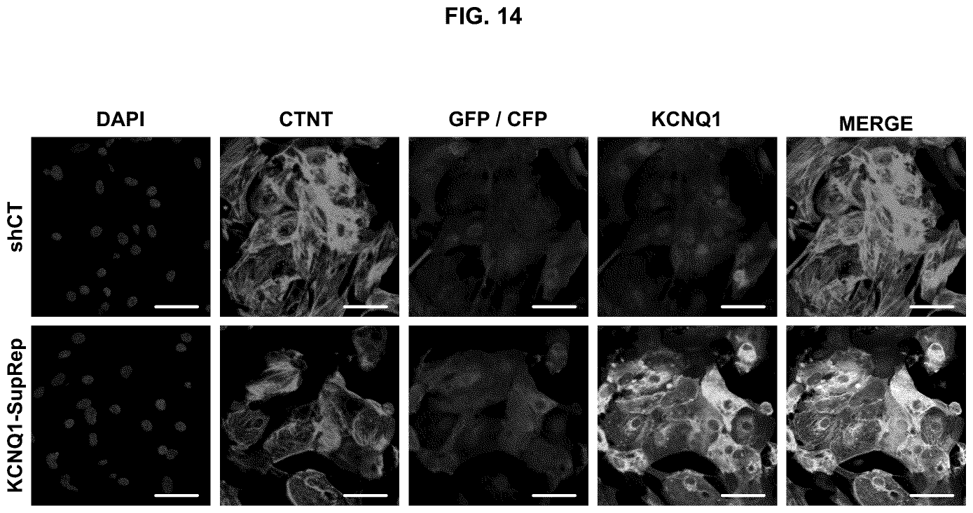

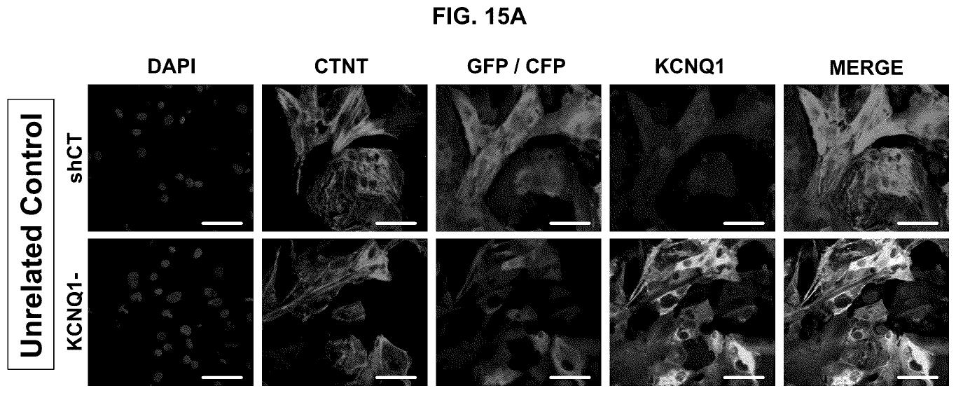

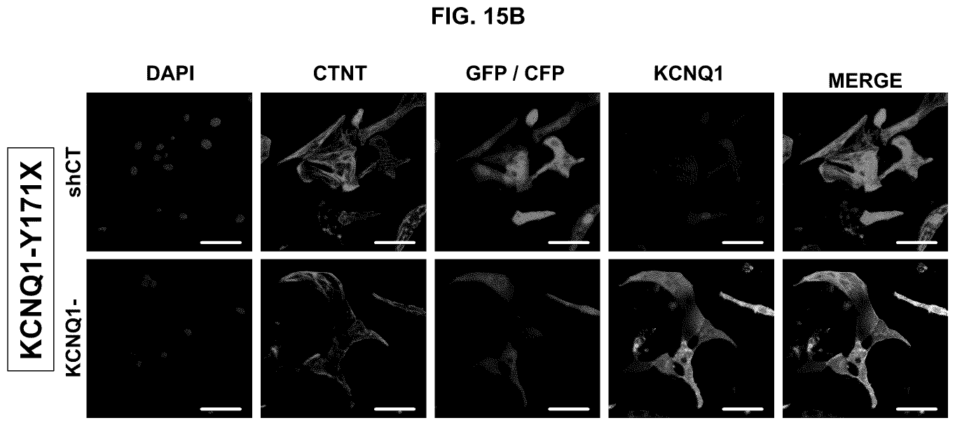

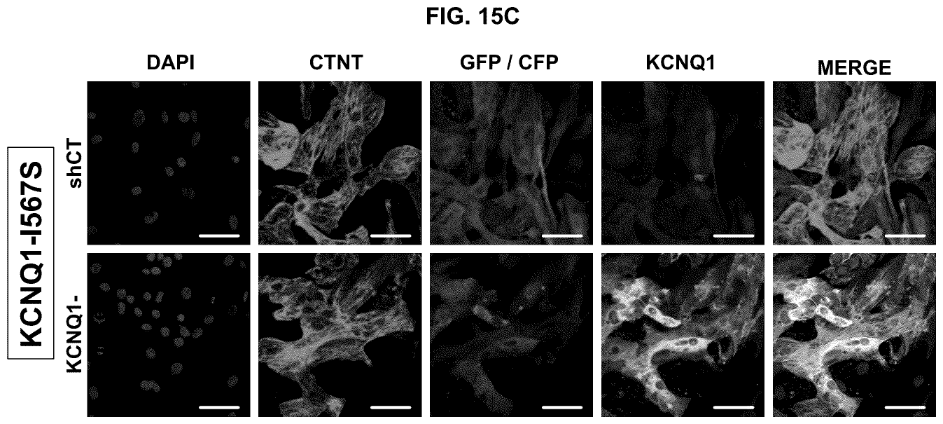

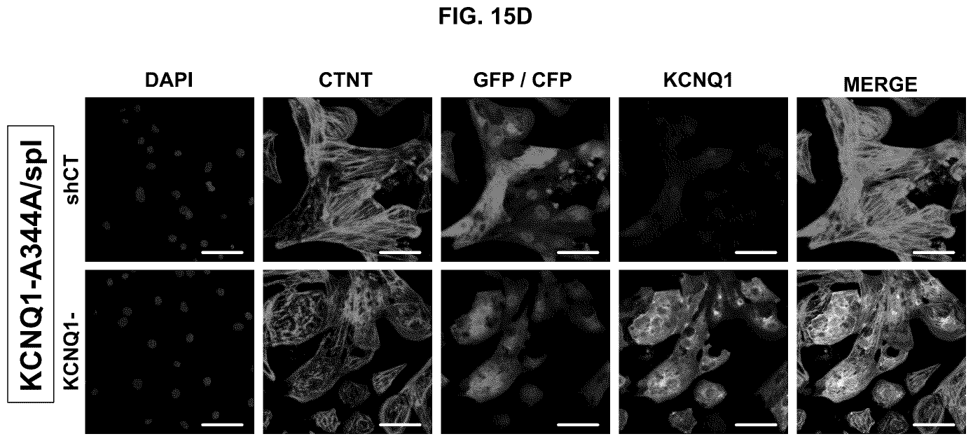

To assess the ability of lentiviral KCNQ1-SupRep to transduce iPSC-CMs and increase WT KCNQ1 expression, unrelated control and LQT1 iPSC-CMs were transduced with lentiviral KCNQ1-SupRep or shCT and evaluated using immunofluorescence microscopy. Cardiac troponin T (cTnT) was used as a marker of cardiomyocytes. Antibodies targeting the lentiviral reporters (turboGFP for shCT or CFP for KCNQ1-SupRep) were used to identify transduced cells, and KCNQ1 was stained to visualize the effects of KCNQ1-SupRep on overall expression of KCNQ1. Results for KCNQ1-V254M iPSC-CMs (FIG. 14) and remaining unrelated control and LQT1 iPSC-CMs (FIGS. 15A-15D) showed high purity cardiomyocytes within the iPSC-CM cultures that had been evenly transduced with lentiviral KCNQ1-SupRep or shCT. At baseline in iPSC-CMs treated with shCT, KCNQ1 was only faintly detectable by confocal microscopy, whereas iPSC-CMs treated with KCNQ1-SupRep displayed robust staining for KCNQ1 (FIGS. 14 and 15A-15D). This suggests that in iPSC-CMs, treatment with KCNQ1-SupRep gene therapy drives substantial overexpression of KCNQ1-shIMM.

### Example 9—KCNQ1-SupRep Gene Therapy Shortens the Cardiac APD in LQT1 iPSC-CMs as Measured by FluoVolt′ Voltage Dye

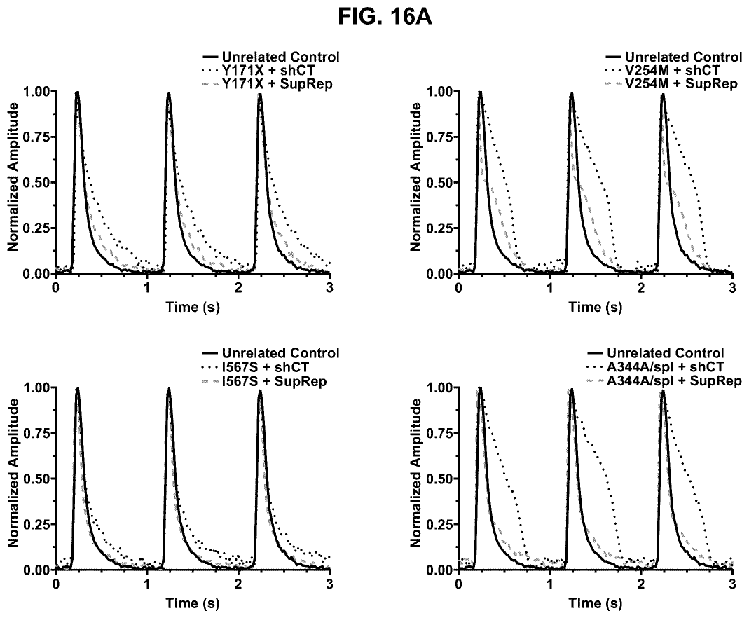

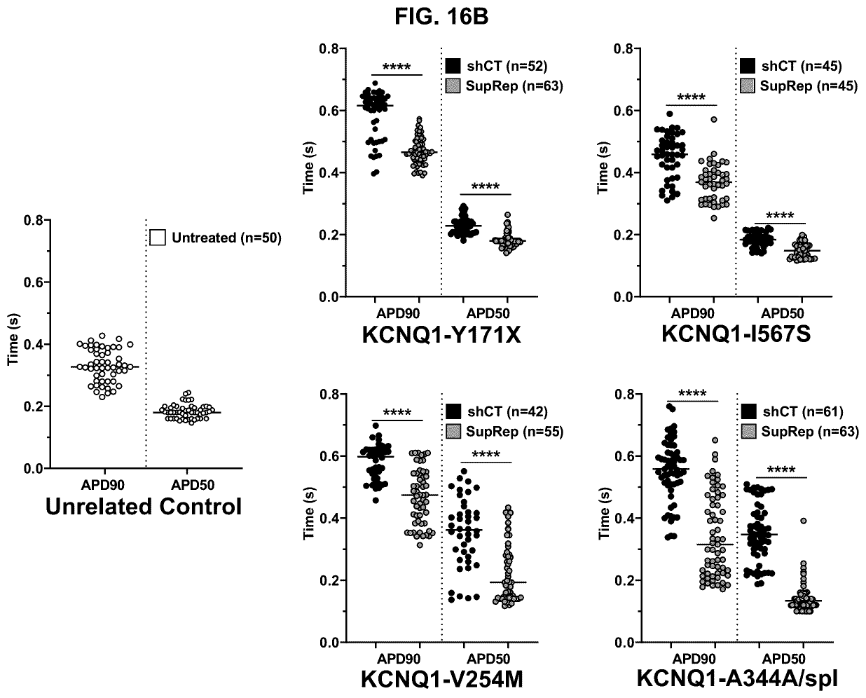

Further studies were conducted to test whether treatment with KCNQ1-SupRep gene therapy is able to rescue the pathognomonic feature of LQT1 by shortening the pathologically prolonged APD. FluoVolt™ voltage dye was used to measure optical action potentials in iPSC-CMs derived from four patients with LQT1 (stemming from KCNQ1-Y171X, -V254M, 4567S, or -A344A/spl) treated with either the lentiviral shCT control or KCNQ1-SupRep gene therapy. The unrelated control was measured without any treatment as a measure for a healthy APD. All iPSC-CMs were paced at 1 Hz during recording to eliminate beat rate-dependent changes to the APD. Representative optical action potentials are shown in FIG. 16A. When treated with shCT, all LQT1 iPSC-CMs had significantly longer APD at 90% repolarization (APD₉₀) and three of the four also had significantly longer APD at 50% repolarization (APD₅₀) compared to untreated unrelated healthy control iPSC-CMs, validating the LQT1 iPSC-CMs as an in vitro model of LQT1.

A full summary of APD₉₀ and APD₅₀ values and APD shortening due to KCNQ1-SupRep is shown in TABLE 5. APD₉₀ and APD₅₀ values were assessed by one-way ANOVA with post-hoc Dunnett's test comparing each KCNQ1 variant treated with shCT or KCNQ1-SupRep to the untreated, unrelated control (brackets in TABLE 5). All four LQT1 iPSC-CMs treated with shCT had significantly longer APD₉₀ than the unrelated control, and two of the three had significantly longer APD₅₀ as well, confirming that these LQT1 lines display prolonged APD—the hallmark feature of LQT1. APD shortening due to KCNQ1-SupRep compared to treatment with shCT was then assessed by unpaired two-tailed student's t-tests at both the APD₉₀ and APD₅₀ levels separately for each variant. KCNQ1-SupRep resulted in statistically significant attenuation of both APD₉₀ and APD₅₀ in all four LQT1 iPSC-CMs (TABLE 5 and FIG. 16B). When treated with KCNQ1-SupRep, the APD₉₀ and APD₅₀ of both LQT1 lines shortened significantly. In particular, the APD₉₀ shortened by 117 ms in KCNQ1-Y171X, by 111 ms in KCNQ1-V254M, by 85 ms in KCNQ1-I567S, and by 210 ms in KCNQ1-A344A/spl (TABLE 5 and FIG. 16B).

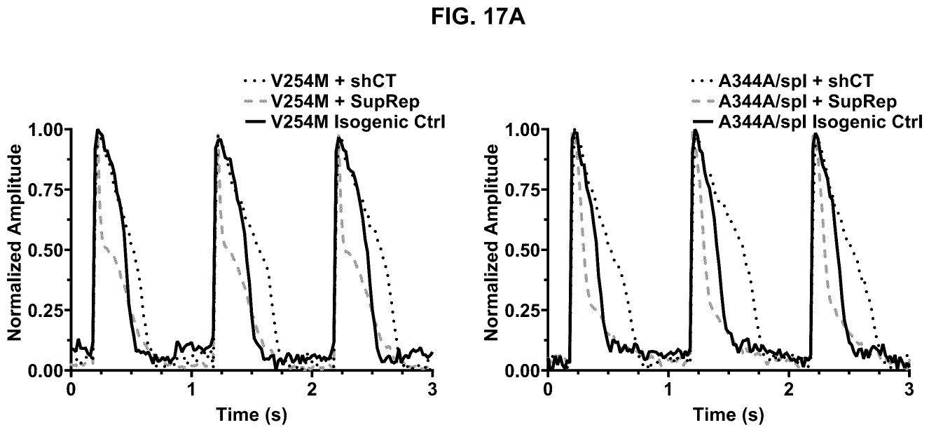

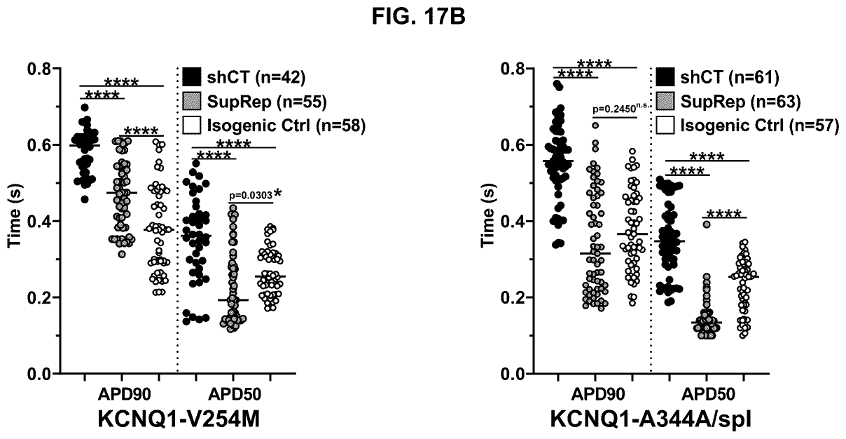

To determine whether the observed APD shortening due to KCNQ1-SupRep represents complete rescue to WT or if the shorter APD values were incomplete or overcorrection, two CRISPR-Cas9 corrected isogenic controls were created from the KCNQ1-V254M and KCNQ1-A344A/spl parent LQT1 iPSC cell lines. When measured by FluoVolt™, and plotted against the shCT and KCNQ1-SupRep treatment data from FIG. 16B, both isogenic controls had significantly shorter APD₉₀ and APD₅₀ compared to their shCT-treated counterparts (FIGS. 17A and 17B).

Isogenic correction of KCNQ1-V254M shortened the APD₉₀ by 200 ms to 380±112 ms (n=58, p<0.0001), and isogenic correction of KCNQ1-A344A/spl shortened the APD₉₀ by 176 ms (n=57, p<0.001). A full summary of the APD₉₀ and APD₅₀ values for KCNQ1-V254M and KCNQ1-A344A/spl with isogenic controls is shown in TABLE 6. Comparing the shortened APD values of the KCNQ1-V254M and KCNQ1-A344A/spl iPSC-CMs treated with KCNQ1-SupRep gene therapy to the APD values of the isogenic controls, there was apparent variability in the actual degree of rescue. In KCNQ1-V254M, there was statistically significant incomplete shortening of the APD₉₀ and concomitant overcorrection of the APD₅₀ while in KCNQ1-A344A/spl the APD₉₀ had complete rescue with no significant difference, but did show overcorrection of the APD₅₀. Despite this variability, treatment with KCNQ1-SupRep gene therapy demonstrated the ability to completely rescue the prolonged action potential in LQT1 iPSC-CMs.

### Example 10—KCNQ1-SupRep Gene Therapy Shortens the Cardiac APD in 3D-Organoid Culture of LQT1 iPSC-CMs

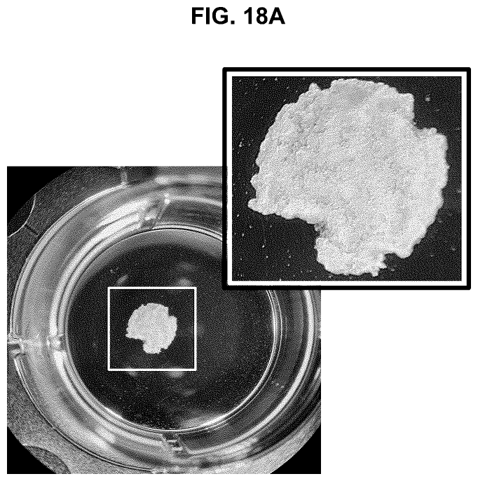

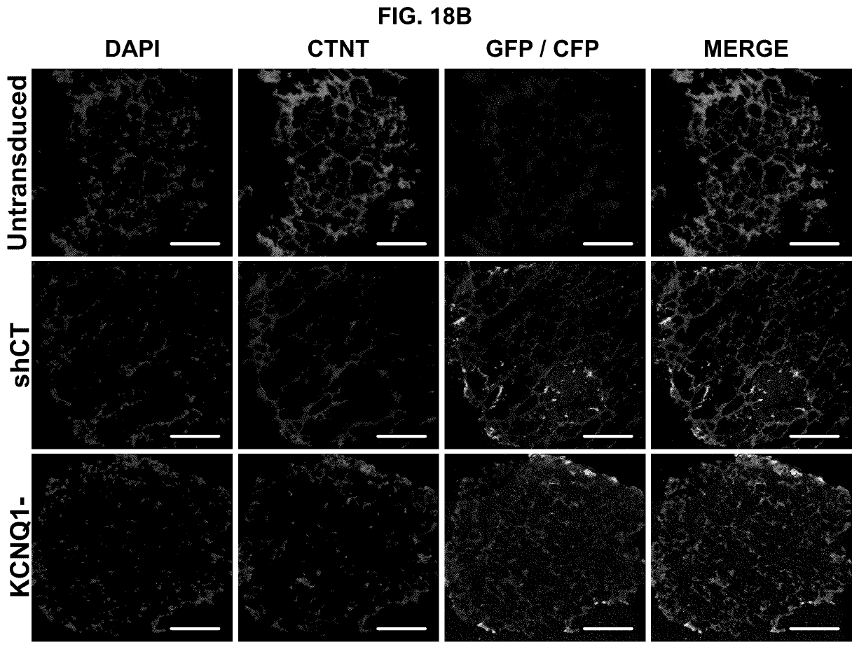

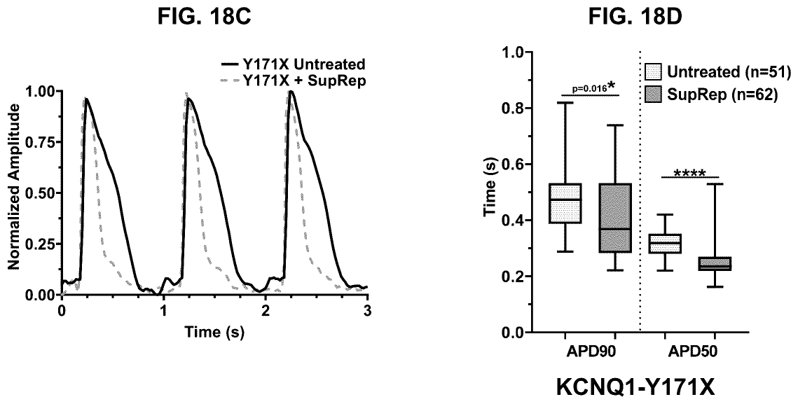

To determine whether the APD-shortening ability of KCNQ1-SupRep is translatable from 2D syncytial monolayer iPSC-CM culture to a three-dimensional environment, LQT1 iPSC-CM 3D-organoids were generated from one of the four LQT1 variants using the KCNQ1-Y171X iPSC-CMs. The KCNQ1-Y171X iPSC-CMs were dissociated and embedded in a MATRIGEL® spheroid mold and allowed to reorganize naturally on the collagenous extracellular architecture to create a 3D-cardiac organoid (FIG. 18A). The organoids were treated with shCT or KCNQ1-SupRep, cryosectioned, and stained for immunofluorescence using cardiac troponin T (cTnT) to mark cardiomyocytes and the lentiviral reporters (turboGFP for shCT and CFP for KCNQ1-SupRep) to mark infected cells. Immunofluorescence revealed networks of cardiomyocytes and prominent staining of turboGFP and CFP, indicating even transduction by shCT and KCNQ1-SupRep (FIG. 18B). The APD of untreated and KCNQ1-SupRep treated organoids were assessed by FluoVolt′, revealing that KCNQ1-SupRep resulted in statistically significant shortening of the APD₉₀ and APD₅₀ (FIGS. 18C and 18D), and suggesting that KCNQ1-SupRep retained APD-shortening ability in a simple 3D organoid environment.

Taken together, the studies described above used two in vitro model systems to engineer and validate the APD-attenuating effect of a hybrid suppression-and-replacement gene therapy construct for LQTS, and LQT1 in particular. The results of these studies indicated that suppression-replacement gene therapy can be used to directly target the pathogenic substrate and ameliorating the resultant disease not only for LQT1 specifically, but also for LQTS in general, and perhaps for almost any sudden death-predisposing autosomal dominant genetic heart disease.

TABLE 2
Summary of subjects selected for generation of iPSCs for iPSC-CM studies

| | | Age at | | | | | | iPSC Source [symbol] |
|---|---|---|---|---|---|---|---|---|
| | | Sample | KCNQ1 | Average QT [symbol] | LQTS-Related | | | Generation |
| Subject | Sex | Collection | Variant(s) | (ms) [Range] | Symptoms | Family History | Treatment | Method |
| LQT1 #1 | Female | 41 | Y171X | No ECG | Asymptomatic | Daughter-JLNS | BB | PBMC: |
| | | | (c.513C > A) | Available | | | | Episomal |
| | | | | | | | | DNA |
| LQT1 #2 | Female | 28 | V254M | 512 [486- [symbol] ] | Near drowning | Mother-near | BB, ICD | Fibroblasts: |
| | | | (c.760G > A) | | (×2) | drowning | | Sendai |
| LQT1 #3 | Female | 59 | [symbol] | 488 [465-512] | Cardiogenic | Sister-syncope | BB, LCSD, | Fibroblasts [symbol] |
| | | | (c.1700T > G) | | syncope, ICD | while swimming (×2) | ICD | Episomal |
| | | | | | storm | Father-sudden | | DNA |
| | | | | | | death (80-years old) | | |
| LQT1 #4 | Male | 12 | A344A/spl | [symbol] [444-604] | Potential | Great-great aunt- | BB, LCSD | Fibroblasts: |
| | | | (c.1032G > A) | | cardiogenic | sudden death (30- | | Sendai |
| | | | | | syncope (×2) | years-old) | | |
| Unrelated | Małe | 47 | — | No ECG | Asymptomatic | — | — | Fibroblasts [symbol] |
| Control | | | | Available | | | | Sendai |

KCNQ1 variants are listed as the resulting change on the protein level with cDNA change in parenthesis.
(QT [symbol] ) Bazett-corrected QT interval;
(ECG) electrocardiogram;
(JLNS) Jervell and Lange-Nielsen syndrome;
(BB) beta-blocker;
(ICD) implantable cardioverter defibrillator;
(PBMC) peripheral blood mononuclear cells. [symbol]
indicates data missing or illegible when filed

TABLE 3A
KCNQ1 shRNA sequences

| | Target sequence | Hairpin | Antisense | KCNQ1 |
|---|---|---|---|---|
| ARNA | (sense)* | Loop | sequence | Location |
| shCT | GCACTACCAGAGCTAA | TCAAGAG | AGTACTATCTGAGTT | Non- |
| | CTCAGATAGTACT | | AGCTCTGGTAGTGC | targeting |
| KCNQ1 | CACTCATTCAGACCGC | TCAAGAG | ATAGCACCTCCATGC | Exon 8-9 |
| sh# 1 (DNA) | ATGGAGGTGCTAT | | GGTCTGAATGAGTG | |
| KCNQ1 | CACUCADUCAGACCGC | UCAAGAG | AUAGCACCUCCAUG | boundary |
| shi#1 (RNA) | AUGGAGGUGCUAU | | CGGUCUGAAUGAGUG | |
| KCNQ1 | TGACTCCTOGAGAGAA | TCAAGAG | GACTGTGAGCATCTT | Exon 10 |
| sh#2 (DNA) | GATGCTCACAGTC | | CTCTCCAGGAGTCA | |
| KCNQ1 | UGACUCCUGGAGAGAA | UCAAGAG | GACUGUGAGCAUCUU | |
| sh#2 (RNA) | GAUGCUCACAGUC | | CUCUCCAGGAGUCA | |
| KCNQ1 | AGTTCTGTGAAACGCT | TCAAGAG | GTGTAACCACTGGAG | Intron 1 |
| sh#3 (DNA) | CCAGTGGTTACAC | | CGTTICACAGAACT | |
| KCNQ1 | ACGGCTATGACAGTTC | UCAAGAG | GUGUAACCACUGGAG | |
| sh#3 (RNA) | TGTAAGGAAGAGC | | CGUUUCACAGAACU | |
| KCNQ1 | ACGGCTATGACAGTTC | TCAAGAG | GCTCTTCCTTACAGAA | Exon |
| sh#4 (DNA) | TGTAAGGAAGAGC | | CTGTCATAGCCGT | 10-11 |
| KCNQ1 | ACGGCUAUGACAGUUC | UCAAGAG | GCUCUUCCUUACAGA | boundary |
| sh#4 (RNA) | UGUAAGGAAGAGC | | ACUGUCAUAGCCGU | |

*shCT = SEQ ID NO: 11
KCNQ1 sh#1 (DNA) = SEQ ID NO: 12; KCNQ1 sh#1 (RNA) = SEQ ID NO: 16
KCNQ1 sh#2 (DNA) = SEQ ID NO: 13: KCNQ1 sh#2 (RNA) = SEQ ID NO: 17
KCNQ1 sb#3 (DNA) = SEQ ID NO: 14; KCNQ1 sh#3 (RNA) = SEQ ID NO: 18
KCNQ1 sh#4 (DNA) = SEQ ID NO: 15; KCNQ1 sh#4 (RNA) = SEQ ID NO: 19

TABLE 3B
KCNQ1 shRNA sequences

| | Target sequence | Hairpin | Antisense | KCNQ1 |
|---|---|---|---|---|
| shRNA | (sense)* | Loop | sequence | Location |
| KCNQ1 | GTTCAAGCTGGACAA | TCAAGAG | TCACCCCATTGTCTT | Exon 10 |
| Sh#5 (DNA) | AGACAATGGGGTGA | | TGTCCAGCTTGAAC | |
| KCNQ1 | GUUCAAGCUGGACAA | UCAAGAG | UCACCCCAUUGUCUU | |
| sh#5 (RNA) | AGACAAUGGGGUGA | | UGUCCAGCUUGAAC | |
| KCNQ1 | GACAGTTCTGTAAGG | TCAAGAG | AGTGTTGGGCTCTTC | Exon |
| sh#6 (DNA) | AAGAGCCCAACACT | | CTTACAGAACTGTC | 10-11 |
| KCNQ1 | GACAGUUCUGUAAGG | UCAAGAG | AGUGUUGGGCUCUUC | |
| sh#6 (RNA) | AAGAGCCCAACACU | | CUUACAGAACUGUC | |
| KCNQ1 | AGACCATCGCCTCCT | TCAAGAG | AAAGACAGAGAACCA | Exon 7 |
| sh#7 (DNA) | GCTTCTCTGTCTTT | | GGAGGCGATGGTCT | |
| KCNQ1 | AGACCAUCGCCUCCU | UCAAGAG | AAAGACAGAGAAGCA | |
| sh#7 (RNA) | GCGUCUCUGUCUUU | | GGAGGCGAUGGUCG | |
| KCNQ1 | CCCAAACCCAAGAAG | TCAAGAG | TTTACCACCACAGAC | Exon |
| sh# 8 (DNA) | TCTGTGGTGGTAAA | | TTCTTGGGTTTGGG | 9-10 |
| KCNQ1 | CGCAAACCCAAGAAG | UCAAGAG | UUUACCACCACAGAC | |
| sh#8 (RNA) | UCUGUGGUGGUAAA | | UUCUUGGGUUUGGG | |

*KCNQ1 sh#5 (DNA) = SEQ ID NO: 36, KCNQ1 sh#5 (RNA) = SEQ ID NO: 40
KCNQ1 sh#6 (DNA) = SEQ ID NO: 37; KCNQ1 sh#6 (RNA) = SEQ ID NO: 41
KCNQ1 sh#7 (DNA) = SEQ ID NO: 38: KCNQ1 sh#7 (RNA) = SEQ ID NO: 42
KCNQ1 sh#8 (DNA) = SEQ ID NO: 39; KCNQ1 sh#8 (RNA) = SEQ ID NO: 43

TABLE 4
qRT-PCR primers

| | | Forward | Reverse | |
|---|---|---|---|---|
| Primer | | Primer | Primer | Location |
| Set | Amplifies: | (5′→3′) | (5′→3′) | (FW, RV) |
| Total | KCNQ1-ALL | GAGCCACAC | GGAGAGAAGAT | Exon 9, |
| | | TCTGCTGTC | GCTCACAGTC | Exon 10 |
| | | (SEQ ID | (SEQ ID | |
| | | NO: 20) | NO: 21) | |
| Allele- | KCNQ1-WT | GACGGCTAT | TGTGAGATGTG | Exon 10, |
| Specific | KCNQ1- | GACAGTTCT | GGTGATGGGTG | Exon 11 |
| Endo- | variants | GTAAGGAAG | TCAGCAGA | |
| genous | | AGC | (SEQ ID | |
| | | (SEQ ID | NO: 23) | |
| | | NO: 22) | | |
| Allele- | KCNQ1- | GATGGATAC | TGTGAGATGTG | Exon 10, |
| Specific | shIMM | GATAGCTCC | GGTGATGGGTG | Exon 11 |
| shIMM | | GTCAGAAAA | TCAGCAGA | |
| | | AGT | (SEQ ID | |
| | | (SEQ ID | NO: 23) | |
| | | NO: 24) | | |
| GAPDH | GAPDH- | ACATCGCTC | TGTAGTTGAGG | Exon 2, |
| | ALL | AGACACCAT | TCAATGAAGGG | Exon 3 |
| | | G | (SEQ ID | |
| | | (SBQ ID | NO: 26) | |
| | | NO: 25) | | |

[symbol]

TABLE 5
Summary of FIGS. 16A and 16B FluoVolt ™ optical action potential data

| | | | | p-value | | | | p-value |
|---|---|---|---|---|---|---|---|---|
| | shCT | SupRep | [symbol] PD₉₀ | (SupRep | shCT | SupRep | [symbol] APD [symbol] | (SupRep |
| iPSC-CMS | ADP₉₀ (ms) | APD₉₀ (ms) | (ms) | v. shCT) | APD [symbol] (ms) | APD [symbol] (ms) | (ms) | v. shCT) |
| Unrelated | [Untreated] | — | — | — | (Untreated] | — | — | — |
| Control | 332 ± 53 (n = 50) | | | | 184 ± 23 (n = [symbol] ) | | | |
| KCNQ1-Y171X | 585 ± 77 (n = 52) | 468 ± 43 (n = 63) | −117 | p < 0.0001**** | 230 ± 26 (n = [symbol] ) | 181 ± 23 (n = 63) | −49 | p < 0.0001**** |
| | p < 0.0001**** | p < 0.0001**** | | | p = 0.0015** | p = 0.9997 [symbol] | | |
| KCNQ1-V254M | 580 ± [symbol] (n = 42) | 469 ± 89 (n = 55) | −111 | p < 0.0001**** | 353 ± 112 (n = 42) | 224 ± 96 (n = 55) | −129 | p < 0.0001**** |
| | p < 0.0001**** | p < 0.0001**** | | | p < 0.0001**** | p = 0.0073** | | |
| KCNQ1-I567S | 452 ± 72 (n = 45) | 367 ± 60 (n = 45) | −85 | p <0 .0001**** | 184 ± 24 (n = 45) | 149 ± 24 (n = 45) | −35 | p < 0.0001**** |
| | p < 0.0001**** | p = [symbol] | | | p > 0.9999 [symbol] | p < 0.0424* | | |
| KCNQ1- | 553 ± [symbol] | 343 ± 133 | −230 | p < 0.0001**** | 350 ± 94 (n = 61) | 142 ± 47 (n = 63) | −208 | p < 0.0001**** |
| A344A/spl | (n = 61) | (n = 63) | | | p < 0.0001**** | p < 0.0033** | | |
| | p < 0.0001**** | p < 0.9757 [symbol] | | | | | | |

APD₉₀ and APD [symbol] values were assessed by one-way ANOVA with post-hoc Dunnett's test to compare each KCNQ1 variant treated with shCT or KCNQ1-SupRep to the untreated, unrelated control (all p-values except those listed in the SupRep v. shCT columns).
All four LQT1 iPSC-CMs treated with shCT had significantly longer APD₉₀ than the unrelated control, and three of the four had significantly longer APD [symbol] as well.
APD shortening due to KCNQ1-SupRep compared to treatment with shCT was assessed by unpaired two-tailed student's [symbol] -tests at both the APD₉₀ and APD [symbol] levels separately for each variant.
KCNQ1-SupRep resulted in statistically significant attenuation of both APD₉₀ and APD [symbol] in all four LQT1 iPSC-CMs.
*p < 0.05, **p < 0.01, ***p < 0.001, ****p < 0.0001, (n.s.) not significant. [symbol]
indicates data missing or illegible when filed

[symbol]

TABLE 6
Summary of FIGS. 17A and 178 FluoVolt ™ optical action potential data

| | | | | p-value | | | | p-value |
|---|---|---|---|---|---|---|---|---|
| | shCT | SupRep | AAPD [symbol] | (SupRep | shCT | SupRep | AAPD [symbol] | (SupRep |
| IPSC-CMS | ADP [symbol] (ms) | APD [symbol] (ms) | (ms) | v. shCT) | APD [symbol] (ms) | APD [symbol] (ms) | (ms) | v. shCT) |
| KCNQ1-V254M | [symbol] ± 56 (n = 42) | 469 ± 89 (n = 55) | −111 | p < 0.0001**** | 353 ± 12 | 224 ± 96 | −129 | p < 0.0001**** |
| | p < 0.0001**** | p < 0.0001**** | | | (n = 42) | (n = 55) | | |
| | | | | | p < 0.0001**** | p = 0.0303* | | |
| isogenic Control | (Untreated) | — | — | — | (Untreated) | — | — | — |
| for V254M | 380 ± 112 | | | | 267 ± 60 | | | |
| | (n = 58) | | | | (n = [symbol] ) | | | |
| KONQ1- | [symbol] ± 98 (0 = 61) | 343 ± 133 | −210 | p < 0.0001**** | 350 ± 94 | 142 ± 47 | −208 | p < 0.0001**** |
| A344A/spl | p < 0.0001**** | (n = 63) | | | (n = 61) | (n = 63) | | |
| | | p = 0.2450 [symbol] | | | p < 0.0001*** | p < 0.0001**** | | |
| isogenic Control | (Untreated) | — | — | — | (Untreated) | — | — | — |
| for A344A/spl | 377 ± 105 | | | | 231 ± 68 | | | |
| | (n = 57) | | | | (n = 57) | | | |

APD [symbol] and APD [symbol] values for KCNQ1-V254M and KCNQ1-A344A/spl were compared to their respective isogenie controls by one-way ANOVA with post-hoc Tukey's test (all p-values except those listed in the SupRep v. shCT columns).
The APD values for the isogenic controls served as a benchmark for the “ideal” rescue of APD for each of the two variants, KCNQ1-V254M and KCNQ1-A344A/spl.
Treatment of the LQT1 iPSC-CMs with KCNQ1-SupRep resulted in shortening of the APD for each set of LQT1 iPSC-CMs tested, bringing the APD closer to the respective isogenic control for each variant.
*p < 0.05, **p < 0.01, ***p < 0.001, ****p < 0.0001, (n.s.) not significant. [symbol]
indicates data missing or illegible when filed

### Example 11—Restoring Normal Cellular Electrophysiology in a Transgenic LQT1 Rabbit Model

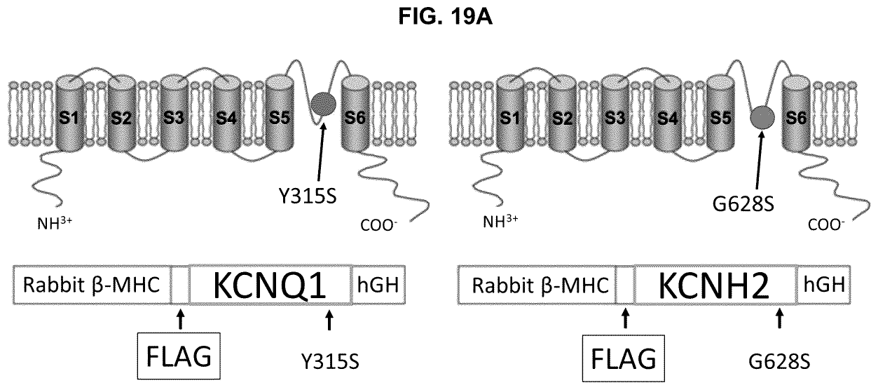

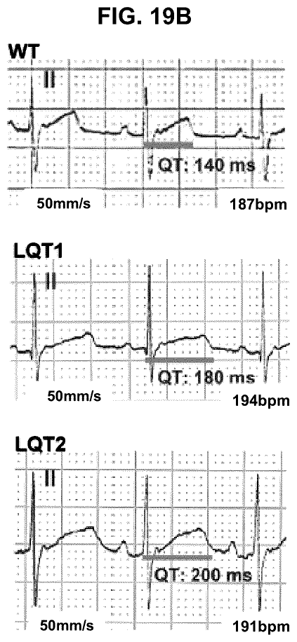

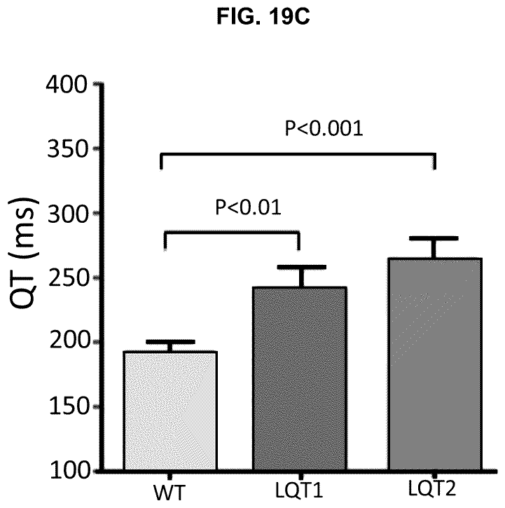

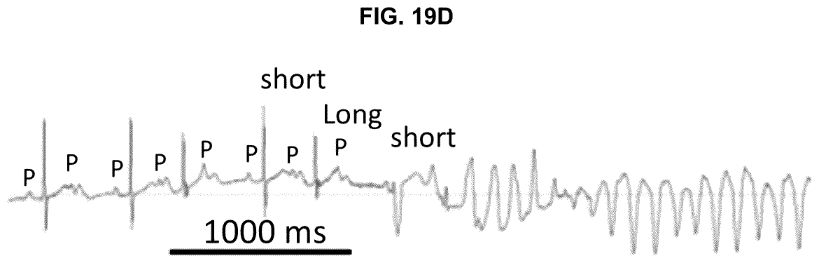

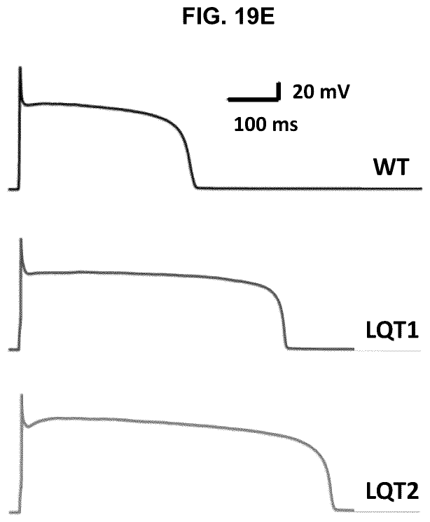

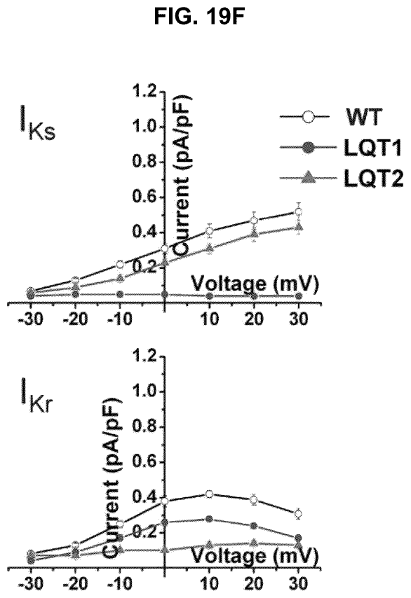

Experiments are conducted to evaluate the effect of AAV9-based gene delivery of KCNQ1-SupRep gene therapy to reverse QT/APD-prolongation and arrhythmia susceptibility in an established humanized rabbit model of LQT1 with an LQT1-causing human pathogenic KCNQ1-p.Y315S variant. Animals are treated with AAV9-KCNQ1-SupRep for whole animal arrhythmia phenotyping and molecular/cellular electrophysiological phenotyping in acutely isolated rabbit ventricular CMs, to determine the effects of AAV9-mediated delivery of the KCNQ1-SupRep vector on restoring normal molecular, cellular, whole heart, and whole animal electrophysiological phenotypes and preventing ventricular arrhythmias. Rabbits and humans share similar K+ currents underlying cardiac repolarization (Nerbonne, J. Physiol., 525(2):285-298 (2000)), such that transgenic rabbit models are useful for investigating human arrhythmogenic diseases with impaired repolarization. The transgenic LQT1 and LQT2 rabbit models for use in these studies selectively over-express either loss-of-function, dominant-negative pore-localizing variants of human KCNQ1 (LQT1, KCNQ1-Y315S, loss of I_{Ks}) or KCNH2 (LQT2, KCNH2-G628S, loss of I_{Kr}) in the heart, respectively. These LQT1 and LQT2 rabbits mimic the human LQTS phenotype with QT-prolongation, spontaneous Torsade-de-Pointes (TdP) ventricular tachycardia, and SCD (FIGS. 19A-19F) (Brunner et al., J. Clin. Invest., 118:2246-2259 (2008); and Odening et al., Heart Rhythm, 9:823-832 (2012)). The KCNQ1-Y315S and KCNH2-G628S mutations are expressed in the rabbit hearts under control of the rabbit beta-myosin heavy chain (β-MyHC) promoter (FIG. 19A) to produce LQT1 and LQT2 phenotypes in the rabbit models, respectively. The rabbits exhibit significant prolongation of QT (FIGS. 19B and 19C), a propensity to develop spontaneous torsades de pointes (TdP) following treatment with ostradiol (FIG. 19D), and action potential duration (FIG. 19E) due to elimination of I_{Ks} or I_{Kr} currents, respectively (FIG. 19F). Detailed methods for generation and phenotypic assessment of the rabbits are described elsewhere (Brunner et al., supra). Given the similarity to the human LQTS phenotype, these models have unique advantages for investigating novel LQTS therapies in vivo and on the whole heart level.

To focus on treatment of the LQT1 transgenic rabbit model, the QTc/APD-attenuating effects of AAV9-KCNQ1-SupRep are investigated in detail in vivo, ex vivo (whole-heart), and in vitro (rabbit cardiomyocyte) in the LQT1 rabbits. The anti-arrhythmic properties of AAV9-KCNQ1-SupRep are assessed ex vivo in Langendorff-perfused LQT1 rabbit hearts in which arrhythmias are facilitated by AV-node ablation and hypokalemia, to evaluate the ability of KCNQ1SupRep gene therapy delivery to reverse the pathogenic LQT1 phenotype in KCNQ1-Y315S transgenic rabbits. Following protocols described elsewhere (Odening et al., Eur Heart J., 40:842-853 (2019)), all experiments are performed in female (f) and male (m) adult rabbits (aged 4-7 months). For in vivo experiments (surgery, surface ECG), rabbits are anesthetized with S-ketamine and xylazine (12.5 mg/kg/3.5 mg/kg IM, followed by IV infusion). After surgery, analgetic therapy with buprenorphine is maintained for 3 days. Beating heart excision (for action potential recordings and arrhythmia assessments in Langendorff-perfused hearts, and cellular patch clamping) are performed after additional injection of heparin (500 IE IV) and thiopental-sodium (40 mg/kg IV). In vivo cardiac phenotyping is performed using surface ECG35 (Odening et al. 2019, supra) on KCNQ1-Y315S transgenic rabbits after AAV9 delivery of KCNQ1-SupRep or AAV9-sham vectors. Similarly, molecular and cellular electrophysiological characterization of AAV9-KCNQ1-SupRep and AAV9-shCT treated rabbits is performed as described elsewhere (Brunner et al., supra; and Odening et al. 2019, supra).

The transgenic LQT1 rabbit expresses two endogenous wild-type rabbit KCNQ1 alleles and a single transgenic human KCNQ1 mutant (p.Y315S) allele. The human and rabbit KCNQ1 cDNA are 73% homologous overall. shRNAs having 100% homology between rabbit and human KCNQ1 (such that both rabbit and human alleles are suppressed simultaneously in the LQT1 rabbit model) are designed and tested, and virus particles are produced.

Analogous experiments are carried out using one or more KCNH2-SupRep constructs in a LQT2 rabbit model.

AAV9-KCNQ1-SupRep gene transfer in isolated LQT1 CMs: The functionality of the AAV9-KCNQ1-SupRep gene transfer is tested in isolated ventricular CMs from LQT1 rabbits before the constructs are tested in LQT1 rabbits in vivo. In particular, left ventricular CMs are obtained from the hearts of transgenic LQT1 rabbits (n=5) by standard collagenase digestion (Brunner et al., supra; and Odening et al. 2019, supra). CMs are maintained in culture for 48 hours, and half of the cell cultures are incubated with AAV9-KCNQ1-SupRep. Functional consequences on cellular APD and I_{Ks} current densities are then analyzed (compared to sham-treated LQT1 CMs) using standard voltage and current mode patch clamping (see below).

AAV9-KCNQ1-SupRep gene transfer in vivo via lateral thoracotomy: For in vivo gene transfer, lateral thoracotomy is performed and AAV9-KCNQ1-SupRep or AAV9-shCT constructs are painted on the epicardial surface of both ventricles and both atria.

Adult LQT1 rabbits of both sexes (LQT1-KCNQ1-SupRep and LQT1-AAV9-shCT controls, split into groups and used for in vivo and ex vivo whole heart experiments or cellular electrophysiology) are anesthetized with S-ketamine and xylazine. Rabbits are intubated to guarantee proper ventilation during open chest surgery, and left lateral thoracotomy is performed. After thorough painting of AAV9-KCNQ1-SupRep or AAV9-shCT on the surface of the whole heart, the chest is closed and the rabbit is awakened. After at least 1-2 weeks of post-surgery recovery, experiments are performed to investigate the electrophysiological consequences of the KCNQ1-SupRep gene therapy in LQT1 rabbits.

12-lead ECG recording in vivo: Adult LQT1-KCNQ1-SupRep (female and male) and LQT1-AAV9-shCT sham-controls (female and male) rabbits are subjected to conventional 12-lead surface ECG recordings to determine the effect of KCNQ1-SupRep gene therapy on restoring normal QT duration and diminishing pro-arrhythmic markers. ECG is performed under general anesthesia with S-ketamine and xylazine, as this anesthetic regimen does not impact cardiac repolarization (Odening et al., Am. J. Physiol. Heart Circ. Physiol., 295:H2264-2272 (2008)). KCNQ1 gene-transfer mediated changes in QT, heart rate corrected QT, and Tpeak-Tend (Tp-e) and beat-to-beat variability of QT (short term variability of the QT interval; STVQT) are calculated to assess changes in spatial and temporal heterogeneity of repolarization.

Monophasic Action Potential (MAP) measurements in Langendorff-perfused hearts ex vivo: MAP is performed as described elsewhere (Odening et al. 2019, supra). Briefly, adult LQT1-KCNQ1-SupRep (female and male) and LQT1-AAV9-shCT sham-control (female and male) rabbits are anesthetized as described above. Following euthanasia with thiopental-sodium (40 mg/kg) IV, hearts are excised rapidly, mounted on a Langendorff-perfusion set-up (IH5, Hugo Sachs Electronic-Harvard Apparatus), retrogradely perfused via the cannulated aorta ascendens with warm (37° C.), pre-oxygenated (95% 02 and 5% CO₂), modified Krebs-Henseleit solution at the constant flow rate of 50 mL/minute. Action potential duration at 90%, 75%, and 30% of repolarization (APD90, 75, 30) is assessed, and AP triangulation (APD90-APD30) and APD restitution (based on APD90 values at 2 and 4 Hz stimulation) are calculated for each LV region.

Arrhythmia experiments in Langendorff-perfused hearts ex vivo: The anti-arrhythmic effect of KCNQ1-SupRep gene therapy is assessed ex vivo in AV-node-ablated Langendorff perfused LQT1-KCNQ1-SupRep (female and male) and LQT1-AAV9-shCT (female and male) hearts, beating spontaneously with stable ventricular escape rhythm (VER) at a constant rate of around 60-80 beats/minute (Hornyik et al., Br. J. Pharmacol., 177:3744-3759 (2020)). After 10 minutes of baseline (arrhythmia-free) recording, hearts are perfused with 2 mM low K⁺ containing KH solution (10 minutes) to provoke arrhythmias. In a second step, 10 μM of IK1-blocker BaCl₂ are added to the 2 mM low K⁺ containing KH solution and perfused (10 minutes) to reduce repolarization reserve and further increase susceptibility to arrhythmia formation. ECGs are recorded continuously and the duration (%) and incidence (average number of events) of arrhythmias are measured off-line. Arrhythmias are defined as ventricular extra beats (VEB), bigeminy, ventricular tachycardia (VT), and ventricular fibrillation (VF). Arrhythmia rates are very high (in the range of 60-80%) in LQT1 hearts, while even in low K⁺ KH combined with BaCl₂, no serious ventricular arrhythmias occur in normal wild type hearts (Hornyik et al., supra).

Electrophysiological recording in rabbit CMs: Left ventricular CMs are obtained from the hearts of KCNQ1SupRep-treated transgenic LQT1 rabbits and sham control transgenic LQT1 rabbits by standard collagenase digestion (Brunner et al., supra; and Odening et al. 2019, supra). Whole cell currents (I_{Ks}, I_{Kr}, Ito, and IK1) and action potentials are recorded using Axopatch 200B patch clamp amplifier (Molecular Devices), digitized at a sampling frequency of 10 kHz with Digidata 1440A interface and acquired with pCLAMP software as described elsewhere (Odening et al., 2019, supra).

Data interpretation: For normally distributed values, Student's t test (unpaired) is used to compare the means of 2 groups, and Mann-Whitney and Wilcoxon matched pairs test are used for values not normally distributed. Fisher's exact test is used for categorical variables such as arrhythmia incidences. In I-V-curves, differences are assessed using repeated-measure ANOVA, complemented by Bonferroni post-hoc analyses. Cellular electrophysiology data are evaluated using pClamp 9.0 and Origin 7.0 software, and results are given as mean±SEM. All other analyses are performed with Prism 5.01 for Windows (Graph-Pad), and their data are presented as mean±SD, with n indicating the number of experiments/animals, tests being 2-tailed, and p<0.05 considered significant. All experiments in the rabbits are performed and analyzed in a blinded fashion.

### Example 12—Materials and Methods for LQT2 SupRep

Cloning of KCNH2-SupRep: WT KCNH2 cDNA (NM_000238.3) was subcloned into pIRES2-EGFP (Clontech; Mountain View, CA) to generate pIRES2-EGFP-KCNH2-WT. The p.G604S and p.N633S variants in pIRES2-EGFP-KCNH2-WT were produced by GenScript (Piscataway, NJ). DNA Sanger sequencing was used to confirm vector integrity. Five custom-designed KCNH2 shRNAs (sh#1-5) were ordered from OriGene (Rockville, MD) in the pGFP-C-shLenti backbone along with a non-targeting scrambled shRNA control (shCT). For the final KCNH2-SupRep gene therapy vector, KCNH2 sh#4 was selected as the lead candidate and is referred to as shKCNH2. A DNA fragment containing ten synonymous variants within the KCNH2 sh#4 (shKCNQ2) target sequence of the KCNH2-WT cDNA: c.2694C>T, c.2697G>C, c.2700G>A, c.2703G>A, c.2706A>T, c.2709G>C, c.2712G>A, c.2715G>C, c.2718G>C, and c.2721C>G (KCNH2: p.D898D, p.T899T, p.E900E, p.Q901Q, p.P902P, p.G903G, p.E904E, p.V905V, p.59065, and p.A907A, respectively) was synthesized and cloned into pIRES2-EGFP-KCNH2-WT to create KCNH2-shIMM (pIRES2-EGFP-KCNH2-shIMM) (GenScript; Piscataway, NJ). KCNH2-shIMM was subcloned into the pGFP-C-shLenti backbone containing shKCNH2 to create the final KCNH2-SupRep.

KCNH2 mammalian expression vectors for patch clamp experiments: Wild-type KCNH2 cDNA was subcloned into pIRES2-EGFP (Clontech, Mountain View, CA) and AAV-P2A CTnC-EGFP (GenScript; Piscataway, NJ) to produce KCNH2-pIRES2-EGFP and KCNH2-AAV-P2A CTnC-EGFP.

TSA 201 and H9C2 cell culture and transfection for patch clamp experiments: TSA 201 and H9C2 cells were cultured in Dulbecco's Modification of Eagle's Medium (DMEM) supplemented with 10% Fetal Bovine Serum (FBS), 1.0% L-glutamine, and 1.2% penicillin/streptomycin solution in a 5% CO₂ incubator at 37° C. Heterologous expression of KCNH2 was accomplished by using 5 μl or 3 μl of Lipofectamine (Invitrogen) to transfect 1.0 μg of pIRES2-KCNH2-EGFP along with 1.0 μg KCNE2-pIRES2-dsRed2 or 1.0 μg KCNH2-AAV-P2A CTnC-EGFP in OPTI-MEM media. The transfected cells were incubated for 48 hours before electrophysiological experiments.

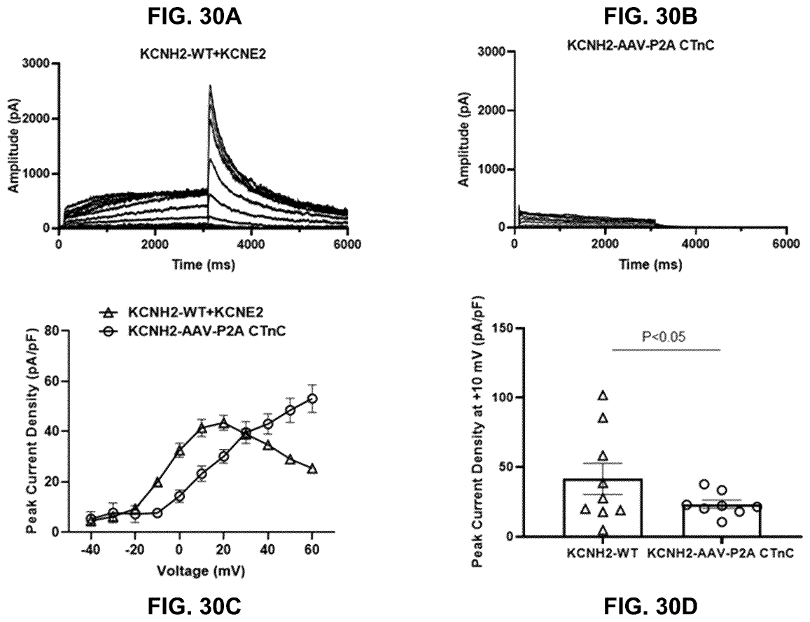

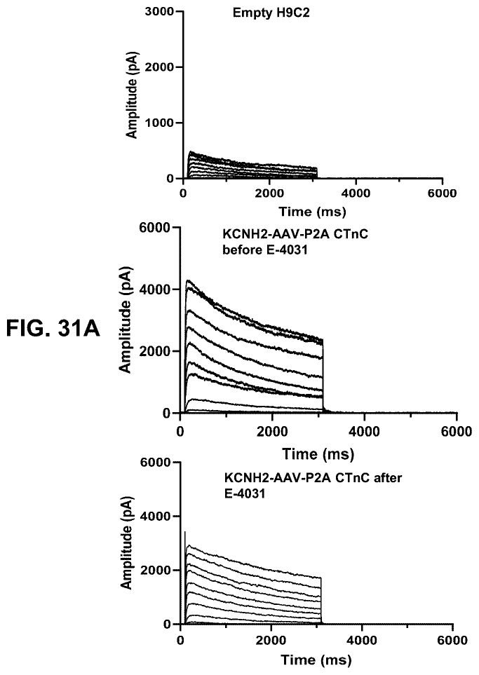

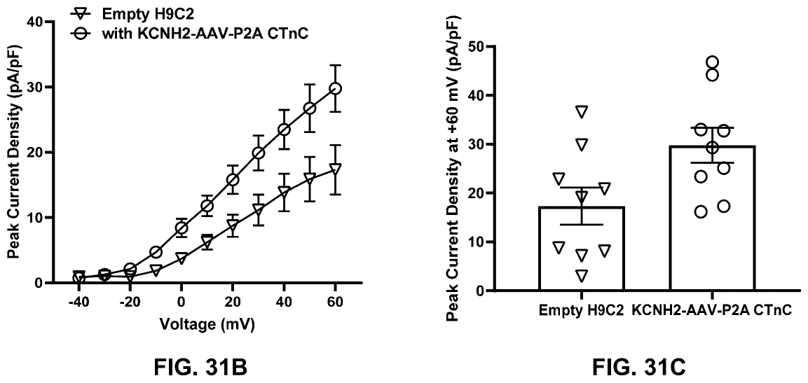

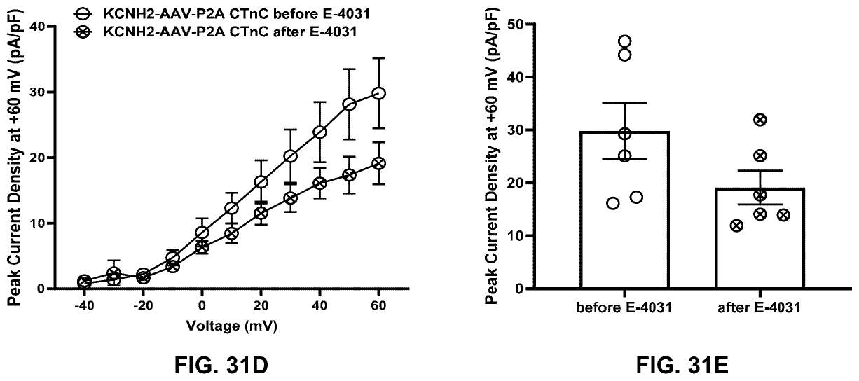

Electrophysiological measurements: A standard whole-cell patch clamp technique was used to measure pIRES2-KCNH2-WT-EGFP with KCNE2-pIRES2-dsRed2 and KCNH2-AAV-P2A CTnC-EGFP currents at room temperature (RT) using an Axopatch 200B amplifier, Digidata 1440A, and pclamp version 10.4 software (Axon Instruments, Sunnyvale, CA). The extracellular (bath) solution contained (mmol/L): 150 NaCl, 5.4 KCl, 1.8 CaCl₂, 1 MgCl₂, 1 Na-Pyruvate, and 15 HEPES. The pH was adjusted to 7.4 with NaOH. The intracellular (pipette) solution contained (mmol/L): 150 KCl, 5 NaCl, 2 CaCl₂, 5 EGTA, 5 MgATP, 10 HEPES, pH adjusted to 7.2 with KOH. Microelectrodes were fire polished to a final resistance of 2-3 MS2 after being pulled using a P-97 puller (Sutter Instruments, Novato, CA). Series resistance was compensated by 80-85%. Currents were filtered at 1 kHz and digitized at 5 kHz with an eight-pole Bessel filter. The voltage dependence of activation was determined using voltage-clamp protocols described for FIGS. 30A and 31A. Data were analyzed using Clampfit (Axon Instruments, Sunnyvale, CA), Excel (Microsoft, Redmond, WA) and graphed with GraphPad Prism 8.3 (GraphPad Software, San Diego, CA).

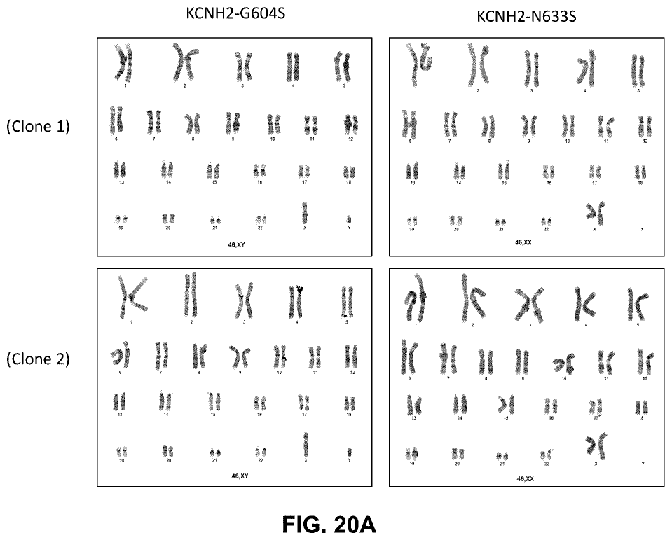

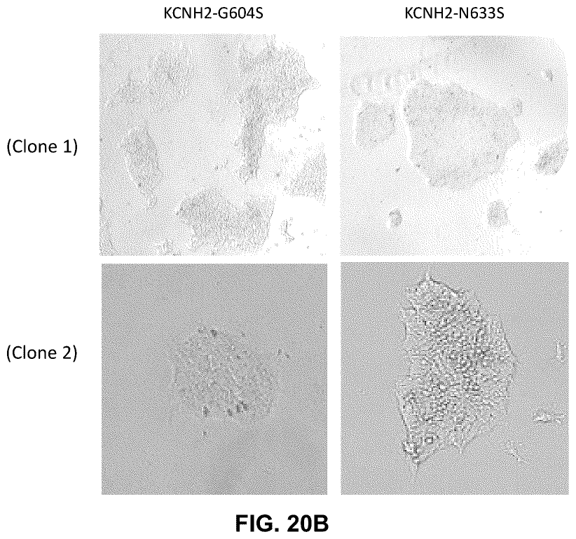

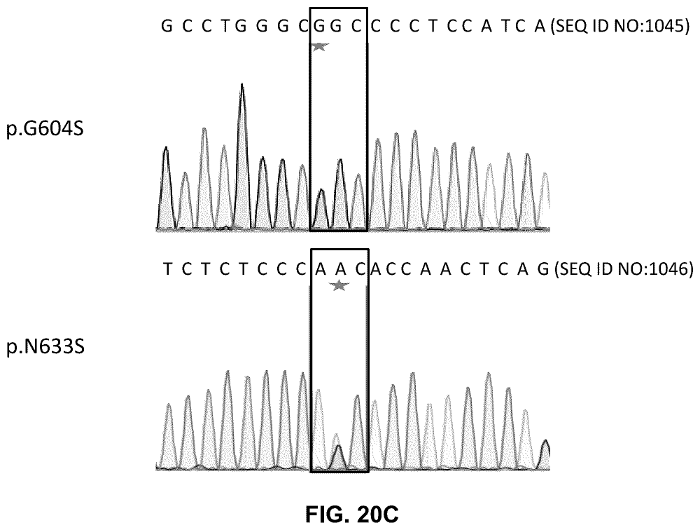

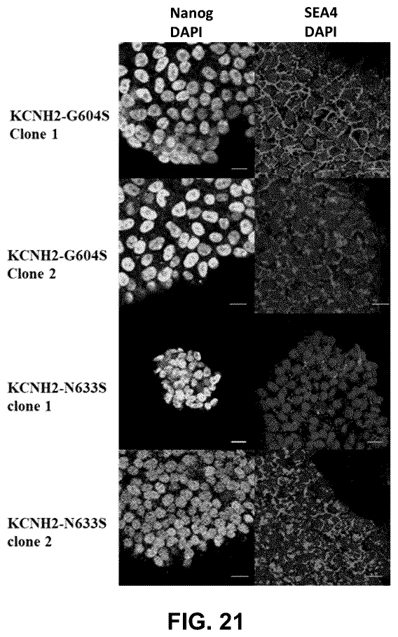

LQT2 Patient Selection for iPSC Generation: All patients were evaluated by a single genetic cardiologist and LQTS specialist. Dermal fibroblasts and peripheral blood mononuclear cells (PBMCs) were collected by 4 mm skin punch biopsy and blood sample, respectively. Samples were obtained from 212 patients with LQT2. For this study, two LQT2 patients (13-year-old male and 12-year-old female) with two different LQT2-causative missense variants were selected based on a strong LQT2 phenotype defined as at least one ECG with QTc greater than 500 ms, positive history of LQTS-related symptoms (syncope, seizure, sudden cardiac arrest), and positive family history of LQTS-related symptoms (TABLE 7). PBMCs or fibroblasts were reprogrammed into induced pluripotent stem cells (iPSCs) by Sendai virus transduction using the Cytotune 2.0 reprogramming kit. Colonies were picked within 21 days post infection with Yamanaka factors. For each variant line, two representative clones were generated, characterized, and analyzed for quality control as described elsewhere (O'Hare et al., Circ Genom Precis Med. 13:466-475 (2020)). Karyotyping for each of the patient-specific iPSC clones was completed by the Mayo Clinic Cytogenetics Laboratory, and all mutant iPSC clones that were tested demonstrated normal karyotypes (FIG. 20A). Genomic DNA was isolated from each iPSC clone (FIG. 20B) and the variant's presence and integrity of the rest of the KCNH2 sequence was confirmed by Sanger sequencing (FIG. 20C). All iPSC clones were confirmed to express Nanog (ThermoFisher PA1-097X) and SSEA-4 (ThermoFisher, MA1-021) pluripotent markers (FIG. 21). All iPSCs were cultured in mTeSR-Plus medium (STEMCELL®) supplemented with 1% antibiotic/antimycotic solution on MATRIGEL®-coated (Corning; Corning, NY) 6 cm culture dishes in a 5% CO₂ incubator at 37° C. At 85% confluence, iPSCs were passaged using ReLeSR (STEMCELL®).

iPSC-CM Culture, Differentiation, and Dissociation: iPSCs were differentiated into cardiomyocytes (CMs) after reaching −85% confluency, using a protocol described elsewhere (Burridge et al., supra; and Mummery et al., supra). On day 0, differentiation was initiated by changing the culture medium from mTeSR-Plus to RPMI 1640 GlutaMAX plus 25 mM HEPES supplemented with B27-minus insulin (RPMI/B27-ins; Thermo) containing 5 μM CHIR99021 (MilliporeSigma; St. Louis, MO). After 48 hours (day 2), the medium was changed to RPMI/B27-ins containing 5 μM IWP-2 (MilliporeSigma). On day 4, the medium was changed back to the RPMI/B27-ins maintenance medium. Spontaneous beating began on days 6-7. From days 10-16, iPSC-CMs were cultured in selection medium containing 500 μg/ml of recombinant human albumin, 217 μg/ml of L-ascorbic acid 2-phosphate, and 5 mM of DL-Lactate in RPMI 1640 medium (without glucose). Post selection, iPSC-CMs were dissociated enzymatically using a STEMdiff cardiomyocyte dissociation kit (STEMCELL) as described elsewhere (Dotzler et al., Circulation. 143:1411-1425 (2021)). After 24 hours, cells were maintained in RPMI/B27-ins medium. For all experiments, cells were used after at least 30 days post differentiation.

Generation of CRISPR-Cas9 Corrected Isogenic Control iPSCs: Genome editing of iPSC cell lines was contracted through Applied Stem Cell (Milpitas, CA). Using CRISPR-Cas9 technology, isogenic “variant corrected” control iPSC cell lines were created for both LQT2 patient cell lines (p.G604S and p.N633S). Briefly, two guide RNAs (gRNAs) for each variant line were designed and validated in vivo. Based on specificity score, cutting efficiency, and off-target profile, one candidate gRNA was chosen for genome editing on each patient iPSC line. A single-stranded oligodeoxynucleotide (ssODN) was designed to be used as a repair template, and a silent mutation in the gRNA binding site was introduced into the ssODN to prevent re-cutting. The LQT2 patient iPSC line was transfected with the gRNA construct and ssODN using a Neon system, and transfected iPSCs were subjected to puromycin selection. Single-cell colonies were picked for genotyping, and two clones with variant correction were expanded for further studies.

TSA201 Cell Culture and Transfection for Western Blot and qRT-PCR: TSA201 cells were maintained at 37° C. using Dulbecco's Modified Eagle Medium (Corning) supplemented with 10% fetal bovine serum, 1% L-glutamine, and 1% penicillin/streptomycin in a 5% CO₂ incubator. For allele-specific qRT-PCR and western blot experiments, 5×10⁵ cells were plated per well in 6-well plates. After 24 hours, cells were co-transfected in maintenance medium using 10 μL Effectene (Qiagen; Hilden, Germany) with 100 fmol (0.3-0.7 μg) equimolar amounts of each plasmid (pIRES2-EGFP-KCNH2-WT or -variant, pGFP-C-shLenti-shKCNH2(#1-#5) or -shCT, KCNH2-shIMM, or pGFP-C-shLenti-KCNH2-SupRep).

Western Blotting: TSA201 cells were co-transfected with KCNH2-WT, -shIMM, or -variants and shKCNH2(#1-5), -shCT, or KCNH2-SupRep as described above. After 48 hours, cells were lysed using 1×RIPA buffer with protease and phosphatase inhibitors. Lysates were chilled on ice for 10 minutes and then sonicated for 10 seconds at 50% amplitude, and the cell debris was pelleted at 21,000 rcf for 15 minutes at 4° C. The supernatant was transferred to a new tube and the protein concentration was measured using the Pierce BCA Protein Assay Kit (ThermoFisher) before mixing 1:1 with loading buffer (2× Laemmli buffer with 1:20 β-mercaptoethanol). Proteins (10 μg/lane) were run on a 4-15% TGX gel (Bio-Rad; Hercules, CA) and transferred to a PVDF membrane using a Trans-Blot Turbo Transfer System (Bio-Rad). After blocking for 1 hour in tris-buffered saline (TB S) with 0.1% Tween-20/3% bovine serum albumin, the membrane was incubated at 4° C. overnight with primary antibodies against KCNH2 (Alomone) and GAPDH housekeeping control (Santa Cruz, sc-376476) at 1:500 and 1:5000 dilutions, respectively, in blocking solution. The membrane was then washed in TBS-T for 3×15 minutes and incubated in secondary antibody HRP-conjugated goat-anti-rabbit (Invitrogen) at a dilution of 1:5000 in blocking solution. After 1 hour, the membrane was washed in TB S-T for 3×15 minutes. Finally, the membrane was incubated in SuperSignal™ West Pico PLUS chemiluminescent ECL substrate (ThermoFisher) and exposed to HyBlot CL autoradiography film (Denville Scientific Inc., E3012). Pixel density was quantified using freely available ImageJ software.

Allele-Specific qRT-PCR: Allele-specific primers were designed for qRT-PCR to specifically amplify total KCNH2, endogenous KCNH2 including KCNH2-WT and -variants, but excluding KCNH2-shIMM, and KCNH2-shIMM, by adapting allele-specific genotyping methods described elsewhere (Rohatgi et al., J Am Coll Cardiol. 2017, 70:453-462; and Priori et al., Heart Rhythm. 2013, 10:1932-1963). For total KCNH2, primers were purchased from IDT (Coralville, IA). For allele-specific primers, two reverse primers spanning the shKCNH2 target site with one complementary to endogenous KCNH2 (allele-specific for KCNH2-WT and -variants) and the other complementary to KCNH2-shIMM (allele-specific for KCNH2-shIMM) were used. A common forward primer was used for both allele-specific forward primers. GAPDH primers (IDT) were used as housekeeping controls. A standard curve was used to correct for PCR amplification bias. TSA201 cells were co-transfected as described above. After 48 hours, RNA was harvested using the RNeasy kit (Qiagen) and measured using the NanoDrop ND-1000 spectrophotometer (Thermo). Complementary DNA (cDNA) was generated by loading 500 ng RNA in the SuperScript IV VILO Master Mix reverse transcription kit (Thermo). Four qRT-PCR reactions were run per sample using the SYBR Green Master Mix kit (Qiagen) with the four sets of primers described above. Data were analyzed using the ΔΔCT method by first normalizing KCNH2 to GAPDH and then comparing the relative fold change to the KCNH2-WT and shCT treatment groups.

Lentivirus Generation and Transduction of iPSC-CMs: Lentivirus was used for application of KCNH2-SupRep or shCT (treatment control) to iPSC-CMs. Lentiviral particles were generated from pGFP-C-shLenti-shKCNH2-shIMM (KCNH2-SupRep) and pGFP-C-shLenti-shCT (shCT), using the pPACKH1 HIV Lentivector Packaging kit (SBI System Biosciences; Palo Alto, CA). After more than 30 days post-induction of differentiation, iPSC-CMs derived from two patients with LQT2 and their respective isogenic controls were dissociated and plated into MATRIGEL®-coated 35 mm dishes with glass-bottom insets for FluoVolt (MatTek; Ashland, MA) as described above. After 48 hours of recovery, iPSC-CMs were transduced with lentiviral particles containing KCNH2-SupRep or shCT. Polybrene (8 μg/mL) infection reagent (MilliporeSigma) was added to increase transduction efficiency and the iPSC-CMs were centrifuged at 250 rcf for 1.5 hours at room temperature in the 35 mm dishes. After 24 hours post-transduction, the medium was exchanged for fresh maintenance medium, RPMI/B27-ins.

Voltage Dye Optical Action Potentials in iPSC-CMs: Voltage dye experiments were conducted between 3-7 days post-transduction of iPSC-CMs with lentiviral particles containing either KCNH2-SupRep or shCT. On the day of imaging, iPSC-CMs were washed with pre-warmed (37° C.) HEPES-buffered Tyrode's solution (Alfa Aesar; Haverhill, MA). Each 35 mm glass-bottom dish was incubated at 37° C. for 20 minutes with 0.125 μL FluoVolt dye, 1.25 μL PowerLoad, and 0.5 mL Tyrode's solution (FluoVolt Membrane Potential kit, Thermo). Excess dye was rinsed thrice with Tyrode's solution, and a final 2 mL of Tyrode's solution was added to the iPSC-CMs for imaging. During imaging, the dishes were kept in a heated 37° C. stage-top chamber (Live Cell Instrument; Seoul, South Korea) with 5% CO₂. Under 40×-water objective magnification using a Nikon Eclipse Ti light microscope (Nikon; Tokyo, Japan), optical action potentials were recorded in 20 second fast time-lapse videos at a rate of 50 frames/second (fps, 20 ms exposure time) with LED illumination at 5% power. iPSC-CMs were paced at 1 Hz (9 ms pulse duration, 25V) using a MyoPacer field stimulator (Ion Optix; Westwood, MA) to eliminate beat-rate dependent effects on the APD. Rectangular regions of interest were drawn over flashing areas of cells for analysis. NIS-Elements software (Nikon) was used to measure the fluorescence intensity over time within each region of interest, resulting in optical action potential traces. The traces were corrected for photobleaching and the amplitude was normalized as change in fluorescence divided by the baseline minimum fluorescence (ΔF/Fₘᵢₙ) using a custom Excel program. In a semi-automated manner, common action potential parameters including APD₉₀, APD₅₀, amplitude, rise time, upstroke velocity, etc. were detected for each individual optical action potential and averaged across all beats within a 20 second trace. The average of all beats within a 20 second trace represented a single data point. For representative traces, the maximum amplitude was further normalized to 1.0 to allow for accurate visualization of APD differences.

Statistics: All statistical analysis was done using GraphPad Prism 9. Individual data points are shown where applicable along with the mean. Differences between group means of normally distributed parameters were assessed using a one-way analysis of variance (ANOVA) for comparisons among >3 groups. For multiple post-hoc ANOVA analyses, Tukey's test was used. A value of P<0.05 was considered statistically significant. For patch clamp experiments, data points are shown as the mean value and bars represent the standard error of the mean. GraphPad Prism 8.3 (GraphPad Software, San Diego, CA) was used for t-test. A Student's t-test was performed to determine statistical significance between two groups. A paired t-test was performed to determine statistical significance before and after E-4031. P<0.05 was considered to be significant.

[symbol]

TABLE 7
Summary of subjects selected for generation of iPSCs for iPSC-CM studies

| | | | | | | | | iPSC |
|---|---|---|---|---|---|---|---|---|
| | | Age at | | Average | | | | Source [symbol] |
| | | Sample | KCN [symbol] 2 | QT [symbol] (ms) | LQTS-Related | Family | | Generation |
| Subject | Sex | Collection | Variant(s) | [Range] | Symptoms | History | Treatment | Method |
| LQT2 #1 | Male | 13.1 | 0604S | [symbol] [439-581] | Aborted cardiac | — | LCSD, | PBMC [symbol] |
| | | | (c.1810G > A) | | arrest, | | Mexiletine, | Episomal |
| | | | | | cardiogenic | | Nadolol | DNA |
| | | | | | syncope | | | |
| LQT2 #2 | Female | 12.6 | N633S | [symbol] 45 [ [symbol] ] | Recurrent | Father passed | Transvenous | Fibroblasts [symbol] |
| | | | (c.1898A > G) | | syncope, Torsade | away from | ICD, LCSD, | Sendai |
| | | | | | de Pointes | SCD | Nadolol | |

KCNH2 variants are listed as the resulting change on the protein level with cDNA change in parenthesis.
QT [symbol] , Bazett-corrected QT interval;
ICD, implantable cardioverter defibrillator;
LCSD, left cardiac sympathic denervation;
PBMC, peripheral blood mononuclear cells;
SCD, sudden cardiac death. [symbol]
indicates data missing or illegible when filed

### Example 13—shRNA Knockdown of KCNH2

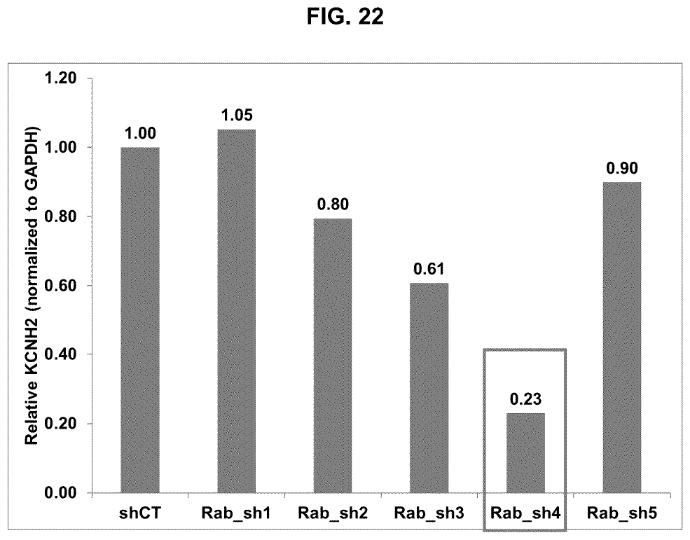

Seventeen (17) unique shRNAs targeting KCNH2 were tested, and one candidate shRNA (designated Rab_sh4) was identified that suppressed the endogenous KCNH2 alleles (both mutant and wild-type) in TSA201 cells with about 80% knockdown efficiency (FIG. 22). The shRNA (5′-CACGGAGCAGCCAGGGGAGGTGTCGGCCT-3; SEQ ID NO:27) (RNA sequence 5′-CACGGAGCAGCCAGGGGAGGUGUCGG CCU-3; SEQ ID NO:28) was completely homologous with the rabbit sequence and the human sequence. The shRNA was designed in a lentivirus backbone (pGFP-C-shLenti) and in an AAV9 backbone (pGFP-A-shAAV). Once this shRNA was identified, a SupRep construct containing the shRNA (SEQ ID NO:28) and an “shRNA-immune” (5′-TACCGAACAACCTGGCGAAGTCTCCGCGT-3; SEQ ID NO:29) version of the KCNH2 cDNA was generated (the shRNA for knocking down the endogenous KCNH2 alleles, and the shRNA-immune for simultaneously providing a replacement wild-type KCNH2 allele). As with KCNQ1, the shIM1V1 sequence had alterations at the wobble base of each codon within the shRNA target sequence, which prevented knockdown by the shRNA, but did not change the encoded amino acid sequence. The SupRep construct was designed in both a lentivirus backbone (pGFP-C-shLenti) and an AAV9 backbone (pGFP-A-shAAV), with five SupRep constructs generated in the lentivirus backbone and five in the AAV9 backbone. These constructs differed in the reporter sequences (P2A, Fusion-GFP, IRES, HA-Tag, and No reporter) that they contained. The 10 total constructs were as follows:
shLenti-SupRep-P2A
shLenti-SupRep-Fusion-GFP
shLenti-SupRep-IRES
shLenti-SupRep-HA Tag
shLenti-SupRep-No Reporter
shAAV-SupRep-P2A
shAAV-SupRep-Fusion-GFP
shAAV-SupRep-IRES
shAAV-SupRep-HA Tag

shAAV-SupRep-No Reporter

The SupRep constructs contained a CMV promoter and a human growth hormone (HGH) polyadenylation signal, but can be modified to include other promoters/enhancers. For example, the CMV promoter can be replaced with a cTnC promoter, which is smaller than the CMV promoter and more cardiac-specific. Additionally, the HGH polyadenylation signal can be replaced with a smaller SV40 terminator sequence. These modifications reduce the size of the SupRep construct and allow it to be packaged into AAV9 with greater efficiency.

### Example 14—SupRep Correction of KCNH2 In Vitro

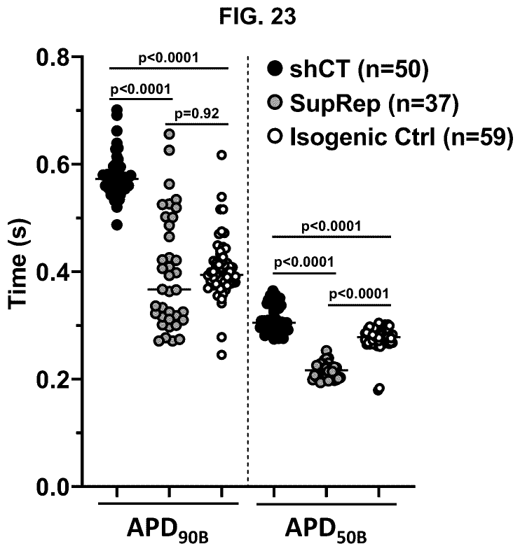

CRISPR-Cas9 corrected isogenic controls were used as a marker for “ideal” correction of the cardiac APD. FluoVolt™ voltage dye was used to measure the cardiac APD in N633S iPSC-CMs and isogenic control iPSC-CMs generated from LQT2 iPSCs (N633S). APD_{90B} and APD_{50B} values for isogenic control treated with shCT and KCNH2-N633S variant treated with shCT or KCNH2-SupRep are plotted in FIG. 23. The isogenic control iPSC-CMs had significantly shorter APD_{90B} and APD_{50B} than the LQT2 iPSC-CMs treated with shCT, indicating that correction of the single pathogenic LQT2 variant in KCNH2 was able to rescue the disease phenotype in vitro. Treatment of LQT2 iPSC-CMs with KCNH2-SupRep resulted in APD_{90B} shortening that was not significantly different from the APD_{90B} of the isogenic control treated with shCT. For KCNH2-N633S, KCNH2-SupRep achieved “ideal” correction of the prolonged APD_{90B} and overcorrected the APD_{50B}.

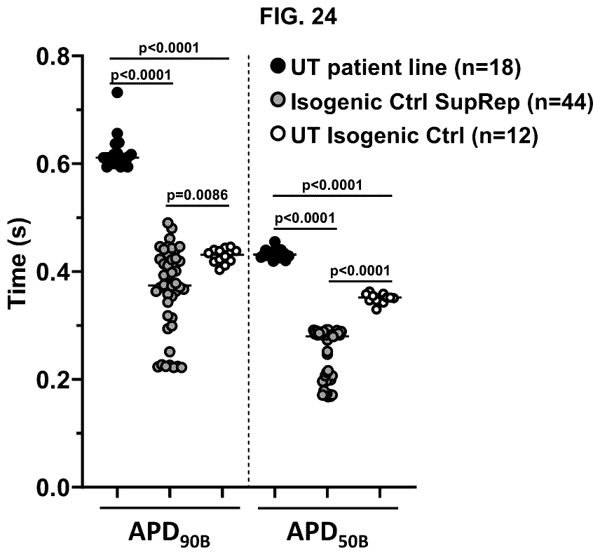

In further studies, CRISPR-Cas9 corrected isogenic controls again served as a marker for correction of cardiac APD. Results from FluoVolt™ voltage dye measurement of cardiac APD in N633S iPSC-CMs and isogenic control iPSC-CMs generated from LQT2 iPSCs (N633S) are plotted in FIG. 24. APD_{90B} and APD_{50B} values for the untreated (UT) KCNH2-N633S variant, the SupRep treated isogenic control, and the untreated (UT) isogenic control are plotted. The treated and untreated isogenic control iPSC-CMs had significantly shorter APD_{90B} and APD_{50B} than the untreated LQT2 iPSC-CMs, again indicating that correction of the single pathogenic LQT2 variant in KCNH2 was able to rescue the disease phenotype in vitro. Treatment of the isogenic control iPSC-CMs with KCNH2-SupRep resulted in overcorrection in APD_{90B} and APD_{50B} shortening, compared to the untreated isogenic control.

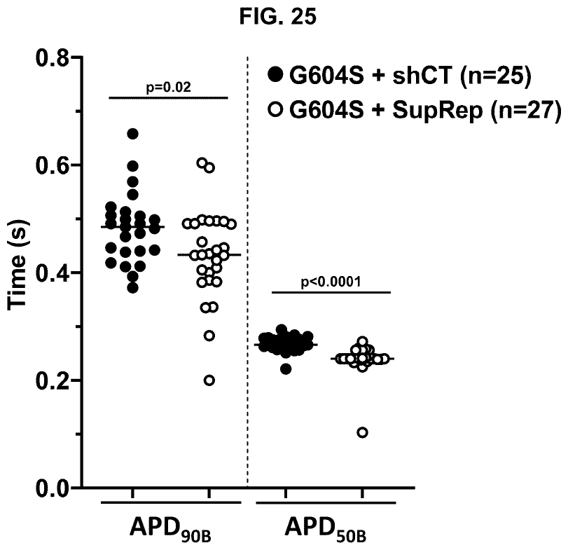

Results from FluoVolt™ voltage dye measurement of cardiac APD in G604S iPSC-CMs are plotted in FIG. 25. APD₉₀ and APD₅₀ values for KCNH2-G604S variant treated with shCT or SupRep are plotted. Treatment of LQT2 iPSC-CMs with SupRep resulted in significant APD₉₀ and APD₅₀ shortening compared to those treated with shCT.

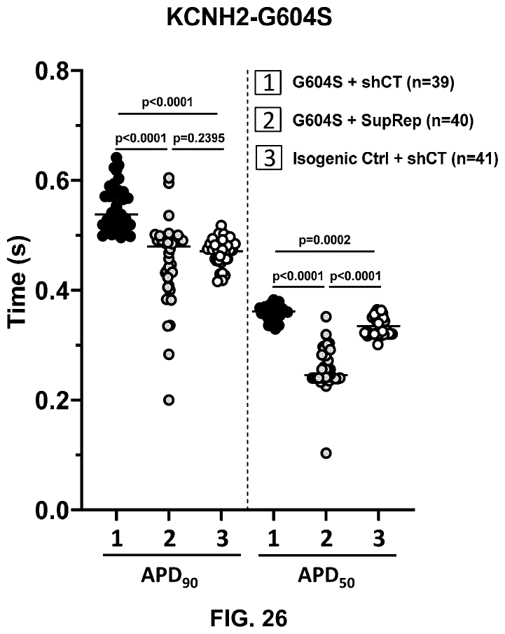

In additional studies, CRISPR-Cas9 corrected isogenic controls served as a marker for “ideal” correction of the cardiac APD. FluoVolt voltage dye measurement of the cardiac APD in G604S iPSC-CMs and isogenic control iPSC-CMs generated from LQT2 iPSCs (G604S) are shown in FIG. 26. APD₉₀ and APD₅₀ values for isogenic controls treated with shCT (3) and KCNH2-G604S variant treated with shCT (1) or KCNH2-SupRep (2) are shown. The isogenic control iPSC-CMs had significantly shorter APD₉₀ and APD₅₀ than the LQT2 iPSC-CMs treated with shCT, indicating that correction of the single pathogenic LQT2 variant in KCNH2 was able to rescue the disease phenotype in vitro. Treatment of LQT2 iPSC-CMs with KCNH2-SupRep resulted in APD₉₀ shortening. For KCNH2-G604S, KCNH2-SupRep overcorrected the prolonged APD₉₀ and APD₅₀ as compared to isogenic control treated with shCT.

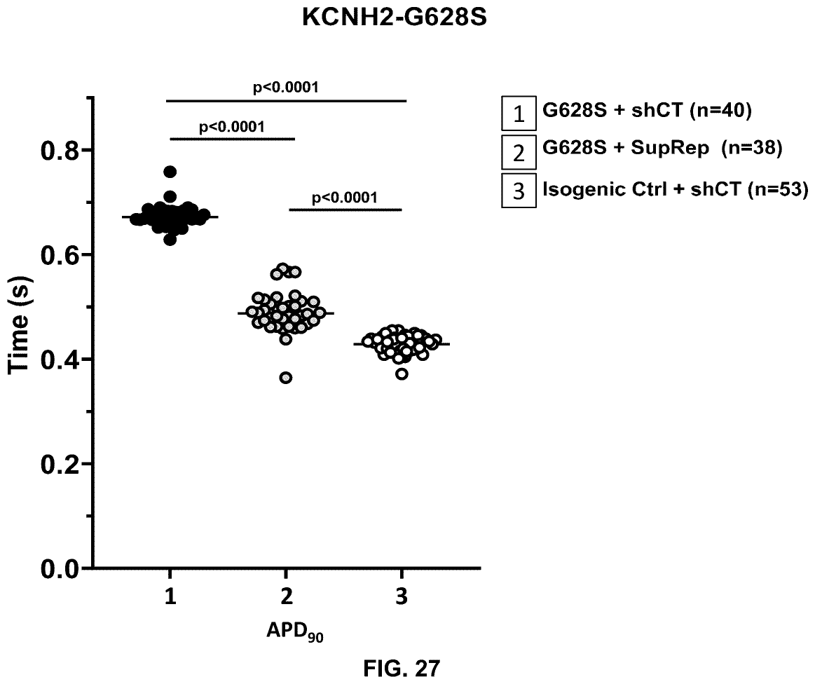

CRISPR-Cas9 also was used to insert KCNH2-G628S into wild type cells that served as isogenic controls that provided a marker for “ideal” cardiac APD. FluoVolt voltage dye measurement of the cardiac APD in G628S iPSC-CMs and isogenic control iPSC-CMs are shown in FIG. 27. APD₉₀ values for isogenic controls treated with shCT (3) and KCNH2-G628S variant treated with shCT (1) or KCNH2-SupRep (2) are shown. The isogenic control iPSC-CMs had significantly shorter APD₉₀ than the LQT2 iPSC-CMs treated with shCT, indicating that insertion of a single pathogenic LQT2 variant in KCNH2 was able to show the disease phenotype in vitro. Treatment of LQT2 iPSC-CMs with KCNH2-SupRep resulted in APD₉₀ shortening. For KCNH2-G628S, KCNH2-SupRep overcorrected the prolonged APD₉₀ as compared to isogenic control treated with shCT.

### Example 15—KCNH2-SupRep Gene Therapy Both Suppresses and Replaces KCNH2-WT

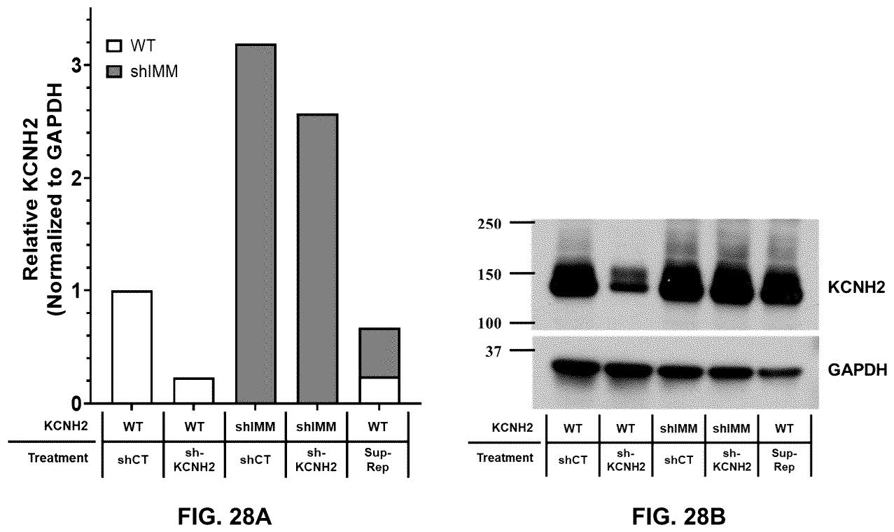

To test whether KCNH2-shIMM is indeed immune to KD by shKCNH2 (sh#4), TSA201 cells were co-transfected with KCNH2-WT or KCNH2-shIMM and shKCNH2. The expression of KCNH2-WT versus KCNH2-shIMM was quantified using allele-specific qRT-PCR. Each sample was run in four separate reactions, using a unique set of allele-specific primers, to quantify (1) total KCNH2, (2) endogenous KCNH2, which included WT and variant-containing alleles, but excluded KCNH2-shIMM, (3) KCNH2-shIMM, and (4) GAPDH as a housekeeping control. Commercial primers were used to amplify total KCNH2. For exclusive amplification of endogenous KCNH2 or KCNH2-shIMM, two reverse primers were designed within the shKCNH2 target site, one complementary to the WT sequence and the other complementary to the unique, modified sequence engineered to create KCNH2-shIMM. A common forward primer was used for both reactions, and a standard curve was used to correct for PCR amplification bias. Results showed that shKCNH2 knocked down KCNH2-WT but not KCNH2-shIMM in TSA201 cells co-transfected with KCNH2-WT or KCNH2-shIMM and shCT, shKCNH2, or KCNH2-SupRep (FIG. 28A). Relative KCNH2 expression normalized to GAPDH was measured by allele-specific qRT-PCR quantifying KCNH2-WT (white) and KCNH2-shIMM (grey). Results were confirmed by western blotting for KCNH2 with GAPDH as a housekeeping control (FIG. 28B).

### Example 16—Validation of Variant-Independent Suppression and Replacement Using KCNH2-SupRep

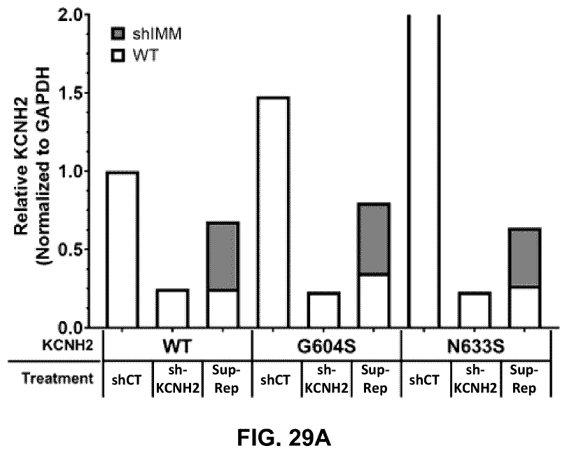

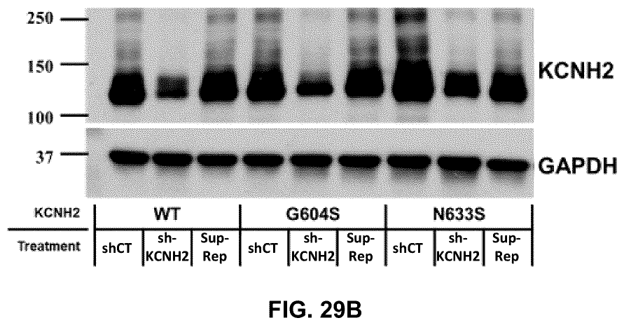

To test whether the KCNH2-SupRep gene therapy knocked down and replaced KCNH2 in a variant independent manner, TSA201 cells were co-transfected with KCNH2-WT or KCNH2-variants and shCT, shKCNH2, or KCNH2-SupRep. Results showed that KCNH2-SupRep knocked down LQT2 disease-causing KCNH2 missense variants and replaced them with KCNH2-shIMM. shKCNH2 knocked down KCNH2 in a variant-independent manner. KCNH2-SupRep knocked down KCNH2 variants via shKCNH2 and expressed KCNH2-shIMM which was knockdown immune. The graph in FIG. 29A shows proportional expression of KCNH2-WT/variants and KCNH2-shIMM detected using allele-specific qRT-PCR to measure KCNH2-WT/variant (white) and KCNH2-shIMM (grey). FIG. 29B shows overall KCNH2 expression (not allele-specific) validated by western blotting with GAPDH as a housekeeping control.

### Example 17—KCNH2-AAV-P2A CTnC-EGFP Generated E-4031 Sensitive Outward Current in H9C2 Cells

To determine whether the cardiac specific KCNH2-AAV-P2A CTnC-EGFP was only expressed in cardiomyocytes and not in non-cardiomyocytes, heterologous expression and patch-clamp studies were performed in TSA201 cells for both KCNH2-pIRES2-EGFP with KCNE2-pIRES2-dsRed2 and KCNH2-AAV-P2A CTnC-EGFP. Co-expression of KCNH2-pIRES2-EGFP along with KCNE2-pIRES2-dsRed2 revealed robust Iₖᵣ current. However, expression of KCNH2-AAV-P2A CTnC-EGFP only exhibited endogenous outward current from TSA201 cells, not typical KCNH2 current (FIGS. 30A and 30B) indicating that KCNH2-AAV-P2A CTnC-EGFP was not expressed in TSA201 cells. Peak current density was significantly smaller lower the voltage range from −10 mV to +20 mV for KCNH2-AAV-P2A CTnC-EGFP expression (FIG. 30C). At +10 mV, the peak current density was 41.4±3.4 pA/pF (KCNH2-pIRES2-EGFP, n=9) and 23.2±3.0 pA/pF (KCNH2-AAV-P2A CTnC-EGFP, n=8, p<0.05 vs. KCNH2-pIRES2-EGFP) (FIG. 30D).

To determine whether cardiac specific KCNH2-AAV-P2A CTnC-EGFP was expressed in cardiomyocytes and could generate KCNH2 current, heterologous expression and patch-clamp studies were performed in H9C2 cells, which are rat neonatal cardiomyocytes. Empty H9C2 cells only exhibited a small outward current (FIG. 31A, upper panel), whereas with KCNH2-AAV-P2A CTnC-EGFP expression, robust outward current was revealed (FIG. 31A, middle panel). This outward current was inhibited by a specific KCNH2 channel blocker (500 nM E-4031) (FIG. 31A, lower panel). The peak current density was significantly increased across the voltage range from −20 mV to +60 mV for KCNH2-AAV-P2A CTnC-EGFP expression (P<0.05 vs. empty H9C2) (FIG. 31B). At +60 mV, the peak current density was 17.3±3.8 pA/pF (empty H9C2, n=9) and 29.8±3.6 pA/pF (KCNH2-AAV-P2A CTnC-EGFP, n=9, p<0.05 vs. empty H9C2) (FIG. 31C). 500 nM E-4031 significantly inhibited KCNH2-AAV-P2A CTnC-EGFP expressed outward current across the voltage range from +10 mV to +60 mV (P<0.05 vs. before E-4031) (FIG. 31D). At +60 mV, peak current density was 29.8±5.4 pA/pF (before E-4031, n=6) and 19.1±3.2 pA/pF (after E-4031, n=6, p<0.05 vs. before E-4031) (FIG. 31E).

### Example 18—KCNH2-SupRep Prolongs the Pathologically Shortened Cardiac APD in SQT1 iPSC-CMs as Measured by FluoVolt Voltage Dye

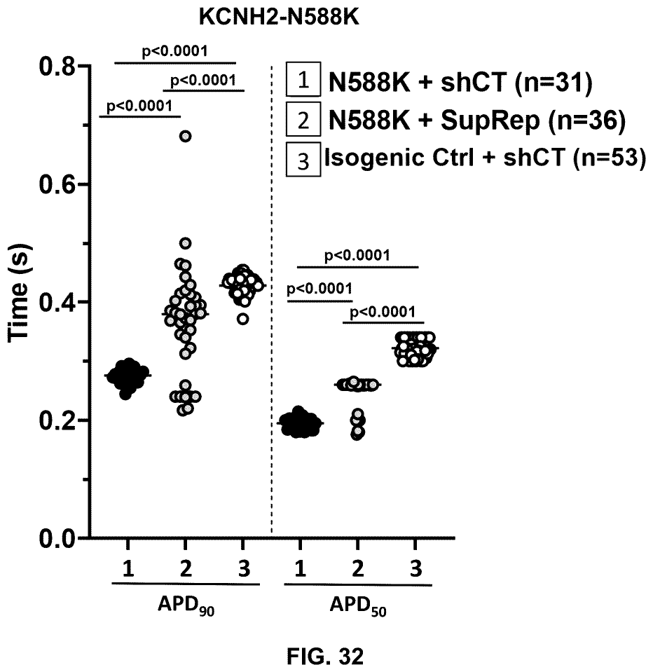

To show that KCNH2-SupRep can rescue both LQT2 and type 1 short QT (SQT1) disease phenotypes, CRISPR-Cas9 was used to insert KCNH2-N588K, a known SQT1 variant, into wildtype cells which serve as the isogenic control (FIG. 32). Isogenic controls served as markers for “ideal” cardiac APD. FluoVolt voltage dye measurement of the cardiac APD in N588K iPSC-CMs and isogenic control iPSC-CMs are plotted in FIG. 32. APD₉₀ and APD₅₀ values for isogenic control treated with shCT and KCNH2-N588K variant treated with shCT (1) or KCNH2-SupRep (2) are shown. The isogenic control iPSC-CMs (3) had significantly longer APD₉₀ and APD₅₀ than the SQT1 iPSC-CMs treated with shCT, which indicated that insertion of a single pathogenic type 1 short QT (SQT1) variant in KCNH2 was able to show the disease phenotype in vitro. Treatment of SQT1 iPSC-CMs with KCNH2-SupRep resulted in APD₉₀ prolongation. For KCNH2-N588K, KCNH2-SupRep corrected the shortened APD₉₀ and APD₅₀ as compared to isogenic control treated with shCT.

### Example 19—Materials and Methods for LQT3 SupRep

LQT3 Patient Selection for iPSC Generation: Patients were evaluated by a genetic cardiologist and LQTS specialist. Dermal fibroblasts and PBMCs were collected by 4 mm skin punch biopsy and blood sample, respectively. Samples were obtained from nearly 1200 patients diagnosed with a variety of inherited cardiac channelopathies and their affected or unaffected family members, including 80 patients with LQT3. For generation of iPSCs, four LQT3 patients bearing mutations resulting in the following changes on the protein level were selected: P1332L, R1623Q, and F1760C (TABLE 8).

Fibroblast/PBMCs Reprogramming into iPSCs and Quality Control: Fibroblasts or PBMCs were reprogrammed by Sendai virus transduction using the CytoTune-iPS 2.0 reprogramming kit (Thermo; Waltham, MA) or electroporation with four episomal DNA plasmids containing the Yamanaka factors: pCXLE-hUL, pCXLE-hSK, pCXLE-hOCT3/4-shp53-F, and pCXWB-EBNA1 (Addgene; Watertown, MA). At least two colonies were picked within 21 days post-induction and clonally expanded. All iPSCs were cultured in mTeSR™1 (STEMCELL®) supplemented with 1% penicillin/streptomycin on MATRIGEL®-coated (Corning) 6 cm culture dishes in a 5% CO₂ incubator at 37° C. At 85% confluence, iPSCs were passaged using ReLeSR (STEMCELL®). Each clone was then karyotyped.

All lines had a normal karyotype. SCN5A variant confirmation was conducted by Sanger sequencing of PCR-amplicons from genomic DNA. Expression of pluripotent markers in all iPSC clones was confirmed by confocal immunofluorescence microscopy using primary antibodies against Oct4 (Thermo, PA5-27438), Nanog (Thermo, PA1-097), Tra-1-60 (Santa Cruz; Dallas, TX; sc-21705), and SSEA-4 (Thermo, MA1-021) at a 1:250 dilution. Secondary antibodies were ALEXA FLUOR® 488 goat-anti-mouse (Thermo, A-11001) and ALEXA FLUOR® 594 goat-anti-rabbit (Thermo, A-11037). Counterstaining with DAPI (Thermo) was used at a 1:2000 dilution from a 5 mg/mL stock. Images were acquired on a Zeiss LSM 980 confocal microscope.

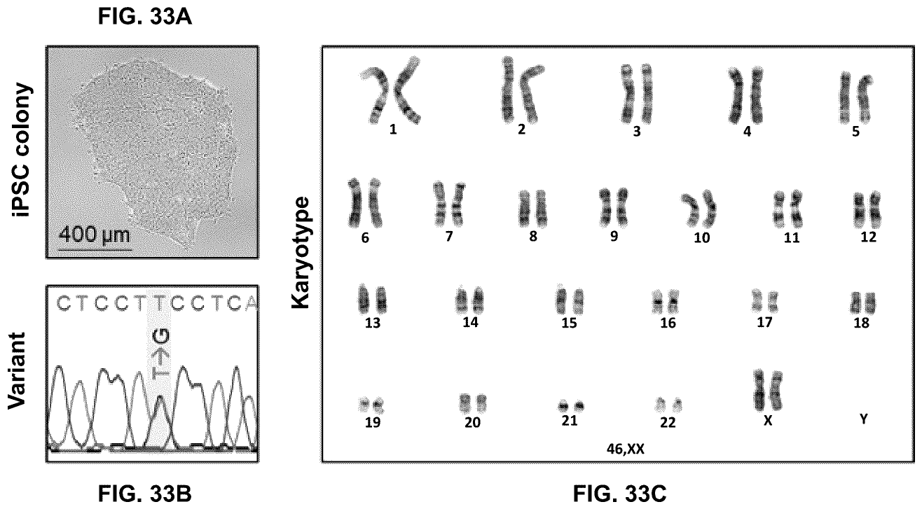

Quality control for iPSCs: Standard quality control assays were performed on SCN5A-F1760C iPSC line, including Sanger sequencing of the LQT3-causative variant, karyotyping, bright field morphology, and immunofluorescence microscopy for pluripotent markers including Tra-1-60, Nanog, SSEA-4, and Oct4 (FIG. 33A-33D). Differentiation of iPSCs was induced by methods described elsewhere to generate spontaneously beating iPSC-CMs (Burridge et al., supra; and Mummery et al., supra). Since the cardiac APD is known to shorten as iPSC-CMs mature over time, all experiments were conducted at least 30 days after the induction of differentiation (Shaheen et al., supra).

iPSC-CM Culture, Differentiation, and Dissociation: When iPSCs were 85% confluent, differentiation into cardiomyocytes (CMs) was induced as described elsewhere (Schwartz 2009, supra; and Schwartz 2013, supra). Differentiation was initiated (day 0) by changing the culture medium to RPMI 1640 GlutaMAX™ plus 25 mM HEPES ((4-(2-hydroxyethyl)-1-piperazineethanesulfonic acid)) supplemented with B27-minus insulin (RPMI/B27-ins) (Thermo) containing 51.1M CHIR99021 (MilliporeSigma; St. Louis, MO). On day 2, the medium was changed to RPMI/B27-ins containing 51.1M IWP-2 (MilliporeSigma). On day 4, the medium was changed back to the maintenance medium RPMI/B27-ins. Spontaneous beating typically began on days 6-7, with expansion to remaining cells by days 10-12. The iPSC-CMs were allowed to mature until at least day 30, changing the media twice per week. After day 30, iPSC-CMs were dissociated enzymatically using STEMdiff™ cardiomyocyte dissociation kit (STEMCELL®). Briefly, cells were rinsed with PBS (without Ca²⁺/Mg²⁺) and placed in dissociation medium for 10 minutes at 37° C., and then deactivated by addition of STEMdiff™ Cardiomyocyte Support Medium (STEMCELL®). Cells were triturated, transferred to a 15 mL conical tube, and pelleted by centrifugation at 300 rcf for 3 minutes. The supernatant was aspirated and the cells suspended in Cardiomyocyte Support Medium before transfer to appropriate MATRIGEL®-coated culture ware. After 24 hours, the medium was changed back to RPMI/B27-ins. Dissociation resulted in a mixture of single cells and small-to-medium sized iPSC-CM clusters, depending on cell density before and after plating. Spontaneous beating generally returned 24 hours after dissociation, with strong electrical coupling and syncytia formation between days 3-7.

CRISPR-Cas9 Corrected Isogenic Control iPSC: Isogenic “variant corrected” control iPSC cell lines were commercially created for the three patient-specific LQT3 cells lines harboring either SCN5A-R1623Q, SCN5A-P1332L, or SCN5A-F1760C mutation. These isogenic controls serve as the gold standard for a possible therapeutic cure, thereby providing a marker for the “ideal” rescue/normalization of the prolonged APD and indicating how close to this ideal did treatment with SCN5A-SupRep gene therapy reach.

Lentivirus Generation and Transduction of iPSC-CMs: Lentivirus was used for application of SCN5A-SupRep to iPSC-CMs (or shCT as a treatment control). Lentiviral particles were generated from shLenti-shSCN5A-shIMM-P2A-GFP (SCN5A-GFP-SupRep) and shLenti-shSCN5A-shIMM-HA (SCN5A-HA-SupRep), using the pPACKH1 HIV Lentivector Packaging kit (SBI System Biosciences; Palo Alto, CA). After reaching at least day 30 post-induction of differentiation, iPSC-CMs patient with LQT3 were dissociated and plated into MATRIGEL®-coated 35 mm dishes with glass-bottom insets for FLUOVOLT™ (MatTek) or 10-well culture reaction slides for immunofluorescence (Marienfeld SUPERIOR™) as described above. After 24-48 hours of recovery, iPSC-CMs were left untreated or were transduced with lentiviral particles containing SCN5A-SupRep. To increase transduction efficiency, Polybrene infection reagent (MilliporeSigma) was added during transduction to a final concentration of 8 μg/mL and the iPSC-CMs were centrifuged at 250 rcf for 1.5 hours at room temperature in the 35 mm dishes. At 24 hours post-transduction, the medium was exchanged for fresh maintenance medium, RPMI/B27-ins.

Voltage Dye Optical Action Potentials in iPSC-CMs: Voltage dye experiments were conducted between 3-7 days post-transduction of iPSC-CMs with lentiviral particles containing SCN5A-SupRep. On the day of imaging, iPSC-CMs were rinsed with pre-warmed (37° C.) HEPES-buffered Tyrode's solution (Alfa Aesar). Using the FLUOVOLT™ Membrane Potential kit (Thermo), 0.125 μL FLUOVOLT™ dye and 1.25 μL PowerLoad were added to 0.5 mL Tyrode's solution for each 35 mm glass-bottom dish and incubated at 37° C. for 20 minutes. Excess dye was removed in three rinses with pre-warmed Tyrode's solution, and a final 2 mL Tyrode's solution was added to the iPSC-CMs for imaging. During imaging, the dishes were kept in a heated 37° C. stage-top chamber (Live Cell Instrument) with 5% CO₂. Using a Nikon Eclipse Ti light microscope (Nikon) under 40×-water objective magnification, optical action potentials were recorded in 20 second fast time-lapse videos at a rate of 50 frames/second (fps, 20 ms exposure time) with LED illumination at 5% power. iPSC-CMs were paced at 1 Hz (9 ms pulse duration, 25V) using a MyoPacer field stimulator (Ion Optix; Westwood, MA) to eliminate beat-rate dependent effects on the APD. Videos were focused on electrically-coupled syncytial areas of iPSC-CMs (clusters and monolayers) since these areas of cells best follow the pacing stimulus and produce the greatest signal-to-noise represented by large changes in fluorescence intensity (often ˜8-12%). For analysis, rectangular regions of interest were drawn over flashing areas of cells, and NIS-Elements software (Nikon) was used to quantify the fluorescence intensity over time within each region of interest, resulting in optical action potential traces. Using a custom Excel-based program, traces were corrected for photobleaching and the amplitude was normalized as change in fluorescence divided by the baseline minimum florescence (ΔF/Fₘᵢₙ). In a semi-automated manner, common action potential parameters including APD₉₀, APD₅₀, amplitude, rise time, upstroke velocity, etc. were detected for each individual optical action potential and averaged across all beats within a 20 second trace. The average of all beats within a 20 second trace represented a single data point. For representative traces, the maximum amplitude was further normalized to 1.0 to allow for accurate visualization of APD differences.

Statistics: GraphPad Prism 9 was used for all statistical analysis and to fit all data for figures. Individual data points are shown wherever practical along with the mean. Error bars represent standard error of the mean (SEM). An unpaired two-tailed student's t-test was performed to determine statistical significance between two groups when indicated. p<0.05 was considered to be significant.

[symbol]

TABLE 8
Summary of subjects selected for generation of iPSCs for iPSC-CM studies

| | | Age at | | Average | LOTS [symbol] | | | IPSC Source [symbol] |
|---|---|---|---|---|---|---|---|---|
| | | Sample | SCN [symbol] | QT [symbol] | Related | Family | | Generation |
| Subject | Sex | Collection | Variant(s) | (ms) | Symptoms | History | Treatment | Method |
| LQT3 #1 | Female | [symbol] | P1332L | 583 | Cardiac arrest at age | — | Nadolol and | PBMC [symbol] |
| | | | (c.3995C > T) | | 25 months | | mexiletine | Episomal |
| | | | | | | | | DNA |
| LQT3 #2 | Male | 3.1 | R1623Q | 480 | Recurrent episodes | Symptomatic | Propranolol, | Fibroblasts [symbol] |
| | | | (c.4868G > A) | | of spontaneously | LQTS in | mexiletine, ICD | Sendai |
| | | | | | [symbol] and ICD- | twin | implant, RCSD, | |
| | | | | | [symbol] VF | brother | LCSD, Heart | |
| | | | | | | | transplant | |
| LQTS #3 | Female | 2 months | F1760C | 680 | Several long | — | Mexiletine, | PBMC [symbol] |
| | | | (c. [symbol] 279T > C) | | episodes of [symbol] | | LCSD, ICD, | Episomal |
| | | | | | de pointes, 3:1 AV | | | DNA |
| | | | | | block | | | |
| | | | | | SCD at age [symbol] | | | |
| | | | | | months | | | |

SCN [symbol] variants are listed as the resulting change on the protein level with cDN A change in parenthesis.
QT [symbol] , Bazett-corrected QT interval;
ICD, implantable cardioverter defibrillator;
PBMC, peripheral blood mononuclear cells;
LCSD, left cardiac sympathie denervation;
LCSD, right cardiac sympathie denervation;
SCD, sudden cardiac death. [symbol]
indicates data missing or illegible when filed

### Example 20—shRNA Knockdown of SCN5A

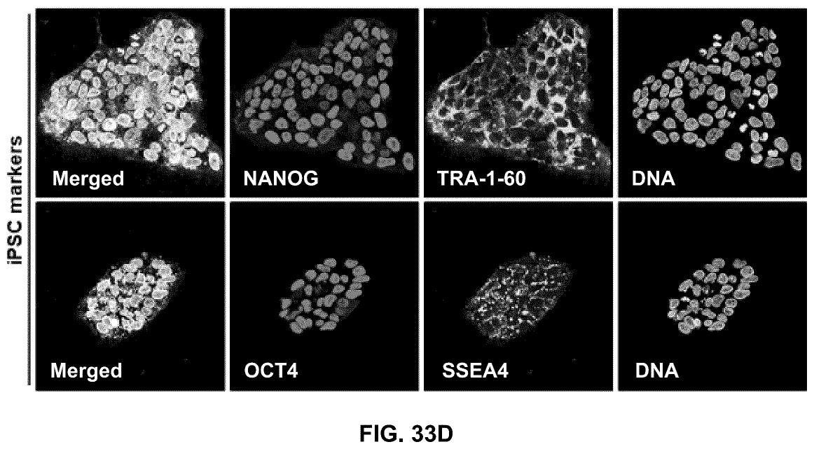

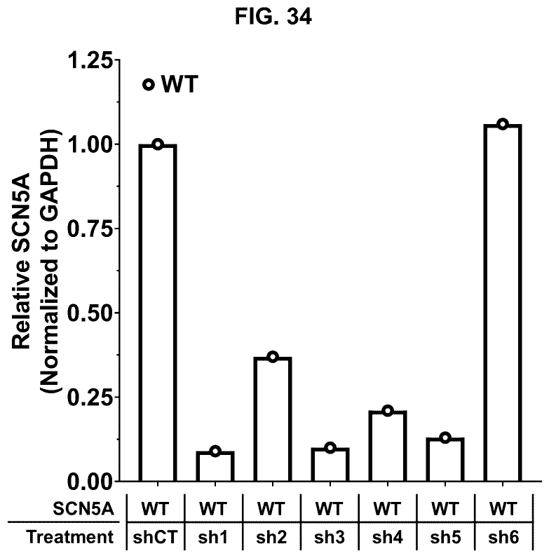

To make SCN5A-SupRep, six candidate SCN5A shRNAs (sh#1-6) in the pGFP-C-shLenti lentiviral backbone were tested. The KD efficiency of each SCN5A shRNA was determined by co-transfecting TSA201 cells with SCN5A-WT and sh#1-6. Expression of SCN5A was measured by quantitative reverse transcription PCR (qRT-PCR, FIG. 34). Of the six shRNAs tested, sh#1, sh#3, sh#4 and sh#5 all resulted in significant KD of SCN5A (mRNA: 78-91% KD). Thus, any of these shRNAs could have been used as part of the final SCN5A-SupRep gene therapy vector. By raw KD, however, SCN5A sh#1 (5′-GGTTCACTCGCTCTTCAACATGCTCATCA-3; SEQ ID NO:30) (RNA sequence 5′-GGUUCACUCGCUCUUCAACAUGCUCAUCA-3; SEQ ID NO:31) provided the strongest KD of SCN5A, suppressing the endogenous SCN5A alleles (both mutant and wild-type) in TSA201 cells with about 91% knockdown efficiency (FIG. 34). Further, at the time of selection, the SCN5A sh#1 target sequence was assessed using the Genome Aggregation Database (gnomAD) and ClinVar, and was found to be devoid of both common genetic polymorphisms and all known pathogenic LQT3-causative mutations that may interfere with KD efficiency. SCN5A sh#1 therefore was selected for the final SCN5A-SupRep and is referred to as “shSCN5A.”

The shRNA was designed in a lentivirus backbone (pGFP-C-shLenti). Once this shRNA was identified, a SupRep construct containing the shRNA (SEQ ID NO:31) and an “shRNA-immune” (5′-CGTACATTCCCTGTTTAATATGCTGATTA-3; SEQ ID NO:32) version of the SCN5A cDNA was generated (the shRNA for knocking down the endogenous SCN5A alleles, and the shRNA-immune for simultaneously providing a replacement wild-type SCN5A allele). As with KCNQ1, the shIMM sequence had alterations at the wobble base of each codon within the shRNA target sequence, which prevented knockdown by the shRNA, but did not change the encoded amino acid sequence. The SupRep construct was designed in a lentivirus backbone (pGFP-C-shLenti), with three SupRep constructs generated in the lentivirus backbone. These constructs differed in the reporter sequences (P2A, HA-Tag, and No reporter) that they contained. The 3 total constructs were as follows:
shLenti-SupRep-P2A
shLenti-SupRep-HA Tag
shLenti-SupRep-No Reporter

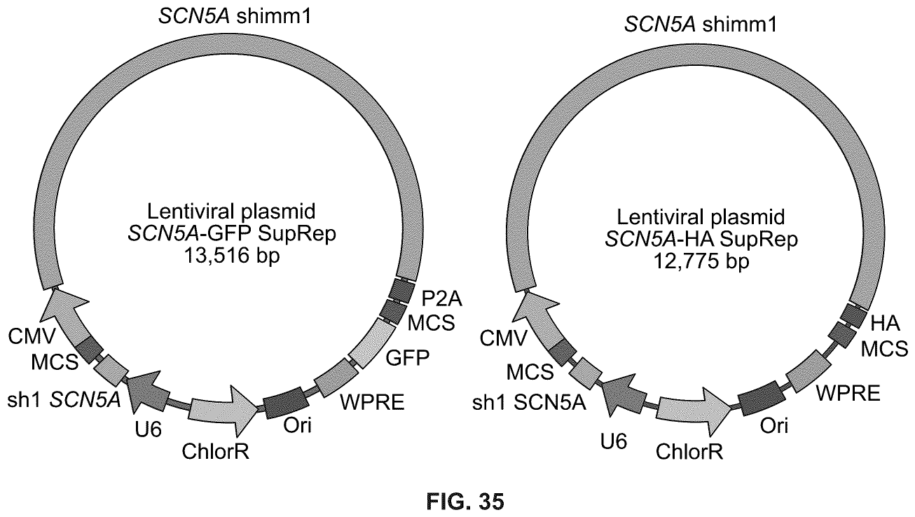

The final SCN5A-SupRep gene therapy vector used in this in vitro study is illustrated in FIG. 35. The SupRep constructs contained a CMV promoter and a human growth hormone (HGH) polyadenylation signal, but can be modified to include other promoters/enhancers. For example, the CMV promoter can be replaced with a cTnC promoter, which is smaller than the CMV promoter and more cardiac-specific. Additionally, the HGH polyadenylation signal can be replaced with a smaller SV40 terminator sequence. These modifications reduce the size of the SupRep construct and allow it to be packaged into AAV9 with greater efficiency.

### Example 21—SCN5A-SupRep Gene Therapy Shortens the Cardiac APD in LQT3 iPSC-CMs as Measured by FLUOVOLT™ Voltage Dye

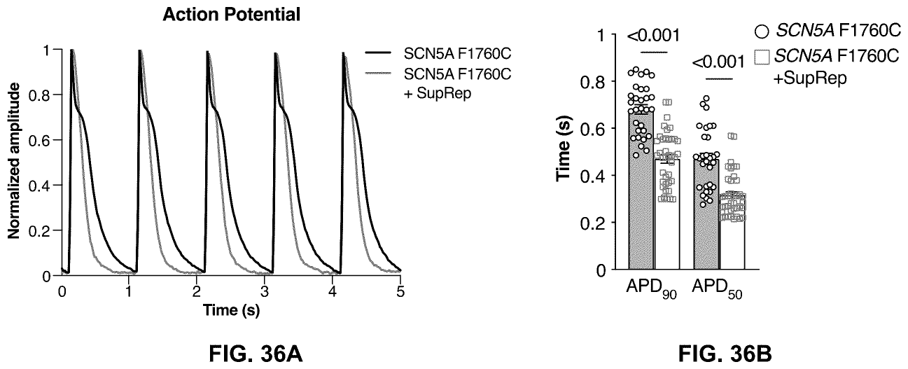

Action potential analyses were conducted to test whether treatment with SCN5A-SupRep gene therapy was able to rescue the pathognomonic feature of LQT3 by shortening the pathologically prolonged APD. FLUOVOLT™ voltage dye was used to measure optical action potentials in iPSC-CMs derived from a patient with LQT3-causing SCN5A-F1760C treated with SCN5A-SupRep gene therapy. All iPSC-CMs were paced at 1 Hz during recording to eliminate beat rate-dependent changes to the APD. Representative optical action potentials are shown in FIG. 36A. When untreated, SCN5A-F1760C iPSC-CMs had a significantly longer APD at 90% repolarization (APD₉₀) and had a significantly longer APD at 50% repolarization (APD₅₀) compared to untreated unrelated healthy control iPSC-CMs, validating the SCN5A-F1760C iPSC-CMs as an in vitro model of LQT3. APD shortening due to SCN5A-SupRep compared to untreated SCN5A-F1760C iPSC-CMs was then assessed by unpaired two-tailed student's t-tests at both the APD₉₀ and APD₅₀ levels separately for each variant. SCN5A-SupRep resulted in statistically significant attenuation of both APD₉₀ and APD₅₀ in SCN5A-F1760C iPSC-CMs (FIG. 36B). When treated with SCN5A-SupRep, the APD₉₀ and APD₅₀ of SCN5A-F1760C lines shortened significantly. These results indicated that suppression-replacement gene therapy is a promising strategy for directly targeting the pathogenic substrate and ameliorating the resultant disease for LQT3.

### Example 22—shRNA Knockdown of MYH7

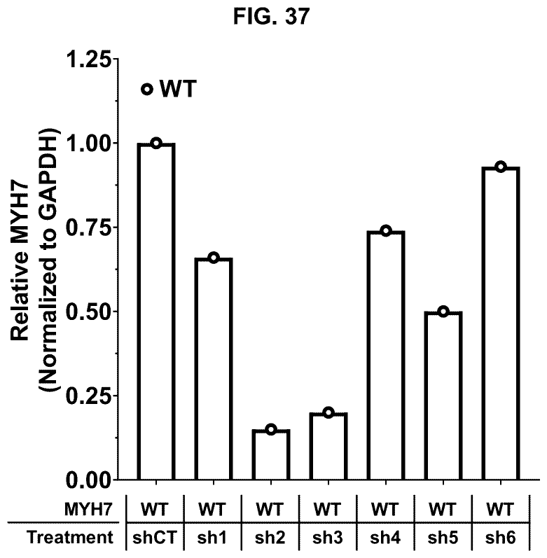

Six (6) unique shRNAs targeting MYH7 were tested, and one candidate shRNA (designated sh2) was identified that suppressed the endogenous MYH7 alleles (both mutant and wild-type) in TSA201 cells with about 85% knockdown efficiency (FIG. 37). The shRNA (5′-GCTGAAAGCAGAGAGAGATTATCACATTT-3; SEQ ID NO:33) (RNA sequence 5′-GCUGAAAGCAGAGAGAGAUUAUCACAUUU-3; SEQ ID NO:34) was completely homologous with the human sequence. The shRNA was designed in a lentivirus backbone (pGFP-C-shLenti). Once this shRNA was identified, a SupRep construct containing the shRNA (SEQ ID NO:34) and an “shRNA-immune” (5′-ACTCAAGGCTGAAAGGGACTACCATATAT-3; SEQ ID NO:35) version of the MYH7 cDNA was generated (the shRNA for knocking down the endogenous MYH7 alleles, and the shRNA-immune for simultaneously providing a replacement wild-type MYH7 allele). As with KCNQ1, the shIMM sequence had alterations at the wobble base of each codon within the shRNA target sequence, which prevented knockdown by the shRNA, but did not change the encoded amino acid sequence. The SupRep construct was designed in a lentivirus backbone (pGFP-C-shLenti), with three SupRep constructs generated in the lentivirus backbone. These constructs differed in the reporter sequences (P2A, HA-Tag, and No reporter) that they contained. The 3 total constructs were as follows:
shLenti-SupRep-P2A
shLenti-SupRep-HA Tag
shLenti-SupRep-No Reporter

The SupRep constructs contained a CMV promoter and a human growth hormone (HGH) polyadenylation signal, but can be modified to include other promoters/enhancers. For example, the CMV promoter can be replaced with a cTnC promoter, which is smaller than the CMV promoter and more cardiac-specific. Additionally, the HGH polyadenylation signal can be replaced with a smaller SV40 terminator sequence. These modifications reduce the size of the SupRep construct and allow it to be packaged into AAV9 with greater efficiency.

### Example 23—Materials and Methods for PKP2 SupRep

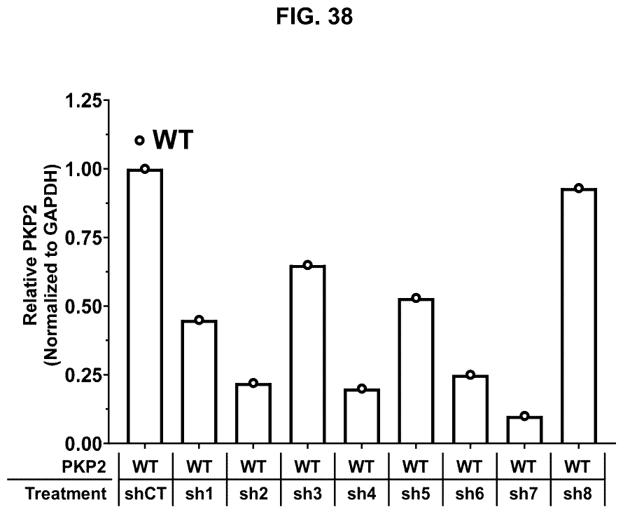

Generation of a PKP2-SupRep gene therapy construct: To make PKP2-SupRep, eight candidate PKP2 shRNAs (sh#1-8) in the pGFP-C-shLenti lentiviral backbone were tested. The KD efficiency of each PKP2 shRNA was determined by co-transfecting TSA201 cells with PKP2-WT and sh#1-8. Expression of PKP2, normalized to GAPDH, was measured by qRT-PCR (FIG. 38). Of the eight shRNAs tested, sh#2, sh#4, sh#6 and sh#7 all resulted in significant KD of PKP2 (mRNA: 75-90% KD). Any of these shRNAs could in theory have been used as part of the final PKP2-SupRep gene therapy vector. To select a final shRNA from the four potential candidates, by raw KD, PKP2 sh#7 (5′-GCAGAGCTCCCAGAGAAATAT-3; SEQ ID NO:52) (RNA sequence 5′-GCAGAGCUCCCAGAGAAAUAU-3; SEQ ID NO:53) provided the strongest KD of PKP2 on both the mRNA (90%) levels. Further, at the time of selection, the PKP2 sh#7 target sequence was assessed using the Genome Aggregation Database (gnomAD) and ClinVar, and was found to be devoid of both common genetic polymorphisms and all known pathogenic ACM-causative mutations that may interfere with KD efficiency. PKP2 sh#7 therefore was selected for the final PKP2-SupRep and is referred to as “shPKP2.”

To create the replacement shRNA-immune version of PKP2, called PKP2-shIMM, ten synonymous variants were introduced into the WT PKP2 cDNA (NM_004572.4) at the wobble base of each codon within the shPKP2 target site (5′-GCTGAACTGCCTGAAAAGTAC-3; SEQ ID NO:990). PKP2-shIMM was then cloned into the shPKP2-containing vector, pGFP-C-shLenti, downstream of the CMV promoter. Three variations of the construct were made: shLenti-SupRep-P2A-GFP, shLenti-SupRep-HA Tag, shLenti-SupRep-No Reporter.

PKP2 Patient Selection for iPSC Generation: Patients were evaluated by a genetic cardiologist. Dermal fibroblasts and PBMCs were collected by 4 mm skin punch biopsy and blood sample, respectively. Samples were obtained from nearly 1200 patients diagnosed with a variety of inherited cardiac channelopathies and their affected or unaffected family members, including 29 patients with PKP2 variants. Four patients with PKP2 variants were selected for generation of iPSCs: R79X, E149X, Q457X, c.2146-1G>C.

Fibroblast/PBMCs Reprogramming into iPSCs and Quality Control: Fibroblasts or PBMCs were reprogrammed by Sendai virus transduction using the CytoTune-iPS 2.0 reprogramming kit (Thermo) or electroporation with four episomal DNA plasmids containing the Yamanaka factors: pCXLE-hUL, pCXLE-hSK, pCXLE-hOCT3/4-shp53-F, and pCXWB-EBNA1 (Addgene; Watertown, MA). At least two colonies were picked within 21 days post-induction and clonally expanded. All iPSCs were cultured in mTeSR™1 (STEMCELL®) supplemented with 1% penicillin/streptomycin on MATRIGEL®-coated (Corning) 6 cm culture dishes in a 5% CO₂ incubator at 37° C. At 85% confluence, iPSCs were passaged using ReLeSR (STEMCELL®). Each clone was then karyotyped.

All lines had a normal karyotype. PKP2 variant confirmation was conducted by Sanger sequencing of PCR-amplicons from genomic DNA. Expression of pluripotent markers in all iPSC clones was confirmed by confocal immunofluorescence microscopy using primary antibodies against Oct4 (Thermo, PAS-27438), Nanog (Thermo, PA1-097), Tra-1-60 (Santa Cruz; Dallas, TX; sc-21705), and SSEA-4 (Thermo, MA1-021) at a 1:250 dilution. Secondary antibodies were ALEXA FLUOR® 488 goat-anti-mouse (Thermo, A-11001) and ALEXA FLUOR® 594 goat-anti-rabbit (Thermo, A-11037). Counterstaining with DAPI (Thermo) was used at a 1:2000 dilution from a 5 mg/mL stock. Images were acquired on a Zeiss LSM 980 confocal microscope.

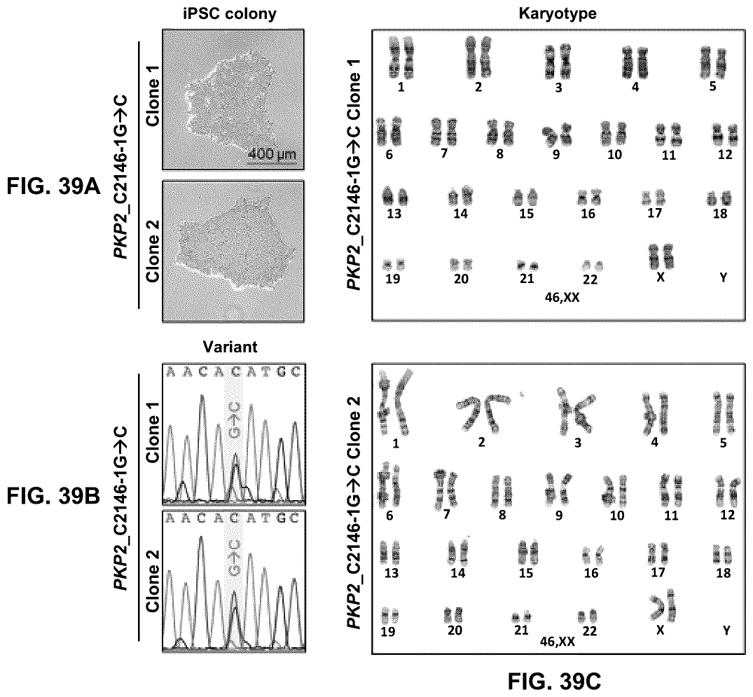

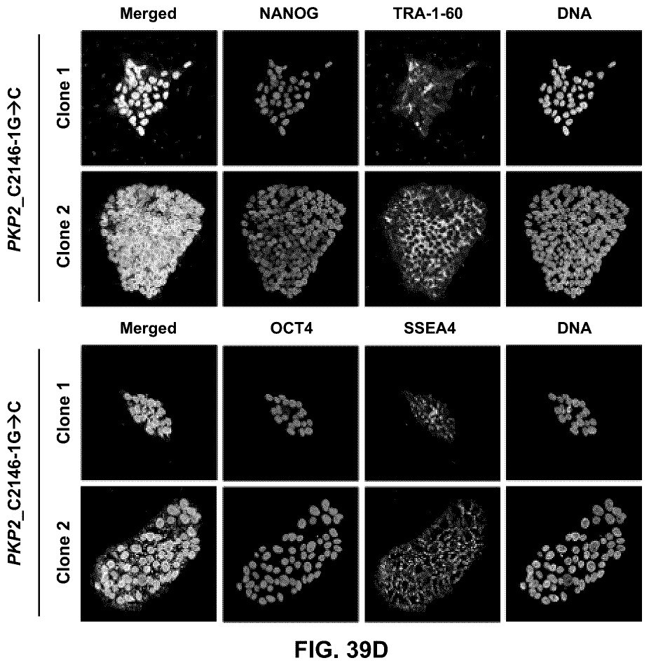

Quality control for iPSCs: Standard quality control assays were performed on c.2146-1G>C iPSC line, including Sanger sequencing of the ACM-causative variant, karyotyping, bright field morphology, and immunofluorescence microscopy for pluripotent markers including Tra-1-60, Nanog, SSEA-4, and Oct4 (FIG. 39A-39D). Differentiation of iPSCs was induced by methods described elsewhere to generate spontaneously beating iPSC-CMs (Burridge et al., supra; and Mummery et al., supra). Since the cardiac APD is known to shorten as iPSC-CMs mature over time, all experiments were conducted at least 30 days after the induction of differentiation (Shaheen et al., supra).

iPSC-CM Culture, Differentiation, and Dissociation: When iPSCs were 85% confluent, differentiation into cardiomyocytes (CMs) was induced as described elsewhere (Schwartz 2009, supra; and Schwartz 2013, supra). Differentiation was initiated (day 0) by changing the culture medium to RPMI 1640 GLUTAMAX™ plus 25 mM HEPES ((4-(2-hydroxyethyl)-1-piperazineethanesulfonic acid)) supplemented with B27-minus insulin (RPMI/B27-ins) (Thermo) containing 51.1M CHIR99021 (MilliporeSigma). On day 2, the medium was changed to RPMI/B27-ins containing 5 μM IWP-2 (Millipore Sigma). On day 4, the medium was changed back to the maintenance medium RPMI/B27-ins. Spontaneous beating typically began on days 6-7, with expansion to remaining cells by days 10-12. The iPSC-CMs were allowed to mature until at least day 30, changing the media twice per week. After day 30, iPSC-CMs were dissociated enzymatically using STEMDIFF™ cardiomyocyte dissociation kit (STEMCELL®). Briefly, cells were rinsed with PBS (without Ca²⁺/Mg²⁺) and placed in dissociation medium for 10 minutes at 37° C., and then deactivated by addition of STEMDIFF™ Cardiomyocyte Support Medium (STEMCELL®). Cells were triturated, transferred to a 15 mL conical tube, and pelleted by centrifugation at 300 rcf for 3 minutes. The supernatant was aspirated, and the cells suspended in Cardiomyocyte Support Medium before transfer to appropriate MATRIGEL®-coated culture ware. After 24 hours, the medium was changed back to RPMI/B27-ins. Dissociation resulted in a mixture of single cells and small-to-medium sized iPSC-CM clusters, depending on cell density before and after plating. Spontaneous beating generally returned 24 hours after dissociation, with strong electrical coupling and syncytia formation between days 3-7.

Lentivirus Generation and Transduction of iPSC-CMs: Lentivirus was used for application of PKP2-SupRep to iPSC-CMs. Lentiviral particles were generated from shLenti-shPKP2-shIMM-P2A-GFP (PKP2-GFP-SupRep) and shLenti-shPKP2-shIMM-HA (PKP2-HA-SupRep), using the pPACKH1 HIV Lentivector Packaging kit (SBI System Biosciences; Palo Alto, CA). After reaching at least day 30 post-induction of differentiation, iPSC-CMs from a patient with ACM were dissociated and plated into MATRIGEL -coated 35 mm dishes with glass-bottom insets for Fluo-4 AM (Invitrogen; cat #F14201) or 10-well culture reaction slides for immunofluorescence (Marienfeld SUPERIOR™) as described above. After 24-48 hours of recovery, iPSC-CMs were left untreated or were transduced with lentiviral particles containing PKP2-SupRep. To increase transduction efficiency, Polybrene infection reagent (MilliporeSigma) was added during transduction to a final concentration of 8 μg/mL and the iPSC-CMs were centrifuged at 250 rcf for 1.5 hours at room temperature in the 35 mm dishes. After 24 hours post-transduction, the medium was exchanged for fresh maintenance medium, RPMI/B27-ins.

Intracellular Calcium Assay in iPSC-CMs: Intracellular calcium assay experiments were conducted between 3-7 days post-transduction of iPSC-CMs with lentiviral particles containing PKP2-SupRep. On the day of imaging, iPSC-CMs were rinsed with pre-warmed (37° C.) HEPES-buffered Tyrode's solution (Alfa Aesar). Fluo-4 AM dye (Invitrogen) was dissolved in 50 μL DMSO, then 5 μL Fluo-4 AM and 2 μL PLURONIC™ F-127 (Invitrogen) were added to 1 mL Tyrode's solution for each 35 mm glass-bottom dish and incubated at 37° C. for 30 minutes. Excess dye was removed in one rinse and two 5-minute washes with pre-warmed Tyrode's solution, and a final 1.5 mL Tyrode's solution was added to the iPSC-CMs for imaging. During imaging, the dishes were kept in a heated 37° C. stage-top chamber (Live Cell Instrument) with 5% CO₂. Using a Nikon Eclipse Ti light microscope (Nikon) under 40X-water objective magnification, calcium transients were recorded in 20 second fast time-lapse videos at a rate of 50 frames/second (fps, 20 ms exposure time) with LED illumination at 5% power. iPSC-CMs were paced at 0.5 Hz (9 ms pulse duration, 25V) using a MyoPacer field stimulator (Ion Optix; Westwood, MA) to eliminate beat-rate dependent effects on the calcium transient. Videos were focused on electrically-coupled syncytial areas of iPSC-CMs (clusters and monolayers) since these areas of cells best follow the pacing stimulus and produce the greatest signal-to-noise represented by large changes in fluorescence intensity. For analysis, rectangular regions of interest were drawn over flashing areas of cells, and NIS-Elements software (Nikon) was used to quantify the fluorescence intensity over time within each region of interest, resulting in traces of calcium transients. Using a custom Excel-based program, traces were corrected for photobleaching and the amplitude was normalized as change in fluorescence divided by the baseline minimum florescence (ΔF/Fₘᵢₙ). In a semi-automated manner, common calcium transient parameters including Ca²⁺ amplitude, 50% and 90% Ca²⁺ transient duration (CTD), peak to 50% and peak to 90% decay, upstroke time, upstroke velocity, Vmax, etc. were detected for each individual calcium transient and averaged across all beats within a 20 second trace, except in case of Ca²⁺ amplitude where the value was taken only for the first beat. For all parameters, except for Ca²⁺ amplitude, the average of all beats within a 20 second trace represented a single data point. Upon recording the baseline measurements, 0.5 ml 400 nM isoproterenol was added to cells to make a final concentration of 100 nM and calcium transient recordings were taken every one minute for a total of 10 minutes. Traces were analyzed the same was as described above for baseline measurement.

Statistics: GraphPad Prism 9 was used for all statistical analysis and to fit all data for figures. Individual data points are shown wherever practical along with the mean. Error bars represent standard error of the mean (SEM). Two-way ANOVA with post-hoc Tukey's test for multiple comparisons also was used. A p<0.05 was considered to be significant.

### Example 24—PKP2-SupRep Gene Therapy Shortens Transient Duration and Decay Time in ACM iPSC-CMs as Measured by Fluo-4 AM

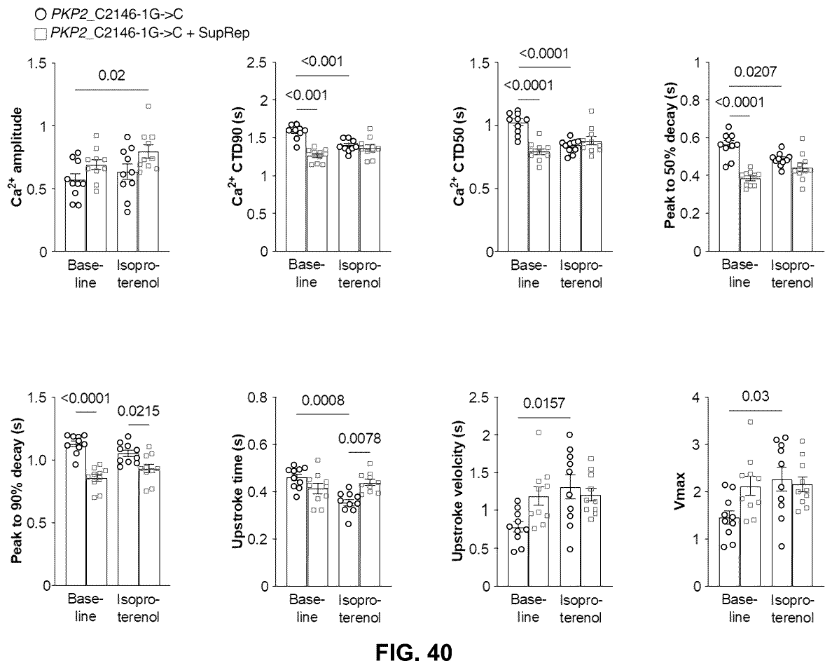

Calcium transient analyses were conducted to test whether treatment with PKP2-SupRep gene therapy was able to rescue the abnormal calcium handling feature of ACM. Fluo-4 AM dye was used to measure calcium transients in iPSC-CMs derived from patient with c.2146-1G>C PKP2 variant treated with PKP2-SupRep gene therapy. All iPSC-CMs were paced at 0.5 Hz during recording to eliminate beat rate-dependent changes to the calcium transient. Prolonged Ca²⁺ decay time is a key pathophysiology of ARVC, and may lead to remodeling of cardiac tissue into myopathic state, such as elevation of fibrosis and aseptic inflammation mediated exacerbation of desmosome alteration. Further, prolongation of Ca²⁺ decay time can accelerate arrhythmic potential through maladaption of sarcolemmal channel functions such as NCX1, LTCC, and Na⁺ channels which elicit DAD and EAD. These studies demonstrated that SupRep successfully rescued arrhythmic potential with one delivery of therapeutic regimen (FIG. 40).

### Example 25—shRNA Knock Down of DSP

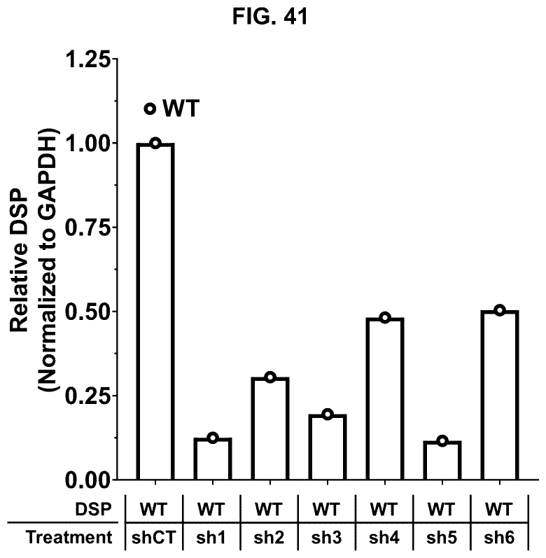

TSA201 cells were co-transfected with DSP-WT and six custom DSP shRNAs (sh1-6) or a non-targeting scrambled shRNA control (shCT). DSP expression normalized to GAPDH was measured by qRT-PCR. sh5 (5′-GCACTACTGCATGATTGACATAG AGAAGA-3; SEQ ID NO:44) (RNA sequence 5′-GCACUACUGCAUGAUUGACA UAGAGAAGA-3; SEQ ID NO:45) had the strongest knockdown by raw value (FIG. 41), with about 88% knockdown efficiency.

### Example 26—shRNA Knock Down of MYBPC3

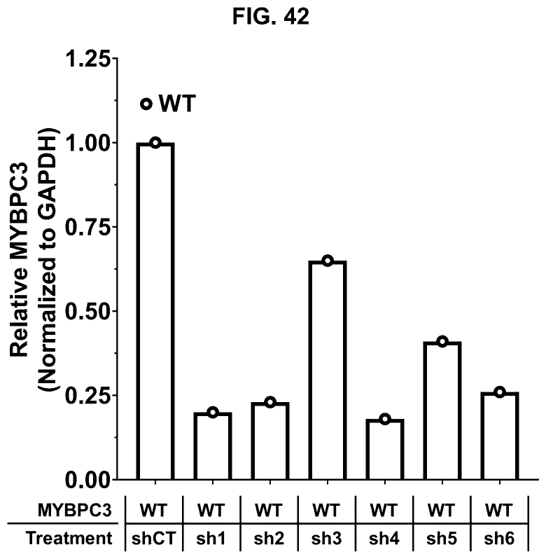

TSA201 cells were co-transfected with MYBPC3-WT and six custom MYBPC3 shRNAs (sh1-6) or non-targeting scramble shRNA control (shCT). MYBPC3 expression normalized to GAPDH was measured by qRT-PCR. sh4 (5′-GGAGGAGACCTTCAAAT ACCGGTTCAAGA-3; SEQ ID NO:46) (RNA sequence 5′-GGAGGAGACCUUCAAA UACCGGUUCAAGA-3; SEQ ID NO:47) had the strongest knockdown by raw value (FIG. 42), with about 82% knockdown efficiency.

### Example 27—shRNA Knock Down of RMB20

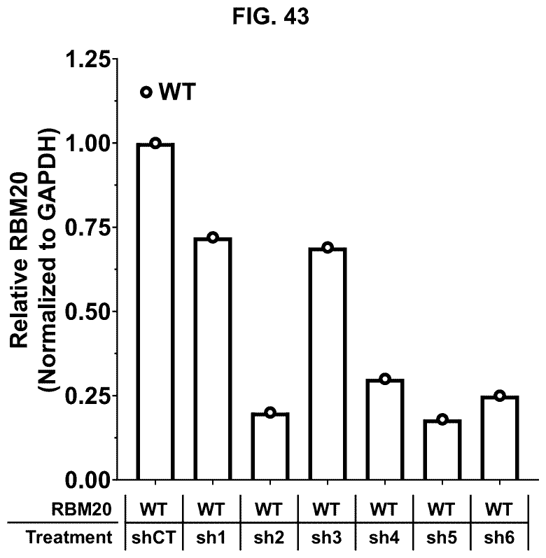

TSA201 cells were co-transfected with RBM20-WT and six custom RBM20 shRNAs (sh1-6) or non-targeting scramble shRNA control (shCT). RBM20 expression normalized to GAPDH was measured by qRT-PCR. sh5 (5′-GGTCATTCACTCAGTC AAGCCCCACATTT-3; SEQ ID NO:48) (RNA sequence 5′-GGUCAUUCACUCAGU CAAGCCCCACAUUU-3; SEQ ID NO:49) had the strongest knockdown by raw value (FIG. 43), with about 82% knockdown efficiency.

### Example 28—shRNA Knock Down of CACNA1C

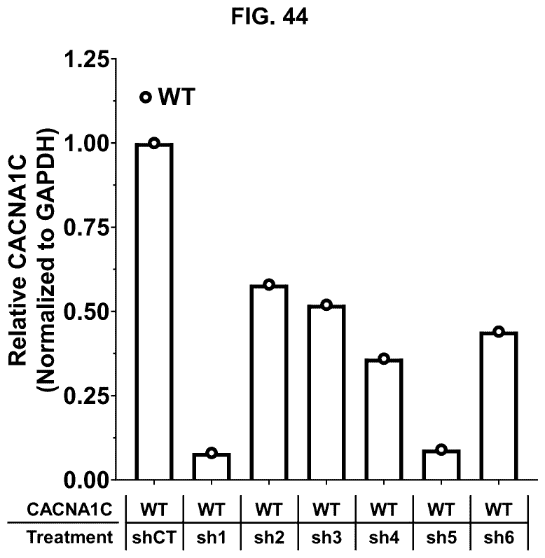

TSA201 cells were co-transfected with CACNA1C-WT and six custom CACNA1C shRNAs (sh1-6) or non-targeting scramble shRNA control (shCT). CACNA1C expression normalized to GAPDH was measured by qRT-PCR. sh1 (5′-GGAACGAGTGGAATATCTCTTTCTCATAA-3; SEQ ID NO:50) (RNA sequence 5′-GGAACGAGUGGAAUAUCUCUUUCUCAUAA-3; SEQ ID NO:51) had the strongest knockdown by raw value (FIG. 44), with about 92% knockdown efficiency.

### Example 29—Testing CALM1 shRNA

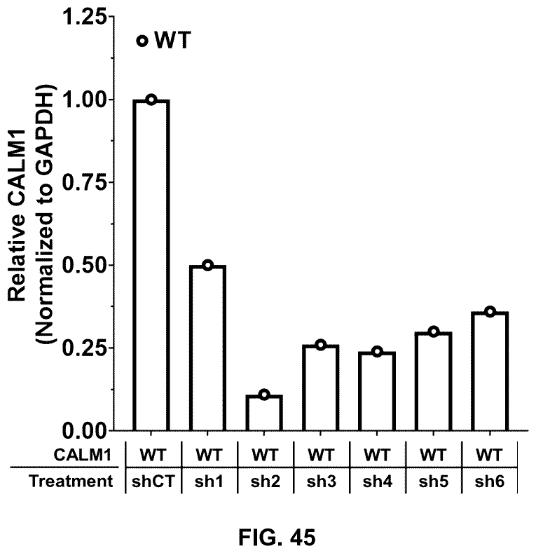

TSA201 cells were co-transfected with CALM1-WT and six custom CALM1 shRNAs (sh1-6) or non-targeting scramble shRNA control (shCT). CALM1 expression normalized to GAPDH was measured by qRT-PCR. Sh2 (5′-GAAAGATACAGATAGTGAAGAAGAA-3; SEQ ID NO:2738) (RNA sequence 5′-GAAAGAUACAGAUAGUGAAGAAGAA-3; SEQ ID NO:2739) had the strongest knockdown by raw value (FIG. 45), with about 89% knockdown efficiency.

### Example 30—Testing CALM2 shRNA

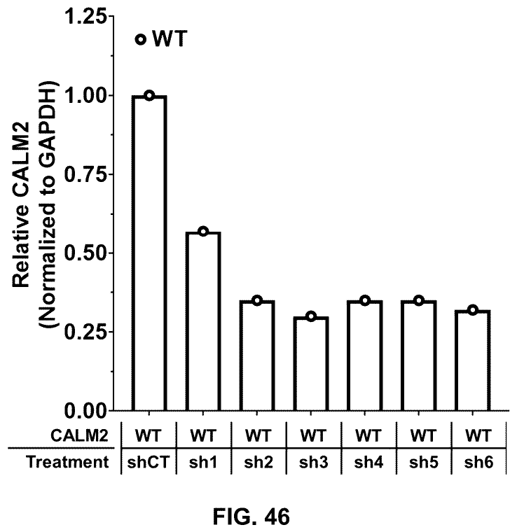

TSA201 cells were co-transfected with CALM2-WT and six custom CALM2 shRNAs (sh1-6) or non-targeting scramble shRNA control (shCT). CALM2 expression normalized to GAPDH was measured by qRT-PCR. Sh3 (5′-GCTGATGGTAATGGCACAATTGACT-3; SEQ ID NO:2740) (RNA sequence 5′-GCUGAUGGUAAUGGCACAAUUGACU-3; SEQ ID NO:2741) had the strongest knockdown by raw value (FIG. 46), with about 70% knockdown efficiency.

### Example 31—Testing CALM3 shRNA

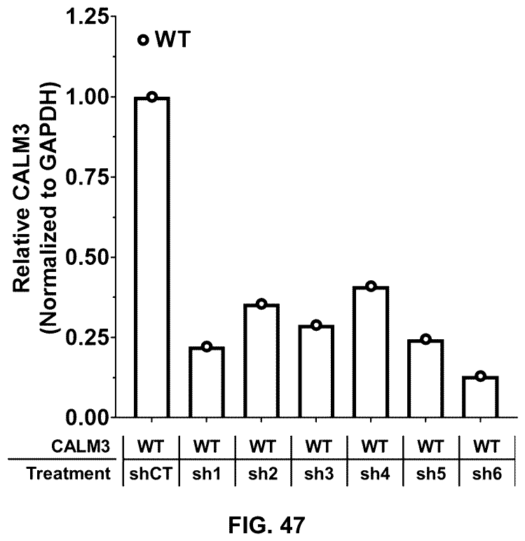

TSA201 cells were co-transfected with CALM3-WT and six custom CALM3 shRNAs (sh1-6) or non-targeting scramble shRNA control (shCT). CALM3 expression normalized to GAPDH was measured by qRT-PCR. Sh6 (5′-GATGAGGAGGTGGATGAGATGATCA-3; SEQ ID NO:2742) (RNA sequence 5′-GAUGAGGAGGUGGAUGAGAUGAUCA-3; SEQ ID NO:2743) had the strongest knockdown by raw value (FIG. 47), with about 87% knockdown efficiency.

### Example 32—Testing KCNJ2 shRNA

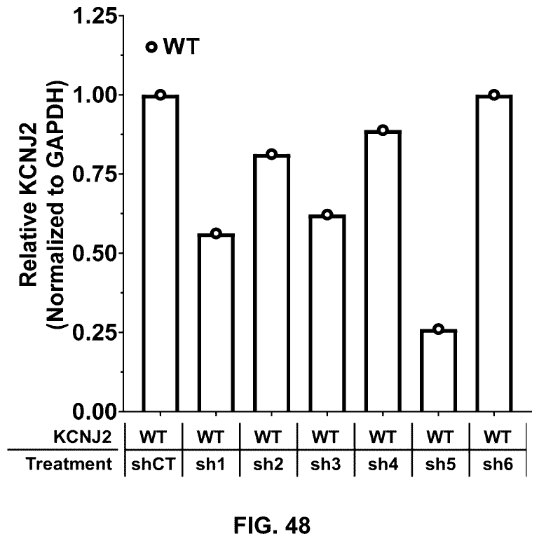

TSA201 cells were co-transfected with KCNJ2-WT and six custom KCNJ2 shRNAs (sh1-6) or non-targeting scramble shRNA control (shCT). KCNJ2 expression normalized to GAPDH was measured by qRT-PCR. Sh5 (5′-GTGCCGTAGCTCTTATCTAGCAAATGAAA-3; SEQ ID NO:2744) (RNA sequence 5′-GUGCCGUAGCUCUUAUCUAGCAAAUGAAA-3; SEQ ID NO:2745) had the strongest knockdown by raw value (FIG. 48), with about 74% knockdown efficiency.

### Example 33—Testing CASQ2 shRNA

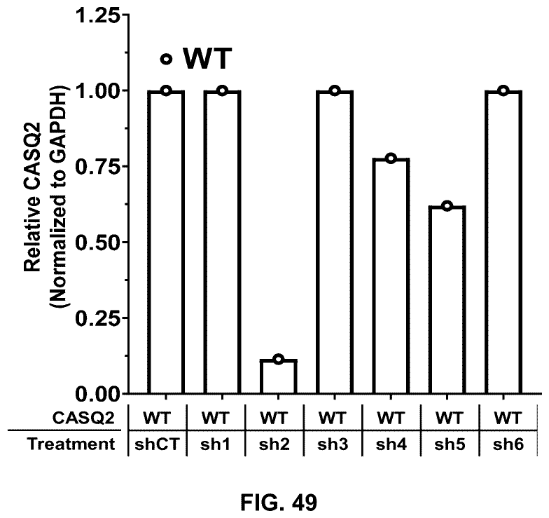

TSA201 cells were co-transfected with CASQ2-WT and six custom CASQ2 shRNAs (sh1-6) or non-targeting scramble shRNA control (shCT). CASQ2 expression normalized to GAPDH was measured by qRT-PCR. Sh2 (5′-AAGGAAGCCTGTATATTCTTA-3; SEQ ID NO:2746) (RNA sequence 5′-AAGGAAGCCUGUAUAUUCUUA-3; SEQ ID NO:2747) had the strongest knockdown by raw value (FIG. 49), with about 89% knockdown efficiency.

### Example 34—Testing DSG2 shRNA

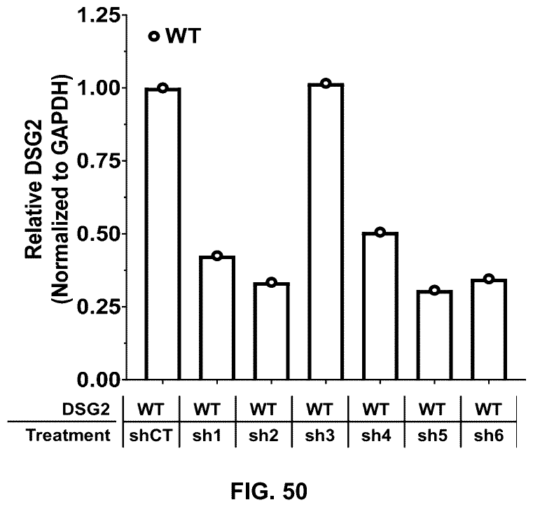

TSA201 cells were co-transfected with DSG2-WT and six custom DSG2 shRNAs (sh1-6) or non-targeting scramble shRNA control (shCT). DSG2 expression normalized to GAPDH was measured by qRT-PCR. Sh5 (5′-GCAGTCTAGTAGGAAGAAATGGAGTAGGA-3; SEQ ID NO:2748) (RNA sequence 5′-GCAGUCUAGUAGGAAGAAAUGGAGUAGGA-3; SEQ ID NO:2749) had the strongest knockdown by raw value (FIG. 50), with about 70% knockdown efficiency.

### Example 35—Testing TNNT2 shRNA

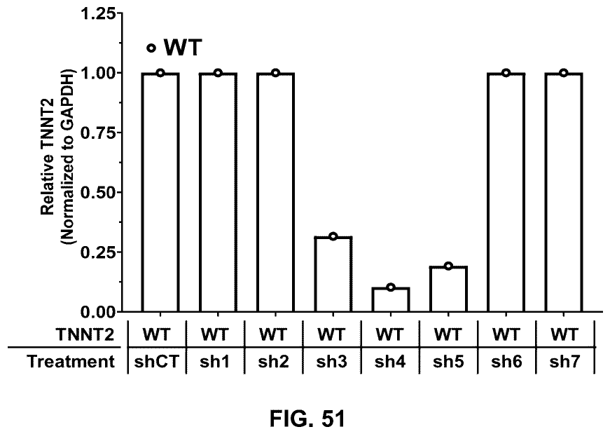

TSA201 cells were co-transfected with TNNT2-WT and seven custom TNNT2 shRNAs (sh1-7) or non-targeting scramble shRNA control (shCT). TNNT2 expression normalized to GAPDH was measured by qRT-PCR. Sh4 (5′-GAAGAAGAAGAGGAAGCAAAG-3; SEQ ID NO:2750) (RNA sequence 5′-GAAGAAGAAGAGGAAGCAAAG-3; SEQ ID NO:2750) had the strongest knockdown by raw value (FIG. 51), with about 90% knockdown efficiency.

### Example 36—Testing TPM1 shRNA

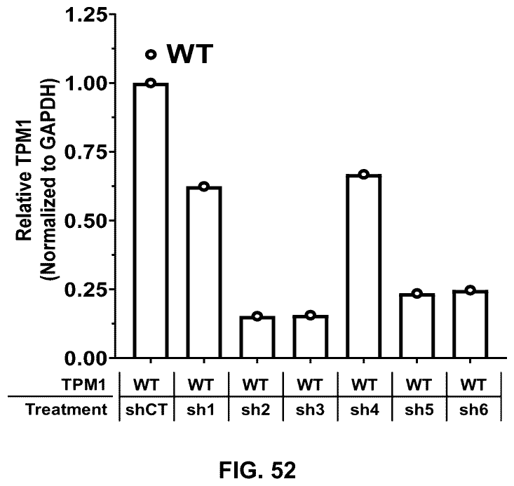

TSA201 cells were co-transfected with TPM1-WT and six custom TPM1 shRNAs (sh1-6) or non-targeting scramble shRNA control (shCT). TPM1 expression normalized to GAPDH was measured by qRT-PCR. Sh2 (5′-AAGCTGAGAAGGCAGCAGATG-3; SEQ ID NO:2751) (RNA sequence 5′-AAGCUGAGAAGGCAGCAGAUG-3; SEQ ID NO:2752) had the strongest knockdown by raw value (FIG. 52), with about 85% knockdown efficiency.

### Example 37—Testing LMNA shRNA

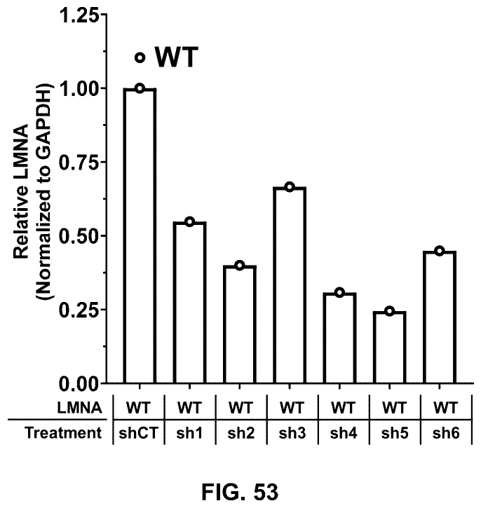

TSA201 cells were co-transfected with LMNA-WT and six custom LMNA shRNAs (sh1-6) or non-targeting scramble shRNA control (shCT). LMNA expression normalized to GAPDH was measured by qRT-PCR. Sh5 (5′-GGCAGATCAAGCGCCAGAATGGAGATGA-3; SEQ ID NO:2753) (RNA sequence 5′-GGCAGAUCAAGCGCCAGAAUGGAGAUGA-3; SEQ ID NO:2754) had the strongest knockdown by raw value (FIG. 53), with about 75% knockdown efficiency.

### Example 38—Testing PLN shRNA

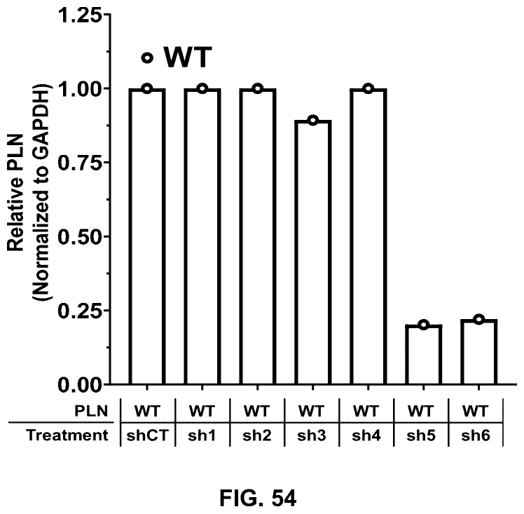

TSA201 cells were co-transfected with LMNA-WT and six custom PLN shRNAs (sh1-6) or non-targeting scramble shRNA control (shCT). PLN expression normalized to GAPDH was measured by qRT-PCR. Sh5 (5′-TGTCTCTTGCTGATCTGTATC-3; SEQ ID NO:2755) (RNA sequence 5′-UGUCUCUUGCUGAUCUGUAUC-3; SEQ ID NO:2756) had the strongest knockdown by raw value (FIG. 54), with about 80% knockdown efficiency.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid construct comprising:
(a) a first nucleotide sequence encoding an RNAi molecule capable of hybridizing to a target sequence encoding an endogenous KCNQ1 polypeptide within a cell and suppressing expression of said endogenous KCNQ1 polypeptide within said cell, and
(b) a second nucleotide sequence encoding a KCNQ1 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNQ1 polypeptide from said second nucleotide sequence within said cell.

2. The nucleic acid construct of claim 1, wherein said first nucleotide sequence comprises the sequence set forth in SEQ ID NO:12, SEQ ID NO:13, SEQ ID NO:15, or SEQ ID NO:36, and wherein said second nucleotide sequence comprises the sequence set forth in SEQ ID NO:9.

3. The nucleic acid construct of claim 1, wherein said first nucleotide sequence comprises the sequence set forth in SEQ ID NO:36 and said second nucleotide sequence comprises the sequence set forth in SEQ ID NO:9.

4. The nucleic acid construct of any one of claims 1 to 3, wherein said first nucleotide sequence is operably linked to a first promoter and said second nucleotide sequence is operably linked to a second promoter.

5. The nucleic acid construct of claim 4, wherein said first and second promoters are the same.

6. The nucleic acid construct of claim 4, wherein said first and second promoters are different.

7. The nucleic acid construct of claim 6, wherein said first promoter is a U6 promoter and said second promoter is a cytomegalovirus immediate-early (CMV) promoter.

8. The nucleic acid construct of any one of claims 1 to 7, further comprising a nucleotide sequence encoding a reporter.

9. The nucleic acid construct of claim 8, wherein said reporter is a fluorescent polypeptide.

10. The nucleic acid construct of claim 8 or claim 9, wherein said nucleotide sequence encoding said reporter is downstream of said second nucleotide sequence encoding said cDNA, and is separated from said second nucleotide sequence by an internal ribozyme entry sequence (IRES) or P2A self-cleaving peptide sequence.

11. The nucleic acid construct of any one of claims 1 to 10, wherein said nucleic acid construct is within a viral vector.

12. The nucleic acid construct of claim 11, wherein said viral vector is an adeno-associated virus (AAV) vector.

13. The nucleic acid construct of claim 12, wherein said AAV vector is an AAV serotype 9 vector or an AAV2/9 vector.

14. The nucleic acid construct of any one of claims 1 to 13, wherein said cell is a cardiomyocyte.

15. A virus particle comprising the nucleic acid construct of any one of claims 1 to 14.

16. A method for treating a mammal having a congenital cardiac disease, said method comprising administering to said mammal a nucleic acid construct comprising:
(a) a first nucleotide sequence encoding an RNAi molecule capable of hybridizing to a target sequence encoding an endogenous KCNQ1 polypeptide within a cell of said mammal and suppressing expression of said endogenous KCNQ1 polypeptide within said cell, and
(b) a second nucleotide sequence encoding a KCNQ1 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNQ1 polypeptide from said second nucleotide sequence within said cell.

17. The method of claim 16, wherein said congenital cardiac disease is long QT syndrome (LQTS) or short QT syndrome (SQTS).

18. The method of claim 16, wherein said congenital cardiac disease is LQT1.

19. The method of any one of claims 16 to 18, wherein said first nucleotide sequence comprises the sequence set forth in SEQ ID NO:12, SEQ ID NO:13, SEQ ID NO:15, or SEQ ID NO:36, and wherein said second nucleotide sequence comprises the sequence set forth in SEQ ID NO:9.

20. The method of any one of claims 16 to 18, wherein said first nucleotide sequence comprises the sequence set forth in SEQ ID NO:36 and said second nucleotide sequence comprises the sequence set forth in SEQ ID NO:9.

21. The method of any one of claims 16 to 20, wherein said first nucleotide sequence is operably linked to a first promoter and said second nucleotide sequence is operably linked to a second promoter.

22. The method of claim 21, wherein said first and second promoters are the same.

23. The method of claim 21, wherein said first and second promoters are different.

24. The method of claim 21, wherein said first promoter is a U6 promoter and said second promoter is a CMV promoter.

25. The method of any one of claims 16 to 24, wherein said nucleic acid construct further comprises a nucleotide sequence encoding a reporter.

26. The method of claim 25, wherein said reporter is a fluorescent polypeptide.

27. The method of claim 25 or claim 26, wherein said nucleotide sequence encoding said reporter is downstream of said second nucleotide sequence encoding said cDNA, and is separated from said second nucleotide sequence by an IRES.

28. The method of any one of claims 16 to 27, wherein said nucleic acid construct is within a viral vector.

29. The method of claim 28, wherein said viral vector is an AAV vector.

30. The method of claim 29, wherein said AAV vector is an AAV serotype 9 vector or an AAV2/9 vector.

31. The method of any one of claims 16 to 30, wherein said cell is a cardiomyocyte.

32. A method for reducing the action potential duration (APD) in cardiac cells within a mammal, said method comprising administering to said mammal a nucleic acid construct comprising:
(a) a first nucleotide sequence encoding an RNAi molecule capable of hybridizing to a target sequence encoding an endogenous KCNQ1 polypeptide within cardiac cells of said mammal and suppressing expression of said endogenous KCNQ1 polypeptide within said cardiac cells, and
(b) a second nucleotide sequence encoding a KCNQ1 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNQ1 polypeptide from said second nucleotide sequence within said cell.

33. The method of claim 32, wherein said first nucleotide sequence comprises the sequence set forth in SEQ ID NO:12, SEQ ID NO:13, SEQ ID NO:15, or SEQ ID NO:36, and wherein said second nucleotide sequence comprises the sequence set forth in SEQ ID NO:9.

34. The method of claim 32, wherein said first nucleotide sequence comprises the sequence set forth in SEQ ID NO:36 and said second nucleotide sequence comprises the sequence set forth in SEQ ID NO:9.

35. The method of any one of claims 32 to 34, wherein said first nucleotide sequence is operably linked to a first promoter and said second nucleotide sequence is operably linked to a second promoter.

36. The method of claim 35, wherein said first and second promoters are the same.

37. The method of claim 35, wherein said first and second promoters are different.

38. The method of claim 37, wherein said first promoter is a U6 promoter and said second promoter is a CMV promoter.

39. The method of any one of claims 32 to 38, wherein said nucleic acid construct is within a viral vector.

40. The method of claim 39, wherein said viral vector is an AAV vector.

41. The method of claim 40, wherein said AAV vector is an AAV serotype 9 vector or an AAV2/9 vector.

42. A method for reducing one or more symptoms of LQTS in a mammal, said method comprising administering to said mammal a nucleic acid construct comprising:
(a) a first nucleotide sequence encoding an RNAi molecule capable of hybridizing to a target sequence encoding an endogenous KCNQ1 polypeptide within a cell of said mammal and suppressing expression of said endogenous KCNQ1 polypeptide within said cell, and
(b) a second nucleotide sequence encoding a KCNQ1 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNQ1 polypeptide from said second nucleotide sequence within said cell.

43. The method of claim 42, wherein said LQTS is LQT1.

44. The method of claim 42 or claim 43, wherein said first nucleotide sequence comprises the sequence set forth in SEQ ID NO:12, SEQ ID NO:13, SEQ ID NO:15, or SEQ ID NO:36, and wherein said second nucleotide sequence comprises the sequence set forth in SEQ ID NO:9.

45. The method of claim 42 or claim 43, wherein said first nucleotide sequence comprises the sequence set forth in SEQ ID NO:36 and said second nucleotide sequence comprises the sequence set forth in SEQ ID NO:9.

46. The method of any one of claims 42 to 45, wherein said first nucleotide sequence is operably linked to a first promoter and said second nucleotide sequence is operably linked to a second promoter.

47. The method of claim 46, wherein said first and second promoters are the same.

48. The method of claim 46, wherein said first and second promoters are different.

49. The method of claim 48, wherein said first promoter is a U6 promoter and said second promoter is a CMV promoter.

50. The method of any one of claims 42 to 49, wherein said nucleic acid construct is within a viral vector.

51. The method of claim 50, wherein said viral vector is an AAV vector.

52. The method of claim 51, wherein said AAV vector is an AAV serotype 9 vector or an AAV2/9 vector.

53. The method of any one of claims 42 to 52, wherein said cell is a cardiomyocyte.

54. A nucleic acid construct comprising:
(a) a first nucleotide sequence encoding an RNAi molecule capable of hybridizing to a target sequence encoding an endogenous KCNH2 polypeptide within a cell and suppressing expression of said endogenous KCNH2 polypeptide within said cell, and
(b) a second nucleotide sequence encoding a KCNH2 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNH2 polypeptide from said second nucleotide sequence within said cell.

55. The nucleic acid construct of claim 54, wherein said first nucleotide sequence comprises the sequence set forth in SEQ ID NO:27 and said second nucleotide sequence comprises the sequence set forth in SEQ ID NO:29.

56. The nucleic acid construct of claim 54 or claim 55, wherein said first nucleotide sequence is operably linked to a first promoter and said second nucleotide sequence is operably linked to a second promoter.

57. The nucleic acid construct of claim 56, wherein said first and second promoters are the same.

58. The nucleic acid construct of claim 56, wherein said first and second promoters are different.

59. The nucleic acid construct of claim 58, wherein said first promoter is a U6 promoter and said second promoter is a CMV promoter.

60. The nucleic acid construct of any one of claims 54 to 59, further comprising a nucleotide sequence encoding a reporter.

61. The nucleic acid construct of claim 60, wherein said reporter is a fluorescent polypeptide.

62. The nucleic acid construct of claim 60 or claim 61, wherein said nucleotide sequence encoding said reporter is downstream of said second nucleotide sequence encoding said cDNA, and is separated from said second nucleotide sequence by an IRES or P2A self-cleaving peptide sequence.

63. The nucleic acid construct of any one of claims 54 to 62, wherein said nucleic acid construct is within a viral vector.

64. The nucleic acid construct of claim 63, wherein said viral vector is an AAV vector.

65. The nucleic acid construct of claim 64, wherein said AAV vector is an AAV serotype 9 vector or an AAV2/9 vector.

66. The nucleic acid construct of any one of claims 54 to 65, wherein said cell is a cardiomyocyte.

67. A virus particle comprising the nucleic acid construct of any one of claims 54 to 66.

68. A method for treating a mammal having a congenital cardiac disease, said method comprising administering to said mammal a nucleic acid construct comprising:
(a) a first nucleotide sequence encoding an RNAi molecule capable of hybridizing to a target sequence encoding an endogenous KCNH2 polypeptide within a cell of said mammal and suppressing expression of said endogenous KCNH2 polypeptide within said cell, and
(b) a second nucleotide sequence encoding a KCNH2 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNH2 polypeptide from said second nucleotide sequence within said cell.

69. The method of claim 68, wherein said congenital cardiac disease is LQTS or SQTS.

70. The method of claim 68, wherein said congenital cardiac disease is LQT2.

71. The method of any one of claims 68 to 70, wherein said first nucleotide sequence comprises the sequence set forth in SEQ ID NO:27 and said second nucleotide sequence comprises the sequence set forth in SEQ ID NO:29.

72. The method of any one of claims 68 to 71, wherein said first nucleotide sequence is operably linked to a first promoter and said second nucleotide sequence is operably linked to a second promoter.

73. The method of claim 72, wherein said first and second promoters are the same.

74. The method of claim 72, wherein said first and second promoters are different.

75. The method of claim 74, wherein said first promoter is a U6 promoter and said second promoter is a CMV promoter.

76. The method of any one of claims 68 to 75, wherein said nucleic acid construct further comprises a nucleotide sequence encoding a reporter.

77. The method of claim 76, wherein said reporter is a fluorescent polypeptide.

78. The method of claim 76 or claim 77, wherein said nucleotide sequence encoding said reporter is downstream of said second nucleotide sequence encoding said cDNA, and is separated from said second nucleotide sequence by an IRES.

79. The method of any one of claims 68 to 78, wherein said nucleic acid construct is within a viral vector.

80. The method of claim 79, wherein said viral vector is an AAV vector.

81. The method of claim 80, wherein said AAV vector is an AAV serotype 9 vector or an AAV2/9 vector.

82. The method of any one of claims 68 to 81, wherein said cell is a cardiomyocyte.

83. A method for reducing the APD in cardiac cells within a mammal, said method comprising administering to said mammal a nucleic acid construct comprising:
(a) a first nucleotide sequence encoding an RNAi molecule capable of hybridizing to a target sequence encoding an endogenous KCNH2 polypeptide within cardiac cells of said mammal and suppressing expression of said endogenous KCNH2 polypeptide within said cardiac cells, and
(b) a second nucleotide sequence encoding a KCNH2 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNH2 polypeptide from said second nucleotide sequence within said cell.

84. The method of claim 83, wherein said first nucleotide sequence comprises the sequence set forth in SEQ ID NO:27 and said second nucleotide sequence comprises the sequence set forth in SEQ ID NO:29.

85. The method of claim 83 or claim 84, wherein said first nucleotide sequence is operably linked to a first promoter and said second nucleotide sequence is operably linked to a second promoter.

86. The method of claim 85, wherein said first and second promoters are the same.

87. The method of claim 85, wherein said first and second promoters are different.

88. The method of claim 87, wherein said first promoter is a U6 promoter and said second promoter is a CMV promoter.

89. The method of any one of claims 83 to 88, wherein said nucleic acid construct is within a viral vector.

90. The method of claim 89, wherein said viral vector is an AAV vector.

91. The method of claim 90, wherein said AAV vector is an AAV serotype 9 vector or an AAV2/9 vector.

92. A method for reducing one or more symptoms of LQTS in a mammal, said method comprising administering to said mammal a nucleic acid construct comprising:
(a) a first nucleotide sequence encoding an RNAi molecule capable of hybridizing to a target sequence encoding an endogenous KCNH2 polypeptide within a cell of said mammal and suppressing expression of said endogenous KCNH2 polypeptide within said cell, and
(b) a second nucleotide sequence encoding a KCNH2 polypeptide, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said KCNH2 polypeptide from said second nucleotide sequence within said cell.

93. The method of claim 92, wherein said LQTS is LQT2.

94. The method of claim 92 or claim 93, wherein said first nucleotide sequence comprises the sequence set forth in SEQ ID NO:27 and said second nucleotide sequence comprises the sequence set forth in SEQ ID NO:29.

95. The method of any one of claims 92 to 94, wherein said first nucleotide sequence is operably linked to a first promoter and said second nucleotide sequence is operably linked to a second promoter.

96. The method of claim 95, wherein said first and second promoters are the same.

97. The method of claim 95, wherein said first and second promoters are different.

98. The method of claim 97, wherein said first promoter is a U6 promoter and said second promoter is a CMV promoter.

99. The method of any one of claims 92 to 98, wherein said nucleic acid construct is within a viral vector.

100. The method of claim 99, wherein said viral vector is an AAV vector.

101. The method of claim 100, wherein said AAV vector is an AAV serotype 9 vector or an AAV2/9 vector.

102. The method of any one of claims 92 to 101, wherein said cell is a cardiomyocyte.

103. A nucleic acid construct for treating a congenital heart disease caused by an endogenous cardiac polypeptide containing one or more mutations causative of said congenital heart disease, wherein said construct comprises:
(a) a first nucleotide sequence encoding an RNAi molecule capable of hybridizing to a target sequence encoding said endogenous cardiac polypeptide within a cell and suppressing expression of said endogenous cardiac polypeptide within said cell, and
(b) a second nucleotide sequence encoding a replacement version of said endogenous cardiac polypeptide that lacks said one or more mutations causative of said congenital heart disease, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said replacement version of said endogenous cardiac polypeptide that lacks said one or more mutations causative of said congenital heart disease from said second nucleotide sequence within said cell.

104. The nucleic acid construct of claim 103, wherein said first nucleotide sequence is operably linked to a first promoter and said second nucleotide sequence is operably linked to a second promoter.

105. The nucleic acid construct of claim 104, wherein said first and second promoters are the same.

106. The nucleic acid construct of claim 104, wherein said first and second promoters are different.

107. The nucleic acid construct of claim 106, wherein said first promoter is a U6 promoter and said second promoter is a CMV promoter.

108. The nucleic acid construct of any one of claims 103 to 107, further comprising a nucleotide sequence encoding a reporter.

109. The nucleic acid construct of claim 108, wherein said reporter is a fluorescent polypeptide.

110. The nucleic acid construct of claim 108 or claim 109, wherein said nucleotide sequence encoding said reporter is downstream of said second nucleotide sequence encoding said cDNA, and is separated from said second nucleotide sequence by an internal ribozyme entry sequence (IRES) or P2A self-cleaving peptide sequence.

111. The nucleic acid construct of any one of claims 103 to 110, wherein said nucleic acid construct is within a viral vector.

112. The nucleic acid construct of claim 111, wherein said viral vector is an AAV vector.

113. The nucleic acid construct of claim 112, wherein said AAV vector is an AAV serotype 9 vector or an AAV2/9 vector.

114. The nucleic acid construct of any one of claims 103 to 113, wherein said cell is a cardiomyocyte.

115. A virus particle comprising the nucleic acid construct of any one of claims 103 to 114.

116. A method for treating a mammal having a congenital cardiac disease, said method comprising administering to said mammal a nucleic acid construct comprising:
(a) a first nucleotide sequence encoding an RNAi molecule capable of hybridizing to a target sequence encoding said endogenous cardiac polypeptide within a cell and suppressing expression of said endogenous cardiac polypeptide within said cell, and
(b) a second nucleotide sequence encoding a replacement version of said endogenous cardiac polypeptide that lacks said one or more mutations causative of said congenital heart disease, wherein said second nucleotide sequence comprises a target sequence identical to said target sequence of said first nucleotide sequence with the exception that said target sequence of said second nucleotide sequence comprises 1 to 13 wobble position variants as compared to said target sequence of said first nucleotide sequence, and wherein said RNAi molecule does not suppress expression of said replacement version of said endogenous cardiac polypeptide that lacks said one or more mutations causative of said congenital heart disease from said second nucleotide sequence within said cell.

117. The method of claim 116, wherein said first nucleotide sequence is operably linked to a first promoter and said second nucleotide sequence is operably linked to a second promoter.

118. The method of claim 117, wherein said first and second promoters are the same.

119. The method of claim 117, wherein said first and second promoters are different.

120. The method of claim 119, wherein said first promoter is a U6 promoter and said second promoter is a CMV promoter.

121. The method of any one of claims 116 to 120, wherein said nucleic acid construct further comprises a nucleotide sequence encoding a reporter.

122. The method of claim 121, wherein said reporter is a fluorescent polypeptide.

123. The method of claim 121 or claim 122, wherein said nucleotide sequence encoding said reporter is downstream of said second nucleotide sequence encoding said cDNA, and is separated from said second nucleotide sequence by an IRES.

124. The method of any one of claims 116 to 123 wherein said nucleic acid construct is within a viral vector.

125. The method of claim 124, wherein said viral vector is an AAV vector.

126. The method of claim 125, wherein said AAV vector is an AAV serotype 9 vector or an AAV2/9 vector.

127. The method of any one of claims 116 to 126, wherein said cell is a cardiomyocyte.
